(19) United States
(12) Patent Application Publication
(10) Pub. No.: US 2024/0279685 A1
(43) Pub. Date: Aug. 22, 2024

(54) METHODS AND COMPOSITIONS FOR PRODUCING VIRAL FUSOSOMES

(71) Applicant: FLAGSHIP PIONEERING INNOVATIONS V, INC., Cambridge, MA (US)

(72) Inventors: Benhur Lee, New York, NY (US); Michael Travis Mee, Montreal (CA); Jagesh V. Shah, Lexington, MA (US); Kyle Marvin Trudeau, Boston, MA (US)

(21) Appl. No.: 18/004,403

(22) PCT Filed: Jul. 6, 2021
(86) PCT No.: PCT/US2021/040501

§ 371 Date: Jan. 5, 2023

Related U.S. Application Data
(60) Provisional application No. 63/048,524, filed on Jul. 6, 2020.

(51) Int. Cl. C12N 15/88; A61K 9/51; C07K 14/005; C12N 5/071; C12N 7/00

(52) U.S. Cl. CPC C12N 15/88; A61K 9/5184; C07K 14/005; C12N 5/0602; C12N 7/00; C12N 2740/16043; C12N 2760/18222

(57) ABSTRACT

The present disclosure provides, at least in part, methods and compositions for producing fusosomes. In some embodiments, a producer cell for producing fusosomes comprises an exogenous or overexpresed cathepsin molecule. In some embodiments, these cells generate an increased level of active fusogen, leading to a higher proportion of fusogenically active fusosomes.

### CROSS-REFERENCE TO RELATED APPLICATIONS

This application claims priority to U.S. provisional application 63/048,524, filed Jul. 6, 2020, the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 18615_2003540_SeqList.TXT, created Jul. 1, 2020, which is 97,896 bytes in size. The information in the electronic format of the Sequence Listing is incorporated by reference in its entirety.

### BACKGROUND

Complex biologics are promising therapeutic candidates for a variety of diseases. However, it is difficult to deliver large biologic agents into a cell because the plasma membrane acts as a barrier between the cell and the extracellular space. There is a need in the art for new methods of delivering complex biologics into cells in a subject.

### SUMMARY

The present disclosure provides, at least in part, methods of making fusosomes that can be used for in vivo delivery. In some embodiments, the method comprises expressing a cathepsin molecule in a producer cell, in order to increase levels of functional fusosomes produced by the cell.

### Enumerated Embodiments

Among the provided embodiments are:

1. A method of producing a plurality of fusosomes, comprising:
(a) providing a modified mammalian producer cell, e.g., a human cell, that comprises:
(i) an elevated level or activity of a mature cathepsin molecule (e.g., cathepsin L or cathepsin B) compared to a corresponding unmodified cell,
(ii) optionally, an exogenous cargo molecule, e.g., a protein or nucleic acid, and
(iii) a henipavirus F protein molecule; and
(iv) a henipavirus G protein molecule;
(b) maintaining (e.g., culturing) the modified mammalian cell under conditions that allow production of a plurality of fusosomes comprising the henipavirus F protein molecule, and the henipavirus G protein molecule.

2. The method of embodiment 1, wherein the cargo molecule comprises a viral nucleic acid (e.g., a lentiviral nucleic acid).

3. The method of embodiment 1, wherein the modified cell has been introduced with the exogenous cargo molecule (e.g., wherein a nucleic acid encoding the exogenous cargo molecule has been introduced into the modified cell).

4. The method of any of the preceding embodiments, wherein the modified cell has been introduced with a cathepsin molecule or a nucleic acid encoding the cathepsin molecule, e.g., under conditions for processing of the cathepsin molecule to the mature cathepsin form.

5. The method of any of the preceding embodiments, wherein the modified cell has been introduced with a cathepsin molecule or a nucleic acid encoding the cathepsin molecule under conditions suitable for expression of the cathepsin molecule.

6. A method of producing a modified mammalian producer cell, the method comprising:
(i) introducing into a mammalian cell a nucleic acid molecule encoding a cathepsin molecule under conditions to increase expression of the mature form of the cathepsin molecule in the mammalian cell;
(ii) optionally, introducing into the mammalian cell an exogenous cargo molecule, e.g., a protein or a nucleic acid;
(iii) introducing into the mammalian cell a henipavirus F protein molecule (e.g., introducing a nucleic acid encoding the henipavirus F protein molecule under conditions suitable for expressing the henipavirus F protein molecule); and
(iv) introducing into the mammalian cell a henipavirus G protein molecule (e.g., introducing a nucleic acid encoding the henipavirus G protein molecule under conditions suitable for expressing the henipavirus G protein molecule),
wherein steps (i)-(iv) can be carried out in any order or one or more of steps (i)-(iv) can be carried out simultaneously.

7. A method of producing a plurality of fusosomes, the method comprising maintaining (e.g., culturing) the modified mammalian cell produced in embodiment 3a under conditions that allow production of a plurality of fusosomes comprising the henipavirus F protein molecule, and the henipavirus G protein molecule.

8. The method of any of the preceding embodiments, further comprising separating at least one of the plurality of fusosomes from the modified cell.

9. The method of any of the preceding embodiments, further comprising:
a) assaying one or more fusosomes from the produced plurality to determine whether one or more (e.g., 2, 3, or more) standards are met, wherein the standard(s) are chosen from:
   i) at least 33%, 35%, 40%, 45%, 50%, 55%, or 60% of henipavirus F protein molecule in the fusosome is active henipavirus F protein; or 1:2, 3:5, 7:10, 4:5, 9:10, or 1:1 1:1;
   ii) the fusosomes have a functional titre of at least about 200,000, 300,000, 400,000, 500,000, 600,000, 700,000, 800,000, 900,000, or 1,000,000 TU/mL, e.g., on 293LX cells, e.g., as measured by detection of a GFP reporter in 293LX cells, e.g., by an assay of Example 1;
   iii) the fusosomes have a functional titre of at least about 200,000, 300,000, 400,000, 500,000, 600,000, 700,000, 800,000, 900,000, or 1,000,000 TU/mL, e.g., on activated T cells, e.g., primary T cells, e.g., Pan-T cells, e.g., as measured by detection of a GFP reporter in the activated T cells, e.g., by an assay of Example 3;
   iv) wherein the produced plurality of fusosomes has a ratio of a titre on target cells to a titre on non-target cells of at least 2:1, 5:1, 10:1, 20:1, 30:1, 40:1, 50:1, 60:1, 70:1, 80:1, 90:1, 100:1, 500:1, 1000:1, 5000:1, 10,000:1, 50,000:1, or 100,000:1, e.g., wherein target cells overexpress a protein bound by the henipavirus G protein molecule and the non-target cells are wild-type, e.g., wherein target cells overexpress CD8 and the non-target cells are wild-type, e.g., in an assay of Example 1;
   v) the fusosomes comprises active henipavirus F protein molecule at a level at least 10%, 20%, 30%, 40%, or 50% greater than the level of active henipavirus F protein molecule in otherwise similar fusosomes produced from a cell without the elevated level or activity of a cathepsin molecule;
b) (optionally) approving the produced plurality of fusosomes or fusosome composition for release if one or more of the standards is met.

10. The method of any of the preceding embodiments, wherein the plurality of fusosomes has 1, 2, 3, 4, 5, 6, or all 7 of the following characteristics:
i) at least 33%, 35%, 40%, 45%, 50%, 55%, or 60% of henipavirus F protein molecule in the fusosome is active henipavirus F protein;
ii) the fusosomes have a functional titre of at least about 200,000, 300,000, 400,000, 500,000, 600,000, 700,000, 800,000, 900,000, or 1,000,000 TU/mL, e.g., on 293LX cells, e.g., as measured by detection of a GFP reporter in 293 XL cells, e.g., by an assay of Example 1;
iii) the fusosomes have a functional titre of at least about 200,000, 300,000, 400,000, 500,000, 600,000, 700,000, 800,000, 900,000, or 1,000,000 TU/mL, e.g., on activated T cells, e.g., primary T cells, e.g., Pan-T cells, e.g., as measured by detection of a GFP reporter in the activated T cells, e.g., by an assay of Example 3;
iv) wherein the produced plurality of fusosomes has a ratio of a titre on target cells to a titre on non-target cells of at least 2:1, 5:1, 10:1, 20:1, 30:1, 40:1, 50:1, 60:1, 70:1, 80:1, 90:1, 100:1, 500:1, 1000:1, 5000:1, 10,000:1, 50,000:1, or 100,000:1, e.g., wherein target cells overexpress a protein bound by the henipavirus G protein molecule and the non-target cells are wild-type, e.g., wherein target cells overexpress CD8 and the non-target cells are wild-type, e.g., in an assay of Example 1;
v) the fusosomes comprises active henipavirus F protein molecule at a level at least 10%, 20%, 30%, 40%, or 50% greater than the level of active henipavirus F protein molecule in otherwise similar fusosomes produced from a cell without the elevated level or activity of a cathepsin molecule.

11. A modified cell produced by the method of embodiment 6.

12. A modified mammalian cell, e.g., a human cell, that comprises:
(i) an elevated level or activity of a mature cathepsin molecule (e.g., cathepsin L or cathepsin B) compared to a corresponding unmodified cell,
(ii) optionally, an exogenous cargo molecule, e.g., a nucleic acid or a protein, e.g., a viral nucleic acid, e.g., a lentiviral nucleic acid, and
(iii) a henipavirus F protein molecule; and
(iv) optionally, a henipavirus G protein molecule.

13. A modified mammalian cell, e.g., a human cell, that comprises:
(i) optionally, an exogenous cargo molecule, e.g., a nucleic acid or a protein, e.g., a viral nucleic acid, e.g., a lentiviral nucleic acid, and
(ii) a henipavirus F protein molecule, wherein at least 50%, 55%, 60%, 65%, 70%, 75%, 80%, or 85% of henipavirus F protein molecule in the cell is active henipavirus F protein; and
(iii) optionally, a henipavirus G protein molecule.

14. A fusosome comprising:
(a) optionally, an exogenous cargo, e.g., a nucleic acid or protein, e.g., a viral nucleic acid (e.g., a lentiviral nucleic acid);
(b) an active henipavirus F protein molecule_comprising a modified F1 form that has a C-terminal truncation of up to 30 contiguous amino acids compared to a wild-type henipavirus protein F1 molecule, wherein at least 33%, 35%, 40%, 45%, 50%, 55%, or 60% of henipavirus F protein molecule in the fusosome is active henipavirus F protein; and
(c) a henipavirus G protein molecule.

15. The fusosome of embodiment 14, wherein the modified F1 form has a C-terminal truncation of 10-30, 15-30, 10-20, or 20-30 amino acids, e.g., 22 or 25 amino acids, contiguous amino acids compared to a wild-type henipavirus F1 protein.

16. A fusosome comprising:
(a) optionally, an exogenous cargo, e.g., a nucleic acid or protein, e.g., a viral nucleic acid (e.g., a lentiviral nucleic acid);
(b) henipavirus F protein molecule, wherein at least 33%, 35%, 40%, 45%, 50%, 55%, or 60% of henipavirus F protein molecule in the fusosome is active henipavirus F protein; and
(c) a henipavirus G protein molecule.

17. The fusosome of any of embodiments 14-16, wherein the henipavirus F protein molecule lacks an endocytosis motif.

18. The fusosome of embodiment 17, wherein the endocytosis motif is a YXXφ motif.

19. The fusosome of embodiment 17 or 18, wherein the endocytosis motif is a YSRL motif.

20. A fusosome comprising:
(a) optionally, an exogenous cargo, e.g., a nucleic acid or protein, e.g., a viral nucleic acid (e.g., a lentiviral nucleic acid);
(b) a henipavirus F protein molecule at least 33%, 35%, 40%, 45%, 50%, 55%, or 60% of henipavirus F protein molecule in the fusosome is active henipavirus F protein; and
(c) a henipavirus G protein molecule;
wherein the henipavirus F protein molecule lacks an endocytosis motif, e.g., a YXXφ motif, e.g., a YRSL motif.

21. The fusosome of embodiment 20, comprising a modified F1 form that has a C-terminal truncation of up to 30 contiguous amino acids compared to a wild-type henipavirus protein F1 molecule.

22. The fusosome of embodiment 21, wherein the henipavirus F protein molecule comprises a truncation of 10-30, 15-30, 10-20, or 20-30 amino acids, e.g., 22 or 25 amino acids, at the C terminus relative to a wild-type henipavirus F protein, e.g., relative to SEQ ID NO:7.

23. A fusosome comprising:
(a) optionally, an exogenous cargo, e.g., a fusosome nucleic acid, e.g., a viral nucleic acid (e.g., a lentiviral nucleic acid);
(b) a henipavirus F protein molecule, wherein at least 33%, 35%, 40%, 45%, 50%, 55%, or 60% of henipavirus F protein molecule in the fusosome is active henipavirus F protein; and
(c) a henipavirus G protein molecule.

24. A pharmaceutical composition comprising a fusosome of any of embodiments 14-23 and, optionally, a pharmaceutically acceptable excipient.

25. A pharmaceutical composition comprising a plurality of fusosomes that comprise:
(a) optionally, an exogenous cargo, e.g., a fusosome nucleic acid, e.g., a viral nucleic acid (e.g., a lentiviral nucleic acid);
(b) a henipavirus F protein molecule, and
(c) a henipavirus G protein molecule,
wherein the pharmaceutical composition has a titre of at least about 200,000, 300,000, 400,000, 500,000, 600,000, 700,000, 800,000, 900,000, or 1,000,000 TU/mL, e.g., on 293LX cells, e.g., as measured by detection of a GFP reporter in 293 XL cells, e.g., by an assay of Example 1.

26. A pharmaceutical composition comprising a plurality of fusosomes that comprise:
(a) optionally, an exogenous cargo, e.g., a fusosome nucleic acid, e.g., a viral nucleic acid (e.g., a lentiviral nucleic acid);
(b) a henipavirus F protein molecule, wherein the henipavirus F protein molecule comprises a modified F1 form that has a C-terminal truncation of up to 30 contiguous amino acids compared to a wild-type henipavirus protein F1 molecule, or wherein the henipavirus F protein molecule lacks an endocytosis motif (e.g., a YXXφ motif, e.g., a YRSL motif), and
(c) a henipavirus G protein molecule, wherein the pharmaceutical composition has a titre of at least about 200,000, 300,000, 400,000, 500,000, 600,000, 700,000, 800,000, 900,000, or 1,000,000 TU/mL, e.g., on 293LX cells, e.g., as measured by detection of a GFP reporter in 293 XL cells, e.g., by an assay of Example 1.

27. A pharmaceutical composition comprising a plurality of fusosomes that comprise:
(a) optionally, an exogenous cargo, e.g., a fusosome nucleic acid, e.g., a viral nucleic acid (e.g., a lentiviral nucleic acid);
(b) a henipavirus F protein molecule, and
(c) a henipavirus G protein molecule,
wherein the pharmaceutical composition has a titre of at least about 200,000, 300,000, 400,000, 500,000, 600,000, 700,000, 800,000, 900,000, or 1,000,000 TU/mL, e.g., on a target cell, e.g., activated T cells, e.g., primary T cells, e.g., Pan-T cells, e.g., as measured by detection of a GFP reporter in activated T cells, e.g., by an assay of Example 3.

28. A method of manufacturing a pharmaceutical composition comprising a plurality of fusosomes, comprising:
a) providing, e.g., producing, a plurality of fusosomes of any of embodiments 14-23, a pharmaceutical composition of any of embodiments 24-27, or fusosomes made by a method of any of embodiments 1-10; and
b) assaying one or more fusosomes from the plurality to determine whether one or more (e.g., 2, 3, or more) standards are met, wherein the standard(s) are chosen from:
   i) at least 33%, 35%, 40%, 45%, 50%, 55%, or 60% of henipavirus F protein molecule in the fusosomes is active henipavirus F protein;
   ii) the pharmaceutical composition has a titre of at least about 200,000, 300,000, 400,000, 500,000, 600,000, 700,000, 800,000, 900,000, or 1,000,000 TU/mL, e.g., on 293LX cells, e.g., as measured by detection of a GFP reporter in 293 XL cells, e.g., by an assay of Example 1;
   iii) the pharmaceutical composition has a titre of at least about 200,000, 300,000, 400,000, 500,000, 600,000, 700,000, 800,000, 900,000, or 1,000,000 TU/mL, e.g., on activated T cells, e.g., primary T cells, e.g., Pan-T cells, e.g., as measured by detection of a GFP reporter in the T cells, e.g., by an assay of Example 3;
   iv) wherein the plurality of fusosomes has a ratio of a titre on target cells to a titre on non-target cells of at least 2:1, 5:1, 10:1, 20:1, 30:1, 40:1, 50:1, 60:1, 70:1, 80:1, 90:1, 100:1, 500:1, 1000:1, 5000:1, 10,000:1, 50,000:1, or 100,000:1, e.g., wherein target cells overexpress a protein bound by the henipavirus G protein molecule and the non-target cells are wild-type, e.g., wherein target cells overexpress CD8 and the non-target cells are wild-type, e.g., in an assay of Example 1;
   v) the fusosomes comprises active henipavirus F protein molecule at a level at least 10%, 20%, 30%, 40%, or 50% greater than the level of active henipavirus F protein molecule in otherwise similar fusosomes produced from a cell without the elevated level or activity of a cathepsin molecule;
c) (optionally) approving the plurality of fusosomes or pharmaceutical composition for release if one or more of the standards is met.

29. A reaction mixture comprising:
a) a plurality of target cells (e.g., human cells, e.g., primary human cells, e.g., cells from a subject), and
b) a plurality of fusosomes of any of embodiments 14-23, a pharmaceutical composition of any of embodiments 24-27, or fusosomes made by a method of any of embodiments 1-10.

30. A target cell (e.g., a human cell, e.g., a primary human cell, e.g., a cell from a subject) comprising:
a) an exogenous cargo molecule (e.g., a sprotein or nucleic acid, e.g., a viral nucleic acid, e.g., a lentiviral nucleic acid), and
b) a henipavirus F protein molecule, wherein at least 33%, 35%, 40%, 45%, 50%, 55%, or 60% of henipavirus F protein molecule in the target cell is active henipavirus F protein; and
c) a henipavirus G protein molecule.

31. A method of delivering an exogenous cargo (e.g., a fusosome nucleic acid, e.g., a viral nucleic acid, e.g., a lentiviral nucleic acid) to a cell (e.g., in vivo or ex vivo), comprising contacting the cell with a plurality of fusosomes of any of embodiments 14-23, a pharmaceutical composition of any of embodiments 24-27, or fusosomes made by a method of any of embodiments 1-10.

32. A method of delivering an exogenous cargo (e.g., a fusosome nucleic acid, e.g., a viral nucleic acid, e.g., a lentiviral nucleic acid) to a subject, comprising administering to the subject an effective number of fusosomes of any of embodiments 14-23, a pharmaceutical composition of any of embodiments 24-27, or fusosomes made by a method of any of embodiments 1-10.

33. The pharmaceutical composition of any of embodiments 24-27, wherein the plurality of fusosomes has a titre of at least about 200,000, 300,000, 400,000, 500,000, 600,000, 700,000, 800,000, 900,000, or 1,000,000 TU/mL, e.g., on 293LX cells, e.g., as measured by detection of a GFP reporter in 293 XL cells, e.g., by an assay of Example 1.

34. The pharmaceutical composition of any of embodiments 24-27, wherein the plurality of fusosomes has a titre of at least about 200,000, 300,000, 400,000, 500,000, 600,000, 700,000, 800,000, 900,000, or 1,000,000 TU/mL, e.g., on activated T cells, e.g., primary T cells, e.g., Pan-T cells, e.g., as measured by detection of a GFP reporter in the T cells, e.g., by an assay of Example 3.

35. The pharmaceutical composition of any of embodiments 24-27, wherein the plurality of fusosomes has a ratio of a titre on target cells to a titre on non-target cells of at least 2:1, 5:1, 10:1, 20:1, 30:1, 40:1, 50:1, 60:1, 70:1, 80:1, 90:1, 100:1, 500:1, 1000:1, 5000:1, 10,000:1, 50,000:1, or 100,000:1, e.g., wherein target cells overexpress a protein bound by the henipavirus G protein molecule and the non-target cells are wild-type, e.g., wherein target cells overexpress CD8 and the non-target cells are wild-type, e.g., in an assay of Example 1.

36. The pharmaceutical composition of any of embodiments 24-27, wherein the plurality of fusosomes comprises active henipavirus F protein molecule at a level at least 10%, 20%, 30%, 40%, or 50% greater than otherwise similar fusosomes produced from a cell without the elevated level or activity of a cathepsin molecule.

37. The method, modified cell, fusosome, or pharmaceutical composition of any of the preceding embodiments, wherein the cathepsin molecule comprises an amino acid sequence of SEQ ID NO: 1 or SEQ ID NO: 2 or a sequence having at least 75, 80, 85, 90, 95, 96, 97, 98, 99, or 100% identity thereto.

38. The method, modified cell, fusosome, or pharmaceutical composition of any of the preceding embodiments, wherein the cathepsin molecule comprises a fusion or chimera.

39. The method, modified cell, fusosome, or pharmaceutical composition of any of the preceding embodiments, wherein the modified cell comprises at least 1,000, 2,000, 3,000, 4,000, 5,000, 6,000, 7,000, 8,000, 9,000, 10,000, 50,000, 100,000, 500,000, or 1,000,000 copies of an exogenous cathepsin molecule.

40. The method, modified cell, fusosome, or pharmaceutical composition of any of the preceding embodiments, wherein the modified cell comprises at least 1,000, 2,000, 3,000, 4,000, 5,000, 6,000, 7,000, 8,000, 9,000, 10,000, 50,000, 100,000, 500,000, or 1,000,000 copies of an total cathepsin L molecules.

41. The method, modified cell, fusosome, or pharmaceutical composition of any of the preceding embodiments, wherein the elevated level of the cathepsin molecule comprises at least 10%, 20%, 25%, 30%, 40%, 50%, 60%, 70%, 80%, 90%, 100%, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1,000-fold, 10,000-fold, 100,000-fold, or more cathepsin molecule than the amount of endogenous cathepsin L in a corresponding unmodified cell.

42. The method, modified cell, fusosome, or pharmaceutical composition of any of the preceding embodiments, wherein the elevated activity of the cathepsin molecule comprises at least 10%, 20%, 25%, 30%, 40%, 50%, 60%, 70%, 80%, 90%, 100%, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1,000-fold, 10,000-fold, 100,000-fold, or greater cathepsin molecule activity per cell than the cathepsin molecule activity of a corresponding unmodified cell, e.g., as measured by an assay of Diederich et al. 2012.

43. The method of making or the modified cell of any of the preceding embodiments, wherein some or all of the cathepsin molecules are situated in the lysosome and/or endosome of the modified cell.

44. The method, modified cell, fusosome, or pharmaceutical composition of any of the preceding embodiments, wherein the fusosome comprises active henipavirus F protein molecule at a level at least 10%, 20%, 30%, 40%, or 50% greater than an otherwise similar fusosome produced from a cell without the elevated level or activity of a cathepsin molecule.

45. The method, modified cell, fusosome, or pharmaceutical composition of any of the preceding embodiments, wherein at least 33%, 35%, 40%, 45%, 50%, 55%, or 60% of henipavirus F protein molecule in the fusosome is active henipavirus F protein.

46. The method, modified cell, or pharmaceutical composition of any of the preceding embodiments, wherein the fusosomes have a functional titre of at least about 200,000, 300,000, 400,000, 500,000, 600,000, 700,000, 800,000, 900,000, or 1,000,000 TU/mL, e.g., on 293LX cells, e.g., as measured by detection of a GFP reporter in 293 XL cells, e.g., by an assay of Example 1.

47. The method, modified cell, or pharmaceutical composition of any of the preceding embodiments, wherein the fusosomes have a functional titre of at least about 200,000, 300,000, 400,000, 500,000, 600,000, 700,000, 800,000, 900,000, or 1,000,000 TU/mL, e.g., on activated T cells, e.g., primary T cells, e.g., Pan-T cells, e.g., as measured by detection of a GFP reporter in the activated T cells, e.g., by an assay of Example 3.

48. The method, modified cell, or pharmaceutical composition of any of the preceding embodiments, wherein the produced plurality of fusosomes has a ratio of a titre on target cells to a titre on non-target cells of at least 2:1, 5:1, 10:1, 20:1, 30:1, 40:1, 50:1, 60:1, 70:1, 80:1, 90:1, 100:1, 500:1, 1000:1, 5000:1, 10,000:1, 50,000:1, or 100,000:1, e.g., wherein target cells overexpress a protein bound by the henipavirus G protein molecule and the non-target cells are wild-type, e.g., wherein target cells overexpress CD8 and the non-target cells are wild-type, e.g., in an assay of Example 1.

49. The method, modified cell, fusosome, or pharmaceutical composition of any of the preceding embodiments, wherein the fusosome comprises a level of total henipavirus protein F that is between 70%-130%, 80%-120%, 90%-110%, 95%-105%, or about 100% of the level of total henipavirus protein F comprised by an otherwise similar fusosome produced from a cell without the elevated level or activity of a cathepsin molecule.

50. The method, modified cell, fusosome, or pharmaceutical composition of any of the preceding embodiments, wherein the henipavirus F protein molecule comprises a Nipah virus or Hendra virus protein F sequence.

51. The method, modified cell, fusosome, or pharmaceutical composition of any of the preceding embodiments, wherein the henipavirus F protein molecule comprises a wild-type Nipah virus amino acid sequence of SEQ ID NO: 7, or a sequence having at least 75, 80, 85, 90, 95, 96, 97, 98, 99, or 100% identity thereto.

52. The method, modified cell, fusosome, or pharmaceutical composition of any of the preceding embodiments, wherein the henipavirus F protein molecule comprises a henipavirus protein F of Table 4.

53. The method, modified cell, fusosome, or pharmaceutical composition of any of the preceding embodiments, wherein the henipavirus F protein molecule comprises a truncation of 10-30, 15-30, 10-20, or 20-30 amino acids, e.g., 22 or 25 amino acids, at the C terminus relative to a wild-type henipavirus F protein, e.g., a protein of Table 4.

54. The method, modified cell, fusosome, or pharmaceutical composition of any of the preceding embodiments, wherein the henipavirus F protein molecule lacks an endocytic motif, e.g., a YXXφ motif, e.g., a YRSL motif.

55. The method, modified cell, fusosome, or pharmaceutical composition of any of the preceding embodiments, wherein the henipavirus F protein molecule comprises a Nipah virus or Hendra virus protein F sequence.

56. The method, modified cell, fusosome, or pharmaceutical composition of any of the preceding embodiments, wherein the henipavirus G protein molecule comprises a wild-type Nipah virus amino acid sequence of SEQ ID NO: 9, or a sequence having at least 75, 80, 85, 90, 95, 96, 97, 98, 99, or 100% identity thereto.

57. The method, modified cell, fusosome, or pharmaceutical composition of any of the preceding embodiments, wherein the henipavirus G protein molecule comprises a truncation of 10-50, 10-40, 20-50, 20-40, 20-30, 30-50, or 30-40 amino acids, e.g., 34 amino acids, at the N terminus, relative to a wild-type henipavirus G protein, e.g., a protein of Table 5.

58. The method, modified cell, fusosome, or pharmaceutical composition of any of the preceding embodiments, wherein the henipavirus G protein molecule comprises one or more mutations (e.g., at least one, two, three, four, five, six, or seven mutations) in a glycosylation site, e.g., an N-linked glycosylation site, e.g., an N-linked glycosylation site in the ectodomain, e.g., a G1, G2, G3, G4, G5, G6, and/or G7 site as described in Biering et al. (2012) J. Virol. 86(22): 11991-12002.

59. The method, modified cell, fusosome, or pharmaceutical composition of any of the preceding embodiments, wherein the henipavirus F protein molecule comprises one or more mutations (e.g., at least one, two, three, or four mutations) in a glycosylation site, e.g., an N-linked glycosylation site, e.g., an F2 (e.g., at N67), F3 (e.g., at N99), F4 (e.g., at N414), and/or F5 (e.g., at N464) site as described in Lee et al. (2011) Trends Microbiol. 19(8): 389-399.

60. The method, modified cell, fusosome, or pharmaceutical composition of any of the preceding embodiments, wherein the henipavirus G protein molecule is a retargeted henipavirus G protein molecule.

61. The method, modified cell, fusosome, or pharmaceutical composition of any of the preceding embodiments, wherein the henipavirus G protein molecule has reduced affinity for EphrinB2 and/or Ephrin B3 compared to a wild-type henipavirus G protein, e.g., wherein the henipavirus G protein molecule comprises a mutation (e.g., a mutation to alanine) at one or more of E501, W504, Q530, and E533.

62. The method, modified cell, fusosome, or pharmaceutical composition of any of the preceding embodiments, wherein the henipavirus G protein molecule further comprises a targeting domain that is exogenous to a wild-type henipavirus G protein.

63. The method, modified cell, fusosome, or pharmaceutical composition of embodiment 62, wherein the targeting domain comprises an antibody molecule.

64. The method, modified cell, fusosome, or pharmaceutical composition of embodiment 62 or 63, wherein the targeting domain binds CD8, CD105, EpCAM, or Gria4.

65. The method, modified cell, fusosome, or pharmaceutical composition of any of the preceding embodiments, wherein the fusosome nucleic acid comprises at least one, e.g., at least two, plasmids.

66. The method, modified cell, fusosome, or pharmaceutical composition of any of the preceding embodiments, wherein the fusosome nucleic acid is not a henipavirus nucleic acid or does not comprise a henipavirus gene.

67. The method, modified cell, fusosome, or pharmaceutical composition of any of the preceding embodiments, wherein the fusosome nucleic acid is not a hendravirus nucleic acid or does not comprise a hendravirus gene.

68. The method, modified cell, fusosome, or pharmaceutical composition of any of the preceding embodiments, wherein the fusosome nucleic acid is a lentiviral nucleic acid.

69. The method, modified cell, fusosome, or pharmaceutical composition of any of the preceding embodiments, wherein the fusosome nucleic acid encodes a therapeutic payload.

70. The method, modified cell, fusosome, or pharmaceutical composition of any of the preceding embodiments, wherein the modified cell is a human cell.

71. The method, modified cell, fusosome, or pharmaceutical composition of any of the preceding embodiments, wherein the modified cell, fusosome, or pharmaceutical composition is produced according to GMP practices.

72. The method, modified cell, fusosome, or pharmaceutical composition of any of the preceding embodiments, wherein the modified cell is a canine cell, primate (e.g., non-human primate, e.g., African green monkey) cell, or murine cell.

73. The method, modified cell, fusosome, or pharmaceutical composition of any of the preceding embodiments, wherein the modified cell is a kidney cell or epithelial cell (e.g., a kidney epithelial cell)

74. The method, modified cell, fusosome, or pharmaceutical composition of any of the preceding embodiments, wherein the modified cell is other than an epithelial cell.

75. The method, modified cell, fusosome, or pharmaceutical composition of any of the preceding embodiments, wherein the modified cell comprises the henipavirus F protein molecule in one or more of the endosome, lysosome, or cell membrane.

76. The method, modified cell, fusosome, or pharmaceutical composition of any of the preceding embodiments, wherein the modified cell comprises the cathepsin molecule in one or more of the endosome, lysosome, or cell membrane.

77. A fusosome comprising:
a) a lipid bilayer comprising a fusogen (e.g., a henipavirus fusogen, e.g., a henipavirus protein G molecule) retargeted to bind CD105; and
b) a lumen comprising a nucleic acid, e.g., a fusosome nucleic acid, e.g., a lentiviral nucleic acid.

78. A fusosome comprising:
a) a lipid bilayer comprising a fusogen (e.g., a henipavirus fusogen, e.g., a henipavirus protein G molecule) retargeted to bind EpCAM; and
b) a lumen comprising a nucleic acid, e.g., a fusosome nucleic acid, e.g., a lentiviral nucleic acid.

79. A fusosome comprising:
a) a lipid bilayer comprising a fusogen (e.g., a henipavirus fusogen, e.g., a henipavirus protein G molecule) retargeted to bind Gria4; and
b) a lumen comprising a nucleic acid, e.g., a fusosome nucleic acid, e.g., a lentiviral nucleic acid.

80. The fusosome of any of the preceding embodiments, wherein one or more of:
i) the fusosome fuses at a higher rate with a target cell than with a non-target cell, e.g., by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold;
ii) the fusosome fuses at a higher rate with a target cell than with another fusosome, e.g., by at least 10%, 20%, 30%, 40%, 50%, 60%, 70%, 80%, or 90%, 2-fold, 3-fold, 4-fold, 5-fold, 10-fold, 20-fold, 50-fold, or 100-fold;
iii) the fusosome fuses with target cells at a rate such that an agent in the fusosome is delivered to at least 10%, 20%, 30%, 40%, 50%, 60%, 70%, 80%, or 90%, of target cells after 24, 48, or 72 hours;
iv) the fusosome delivers the nucleic acid to a target cell at a higher rate than to a non-target cell, e.g., by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold;
v) the fusosome delivers the nucleic acid to a target cell at a higher rate than to another fusosome, e.g., by at least 10%, 20%, 30%, 40%, 50%, 60%, 70%, 80%, or 90%, 2-fold, 3-fold, 4-fold, 5-fold, 10-fold, 20-fold, 50-fold, or 100-fold; or
vi) the fusosome delivers the nucleic acid to a target cell at a rate such that an agent in the fusosome is delivered to at least 10%, 20%, 30%, 40%, 50%, 60%, 70%, 80%, or 90%, of target cells after 24, 48, or 72 hours.

81. The fusosome of any of the preceding embodiments, wherein the nucleic acid comprises one or more of (e.g., all of) the following nucleic acid sequences: 5′ LTR (e.g., comprising U5 and lacking a functional U3 domain), Psi packaging element (Psi), Central polypurine tract (cPPT) Promoter operatively linked to the payload gene, e.g. nucleic acid encoding the exogenous agent, payload gene, e.g. nucleic acid encoding the exogenous agent (optionally comprising an intron before the open reading frame), Poly A tail sequence, WPRE, and 3′ LTR (e.g., comprising U5 and lacking a functional U3).

82. The fusosome of any of the preceding embodiments, which comprises one or more of (e.g., all of) a polymerase (e.g., a reverse transcriptase, e.g., pol or a portion thereof), an integrase (e.g., pol or a portion thereof, e.g., a functional or non-functional variant), a matrix protein (e.g., gag or a portion thereof), a capsid protein (e.g., gag or a portion thereof), a nucleocaspid protein (e.g., gag or a portion thereof), and a protease (e.g., pro).

83. The fusosome of any of the preceding embodiments, wherein, when the fusosome is administered to a subject, one or more of:
i) less than 10%, 5%, 4%, 3%, 2%, or 1% of the exogenous agent detectably present in the subject is in non-target cells;
ii) at least 90%, 95%, 96%, 97%, 98%, or 99% of the cells of the subject that detectably comprise the exogenous agent, are target cells (e.g., cells of a single cell type, e.g., T cells);
iii) less than 1,000,000, 500,000, 200,000, 100,000, 50,000, 20,000, or 10,000 cells of the cells of the subject that detectably comprise the exogenous agent are non-target cells;
iv) average levels of the exogenous agent in all target cells in the subject are at least 100-fold, 200-fold, 500-fold, or 1,000-fold higher than average levels of the exogenous agent in all non-target cells in the subject; or
v) the exogenous agent is not detectable in any non-target cell in the subject.

84. The fusosome of any of the preceding embodiments, wherein the re-targeted fusogen comprises a sequence chosen from Nipah virus F and G proteins, measles virus F and H proteins, tupaia paramyxovirus F and H proteins, paramyxovirus F and G proteins or F and H proteins or F and HN proteins, Hendra virus F and G proteins, Henipavirus F and G proteins, Morbilivirus F and H proteins, respirovirus F and HN protein, a Sendai virus F and HN protein, rubulavirus F and HN proteins, or avulavirus F and HN proteins, or a derivative thereof, or any combination thereof.

85. The fusosome of any of the preceding embodiments, wherein the fusogen comprises a domain of at least 40, 50, 60, 80, 100, 200, 300, 400, 500, or 600 amino acids in length having at least 80%, 85%, 90%, 95%, 96%, 97%, 98%, or 99% sequence identity to a wild-type paramyxovirus fusogen, e.g., a sequence of Table 4 or Table 5.

86. The fusosome of embodiment 85, wherein the paramyxovirus is a Nipah virus, e.g., a henipavirus.

87. The fusosome of any of the preceding embodiments, wherein the target cell is a cancer cell and the non-target cell is a non-cancerous cell.

88. The fusosome of any of the preceding embodiments, which does not deliver nucleic acid to a non-target cell, e.g., an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell.

89. The fusosome of any of the preceding embodiments, wherein less than 10%, 5%, 2.5%, 1%, 0.5%, 0.1%, 0.01%, 0.001%, 0.0001%, 0.00001%, or 0.000001% of a non-target cell type (e.g., one or more of an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell) comprise the nucleic acid, e.g., retroviral nucleic acid, e.g., using quantitative PCR.

90. The fusosome of any of the preceding embodiments, wherein the target cells comprise 0.00001-10, 0.0001-10, 0.001-10, 0.01-10, 0.1-10, 0.5-5, 1-4, 1-3, or 1-2 copies of the nucleic acid, e.g., retroviral nucleic acid or a portion thereof, per host cell genome, e.g., wherein copy number of the nucleic acid is assessed after administration in vivo.

91. The fusosome of any of the preceding embodiments, wherein:
less than 10%, 5%, 2.5%, 1%, 0.5%, 0.1%, 0.01% of the non-target cells (e.g., an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell) comprise the exogenous agent; or
the exogenous agent (e.g., protein) is not detectably present in a non-target cell, e.g an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell.

92. The fusosome of any of the preceding embodiments, wherein the fusosome delivers the nucleic acid, e.g., retroviral nucleic acid, to a target cell, e.g., a T cell, a CD3+ T cell, a CD4+ T cell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+B cell, a CD19+B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

93. The fusosome of any of the preceding embodiments, wherein at least 0.00001%, 0.0001%, 0.001%, 0.001%, 0.01%, 0.1%, 1%, 2%, 5%, 10%, 20%, 30%, 40%, 50%, 60%, 70%, 80%, or 90% of target cells (e.g., one or more of a T cell, a CD3+ T cell, a CD4+ T cell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+B cell, a CD19+B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell) comprise the nucleic acid, e.g., using quantitative PCR.

94. The fusosome of any of the preceding embodiments, wherein at least 0.00001%, 0.0001%, 0.001%, 0.001%, 0.01%, 0.1%, 1%, 2%, 5%, 10%, 20%, 30%, 40%, 50%, 60%, 70%, 80%, or 90% of target cells (e.g., a T cell, a CD3+ T cell, a CD4+ T cell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+B cell, a CD19+B cell, a cancer cell, a CD133+ cancer cell, an EpCAM+ cancer cell, a CD19+ cancel cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell) comprise the exogenous agent.

95. The fusosome of any of the preceding embodiments, wherein, upon administration, the ratio of target cells comprising the nucleic acid to non-target cells comprising the nucleic acid is at least 1.5, 2, 3, 4, 5, 10, 25, 50, 100, 500, 1000, 5000, 10,000, e.g., according to a quantitative PCR assay.

96. The fusosome of any of the preceding embodiments, wherein the ratio of the average copy number of nucleic acid or a portion thereof in target cells to the average copy number of nucleic acid or a portion thereof in non-target cells is at least 1.5, 2, 3, 4, 5, 10, 25, 50, 100, 500, 1000, 5000, 10,000, e.g., according to a quantitative PCR assay.

97. The fusosome of any of the preceding embodiments, wherein the ratio of the median copy number of of nucleic acid or a portion thereof in target cells to the median copy number of nucleic acid or a portion thereof in non-target cells is at least 1.5, 2, 3, 4, 5, 10, 25, 50, 100, 500, 1000, 5000, 10,000, e.g., according to a quantitative PCR assay.

98. The fusosome of any of the preceding embodiments, wherein the ratio of target cells comprising the exogenous RNA agent to non-target cells comprising the exogenous RNA agent is at least 1.5, 2, 3, 4, 5, 10, 25, 50, 100, 500, 1000, 5000, 10,000, e.g., according to a reverse transcription quantitative PCR assay.

99. The fusosome of any of the preceding embodiments, wherein the ratio of the average exogenous RNA agent level of target cells to the average exogenous RNA agent level of non-target cells is at least 1.5, 2, 3, 4, 5, 10, 25, 50, 100, 500, 1000, 5000, 10,000, e.g., according to a reverse transcription quantitative PCR assay.

100. The fusosome of any of the preceding embodiments, wherein the ratio of the median exogenous RNA agent level of target cells to the median exogenous RNA agent level of non-target cells is at least 1.5, 2, 3, 4, 5, 10, 25, 50, 100, 500, 1000, 5000, 10,000, e.g., according to a reverse transcription quantitative PCR assay.

101. The fusosome of any of the preceding embodiments, wherein the ratio of target cells comprising the exogenous protein agent to non-target cells comprising the exogenous protein agent is at least 1.5, 2, 3, 4, 5, 10, 25, 50, 100, 500, 1000, 5000, 10,000, e.g., according to a FACS assay.

102. The fusosome of any of the preceding embodiments, wherein the ratio of the average exogenous protein agent level of target cells to the average exogenous protein agent level of non-target cells is at least 1.5, 2, 3, 4, 5, 10, 25, 50, 100, 500, 1000, 5000, 10,000, e.g., according to a FACS assay.

103. The fusosome of any of the preceding embodiments, wherein the ratio of the median exogenous protein agent level of target cells to the median exogenous protein agent level of non-target cells is at least 1.5, 2, 3, 4, 5, 10, 25, 50, 100, 500, 1000, 5000, 10,000, e.g., according to a FACS assay.

104. The fusosome of any of the preceding embodiments, which comprises one or both of:
i) an exogenous or overexpressed immunosuppressive protein on the lipid bilayer, e.g., envelope; and
ii) an immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell.

105. The fusosome of any of the preceding embodiments, which comprises one or more of:
i) a first exogenous or overexpressed immunosuppressive protein on the lipid bilayer, e.g., envelope, and a second exogenous or overexpressed immunosuppressive protein on the lipid bilayer, e.g., envelope;
ii) a first exogenous or overexpressed immunosuppressive protein on the lipid bilayer, e.g., envelope, and a second immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell; or
iii) a first immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell and a second immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell.

106. The fusosome of any of the preceding embodiments, wherein the nucleic acid comprises one or more insulator elements.

107. The fusosome of any of the preceding embodiments, which, when administered to a subject (e.g., a human subject or a mouse), one or more of:
i) the fusosome does not produce a detectable antibody response (e.g., after a single administration or a plurality of administrations), or antibodies against the fusosome are present at a level of less than 10%, 5%, 4%, 3%, 2%, or 1% above a background level, e.g., by a FACS antibody detection assay);
ii) the fusosome does not produce a detectable cellular immune response (e.g., T cell response, NK cell response, or macrophage response), or a cellular immune response against the fusosome is present at a level of less than 10%, 5%, 4%, 3%, 2%, or 1% above a background level, e.g., by a PBMC lysis assay, by an NK cell lysis assay, by a CD8 killer T cell lysis assay, by a macrophage phagocytosis assay;
iii) the fusosome does not produce a detectable innate immune response, e.g., complement activation (e.g., after a single administration or a plurality of administrations), or the innate immune response against the fusosome is present at a level of less than 10%, 5%, 4%, 3%, 2%, or 1% above a background level, e.g., by a complement activity assay;
iv) less than 90%, 80%, 70%, 60%, 50%, 40%, 30%, 20%, 10%, 5%, 4%, 3%, 2%, 1%, 0.5%, 0.2%, 0.1%, 0.05%, 0.02%, 0.01%, 0.005%, 0.002%, or 0.001% of fusosomes are inactivated by serum, e.g., by a serum inactivation assay;
v) a target cell that has received the exogenous agent from the fusosome does not produce a detectable antibody response (e.g., after a single administration or a plurality of administrations), or antibodies against the target cell are present at a level of less than 10%, 5%, 4%, 3%, 2%, or 1% above a background level, e.g., by a FACS antibody detection assay; or
vi) a target cell that has received the exogenous agent from the fusosome do not produce a detectable cellular immune response (e.g., T cell response, NK cell response, or macrophage response), or a cellular response against the target cell is present at a level of less than 10%, 5%, 4%, 3%, 2%, or 1% above a background level, e.g., by a macrophage phagocytosis assay, by a PBMC lysis assay, by an NK cell lysis assay, or by a CD8 killer T cell lysis assay.

108. The fusosome of any of the preceding embodiments, wherein one or more of (e.g., 2 or all 3 of) the following apply: the fusosome is a retroviral vector, the lipid bilayer is comprised by an envelope, e.g., a viral envelope, and the nucleic acid is a retroviral nucleic acid.

109. The fusosome of embodiment 107 or 108, wherein the background level is the corresponding level in the same subject prior to administration of the particle or vector.

110. The fusosome of any of embodiments 107-109, wherein the immunosuppressive protein is a complement regulatory protein or CD47.

111. The fusosome of any of embodiments 107-110, wherein the immunostimulatory protein is an MHC (e.g., HLA) protein.

112. The fusosome of any of embodiments 107-111, wherein one or both of: the first exogenous or overexpressed immunosuppressive protein is other than CD47, and the second immunostimulatory protein is other than MHC.

113. The fusosome of any of the preceding embodiments, wherein MHC I (e.g., HLA-A, HLA-B, or HLA-C) or MHC II (e.g., HLA-DP, HLA-DM, HLA-DOA, HLA-DOB, HLA-DQ, or HLA-DR) is absent from the fusosome or present at reduced levels (e.g., reduced by at least 10%, 20%, 30%, 40%, 50%, 60%, 70%, 80%, or 90%) compared to a fusosome generated from an otherwise similar, unmodified source cell.

114. The fusosome of any of the preceding embodiments, which comprises one or both of: (i) an exogenous or overexpressed immunosuppressive protein or (ii) an immunostimulatory protein that is absent or present at reduced levels (e.g., reduced by at least 10%, 20%, 30%, 40%, 50%, 60%, 70%, 80%, or 90%) compared to a fusosome generated from an otherwise similar, unmodified source cell.

115. The fusosome of any of the preceding embodiments, wherein the fusosome is in circulation at least 0.5, 1, 2, 3, 4, 6, 12, 18, 24, 36, or 48 hours after administration to the subject.

116. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 30 minutes after administration.

117. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 1 hour after administration.

118. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 2 hours after administration.

119. The fusosome of any of the preceding embodiments, wherein at least odiments 0.001%, 0.01%, 0.1%, 1%, 10%, 20%, 30%, 40%, 50%, 60%, 70%, 80%, 90%, or 100% of fusosomes are in circulation 4 hours after administration.

120. The fusosome of any of the preceding embodiments, wherein at least 0.001%, 0.01%, 0.1%, 1%, 10% 20%, 30%, 40%, 50%, 60%, 70%, 80%, 90%, or 100% of fusosomes are in circulation 8 hours after administration.

121. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 12 hours after administration.

122. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 18 hours after administration.

123. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 24 hours after administration.

124. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 36 hours after administration.

125. The fusosome of any of the preceding embodiments, wherein at least 0.001%, 0.01%, 0.1%, 1%, 10%, 20%, 30%, 40%, 50%, 60%, 70%, 80%, 90%, or 100% of fusosomes are in circulation 48 hours after administration.

126. The fusosome of any of the preceding embodiments, which has a reduction in immunogenicity as measured by a reduction in humoral response following one or more administration of the fusosome to an appropriate animal model, e.g., an animal model described herein, compared to reference retrovirus, e.g., an unmodified fusosome otherwise similar to the fusosome.

127. The fusosome of any of the preceding embodiments, wherein the reduction in humoral response is measured in a serum sample by an anti-cell antibody titre, e.g., anti-retroviral antibody titre, e.g., by ELISA.

128. The fusosome of any of the preceding embodiments, wherein a serum sample from animals administered the fusosome has a reduction of 1%, 5%, 10%, 20%, 30%, 40%, 50%, 60%, 70%, 80%, 90%, or more of an anti-fusosome antibody titer compared to the serum sample from a subject administered an unmodified cell.

129. The fusosome of any of the preceding embodiments, wherein a serum sample from a subject administered the fusosome has an increased anti-cell antibody titre, e.g., increased by 1%, 2%, 5%, 10%, 20%, 30%, or 40% from baseline, e.g., wherein baseline refers to serum sample from the same subject before administration of the fusosome.

130. The fusosome of any of the preceding embodiments, wherein:
the subject to be administered the fusosome has, or is known to have, or is tested for, a pre-existing antibody (e.g., IgG or IgM) reactive with the fusosome;
the subject to be administered the fusosome does not have detectable levels of a pre-existing antibody reactive with the fusosome;
   a subject that has received the fusosome has, or is known to have, or is tested for, an antibody (e.g., IgG or IgM) reactive with the fusosome;
the subject that received the fusosome (e.g., at least once, twice, three times, four times, five times, or more) does not have detectable levels of antibody reactive with the fusosome; or
levels of antibody do not rise more than 1%, 2%, 5%, 10%, 20%, or 50% between two timepoints, the first timepoint being before the first administration of the fusosome, and the second timepoint being after one or more administrations of the fusosome.

131. The fusosome of any of the preceding embodiments, wherein the fusosome is a retroviral vector produced from cells that express exogenous or overexpressed HLA-G or HLA-E, e.g., cells that are transfected with a nucleic acid encoding HLA-G or HLA-E.

132. The fusosome of any of the preceding embodiments, wherein the fusosome is a retroviral vector generated from NMC-HLA-G cells and has a decreased percentage of lysis, e.g., PBMC mediated lysis, NK cell mediated lysis, and/or CD8+ T cell mediated lysis, at specific timepoints as compared to retroviral vectors generated from NMCs or NMC-empty vector.

133. The fusosome of any of the preceding embodiments, wherein the modified fusosome evades phagocytosis by macrophages.

134. The fusosome of any of the preceding embodiments, wherein the fusosome is produced from cells that express exogenous or overexpressed CD47, e.g., transfected with a nucleic acid encoding CD47.

135. The fusosome of any of the preceding embodiments, wherein the fusosome is a retroviral vector and wherein the phagocytic index is reduced when macrophages are incubated with retroviral vectors derived from NMC-CD47, versus those derived from NMC, or NMC-empty vector.

136. The fusosome of any of the preceding embodiments, which has a reduction in macrophage phagocytosis, e.g., a reduction of 1%, 5%, 10%, 20%, 30%, 40%, 50%, 60%, 70%, 80%, 90%, or more in macrophage phagocytosis compared to a reference fusosome, e.g., an unmodified fusosome otherwise similar to the fusosome, wherein the reduction in macrophage phagocytosis is determined by assaying the phagocytosis index in vitro.

137. The fusosome of any of the preceding embodiments, wherein a composition comprising a plurality of the fusosomes has a phagocytosis index of 0, 1, 10, 100, or more, when incubated with macrophages in an in vitro assay of macrophage phagocytosis.

138. The fusosome of any of the preceding embodiments, which is modified and has reduced complement activity compared to an unmodified retroviral vector.

139. The fusosome of any of the preceding embodiments, which is produced from cells comprising an exogenous or overexpressed complement regulatory protein (e.g., DAF), e.g., from cells transfected with a nucleic acid encoding a complement regulatory protein, e.g., DAF.

140. The fusosome of any of the preceding embodiments, wherein the fusosome is a retroviral vector, and wherein the dose of retroviral vector at which 200 pg/ml of C3a is present is greater for the modified retroviral vector (e.g., HEK293-DAF) incubated with corresponding mouse sera (e.g., HEK-293 DAF mouse sera) than for the reference retroviral vector (e.g., HEK293 retroviral vector) incubated with corresponding mouse sera (e.g., HEK293 mouse sera).

141. The fusosome of any of the preceding embodiments, wherein wherein the fusosome is a retroviral vector, and wherein the dose of retroviral vector at which 200 pg/ml of C3a is present is greater for for the modified retroviral vector (e.g., HEK293-DAF) incubated with naive mouse sera than for the reference retroviral vector (e.g., HEK293 retroviral vector) incubated with naive mouse sera.

142. The fusosome of any of the preceding embodiments, which is resistant to complement mediated inactivation in patient serum 30 minutes after administration.

143. The fusosome of any of the preceding embodiments, wherein at least 0.001%, 0.01%, 0.1%, 10%, 1%, 20%, 30%, 40%, 50%, 60%, 70%, 80%, 90%, or 100% of fusosomes are resistant to complement mediated inactivation.

144. The fusosome of any of the preceding embodiments, wherein the complement regulatory protein comprises one or more of proteins that bind decay-accelerating factor (DAF, CD55), e.g. factor H (FH)-like protein-1 (FHL-1), e.g. C4b-binding protein (C4BP), e.g. complement receptor 1 (CD35), e.g. Membrane cofactor protein (MCP, CD46), e.g., Protectin (CD59), e.g. proteins that inhibit the classical and alternative complement pathway CD/C5 convertase enzymes, e.g. proteins that regulate MAC assembly.

145. The fusosome of any of the preceding embodiments, which is produced by cells with a reduced level of MHC I, e.g., from cells transfected with a DNA coding for an shRNA targeting MHC class I, e.g., wherein retroviral vectors derived from NMC-shMHC class I has lower expression of MHC class I compared to NMCs and NMC-vector control.

146. The fusosome of any of the preceding embodiments, wherein a measure of immunogenicity for fusosomes (e.g., retroviral vectors) is serum inactivation.

147. The fusosome of any of the preceding embodiments, wherein the percent of cells which receive the exogenous agent is not different between fusosome samples that have been incubated with serum and heat-inactivated serum from fusosome naïve mice.

148. The fusosome of any of the preceding embodiments, wherein the percent of cells which receive the exogenous agent is not different between fusosome samples that have been incubated with serum from fusosome naïve mice and no-serum control incubations.

149. The fusosome of any of the preceding embodiments, wherein the percent of cells which receive the exogenous agent is less in fusosome samples that have been incubated with positive control serum than in fusosome samples that have been incubated with serum from fusosome naïve mice.

150. The fusosome of any of the preceding embodiments, wherein a modified retroviral vector, e.g., modified by a method described herein, has a reduced (e.g., reduced compared to administration of an unmodified retroviral vector) serum inactivation following multiple (e.g., more than one, e.g., 2 or more), administrations of the modified retroviral vector.

151. The fusosome of any of the preceding embodiments, which is not inactivated by serum following multiple administrations.

152. The fusosome of any of the preceding embodiments, wherein a measure of immunogenicity for fusosome is serum inactivation, e.g., after multiple administrations.

153. The fusosome of any of the preceding embodiments, wherein the percent of cells which receive the exogenous agent is not different between fusosome samples that have been incubated with serum and heat-inactivated serum from mice treated with modified (e.g., HEK293-HLA-G) fusosome.

154. The fusosome of any of the preceding embodiments, wherein the percent of cells which receive the exogenous agent is not different between fusosome samples that have been incubated with serum from mice treated 1, 2, 3, 5 or 10 times with modified (e.g., HEK293-HLA-G) retroviral vectors.

155. The fusosome of any of the preceding embodiments, wherein the percent of cells which receive the exogenous agent is not different between fusosome samples that have been incubated with serum from mice treated with vehicle and from mice treated with modified (e.g., HEK293-HLA-G) fusosomes.

156. The fusosome of any of the preceding embodiments, wherein the percent of cells which receive the exogenous agent is less for fusosomes derived from a reference cell (e.g., HEK293) than for modified (e.g., HEK293-HLA-G) fusosomes.

157. The fusosome of any of the preceding embodiments, wherein a measure of immunogenicity for a fusosome is antibody responses.

158. The fusosome of any of the preceding embodiments, wherein a subject that receives a fusosome described herein has pre-existing antibodies which bind to and recognize the fusosome.

159. The fusosome of any of the preceding embodiments, wherein serum from fusosome-naïve mice shows more signal (e.g., fluorescence) than the negative control, e.g., serum from a mouse depleted of IgM and IgG, e.g., indicating that immunogenicity has occurred.

160. The fusosome of any of the preceding embodiments, wherein serum from fusosome-naïve mice shows similar signal (e.g., fluorescence) compared to the negative control, e.g., indicating that immunogenicity did not detectably occur.

161. The fusosome of any of the preceding embodiments, which comprises a modified retroviral vector, e.g., modified by a method described herein, and which has a reduced (e.g., reduced compared to administration of an unmodified retroviral vector) humoral response following multiple (e.g., more than one, e.g., 2 or more), administrations of the modified retroviral vector.

162. The fusosome of any of the preceding embodiments, wherein humoral response is assessed by determining a value for the level of anti-fusosome antibodies (e.g., IgM, IgG1, and/or IgG2 antibodies).

163. The fusosome of any of the preceding embodiments, wherein modified (e.g., NMC-HLA-G) fusosomes, e.g., retroviral vectors, have decreased anti-viral IgM or IgG1/2 antibody titers (e.g., as measured by fluorescence intensity on FACS) after injections, as compared to a control, e.g., NMC retroviral vectors or NMC-empty retroviral vectors.

164. The fusosome of any of the preceding embodiments, wherein recipient cells are not targeted by an antibody response, or an antibody response will be below a reference level.

165. The fusosome of any of the preceding embodiments, wherein signal (e.g., mean fluorescence intensity) is similar for recipient cells from mice treated with retroviral vectors and mice treated with PBS.

166. The fusosome of any of the preceding embodiments, wherein a measure of the immunogenicity of recipient cells is the macrophage response.

167. The fusosome of any of the preceding embodiments, wherein recipient cells are not targeted by macrophages, or are targeted below a reference level.

168. The fusosome of any of the preceding embodiments, wherein the phagocytic index is similar for recipient cells derived from mice treated with fusosomes and mice treated with PBS.

169. The fusosome of any of the preceding embodiments, wherein a measure of the immunogenicity of recipient cells is the PBMC response.

170. The fusosome of any of the preceding embodiments, wherein recipient cells do not elicit a PBMC response.

171. The fusosome of any of the preceding embodiments, wherein the percent of CD3+/CMG+ cells is similar for recipient cells derived from mice treated with fusosome and mice treated with PBS.

172. The fusosome of any of the preceding embodiments, wherein a measure of the immunogenicity of recipient cells is the natural killer cell response.

173. The fusosome of any of the preceding embodiments, wherein recipient cells do not elicit a natural killer cell response or elicit a lower natural killer cell response, e.g., lower than a reference value.

174. The fusosome of any of the preceding embodiments, wherein the percent of CD3+/CMG+ cells is similar for recipient cells derived from mice treated with fusosome and mice treated with PBS.

175. The fusosome of any of the preceding embodiments, wherein a measure of the immunogenicity of recipient cells is the CD8+ T cell response.

176. The fusosome of any of the preceding embodiments, wherein recipient cells do not elicit a CD8+ T cell response or elicit a lower CD8+ T cell response, e.g., lower than a reference value.

177. The fusosome of any of the preceding embodiments, wherein the percent of CD3+/CMG+ cells is similar for recipient cells derived from mice treated with fusosome and mice treated with PBS.

178. The fusosome of any of the preceding embodiments, wherein the fusogen is a re-targeted fusogen.

179. The fusosome of any of the preceding embodiments, which comprises a retroviral nucleic acid that encodes one or both of: (i) a positive target cell-specific regulatory element operatively linked to a nucleic acid encoding an exogenous agent, or (ii) a non-target cell-specific regulatory element operatively linked to the nucleic acid encoding the exogenous agent.

180. The fusosome of embodiment 179, wherein the nucleic acid comprises two insulator elements, e.g., a first insulator element upstream of a region encoding the exogenous agent and a second insulator element downstream of a region encoding the exogenous agent, e.g., wherein the first insulator element and second insulator element comprise the same or different sequences.

181. The fusosome of any of embodiments 179-180, wherein variation in the median exogenous agent level in a sample of cells isolated after administration of the fusosome to the subject at a first timepoint is at least, less than, or about 10,000%, 5,000%, 2,000%, 1,000%, 500%, 200%, 100%, 50%, 20%, 10%, or 5% of the median exogenous agent level in a sample of cells isolated after administration of the fusosome to the subject at a second, later timepoint.

182. The fusosome of embodiment 181, wherein the median expression level per cell is assessed only in cells that have a retroviral genome copy number of at least 1.0.

183. The fusosome of any of embodiments 179-182, wherein at least 10%, 20%, 30%, 40%, 50%, 60%, 70%, 80%, or 90% of target cells in the subject detectably comprise the exogenous agent.

184. The fusosome of any of embodiments 181-182, wherein the median payload gene expression level is assessed across cells isolated from the subject 7 days, 14 days, 28 days, 56 days, 112 days, 365 days, 730 days, 1095 days after administration of the fusosome to the subject.

185. The fusosome of any of embodiments 179-184, wherein at least 10%, 20%, 30%, 40%, 50%, 60%, 70%, 80%, or 90% of target cells in the subject that detectably comprised the exogenous agent at a first time point still detectably comprise the exogenous agent at a second, later timepoint, e.g., wherein the first time point is 7 days, 14 days, 28 days, 56 days, 112 days, 365 days, 730 days, 1095 days after administration of the fusosome to the subject.

186. The fusosome of embodiment 185, wherein the second time point is 7 days, 14 days, 28 days, 56 days, 112 days, 365 days, 730 days, 1095 days after the first time point.

187. The fusosome of any of embodiments 179-186, which is not genotoxic or does not increase the rate of tumor formation in target cells compared to target cells not treated with the fusosome.

188. The fusosome of any of embodiments 179-187, wherein the median exogenous agent level is assessed in a population of cells from a subject that has received the fusosome.

189. The fusosome of any of embodiments 179-188, wherein the median exogenous agent level assessed in populations of cells collected (e.g., isolated) from the subject at different days post administration is less than about 10,000% 1000%, 100%, or 10%, e.g., 10,000%-1000%, 1000%-100%, or 100%-10% different from the median exogenous agent level in the population of cells assessed at day 7, day 14, day 28, or day 56, wherein the cells in the population have a vector copy number of at least 1.0.

190. The fusosome of any of embodiments 179-189, wherein exogenous agent level is assessed across cells from a subject that has received the fusosome.

191. The fusosome of any of embodiments 179-190, wherein the percent of cells comprising the exogenous agent is assessed in a plurality of cells collected (e.g., isolated) from the subject 7 days, 14 days, 28 days, 56 days, 112 days, 365 days, 730 days, 1095 days after administration of the fusosome.

192. The fusosome of any of embodiments 179-191, wherein the difference in the percent of cells comprising the exogenous agent assessed in cells isolated at two different days post administration is less than 1%, 5%, 10%, 20%, 50%, 75%, 100%, 150%, 200%, 250%, 300%, 400%, 500%, 750%, 1000%, 1500%, or 2000%.

193. The fusosome of any of embodiments 179-192, wherein the percent of target cells that are positive for the exogenous agent is similar across cells collected at 7 days, 14 days, 28 days, 56 days, 112 days, 365 days, 730 days, or 1095 days.

194. The fusosome of any of embodiments 179-193, wherein:
at least as many target cells are positive for the exogenous agent 14 days, 28 days, 56 days, 112 days, 365 days, 730 days, or 1095 days as at 7 days;
at least as many target cells are positive for the exogenous agent at 28 days, 56 days, 112 days, 365 days, 730 days, or 1095 days as at 14 days;
at least as many target cells are positive for the exogenous agent at 56 days, 112 days, 365 days, 730 days, or 1095 days as at 28 days;
at least as many target cells are positive for the exogenous agent at 112 days, 365 days, 730 days, or 1095 days as at 56 days;
at least as many target cells are positive for the exogenous agent at 365 days, 730 days, or 1095 days as at 112 days;
at least as many target cells are positive for the exogenous agent at 730 days or 1095 days as at 365 days; or
at least as many target cells are positive for the exogenous agent at 1095 days as at 730 days.

195. The fusosome of any of embodiments 179-194, wherein:
the median exogenous agent level in target cells that comprise the exogenous agent is similar in cells collected at 7 days, 14 days, 28 days, 56 days, 112 days, 365 days, 730 days, or 1095 days;
the median exogenous agent level in target cells that comprise the exogenous agent at 14 days, 28 days, 56 days, 112 days, 365 days, 730 days, or 1095 days is at least as high as at 7 days;
the median exogenous agent level in target cells that comprise the exogenous agent at 28 days, 56 days, 112 days, 365 days, 730 days, or 1095 days is at least as high as at 14 days;
the median exogenous agent level in target cells that comprise the exogenous agent at 56 days, 112 days, 365 days, 730 days, or 1095 days is at least as high as at 28 days;
the median exogenous agent level in target cells that comprise the exogenous agent at 112 days, 365 days, 730 days, or 1095 days is at least as high as at 56 days;
the median exogenous agent level in target cells that comprise the exogenous agent at 365 days, 730 days, or 1095 days is at least as high as at 112 days;
the median exogenous agent level in target cells that comprise the exogenous agent at 730 days, or 1095 days is at least as high as at 365 days; or
the median exogenous agent level in target cells that comprise the exogenous agent at 1095 days is at least as high as at 730 days.

196. A method of delivering an exogenous agent to a subject (e.g., a human subject) comprising administering to the subject a fusosome of any of the preceding embodiments, thereby delivering the exogenous agent to the subject.

197. A method of modulating a function, in a subject (e.g., a human subject), target tissue or target cell, comprising contacting, e.g., administering to, the subject, the target tissue or the target cell a fusosome of any of the preceding embodiments.

198. The method of embodiment 197, wherein the target tissue or the target cell is present in a subject.

199. A method of treating or preventing a disorder, e.g., a cancer, in a subject (e.g., a human subject) comprising administering to the subject a fusosome of any of the preceding embodiments.

200. A method of making a fusosome of any of the preceding embodiments, comprising:
a) providing a source cell that comprises the nucleic acid and the fusogen (e.g., re-targeted fusogen);
b) culturing the source cell under conditions that allow for production of the fusosome, and
c) separating, enriching, or purifying the fusosome from the source cell, thereby making the fusosome.

201. The method of any of the preceding embodiments, wherein the source cell for producing the fusosome lacksa fusogen receptor or wherein the fusogen receptor is present at reduced levels (e.g., reduced by at least 10%, 20%, 30%, 40%, 50%, 60%, 70%, 80%, or 90%) compared to an otherwise similar, unmodified source cell.

202. The source cell of embodiment 201, wherein the fusogen causes fusion of the fusosome with the target cell upon binding to the fusogen receptor.

203. The source cell of any of embodiments 201-202, which binds to the second similar source cell, e.g., the fusogen of the source cell binds to the fusogen receptor on the second source cell.

204. A population of source cells of any of embodiments 201-203.

205. The method of any of embodiments 201-204, wherein wherein less than 10%, 5%, 4%, 3%, 2%, or 1% of source cells are multinucleated.

206. The method of any of embodiments 201-205, wherein a source cell is modified to have reduced fusion (e.g., to not fuse) with other source cells during manufacturing of a fusosome described herein.

207. The method of any of embodiments 201-206, wherein the fusogen (e.g., re-targeted fusogen) does not bind to a protein comprised by a source cell, e.g., to a protein on the surface of the source cell.

208. The method of any of embodiments 201-207, wherein the fusogen (e.g., re-targeted fusogen) binds to a protein comprised by a source cell, but does not fuse with the cell.

209. The method of any of embodiments 201-208, wherein the fusogen does not induce fusion with a source cell.

210. The method of any of embodiments 201-209, wherein the source cell does not express a protein (e.g., an antigen) that binds the fusogen.

211. The method of any of embodiments 201-210, wherein a plurality of source cells do not form a syncytium when expressing the fusogen, or less than 50%, 40%, 30%, 20%, 10%, 5%, 4%, 3%, 2%, or 1% of cells in the population are multinucleated (e.g., comprise two or more nuclei).

212. The method of any of embodiments 201-211, wherein a plurality of source cells do not form a syncytium when producing fusosomes, or less than 50%, 40%, 30%, 20%, 10%, 5%, 4%, 3%, 2%, or 1% of cells in the population are multinucleated.

213. The method any of embodiments 201-212, wherein less than 50%, 40%, 30%, 20%, 10%, 5%, 4%, 3%, 2%, or 1% of the nuclei in the population are in syncytia.

214. The method of any of embodiments 201-213, wherein at least 50%, 60%, 70%, 80%, 90%, 95%, 96%, 97%, 98%, 99% of nuclei in the population are in uninuclear cells.

215. The method of any of embodiments 201-214, wherein the percentage of cells that are multinucleated is lower in a population of the modified source cells compared to an otherwise similar population of unmodified source cell, e.g., lower by at least 10%, 20%, 30%, 40%, 50%, 60%, 70%, 80%, 90%, 95%, 96%, 97%, 98%, or 99%.

216. The method of any of embodiments 201-215, wherein the percent of nuclei present in syncytia is lower in a population of the modified source cells compared to an otherwise similar population of unmodified source cell, e.g., lower by at least 10%, 20%, 30%, 40%, 50%, 60%, 70%, 80%, 90%, 95%, 96%, 97%, 98%, or 99%.

217. The method of any of embodiments 201-216, wherein multinucleated cells (e.g., cells having two or more nuclei) are detected by a microscopy assay, e.g., using a DNA stain.

218. The method of any of embodiments 201-217, wherein the functional fusosomes (e.g., viral particles) obtained from the modified source cells is at least 10%, 20%, 40%, 40%, 50%, 60%, 70%, 8-%, 90%, 2-fold, 5-fold, or 10-fold greater than the number of fusosomes obtained from otherwise similar unmodified source cells.

219. The fusosome of any of the preceding embodiments, which lacks a fusogen receptor or comprises a fusogen receptor that is present at reduced levels (e.g., reduced by at least 10%, 20%, 30%, 40%, 50%, 60%, 70%, 80%, or 90%) compared to an unmodified fusosome from an otherwise similar source cell.

220. The method of any of embodiments 201-219, which comprises knocking down or knocking out the fusogen receptor in the source cell or a precursor thereof.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Jul. 6, 2020. When one gene or protein references a plurality of sequence accession numbers, all of the sequence variants are encompassed. In addition, the materials, methods, and examples are illustrative only and not intended to be limiting.

### BRIEF DESCRIPTION OF DRAWINGS

FIGS. 1A-1B are a series of diagrams showing an overview of henipavirus F protein processing. FIG. 1A shows the inactive precursor (F₀) that results from initial translation of henipavirus F protein, which is trafficked to the plasma membrane (PM) and then recycled to endosomes and lysosomes for cleavage by cathepsin L into the fusion active F₁/F₂ subunits. The active form complexes with protein G and initiates membrane fusion. FIG. 1B shows two motifs (Y(525)RSL and Y(542)Y) in the henipavirus F protein cytoplasmic tail that were identified as important for henipavirus F protein endocytosis and exposure to cathepsin L for cleavage. These motifs are missing in the NivFd22 truncated protein that was used to enhance lentiviral-pseudo-typing.

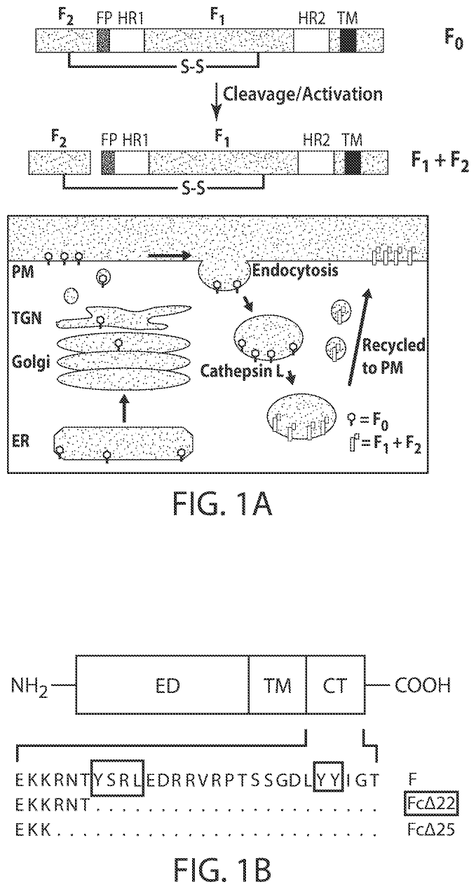

FIGS. 2A-2B are data from a series of experiments showing titres for fusosomes targeting CD8 or other cell surface markers following overexpression of cathepsin L. FIG. 2A shows quantification of functional viral titres measured after transduction of CD8 overexpressing cells, as described in Example 1. FIG. 2B shows quantification of viral titres on target cells transduced with Niv protein G constructs having targeting moieties recognizing different cell surface moieties, with or without overexpressed cathepsin L, as was described in Example 2.

FIGS. 3A-3B show quantification of functional titres for fusosomes targeting CD8 on PanT cells. Pan T cells were transduced with either concentrated (FIG. 3A) or crude (FIG. 3B) pseudotyped lentivirus lysates as described in Example 3. From left to right, each bar indicates: 1) no HA on NivF and no CathL overexpression; Xfect transfection reagent; 2) HA on NivF and no CathL overexpression; Xfect transfection reagent; 3) no HA on NivF and CathL overexpression; Xfect transfection reagent; and 4) HA on NivF and CathL overexpression; Xfect transfection reagent.

FIG. 4 shows quantification by flow cytometry of transduced Pan T cells that are positive for GFP. GFP expression in the Pan T cells indicates successful transduction of the target cells by the fusosome. Pan T cells were transduced with pseudotyped lentivirus lysates isolated from 293LX producer cells that were transfected as described in Example 3 and Table 6. The flow cytometry plots show, on the X axis, GFP levels, indicating successful transduction of Pan T cells, and on the Y axis, CD8 levels, indicating which cells in the population are positive for CD8 and therefore targeted by the fusosomes. All experiments used a pseudotyped lentivirus dilution of 0.04. The percentage of the double-positive CD8 and GFP cells for each transduction shown in FIG. 4 is included in Table 7.

FIGS. 5A-5C show the effects of overexpression of cathepsin L on the processing of henipavirus F protein in producer cells and their respective isolated pseudotyped lentivirus sample. 293LX producer cells were transfected as previously described to produce CD8-targeted Nipah G and F pseudotyped lentiviral vectors. Their respective supernatants containing the pseudotyped lentiviruses were isolated, as described in Example 3 and Table 6. In FIG. 5A Western blot band intensity for the F₀ precursor inactive protein and the cleaved, fusion active F₁ subunit detected in the producer cells and pseudotyped lentivirus samples were quantified, as in Example 4A. The X axis indicates the sample (Producer cells − CathL and + CathL, LVs − CathL and + CathL), and the Y axis indicates the AUC (area under curve) of the protein signal intensity from the Western blot. Producer cells − CathL shows AUC values of approximately 12-13,000 for both F₀ and F₁; Producer cells + CathL shows AUC values of approximately 2,000 for F₀ and 9,000 for F₁; LV − CathL shows AUC values of approximately 20,000 for F₀ and 9,000 for F₁; and LV+ CathL shows AUC values of approximately 12,000 for both F₀ and F₁. In FIG. 5B, the percent of the cleaved F₁ subunit to total F protein (F₁+F₀) was determined, as in Example 4A. The X axis indicates the sample (Producer cells − CathL and + CathL, LVs − and + CathL) and the Y axis indicates the percent of the cleaved F₁ subunit to total F protein. The percent of the cleaved F₁ subunit to total F protein was approximately 45% for Producer cells − CathL, approximately 80% for Producer cells + CathL, approximately 30% for LVs − CathL, and approximately 50% for LVs+ CathL. In FIG. 5C, there is a schematic of the henipavirus F protein, that shows the active F₁/F₂ subunits with the cleavage site, as well as the entire inactive F₀ subunit for reference.

FIG. 6 measures mature (proteolytically-processed) cathepsin L production and processing in producer cell samples. 293LX producer cells were transfected and their respective supernatants containing the pseudotyped lentiviruses were isolated, as described in Example 3 and Table 6. Producer cells were lysed to obtain protein samples and these were analyzed by Western blot with an anti-cathepsin L antibody, as described in Example 4B. The image shows the protein band corresponding to mature cathepsin L.

FIG. 7 measures p24 production in isolated pseudotyped lentivirus samples. 293LX producer cells were transfected and their respective supernatants containing the pseudotyped lentiviruses were isolated, as described in Example 3 and Table 6. Pseudotyped lentivirus samples were analyzed by Western blot with an anti-p24 antibody, as described in Example 4C.

FIG. 8 measures henipavirus G protein expression in producer cell samples. 293LX producer cells were transfected and their respective supernatants containing the pseudotyped lentiviruses were isolated, as described in Example 3 and Table 6. Producer cells were lysed to obtain protein samples and these were analyzed by Western blot with an anti-henipavirus G protein antibody, as in Example 5.

### DETAILED DESCRIPTION

The present disclosure provides, at least in part, fusosome methods and compositions for in vivo delivery. In particular, the disclosure provides methods for producing a plurality of fusosomes, using mammalian producer cells comprising an elevated level or activity of a mature cathepsin molecule (e.g., cathepsin L or cathepsin B), a henipavirus F protein, a henipavirus G protein, and optionally an exogenous cargo molecule.

### Definitions

Terms used in the claims and specification are defined as set forth below unless otherwise specified.

As used herein, the term “antibody molecule” refers to a polypeptide that comprises sufficient sequence(s) from an immunoglobulin heavy chain variable region and/or sufficient sequence(s) from an immunoglobulin light chain variable region to provide antigen specific binding. An antibody molecule may comprise a full length antibody and/or a fragment thereof, e.g., a Fab fragment, that support antigen binding. In some embodiments an antibody molecule will comprise heavy chain CDR1, CDR2, and CDR3 sequences and light chain CDR1, CDR2, and CDR3 sequences. Antibody molecules include, for example, human, humanized, CDR-grafted antibodies and antigen binding fragments thereof. In some embodiments, an antibody molecule comprises a protein that comprises at least one immunoglobulin variable region segment, e.g., an amino acid sequence that provides an immunoglobulin variable domain or immunoglobulin variable domain sequence. Examples of antibody molecules include, but are not limited to, humanized antibody molecules, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); Anticalins®; Nanobodies®; minibodies; BiTE® s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR® s.

As used herein, “cargo molecule” refers to a molecule (e.g., a nucleic acid molecule or a polypeptide, e.g., a protein) that is comprised by a fusosome. In some embodiments, a cargo molecule is packaged into a fusosome by a cell, e.g., a source cell as described herein. In some embodiments, a cargo molecule is an exogenous agent relative to the fusosome or the source cell.

As used herein, the term “cathepsin molecule” refers to a molecule having a structure and/or function of a cathepsin (e.g., cathepsin B or cathepsin L, e.g., as described herein). A cathepsin molecule can, in some embodiments, be a cysteine protease. In some embodiments, a cathepsin molecule comprises the amino acid sequence of a cathepsin protein as described herein (e.g., a cathepsin B or cathepsin L, e.g., a human cathepsin B or human cathepsin L). In some embodiments, an elevated level or activity of cathepsin molecules can result in increased functional titres of fusosomes (e.g., as described in Example 3), for example, by increasing F protein (e.g., Henipavirus F protein) processing (without being bound by theory). In some embodiments, a cathepsin molecule increases the ratio of active F protein (e.g., measured by F₁ level) to inactive F protein (F₀) in a producer cell, e.g., as described in Example 4. As used herein, “total cathepsin molecules” generally refers to the total number of cathepsin molecules in a cell, e.g., a source cell. Total cathepsin molecules may include, in some instances, both cathepsin molecules exogenous to the cell as well as cathepsin molecules endogenous to the cell. As used herein, an “exogenous cathepsin molecule” is a cathepsin molecule that is exogenous relative to the fusosome, source cell, and/or target cell. In some embodiments, the exogenous cathepsin molecule comprises one or more differences (e.g., mutations) relative to a wild-type cathepsin molecule (e.g., expressed by the source cell, e.g., producer cell). In some embodiments, the exogenous cathepsin molecule has the sequence of a wild-type cathepsin molecule, e.g., and is expressed by a nucleic acid molecule provided to the source cell (e.g., producer cell) exogenously.

As used herein, “fusosome” refers to a bilayer of amphipathic lipids enclosing a lumen or cavity and a fusogen that interacts with the amphipathic lipid bilayer. In embodiments, the fusosome comprises a nucleic acid. In some embodiments, the fusosome is a membrane enclosed preparation. In some embodiments, the fusosome is derived from a source cell.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “fusogen receptor” refers to an entity (e.g., a protein) comprised by a target cell, wherein binding of a fusogen on a fusosome (e.g., retrovirus) to a fusogen receptor on a target cell promotes delivery of a nucleic acid (e.g., retroviral nucleic acid) (and optionally also an exogenous agent encoded therein) to the target cell.

As used herein, an “insulator element” refers to a nucleotide sequence that blocks enhancers or prevents heterochromatin spreading. An insulator element can be wild-type or mutant.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a fusosome, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, the term “henipavirus F protein molecule” refers to a polypeptide having a structure and/or function of a henipavirus fusion protein (e.g., encoded by a henipavirus F gene). In some embodiments, a henipavirus F protein molecule is involved in (e.g., induces, e.g., in combination with a henipavirus G protein molecule) fusion between the membrane of the fusosome and the membrane of a target cell. In some embodiments, a henipavirus F protein molecule is part of a trimer of polypeptides, e.g., a homotrimer. In some embodiments, a fusosome comprises a plurality of henipavirus F protein molecules on its surface. In some embodiments, a henipavirus F protein molecule has at least 85%, 90%, 95%, 96%, 97%, 98%, 99%, or 100% sequence identity to a henipavirus F protein, or to a protein encoded by a henipavirus F gene. In some embodiments, a henipavirus F protein molecule is capable of promoting fusion between a fusosome membrane and the membrane of a target cell (e.g., in combination with a henipavirus G protein molecule). A henipavirus F protein molecule may be active or inactive. Typically, a henipavirus F protein is produced as an inactive form and then processed into the active form. More specifically, a henipavirus F protein is typically produced as an F0 chain and then cleaved to produce the F₁ and F₂ chains (which are connected to each other by a disulfide bridge) and are active. An “active” henipavirus F protein molecule, as used herein, refers to a henipavirus F protein molecule that comprises an F₁ chain, e.g., which has been produced by cleavage of an F0 chain to produce an F₁ and F₂ chains. An “inactive” henipavirus F protein molecule, as used herein, refers to a henipavirus F protein molecule having a F0 chain “Total” henipavirus protein F includes both active and inactive henipavirus protein F.

As used herein, the term “henipavirus G protein molecule” refers to a polypeptide having a structure and/or function of a henipavirus G protein (e.g., encoded by a henipavirus G gene). In some embodiments, a henipavirus G protein molecule is capable of binding to a polypeptide on the surface of a target cell. In some embodiments, a fusosome comprises a plurality of henipavirus G protein molecules on its surface. In some embodiments, a henipavirus G protein molecule has at least 85%, 90%, 95%, 96%, 97%, 98%, 99%, or 100% sequence identity to a henipavirus G protein, or to a protein encoded by a henipavirus G gene. In some embodiments, the henipavirus G protein molecule is a fusion protein, e.g., comprising a heterologous targeting moiety. In some embodiments, the henipavirus G protein molecule is a re-targeted fusogen.

The term “pharmaceutically acceptable” as used herein, refers to excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “positive target cell-specific regulatory element” (or positive TCSRE) refers to a nucleic acid sequence that increases the level of an exogenous agent in a target cell compared to in a non-target cell, wherein the nucleic acid encoding the exogenous agent is operably linked to the positive TCSRE. In some embodiments, the positive TCSRE is a functional nucleic acid sequence, e.g., the positive TCSRE can comprise a promoter or enhancer. In some embodiments, the positive TCSRE encodes a functional RNA sequence, e.g., the positive TCSRE can encode a splice site that promotes correct splicing of the RNA in the target cell. In some embodiments, the positive TCSRE encodes a functional protein sequence, or the positive TCSRE can encode a protein sequence that promotes correct post-translational modification of the protein. In some embodiments, the positive TCSRE decreases the level or activity of a downregulator or inhibitor of the exogenous agent.

As used herein, a “non-target cell-specific regulatory element” (or NTCSRE) refers to a nucleic acid sequence that decreases the level of an exogenous agent in a non-target cell compared to in a target cell, wherein the nucleic acid encoding the exogenous agent is operably linked to the NTCSRE. In some embodiments, the NTCSRE is a functional nucleic acid sequence, e.g., a miRNA recognition site that causes degradation or inhibition of the retroviral nucleic acid in a non-target cell. In some embodiments, the nucleic acid sequence encodes a functional RNA sequence, e.g., the nucleic acid encodes an miRNA sequence present in an mRNA encoding an exogenous protein agent, such that the mRNA is degraded or inhibited in a non-target cell. In some embodiments, the NTCSRE increases the level or activity of a downregulator or inhibitor of the exogenous agent. The terms “negative TCSRE” and “NTCSRE” are used interchangeably herein.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “target cell” refers to a cell of a type to which it is desired that a fusosome (e.g., lentiviral vector) deliver an exogenous agent. In embodiments, a target cell is a cell of a specific tissue type or class, e.g., an immune effector cell, e.g., a T cell. In some embodiments, a target cell is a diseased cell, e.g., a cancer cell.

As used herein a “non-target cell” refers to a cell of a type to which it is not desired that a fusosome (e.g., lentiviral vector) deliver an exogenous agent. In some embodiments, a non-target cell is a cell of a specific tissue type or class. In some embodiments, a non-target cell is a non-diseased cell, e.g., a non-cancerous cell.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

### Cathespins

Described herein are methods and compositions involving fusosomes comprising an elevated level or activity of a cathepsin molecule, e.g., a mature cathepsin molecule. In some embodiments, the cathepsin molecule is cathepsin L or cathepsin B. Generally, cathepsins are protease enzymes commonly active in organelles characterized by low pH (e.g., low relative to cytosolic pH), such as lysosomes. In some instances, cathepsins, such as cathepsin L and cathepsin B, are cysteine proteases involved in intracellular proteolysis (e.g., lysosomal proteolysis).

In some embodiments, a cathepsin molecule is initially produced as a preproenzyme, generally referred to as a procathepsin, which is subsequently processed in the cell into a “mature” cathepsin molecule. A mature cathepsin molecule may include, in some embodiments, a heavy chain polypeptide and a light chain polypeptide. The mature cathepsin can exist as a single chain form (e.g. of about 28 kDa) and/or as a two-chain form of a heavy and light chain, (e.g. of about 24 and 4 kDa, respectively). In some embodiments, the heavy chain polypeptide and the light chain polypeptide are linked, e.g., by one or more disulfides. In some embodiments, a mature cathepsin molecule comprises the amino acid sequence of a cathepsin L1 protein, e.g., a human cathepsin L1 protein (e.g., the amino acid sequence of SEQ ID NO: 1 below). In some embodiments, a cathepsin molecule has at least 85%, 90%, 95%, 96%, 97%, 98%, 99%, or 100% sequence identity to the exemplary cathepsin L1 sequence of SEQ ID NO: 1. In some embodiments, a mature cathepsin molecule comprises the amino acid sequence of a cathepsin B protein, e.g., a human cathepsin B protein (e.g., the amino acid sequence of SEQ ID NO: 2 below). In some embodiments, a cathepsin molecule has at least 85%, 90%, 95%, 96%, 97%, 98%, 99%, or 100% sequence identity to the exemplary cathepsin B sequence of SEQ ID NO: 2.

Exemplary Cathepsin L1 Sequence (SEQ ID NO: 1):
MDYAFQYVQDNGGLDSEESYPYEATEESCKYNPKYSVANDTGFVDIPKQ
EKALMKAVATVGPISVAIDAGHESFLFYKEGIYFEPDCSSEDMDHGVLV
VGYGFESTESDNNKYWLVKNSWGEEWGMGGYVKMAKDRRNHCGIASAAS
YPTV
Exemplary Cathepsin B Sequence (SEQ ID NO: 2):
MHGNNGHSVPPSKRSETRAPVAPAGCNGGYPAEAWNFWTRKGLVSGGLY
ESHVGCRPYSIPPCEHHVNGSRPPCTGEGDTPKCSKICEPGYSPTYKQD
KHYGYNSYSVSNSEKDIMAEIYKNGPVEGAFSVYSDELLYKSGVYQHVT
GEMMGGHAIRILGWGVENGTPYWLVANSWNTDWGDNGFFKILRGQDHCG
IESEVVAGIPRTDQYWEKI

In some embodiments, a mature cathepsin molecule comprises the amino acid sequence of a cathepsin L1 protein, e.g., a human cathepsin L1 protein (e.g., the amino acid sequence of SEQ ID NO: 37). In some embodiments, a cathepsin molecule has at least 85%, 90%, 95%, 96%, 97%, 98%, 99%, or 100% sequence identity to the exemplary cathepsin L1 sequence of SEQ ID NO: 37.

In some embodiments, a mature cathepsin molecule comprises the amino acid sequence of a cathepsin B protein, e.g., a human cathepsin B protein (e.g., the amino acid sequence of SEQ ID NO: 38). In some embodiments, a cathepsin molecule has at least 85%, 90%, 95%, 96%, 97%, 98%, 99%, or 100% sequence identity to the exemplary cathepsin B sequence of SEQ ID NO: 38.

In some embodiments, a mature cathepsin molecule comprises the amino acid sequence of a cathepsin B protein, e.g., a human cathepsin B protein (e.g., the amino acid sequence of SEQ ID NO: 39). In some embodiments, a cathepsin molecule has at least 85%, 90%, 95%, 96%, 97%, 98%, 99%, or 100% sequence identity to the exemplary cathepsin B sequence of SEQ ID NO: 39.

In some embodiments, a nucleic acid encoding a cathepsin molecule is introduced into a host cell used in connection with producing a fusosme as provided herein. For instance, a nucleic acid encoding a cathepsin (e.g. a cathepsin L or cathepsin B) is introduced into a packaging cell line (a producer cell) used in connection with methods of producing retroviral vectors as described below. In some embodiments, the nucleic acid molecule encodes a propeptide form of acathepsin that includes the coding sequence for mature cathepsin. Upon cleavage of the propeptide a mature cathepsin is produced. In other embodiments, the nucleic acid molecule encodes mature cathepin, e.g. set forth in SEQ ID NO:1 SEQ ID NO:2, SEQ ID NO: 37, SEQ ID NO: 38, or SEQ ID NO: 39. In some embodiments, the nucleic acid encodes a cathepsin L that exhibits at least 85%, 90%, 95%, 98% or more sequence identity to SEQ ID NO:1 In some embodiments, the nucleic acid encodes a cathepsin L set forth in SEQ ID NO:1. In some embodiments, the nucleic acid encodes a cathepsin L that exhibits at least 85%, 90%, 95%, 98% or more sequence identity to SEQ ID NO:37 In some embodiments, the nucleic acid encodes a cathepsin L set forth in SEQ ID NO:37. In some embodiments, the nucleic acid molecules encodes a cathepsin B that exhibits at least 85%, 90%, 95%, 98% or more sequence identity to SEQ ID NO:2. In some embodiments, the nucleic acid molecules encodes a cathepsin B as set forth in SEQ ID NO:2. In some embodiments, the nucleic acid encodes a cathepsin L that exhibits at least 85%, 90%, 95%, 98% or more sequence identity to SEQ ID NO:38. In some embodiments, the nucleic acid encodes a cathepsin L set forth in SEQ ID NO:38. In some embodiments, the nucleic acid molecules encodes a cathepsin B that exhibits at least 85%, 90%, 95%, 98% or more sequence identity to SEQ ID NO:39. In some embodiments, the nucleic acid molecules encodes a cathepsin B as set forth in SEQ ID NO:39.

In some embodiments, the producer cell is a mammalian cell. Any suitable cell line can be employed as a producer or packaging cell line in accord with producing fusosome, e.g. retroviral vector particles, such as a lentiviral vector. In some embodiments, the cell line includes mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a cathepsin molecule is expressed in a host cell (e.g., a producer cell for producing fusosomes, as described herein). Any suitable method for expressing an exogenous polypeptide can be used to express a cathepsin molecule in such a host cell.

In some embodiments, a cathepsin molecule is expressed transiently in a host cell, e.g., by transfecting the host cell with a nucleic acid construct comprising a sequence encoding the cathepsin molecule, e.g., under the control of a suitable promoter (e.g., a constitutive promoter or an inducible promoter). In some embodiments, the nucleic acid molecule is introduced for episomal delivery to the cell. Methods that provide a transgene as an episome include delivery with an expression plasmid, a virus-like particle, or an adenovirus (AAV).

In some embodiments, expression is achieved using a site-specific activator of a cathepsin locus in the cell, e.g. a mammalian cell. For instance, a fusion protein may be introduced into the cell comprising a site-specific binding domain specific for the cathepsin gene (e.g. CTSB or CTSL) and a transcriptional activator. In some of any embodiments, the site-specific binding domain is selected from the group consisting of: zinc fingers, transcription activation like (TAL) effectors, meganucleases, and CRISPR/Cas9 system components, or a modified form thereof. In some of any embodiments, the encoded regulatory factor is a zinc finger transcription factor (ZF-TF). In some of any embodiments, the site-specific binding domain is a CRISPR/Cas system, wherein the CRISPR/Cas system comprises a modified Cas nuclease that lacks nuclease activity and a guide RNA (gRNA). In some of any embodiments, the modified nuclease is a catalytically dead Cas9 (dCas9). In some of any embodiments, the transcriptional activator is selected from Herpes simplex-derived transactivation domain, Dnmt3a methyltransferase domain, p65, VP16, and VP64. In some of any embodiments, the transcriptional activator is the tripartite activator VP64-p65-Rta (VPR).

In some embodiments, a cathepsin molecule is introduced into the cell under conditions for stable expression of the cathepsin. For instance, in some embodiments, a cathepsin molecule is introduced into the cell for integration into the chromosome of the cells. Any of a variety of methods can be used for stable integration of a delivered nucleic acid molecule into a cell. In some embodiments, a nucleic acid encoding a cathepsin is delivered to a cell using a lentiviral vector. In other embodiments, a nucleic acid encoding a cathepsin is delivered to the cell by targeted integration to a chosen locus in the cell.

Methods for targeted integration are known. For instance, any of a variety of site-specific nucleases can be used to mediate targeted cleavage of host cell DNA to bias insertion into a chosen genomic locus (see, e.g. U.S. Pat. No. 7,888,121 and U.S. Patent Publication No. 201 10301073). Specific nucleases can be used that cleave within or near the endogenous locus and the transgene can be integrated at or near the site of cleavage through homology directed repair (HDR) or by end capture during non-homologous end joining (NHIEJ). The integration process is influenced by the use or non-use of regions of homology on the transgene donors. These regions of chromosomal homology on the donor flank the transgene cassette and are homologous to the sequence of the endogenous locus at the site of cleavage.

In some embodiments, the target locus is a non-cognate locus, such as one chosen due to a desired beneficial property. In some cases, the nucleic acid encoding a cathepsin may be inserted into a specific “safe harbor” location in the genome that may either utilize the promoter found at that safe harbor locus, or allow the expressional regulation of the transgene by an exogenous promoter that is fused to the transgene prior to insertion. Several such “safe harbor” loci have been described, including the AAVS1 (also known as PPP1R12C) and CCR5 genes in human cells, Rosa26 and albumin (see co-owned U.S. Patent Publication Nos. 20080299580, 20080159996 and 201000218264 and U.S. application Ser. Nos. 13/624,193 and 13/624,217). As described above, nucleases specific for the safe harbor can be utilized such that the transgene construct is inserted by either HDR- or NHEJ-driven processes.

In some embodiments, other components used for producing the fusosome also may be expressed in the producer or packaging cell, such as described below, e.g. for retroviral or viral-like particle production methods. In some embodiments, a transfer vector may be employed that is a retroviral (e.g. lentiviral) transfer plasmid encoding a transgene (e.g. exogenous agent) of interest in which the transgene sequence is flanked by long terminal repeat (LTR) sequences to facilitate integration of the transfer plasmid sequences into the host genome, and otherwise lacks viral sequences so that it is replication defective. The transfer vector may then be introduced into a packaging cell line containing the gag, pol, and env genes, but without the LTR and packaging components. The recombinant retroviral particles are secreted into the culture media, then collected, optionally concentrated, and used for gene transfer.

In particular embodiments, the packaging cell line contains genes encoding a henipavirus F protein molecule (e.g. any as described) and a henipavirus G protein molecule (e.g. any as described), such that the retroviral vector (e.g. lentiviral vector) is pseudotyped with envelope proteins from a henipavirus. In particular embodiments, the packaging cell line contains genes encoding a henipavirus F protein molecule (e.g. any as described) and a henipavirus G protein molecule (e.g. any as described), such that the virus-like particle (e.g. lentiviral-like particle) is pseudotyped with envelope proteins from a henipavirus. In some embodiments, the henipavirus is Nipah virus. As described herein, the G protein molecule may modified to incorporate targeting/binding ligands to re-target the psuedotyped fusome (e.g. lentiviral vector or virus-like particle) to any desired target cell. In some embodiments, production of the retroviral particle (e.g. lentiviral vector or lentivirus-like vector) using the provided producer cells that exhibit elevated or increased expression of a cathepsin (such as due to deliver of an exogenous nucleic acid encoding the cathepsin) results in retroviral vectors pseudotyped with the F and G protein molecules in which expression of the active F protein is increased due to improved F protein processing.

In some embodiments, an elevated level or activity of cathepsin molecules can promote increased functional titres of fusosomes (e.g., as described in Examples 1-3), for example, by increasing F protein (e.g., Henipavirus F protein) processing. For example, a cathepsin molecule may increase the ratio of active F protein (F₁+F₂) to inactive F protein (F₀) in a producer cell, e.g., as described in Example 4. In some embodiments, the ratio of active to inactive F protein is increased by reducing the levels of inactive protein, e.g., as described in Example 4.

### Fusosomes, e.g., Cell-Derived Fusosomes

Fusosomes can take various forms. Generally, a fusosome described herein comprises an elevated activity and/or elevated level of a cathepsin molecule (e.g., as described herein). In some embodiments, the fusosome comprises a henipavirus F protein molecule and a henipavirus G protein molecule. In some embodiments, a fusosome described herein is derived from a source cell (e.g., a producer cell as described herein). A fusosome may comprise, e.g., an extracellular vesicle, a microvesicle, a nanovesicle, an exosome, an apoptotic body (from apoptotic cells), a microparticle (which may be derived from, e.g., platelets), an ectosome (derivable from, e.g., neutrophiles and monocytes in serum), a prostatosome (obtainable from prostate cancer cells), a cardiosome (derivable from cardiac cells), or any combination thereof. In some embodiments, a fusosome is released naturally from a source cell, and in some embodiments, the source cell is treated to enhance formation of fusosomes. In some embodiments, the fusosome is between about 10-10,000 nm in diameter, e.g., about 30-100 nm in diameter. In some embodiments, the fusosome comprises one or more synthetic lipids.

In some embodiments, the fusosome is or comprises a virus, e.g., a retrovirus, e.g., a lentivirus. In some embodiments, a fusosome comprising a lipid bilayer comprises a retroviral vector comprising an envelope. For instance, in some embodiments, the fusosome's bilayer of amphipathic lipids is or comprises the viral envelope. The viral envelope may comprise a fusogen, e.g., a fusogen that is endogenous to the virus or a pseudotyped fusogen. In some embodiments, the fusosome's lumen or cavity comprises a viral nucleic acid, e.g., a retroviral nucleic acid, e.g., a lentiviral nucleic acid. The viral nucleic acid may be a viral genome. In some embodiments, the fusosome further comprises one or more viral non-structural proteins, e.g., in its cavity or lumen.

Fusosomes may have various properties that facilitate delivery of a payload, such as, e.g., a desired transgene or exogenous agent, to a target cell. For instance, in some embodiments, the fusosome and the source cell together comprise nucleic acid(s) sufficient to make a particle that can fuse with a target cell. In embodiments, these nucleic acid(s) encode proteins having one or more of (e.g., all of) the following activities: gag polyprotein activity, polymerase activity, integrase activity, protease activity, and fusogen activity.

Fusosomes may also comprise various structures that facilitate delivery of a payload to a target cell. For instance, in some embodiments, the fusosome (e.g., virus, e.g., retrovirus, e.g., lentivirus) comprises one or more of (e.g., all of) the following proteins: gag polyprotein, polymerase (e.g., pol), integrase (e.g., a functional or non-functional variant), protease, and a fusogen. In some embodiments, the fusosome further comprises rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the fusosome nucleic acid (e.g., retroviral nucleic acid) comprises one or more of (e.g., all of) the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the fusosome nucleic acid (e.g., retroviral nucleic acid) further comprises one or more insulator element. In some embodiments the fusosome nucleic acid (e.g., retroviral nucleic acid) further comprises one or more miRNA recognition sites. In some embodiments, one or more of the miRNA recognition sites are situated downstream of the poly A tail sequence, e.g., between the poly A tail sequence and the WPRE.

In some embodiments, a fusosome provided herein is administered to a subject, e.g., a mammal, e.g., a human. In such embodiments, the subject may be at risk of, may have a symptom of, or may be diagnosed with or identified as having, a particular disease or condition (e.g., a disease or condition described herein). In one embodiment, the subject has cancer. In one embodiment, the subject has an infectious disease. In some embodiments, the fusosome contains nucleic acid sequences encoding an exogenous agent for treating the disease or condition.

### Fusosome Components and Helper Cells

In some embodiments, the fusosome nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the fusosome nucleic acid further comprises one or more of a cPPT, a WPRE, and/or an insulator element.

In some embodiments, a fusosome comprises one or more elements of a retrovirus. A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLy), spumavirus, Friend murine leukemia virus, Murine Stem Cell Virus (MSCV) and Rous Sarcoma Virus (RSV)) and lentivirus. In some embodiments the retrovirus is a Gammaretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus.

In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. A viral vector can comprise, e.g., a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). A viral vector can comprise, e.g., a virus or viral particle capable of transferring a nucleic acid into a cell, or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′)R-U5-one or more first nucleotide sequences-U3-R(3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a fusosome nucleic acid (e.g., a retroviral nucleic acid, e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail, for example, in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

In some embodiments, the retrovial nucleic acid is devoid of all non-structual genes. In some embodiments, the fusosome is a viral-like particle (VLP) that is derived from virus. In some embodiments, the viral envelope may comprise a fusogen, e.g., a fusogen that is endogenous to the virus or a pseudotyped fusogen. The VLPS include those derived from retroviruses or lentiviruses. While VLPs mimic native virion structure, they lack the viral genomic information necessary for independent replication within a host cell. Therefore, in some aspects, VLPs are non-infectious. In particular embodiments, a VLP does not contain a viral genome. In some embodiments, the VLP's bilayer of amphipathic lipids is or comprises the viral envelope. In some embodiments, a VLP contains at least one type of structural protein from a virus. In most cases this protein will form a proteinaceous capsid. In some cases the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the fusosome comprises supramolecular complexes formed by viral proteins that self-assemble into capsids. In some embodiments, the fusosome is a virus-like particle derived from viral capsid proteins. In some embodiments, the fusosome is a virus-like particle derived from viral nucleocapsid proteins. In some embodiments, the fusosome comprises nucleocapsid-derived proteins that retain the property of packaging nucleic acids. In some embodiments, the fusosomes, such as virus-like particles, comprises only viral structural glycoproteins among proteins from the viral genome. In some embodiments, the fusosome does not contain a viral genome.

In some embodiments, the fusosome packages nucleic acids from host cells during the expression process, such as a nucleic acid encoding an exogenous agent. In some embodiments, the nucleic acids do not encode any genes involved in virus replication. In particular embodiments, the fusosome is a virus-like particle, e.g. retrovirus-like particle such as a lentivirus-like particle, that is replication defective.

In some embodiments, the fusosome is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the fusosome could be used to deliver therapeutic RNA, in which case functional integrase and/or reverse transcriptase is not required. In some embodiments, the fusosome could also be used to deliver a therapeutic gene of interest, in which case pol is typically included.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

Many viruses, including HIV and other lentiviruses, use a large number of rare codons and by changing these to correspond to commonly used mammalian codons, increased expression of the packaging components in mammalian producer cells can be achieved.

Codon optimization has a number of other advantages. By virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

Derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

Due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

The strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

As described above, the packaging components for a retroviral vector can include expression products of gag, pol and env genes. In addition, packaging can utilize a short sequence of 4 stem loops followed by a partial sequence from gag and env as a packaging signal. Thus, inclusion of a deleted gag sequence in the retroviral vector genome (in addition to the full gag sequence on the packaging construct) can be used. In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

The retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

The gag and pol coding sequences are generally organized as the Gag-Pol Precursor in native lentivirus. The gag sequence codes for a 55-kD Gag precursor protein, also called p55. The p55 is cleaved by the virally encoded protease4 (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

Native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In various examples, a fusosome nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

A packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, fusosome nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The fusosome nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a fusosome nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### Vectors Engineered to Remove Splice Sites

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read-through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

### Retroviral Production Methods

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self cleaving viral peptides.

Packaging cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

A source cell line includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

### Packaging Plasmids and Cell Lines

In some embodiments, the source cell used as a packaging cell line comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments, expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or fusosome may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

### Strategies for Packaging a Retroviral Nucleic Acid

Typically, modern retroviral vector systems consist of viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A viral vector particle which comprises a sequence that is devoid of or lacking viral RNA may be the result of removing or eliminating the viral RNA from the sequence. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The vector particles could be used to deliver therapeutic RNA, in which case functional integrase and/or reverse transcriptase is not required. These vector particles could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognising a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U1 small nuclear ribonucleoprotein particle, a Nova protein, a TF111A protein, a TIS11 protein, a trp RNA-binding attenuation protein (TRAP) or a pseudouridine synthase.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretrovirus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see, e.g., WO2018023094A1.

In some embodiments, the assembly of a fusosome (i.e., a VLP) is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral vector as described above can be detected by any suitable technique known in the art. Examples of such techniques include, e.g., electron microscopy, dynamic light scattering, selective chromatographic separation and/or density gradient centrifugation.

Repression of a Gene Encoding an Exogenous Agent in a Source Cell (Over-)expressed protein in the source cell may have an indirect or direct effect on vector virion assembly and/or infectivity. Incorporation of the exogenous agent into vector virions may also impact downstream processing of vector particles.

In some embodiments, a tissue-specific promoter is used to limit expression of the exogenous agent in source cells. In some embodiments, a heterologous translation control system is used in eukaryotic cell cultures to repress the translation of the exogenous agent in source cells. More specifically, the retroviral nucleic acid may comprise a binding site operably linked to the gene encoding the exogenous agent, wherein the binding site is capable of interacting with an RNA-binding protein such that translation of the exogenous agent is repressed or prevented in the source cell.

In some embodiments, the RNA-binding protein is tryptophan RNA-binding attenuation protein (TRAP), for example bacterial tryptophan RNA-binding attenuation protein. The use of an RNA-binding protein (e.g. the bacterial trp operon regulator protein, tryptophan RNA-binding attenuation protein, TRAP), and RNA targets to which it binds, will repress or prevent transgene translation within a source cell. This system is referred to as the Transgene Repression In vector Production cell system or TRIP system.

In embodiments, the placement of a binding site for an RNA binding protein (e.g., a TRAP-binding sequence, tbs) upstream of the NOI translation initiation codon allows specific repression of translation of mRNA derived from the internal expression cassette, while having no detrimental effect on production or stability of vector RNA. The number of nucleotides between the tbs and translation initiation codon of the gene encoding the exogenous agent may be varied from 0 to 12 nucleotides. The tbs may be placed downstream of an internal ribosome entry site (IRES) to repress translation of the gene encoding the exogenous agent in a multicistronic mRNA.

### Kill Switch Systems and Amplification

In some embodiments, a polynucleotide or cell harboring the gene encoding the exogenous agent utilizes a suicide gene, e.g., an inducible suicide gene, to reduce the risk of direct toxicity and/or uncontrolled proliferation. In specific aspects, the suicide gene is not immunogenic to the host cell harboring the exogenous agent. Examples of suicide genes include caspase-9, caspase-8, or cytosine deaminase. Caspase-9 can be activated using a specific chemical inducer of dimerization (CID).

In certain embodiments, vectors comprise gene segments that cause target cells, e.g., immune effector cells, e.g., T cells, to be susceptible to negative selection in vivo. For instance, the transduced cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, and bacterial cytosine deaminase, (Mullen et al., Proc. Natl. Acad. Sci. USA. 89:33 (1992)).

In some embodiments, transduced cells, e.g., immune effector cells, such as T cells, comprise a polynucleotide further comprising a positive marker that enables the selection of cells of the negative selectable phenotype in vitro. The positive selectable marker may be a gene which, upon being introduced into the target cell, expresses a dominant phenotype permitting positive selection of cells carrying the gene. Genes of this type include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

In some embodiments, the positive selectable marker and the negative selectable element are linked such that loss of the negative selectable element necessarily also is accompanied by loss of the positive selectable marker. For instance, the positive and negative selectable markers can be fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 1 1:3374-3378, 1991. In addition, in embodiments, the polynucleotides encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT U591/08442 and PCT/US94/05601, describing the use of bifunctional selectable fusion genes derived from fusing a dominant positive selectable markers with negative selectable markers.

Suitable positive selectable markers can be derived from genes selected from the group consisting of hph, nco, and gpt, and suitable negative selectable markers can be derived from genes selected from the group consisting of cytosine deaminase, HSV-I TK, VZV TK, HPRT, APRT and gpt. Other suitable markers are bifunctional selectable fusion genes wherein the positive selectable marker is derived from hph or neo, and the negative selectable marker is derived from cytosine deaminase or a TK gene or selectable marker.

### Strategies for Remulating Lentiviral Integration

Retroviral and lentiviral nucleic acids are disclosed which are lacking or disabled in key proteins/sequences so as to prevent integration of the retroviral or lentiviral genome into the target cell genome. For instance, viral nucleic acids lacking each of the amino acids making up the highly conserved DDE motif (Engelman and Craigie (1992) J. Virol. 66:6361-6369; Johnson et al. (1986) Proc. Natl. Acad. Sci. USA 83:7648-7652; Khan et al. (1991) Nucleic Acids Res. 19:851-860) of retroviral integrase enables the production of integration defective retroviral nucleic acids.

For instance, in some embodiments, a retroviral nucleic acid herein comprises a lentiviral integrase comprising a mutation that causes said integrase to be unable to catalyze the integration of the viral genome into a cell genome. In some embodiments, said mutations are type I mutations which affect directly the integration, or type II mutations which trigger pleiotropic defects affecting virion morphogenesis and/or reverse transcription. Illustrative non-limitative examples of type I mutations are those mutations affecting any of the three residues that participate in the catalytic core domain of the integrase: DX₃₉₋₅₈DX₃₅E (D64, D116 and E152 residues of the integrase of the HIV-1). In a particular embodiment, the mutation that causes said integrase to be unable to catalyze the integration of the viral genome into a cell genome is the substitution of one or more amino acid residues of the DDE motif of the catalytic core domain of the integrase, preferably the substitution of the first aspartic residue of said DEE motif by an asparagine residue. In some embodiment the retroviral vector does not comprise an integrase protein.

In some embodiments the retrovirus integrates into active transcription units. In some embodiments the retrovirus does not integrate near transcriptional start sites, the 5′ end of genes, or DNAse1 cleavage sites. In some embodiments the retrovirus integration does not active proto-oncogenes or inactive tumor suppressor genes. In some embodiments the retrovirus is not genotoxic. In some embodiments the lentivirus integrates into introns.

In some embodiments, the retroviral nucleic acid integrates into the genome of a target cell with a particular copy number. The average copy number may be determined from single cells, a population of cells, or individual cell colonies. Exemplary methods for determining copy number include polymerase chain reaction (PCR) and flow cytometry.

In some embodiments, DNA encoding the exogenous agent is integrated into the genome. In some embodiments DNA encoding the exogenous agent is maintained episomally. In some embodiments the ratio of integrated to episomal DNA encoding the exogenous agent is at least 0.01, 0.1, 0.5, 1.0, 2, 5, 10, 100.

In some embodiments, DNA encoding the exogenous agent is linear. In some embodiments DNA encoding the exogenous agent is circular. In some embodiments the ratio of linear to circular copies of DNA encoding the exogenous agent is at least 0.01, 0.1, 0.5, 1.0, 2, 5, 10, 100.

In embodiments the DNA encoding the exogenous agent is circular with 1 LTR. In some embodiments the DNA encoding the exogenous agent is circular with 2 LTRs. In some embodiments the ratio of circular, 1 LTR-comprising DNA encoding the exogenous agent to circular, 2 LTR-comprising DNA encoding the exogenous agent is at least 0.1, 0.5, 1.0, 2, 5, 10, 20, 50, 100.

### Maintenance of an Episomal Virus

In retroviruses deficient in integration, circular cDNA off-products of the retrotranscription (e.g., 1-LTR and 2-LTR) can accumulate in the cell nucleus without integrating into the host genome (see Yinez-Munoz R J et al., Nat. Med. 2006, 12: 348-353). Like other exogenous DNA those intermediates can then integrate in the cellular DNA at equal frequencies (e.g., 10³ to 10⁵/cell).

In some embodiments, episomal retroviral nucleic acid does not replicate. Episomal virus DNA can be modified to be maintained in replicating cells through the inclusion of eukaryotic origin of replication and a scaffold/matrix attachment region (S/MAR) for association with the nuclear matrix.

Thus, in some embodiments, a retroviral nucleic acid described herein comprises a eukaryotic origin of replication or a variant thereof. Examples of eukaryotic origins of replication of interest are the origin of replication of the β-globin gene as have been described by Aladjem et al (Science, 1995, 270: 815-819), a consensus sequence from autonomously replicating sequences associated with alpha-satellite sequences isolated previously from monkey CV-1 cells and human skin fibroblasts as has been described by Price et al Journal of Biological Chemistry, 2003, 278 (22): 19649-59, the origin of replication of the human c-myc promoter region has have been described by McWinney and Leffak (McWinney C. and Leffak M., Nucleic Acid Research 1990, 18(5): 1233-42). In embodiments, the variant substantially maintains the ability to initiate the replication in eukaryotes. The ability of a particular sequence of initiating replication can be determined by any suitable method, for example, the autonomous replication assay based on bromodeoxyuridine incorporation and density shift (Araujo F. D. et al., supra; Frappier L. et al., supra).

In some embodiments, the retroviral nucleic acid comprises a scaffold/matrix attachment region (S/MAR) or variant thereof, e.g., a non-consensus-like AT-rich DNA element several hundred base pairs in length, which organizes the nuclear DNA of the eukaryotic genome into chromatin domains, by periodic attachment to the protein scaffold or matrix of the cell nucleus. They are typically found in non-coding regions such as flanking regions, chromatin border regions, and introns. Examples of S/MAR regions are 1.8 kbp S/MAR of the human IFN-7 gene (hIFN-γ^{large}) as described by Bode et al (Bode J. et al., Science, 1992, 255: 195-7), the 0.7 Kbp minimal region of the S/MAR of the human IFN-7 gene (hIFN-γ^{short}) as has have been described by Ramezani (Ramezani A. et al., Blood 2003, 101: 4717-24), the 0.2 Kbp minimal region of the S/MAR of the human dehydrofolate reductase gene (hDHFR) as has been described by Mesner L. D. et al., Proc Natl Acad Sci USA, 2003, 100: 3281-86). In embodiments, the functionally equivalent variant of the S/MAR is a sequence selected based on the set six rules that together or alone have been suggested to contribute to S/MAR function (Kramer et al (1996) Genomics 33, 305; Singh et al (1997) Nucl. Acids Res 25, 1419). These rules have been merged into the MAR-Wiz computer program freely available at genomecluster.secs.oakland.edu/MAR-Wiz. In embodiments, the variant substantially maintains the same functions of the S/MAR from which it derives, in particular, the ability to specifically bind to the nuclear the matrix. The skilled person can determine if a particular variant is able to specifically bind to the nuclear matrix, for example by the in vitro or in vivo MAR assays described by Mesner et al. (Mesner L. D. et al, supra). In some embodiments, a specific sequence is a variant of a S/MAR if the particular variant shows propensity for DNA strand separation. This property can be determined using a specific program based on methods from equilibrium statistical mechanics. The stress-induced duplex destabilization (SIDD) analysis technique “[ . . . ] calculates the extent to which the imposed level of superhelical stress decreases the free energy needed to open the duplex at each position along a DNA sequence. The results are displayed as an SIDD profile, in which sites of strong destabilization appear as deep minima [ . . . ]” as defined in Bode et al (2005) J. Mol. Biol. 358,597. The SIDD algorithm and the mathematical basis (Bi and Benham (2004) Bioinformatics 20, 1477) and the analysis of the SIDD profile can be performed using the freely available internet resource at WebSIDD (www.genomecenter.ucdavis.edu/benham). Accordingly, in some embodiment, the polynucleotide is considered a variant of the S/MAR sequence if it shows a similar SIDD profile as the S/MAR.

### Fusogens and Pseudotyping

Fusogens, which include, e.g., viral envelope proteins (env), generally determine the range of host cells which can be infected and transformed by fusosomes. In some embodiments, a fusosome herein comprises a henipavirus F protein molecule and a henipavirus G protein molecule. In some embodiments, the henipavirus F protein molecule and/or the henipavirus G protein molecule contribute to fusosome fusion with the cell membrane. For example, a henipavirus F protein molecule may mediate fusion between the membrane of the fusosome and the cell membrane, e.g., of a desired target cell. A henipavirus G protein may, for example, bind to a molecule (e.g., a polypeptide) on the surface of the target cell.

Illustrative examples of retroviral-derived env genes which can also be employed as fusogens include, but are not limited to: MLV envelopes, 10A1 envelope, BAEV, FeLV-B, RD 114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include, FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV. In the case of lentiviruses, such as HIV-1, HIV-2, SIV, FIV and EIV, the native env proteins include gp41 and gp120. In some embodiments, the viral env proteins expressed by source cells described herein are encoded on a separate vector from the viral gag and pol genes, as has been previously described.

In some embodiments, envelope proteins for display on a fusosome include, but are not limited to any of the following sources: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

A fusosome or pseudotyped virus generally has a modification to one or more of its envelope proteins, e.g., an envelope protein is substituted with an envelope protein from another virus. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In some embodiments, lentiviral envelope proteins are pseudotyped with VSV-G. In one embodiment, source cells produce recombinant retrovirus, e.g., lentivirus, pseudotyped with the VSV-G envelope glycoprotein. In some embodiments, a source cell described herein produces a fusosome, e.g., recombinant retrovirus, e.g., lentivirus, pseudotyped with the VSV-G glycoprotein.

Furthermore, a fusogen or viral envelope protein can be modified or engineered to contain polypeptide sequences that allow the transduction vector to target and infect host cells outside its normal range or more specifically limit transduction to a cell or tissue type. For example, the fusogen or envelope protein can be joined in-frame with targeting sequences, such as receptor ligands, antibodies (using an antigen-binding portion of an antibody or a recombinant antibody-type molecule, such as a single chain antibody), and polypeptide moieties or modifications thereof (e.g., where a glycosylation site is present in the targeting sequence) that, when displayed on the transduction vector coat, facilitate directed delivery of the virion particle to a target cell of interest. Furthermore, envelope proteins can further comprise sequences that modulate cell function. Modulating cell function with a transducing vector may increase or decrease transduction efficiency for certain cell types in a mixed population of cells. For example, stem cells could be transduced more specifically with envelope sequences containing ligands or binding partners that bind specifically to stem cells, rather than other cell types that are found in the blood or bone marrow. Non-limiting examples are stem cell factor (SCF) and Flt-3 ligand. Other examples, include, e.g., antibodies (e.g., single-chain antibodies that are specific for a cell-type), and essentially any antigen (including receptors) that binds tissues as lung, liver, pancreas, heart, endothelial, smooth, breast, prostate, epithelial, vascular cancer, etc.

### Exemplary Fusogens

In some embodiments, the fusosome includes one or more fusogens, e.g., to facilitate the fusion of the fusosome to a membrane, e.g., a cell membrane. In some embodiments, the one or more fusogens comprises a henipavirus F protein molecule (e.g., an active henipavirus F protein molecule) and/or a henipavirus G protein molecule.

In some embodiments, the retroviral vector or fusosome comprises one or more fusogens on its envelope to target a specific cell or tissue type. Fusogens include without limitation protein based, lipid based, and chemical based fusogens. In some embodiments, the retroviral vector or fusosome includes a first fusogen which is a protein fusogen and a second fusogen which is a lipid fusogen or chemical fusogen. The fusogen may bind a fusogen binding partner on a target cells' surface. In some embodiments, the fusosome comprising the fusogen will integrate the membrane into a lipid bilayer of a target cell.

In some embodiments, one or more of the fusogens described herein may be included in the fusosome.

### Protein Fusogens

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof. In some embodiments, the protein fusogens comprise a henipavirus F protein molecule (e.g., an active henipavirus F protein molecule) and/or a henipavirus G protein molecule.

In some embodiments, the fusogen results in mixing between lipids in the retroviral vector or fusosome and lipids in the target cell. In some embodiments, the fusogen results in formation of one or more pores between the interior of the retroviral vector or fusosome and the cytosol of the target cell.

### Mammalian Proteins

In some embodiments, the fusogen may include a mammalian protein, see, e.g., Table 1. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/naturel2343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells (see Table 2), any protein with fusogen properties (see Table 3), a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

TABLE 1
Non-limiting examples of human and non-human fusogens.
Human and Non-Human Fusogen Classes

| | Uniprot Protein | |
|---|---|---|
| Fusogen Class | Family ID | # of sequences |
| EFF-AFF | PF14884 | 191 |
| SNARE | PF05739 | 5977 |
| DC-STAMP | PF07782 | 633 |
| ENV | PF00429 | 312 |

TABLE 2
Genes that encode proteins with fusogen properties.
Human genes with the gene ontology annotation of:
Syncytium formation by plasma
membrane fusion proteins

| ID | Symbol |
|---|---|
| A0A024R0I0 | DYRK1B |
| A0A024R1N1 | MYH9 |
| A0A024R2D8 | CAV3 |
| A0A096LNV2 | FER1L5 |
| A0A096LPA8 | FER1L5 |
| A0A096LPB1 | FER1L5 |
| A0AVI2 | FER1L5 |
| A6NI61 | TMEM8C (myomaker) |
| B3KSL7 | — |
| B7ZLI3 | FER1L5 |
| H0YD14 | MYOF |
| O43184 | ADAM12 |
| O60242 | ADGRB3 |
| O60500 | NPHS1 |
| O95180 | CACNA1H |
| O95259 | KCNH1 |
| P04628 | WNT1 |
| P15172 | MYOD1 |
| P17655 | CAPN2 |
| P29475 | NOS1 |
| P35579 | MYH9 |
| P56539 | CAV3 |
| Q2NNQ7 | FER1L5 |
| Q4KMG0 | CDON |
| Q53GL0 | PLEKHO1 |
| Q5TCZ1 | SH3PXD2A |
| Q6YHK3 | CD109 |
| Q86V25 | VASH2 |
| Q99697 | PITX2 |
| Q9COD5 | TANC1 |
| Q9H295 | DCSTAMP |
| Q9NZM1 | MYOF |
| Q9Y463 | DYRK1B |

TABLE 3
Human Fusogen Candidates

| Fusogen Class | Gene ID |
|---|---|
| SNARE | O15400 |
| | Q16623 |
| | K7EQB1 |
| | Q86Y82 |
| | E9PN33 |
| | Q96NA8 |
| | H3BT82 |
| | Q9UNK0 |
| | P32856 |
| | Q13190 |
| | O14662 |
| | P61266 |
| | O43752 |
| | O60499 |
| | Q13277 |
| | B7ZBM8 |
| | AOAVG3 |
| | Q12846 |
| DC-STAMP | Q9H295 |
| | Q5T1A1 |
| | Q5T197 |
| | E9PJX3 |
| | Q9BR26 |
| ENV | Q9UQF0 |
| | Q9N2K0 |
| | P60507 |
| | P60608 |
| | B6SEH9 |
| | P60508 |
| | B6SEH8 |
| | P61550 |
| | P60509 |
| | Q9N2J8 |
| Muscle Fusion | HOY5B2 |
| (Myomaker) | |
| | H7C1S0 |
| | Q9HCN3 |
| | A6NDV4 |
| | K4DI83 |
| Muscle Fusion | NP_001302423.1 |
| (Myomixer) | |
| | ACT64390.1 |
| | XP_018884517.1 |
| | XP_017826615.1 |
| | XP_020012665.1 |
| | XP_017402927.1 |
| | XP_019498363.1 |
| | ELW65617.1 |
| | ERE90100.1 |
| | XP_017813001.1 |
| | XP_017733785.1 |
| | XP_017531750.1 |
| | XP_020142594.1 |
| | XP_019649987.1 |
| | XP_019805280.1 |
| | NP_001170939.1 |
| | NP_001170941.1 |
| | XP_019590171.1 |
| | XP_019062106.1 |
| | EPQ04443.1 |
| | EPY76709.1 |
| | XP_017652630.1 |
| | XP_017459263.1 |
| | OBS58441.1 |
| | XP_017459262.1 |
| | XP_017894180.1 |
| | XP_020746447.1 |
| | ELK00259.1 |
| | XP_019312826.1 |
| | XP_017200354.1 |
| | BAH40091.1 |
| HA | P03452 |
| | Q9Q0U6 |
| | P03460 |
| GAP JUNCTION | P36382 |
| | P17302 |
| | P36383 |
| | P08034 |
| | P35212 |
| Other | FGFRL1 |
| | GAPDH |

In some embodiments, the retroviral vector or fusosome comprises a curvature-generating protein, e.g., Epsin1, dynamin, or a protein comprising a BAR domain. See, e.g., Kozlov et. al., CurrOp StrucBio 2015, Zimmerberg et. al.. Nat Rev 2006, Richard et al, Biochem J 2011.

### Non-mammalian Proteins

### Viral Proteins

In some embodiments, the fusogen may include a non-mammalian protein, e.g., a viral protein. In some embodiments, a viral fusogen is a henipavirus F protein (e.g., an active henipavirus F protein). In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatatis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof, human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glyoproteins F₁ and F₂; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and p sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

In some embodiments the fusogen is a paramyxovirus fusogen. In some embodiments, the fusogen is a henipavirus fusogen, such as from any virus set forth in Table 3A. In some embodiments the fusogen is a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein.

In some embodiments, the fusogen is a poxviridae fusogen.

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

TABLE 3A
Exemplary Members of the Genus Henipavirus

| Species | Virus |
|---|---|
| Cedar henipavirus | Cedar virus (CedV) |
| Ghanaian bat henipavirus | Kumasi virus (KV) |
| Hendra henipavirus | Hendra virus (HeV) |
| Mojiang henipavirus | Mòjiāng virus (MojV) |
| Nipah henipavirus | Nipah virus (NiV) |

In some embodiments, the fusogen comprises a protein with a hydrophobic fusion peptide domain. In some embodiments, the fusogen comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein or a bat Paramyxovirus F protein or a biologically active portion thereof.

Table 4 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F0 precursors containing a signal peptide (e.g. corresponding to amino acid residues 1-26 of SEQ ID NO:7). Following cleavage of the signal peptide, the mature F0 (e.g., SEQ ID NO: 13) is transported to the cell surface, then endocytosed and cleaved by cathepsin L (e.g. between amino acids 109-110 of SEQ ID NO:7) into the mature fusogenic subunits F₁ (e.g. corresponding to amino acids 110-546 of SEQ ID NO:7; set forth in SEQ ID NO:15) and F₂ (e.g. corresponding to amino acid residues 27-109 of SEQ ID NO:7; set forth in SEQ ID NO:14). The F₁ and F₂ subunits are associated by a disulfide bond and recycled back to the cell surface. The F₁ subunit contains the fusion peptide domain located at the N terminus of the F₁ subunit (e.g. .g. corresponding to amino acids 110-129 of SEQ ID NO:7) where it is able to insert into a cell membrane to drive fusion. In particular cases, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided fusosomes, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

In some embodiments, the F protein is encoded by a nucleotide sequence that encodes the sequence set forth by any one of SEQ ID NOs: 3-7 or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 3-7. In particular embodiments, the F protein or the functionally active variant or biologically active portion thereof retains fusogenic activity in conjunction with a Henipavirus G protein, such as a G protein set forth in Table 5 (e.g. NiV-G or HeV-G). Fusogenic activity includes the activity of the F protein in conjunction with a Henipavirus G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:7).

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NOs: 3-7, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO: 3-7.

In some embodiments, a henipavirus F protein molecule described herein comprises an amino acid sequence of Table 4 (e.g., any of SEQ ID NOS: 3-7), or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 100, 200, 300, 400, 500, or 600 amino acids in length. For instance, in some embodiments, a henipavirus F protein molecule described herein comprises an amino acid sequence having at least 80% identity to any amino acid sequence of Table 4. In some embodiments, a henipavirus F protein molecule described herein comprises an amino acid sequence having at least 80% identity to SEQ ID NO: 3. In some embodiments, a henipavirus F protein molecule described herein comprises an amino acid sequence having at least 80% identity to SEQ ID NO: 4. In some embodiments, a henipavirus F protein molecule described herein comprises an amino acid sequence having at least 80% identity to SEQ ID NO: 5. In some embodiments, a henipavirus F protein molecule described herein comprises an amino acid sequence having at least 80% identity to SEQ ID NO: 6. In some embodiments, a henipavirus F protein molecule described herein comprises an amino acid sequence having at least 80% identity to SEQ ID NO: 7. In some embodiments, a nucleic acid sequence described herein encodes an amino acid sequence of Table 4, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 40, 50, 60, 80, 100, 200, 300, 400, 500, or 600 amino acids in length.

TABLE 4
Henipavirus F sequences. Column 1, Genbank ID includes the Genbank ID of the
whole genome sequence of the virus that is the centroid sequence of the cluster. Column 2,
Nucleotides of CDS provides the nucleotides corresponding to the CDS of the gene in the
whole genome. Column 3, Full Gene Name, provides the full name of the gene including
Genbank ID, virus species, strain, and protein name. Column 4, Sequence, provides the
amino acid sequence of the gene. Column 5, SEQ ID number

| Genbank | Nucleotides | Full Gene | | SEQ |
|---|---|---|---|---|
| ID | of CDS | Name | Sequence | ID |
| AF017149 | 6618-8258 | gb:AF017149\| | MATQEVRLKCLLCGIIVLVLSLEGLGILHYEKLSKIGLVKGITR | 3 |
| | | Organism: | KYKIKSNPLTKDIVIKMIPNVSNVSKCTGTVMENYKSRLTGI | |
| | | Hendra virus\| | LSPIKGAIELYNNNTHDLVGDVKLAGVVMAGIAIGIATAAQ | |
| | | Strain Name: | ITAGVALYEAMKNADNINKLKSSIESTNEAVVKLQETAEKT | |
| | | UNKNOWN- | VYVLTALQDYINTNLVPTIDQISCKQTELALDLALSKYLSDLL | |
| | | AF017149\| | FVFGPNLQDPVSNSMTIQAISQAFGGNYETLLRTLGYATED | |
| | | Protein Name: | FDDLLESDSIAGQIVYVDLSSYYIIVRVYFPILTEIQQAYVQEL | |
| | | fusion\| | LPVSFNNDNSEWISIVPNFVLIRNTLISNIEVKYCLITKKSVIC | |
| | | Gene Symbol: | NQDYATPMTASVRECLTGSTDKCPRELVVSSHVPRFALSG | |
| | | F | GVLFANCISVTCQCQTTGRAISQSGEQTLLMIDNTTCTTVV | |
| | | | LGNIIISLGKYLGSINYNSESIAVGPPVYTDKVDISSQISSMN | |
| | | | QSLQQSKDYIKEAQKILDTVNPSLISMLSMIILYVLSIAALCIG | |
| | | | LITFISFVIVEKKRGNYSRLDDRQVRPVSNGDLYYIGT | |
| JQ001776 | 6129-8166 | gb: | MSNKRTTVLIIISYTLFYLNNAAIVGFDFDKLNKIGVVQGRV | 4 |
| | | JQ001776: | LNYKIKGDPMTKDLVLKFIPNIVNITECVREPLSRYNETVRRL | |
| | | 6129-8166\| | LLPIHNMLGLYLNNTNAKMTGLMIAGVIMGGIAIGIATAA | |
| | | Organism: | QITAGFALYEAKKNTENIQKLTDSIMKTQDSIDKLTDSVGTS | |
| | | Cedar virus\| | ILILNKLQTYINNQLVPNLELLSCRQNKIEFDLMLTKYLVDL | |
| | | Strain Name: | MTVIGPNINNPVNKDMTIQSLSLLFDGNYDIMMSELGYTP | |
| | | CG1a\| | QDFLDLIESKSITGQIIYVDMENLYVVIRTYLPTLIEVPDAQIY | |
| | | Protein Name: | EFNKITMSSNGGEYLSTIPNFILIRGNYMSNIDVATCYMTKA | |
| | | fusion | SVICNQDYSLPMSQNLRSCYQGETEYCPVEAVIASHSPRFA | |
| | | glycoprotein\| | LTNGVIFANCINTICRCQDNGKTITQNINQFVSMIDNSTCN | |
| | | Gene Symbol: | DVMVDKFTIKVGKYMGRKDINNINIQIGPQIIIDKVDLSNEI | |
| | | F | NKMNQSLKDSIFYLREAKRILDSVNISLISPSVQLFLIIISVLSFI | |
| | | | ILLIIIVYLYCKSKHSYKYNKFIDDPDYYNDYKRERINGKASKS | |
| | | | NNIYYVGD | |
| NC_025352 | 5950-8712 | gb: | MALNKNMFSSLFLGYLLVYATTVQSSIHYDSLSKVGVIKGLT | 5 |
| | | NC_025352: | YNYKIKGSPSTKLMVVKLIPNIDSVKNCTQKQYDEYKNLVR | |
| | | 5950-8712\| | KALEPVKMAIDTMLNNVKSGNNKYRFAGAIMAGVALGVA | |
| | | Organism: | TAATVTAGIALHRSNENAQAIANMKSAIQNTNEAVKQLQL | |
| | | Mojiang virus\| | ANKQTLAVIDTIRGEINNNIIPVINQLSCDTIGLSVGIRLTQYY | |
| | | Strain Name: | SEIITAFGPALQNPVNTRITIQAISSVFNGNFDELLKIMGYTS | |
| | | Tongguan1\| | GDLYEILHSELIRGNIIDVDVDAGYIALEIEFPNLTLVPNAVV | |
| | | Protein Name: | QELMPISYNIDGDEWVTLVPRFVLTRTTLLSNIDTSRCTITD | |
| | | fusion protein\| | SSVICDNDYALPMSHELIGCLQGDTSKCAREKVVSSYVPKF | |
| | | Gene Symbol: | ALSDGLVYANCLNTICRCMDTDTPISQSLGATVSLLDNKRC | |
| | | F | SVYQVGDVLISVGSYLGDGEYNADNVELGPPIVIDKIDIGN | |
| | | | QLAGINQTLQEAEDYIEKSEEFLKGVNPSIITLGSMVVLYIF | |
| | | | MILIAIVSVIALVLSIKLTVKGNVVRQQFTYTQHVPSMENIN | |
| | | | YVSH | |
| NC_025256 | 6865-8853 | gb: | MKKKTDNPTISKRGHNHSRGIKSRALLRETDNYSNGLIVEN | 6 |
| | | NC_025256: | LVRNCHHPSKNNLNYTKTQKRDSTIPYRVEERKGHYPKIKH | |
| | | 6865-8853\| | LIDKSYKHIKRGKRRNGHNGNIITIILLLILILKTQMSEGAIHYE | |
| | | Organism: | TLSKIGLIKGITREYKVKGTPSSKDIVIKLIPNVTGLNKCTNIS | |
| | | Bat | MENYKEQLDKILIPINNIIELYANSTKSAPGNARFAGVIIAGV | |
| | | Paramyxovirus | ALGVAAAAQITAGIALHEARQNAERINLLKDSISATNNAVA | |
| | | Eid_hel/ | ELQEATGGIVNVITGMQDYINTNLVPQIDKLQCSQIKTALDI | |
| | | GH-M74a/GHA/ | SLSQYYSEILTVFGPNLQNPVTTSMSIQAISQSFGGNIDLLL | |
| | | 2009\| | NLLGYTANDLLDLLESKSITGQITYINLEHYFMVIRVYYPIMT | |
| | | Strain Name: | TISNAYVQELIKISFNVDGSEWVSLVPSYILIRNSYLSNIDISE | |
| | | BatPV/Eid_hel/ | CLITKNSVICRHDFAMPMSYTLKECLTGDTEKCPREAVVTS | |
| | | GH-M74a/GHA/ | YVPRFAISGGVIYANCLSTTCQCYQTGKVIAQDGSQTLMMI | |
| | | 2009\| | DNQTCSIVRIEEILISTGKYLGSQEYNTMHVSVGNPVFTDKL | |
| | | Protein Name: | DITSQISNINQSIEQSKFYLDKSKAILDKINLNLIGSVPISILFIIA | |
| | | fusion protein\| | ILSLILSIITFVIVMIIVRRYNKYTPLINSDPSSRRSTIQDVYIIPN | |
| | | Gene Symbol: | PGEHSIRSAARSIDRDRD | |
| | | F | | |
| UniProt | | FUS_NIPAV | MVVILDKRCYCNLLILILMISECSVGILHYEKLSKIGLVKGVTR | 7 |
| ID: | | Fusion | KYKIKSNPLTKDIVIKMIPNVSNMSQCTGSVMENYKTRLNG | |
| Q9IH63 | | glycoprotein F0 | ILTPIKGALEIYKNNTHDLVGDVRLAGVIMAGVAIGIATAAQ | |
| | | OS = Nipah virus | ITAGVALYEAMKNADNINKLKSSIESTNEAVVKLQETAEKT | |
| | | | VYVLTALQDYINTNLVPTIDKISCKQTELSLDLALSKYLSDLLF | |
| | | | VFGPNLQDPVSNSMTIQAISQAFGGNYETLLRTLGYATEDF | |
| | | | DDLLESDSITGQIIYVDLSSYYIIVRVYFPILTEIQQAYIQELLPV | |
| | | | SFNNDNSEWISIVPNFILVRNTLISNIEIGFCLITKRSVICNQD | |
| | | | YATPMTNNMRECLTGSTEKCPRELVVSSHVPRFALSNGVL | |
| | | | FANCISVTCQCQTTGRAISQSGEQTLLMIDNTTCPTAVLGN | |
| | | | VIISLGKYLGSVNYNSEGIAIGPPVFTDKVDISSQISSMNQSL | |
| | | | QQSKDYIKEAQRLLDTVNPSLISMLSMIILYVLSIASLCIGLIT | |
| | | | FISFIIVEKKRNTYSRLEDRRVRPTSSGDLYYIGT | |

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 3-7. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F0 precursor. In some embodiments, the F0 precursor is inactive. In some embodiments, the cleavage of the F0 precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F0 precursor is encoded by a sequence of nucleotides encoding the sequence set forth in SEQ ID NO: 7. The encoding nucleic acid can encode a signal peptide sequence that has the sequence MVVILDKRCY CNLLILILMI SECSVG (SEQ ID NO: 16). In some embodiments, the F protein has the sequence set forth in SEQ ID NO:13. In some examples, the F protein is cleaved into an F1 subunit comprising the sequence set forth in SEQ ID NO:15 and an F2 subunit comprising the sequence set forth in SEQ ID NO: 14.

In some embodiments, the F protein or the functionally active variant or the biologically active portion thereof includes an F1 subunit that has the sequence set forth in SEQ ID NO: 15, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15.

In some embodiments, the F protein or the functionally active variant or biologically active portion thereof includes an F2 subunit that has the sequence set forth in SEQ ID NO: 14, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14.

In some embodiments, the F protein is a mutant NiV-F protein that is a biologically active portion thereof that comprises a 22 amino acid truncation at or near the C-terminus of the wild-type NiV-F protein (SEQ ID NOs:7 or 13). In some embodiments, the NiV-F protein is encoded by a nucleotide sequence that encodes the sequence set forth in SEQ ID NO: 17. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17. In some embodiments, the NiV-F protein has the amino acid sequence set forth in SEQ ID NO: 17. In some embodiments, the NiV-F protein has the amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17.

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein or a biologically active portion thereof. Table 5 provides non-limiting examples of G proteins.

The attachment G proteins are type II transmembrane glycoproteins containing an N-terminal cytoplasmic tail (e.g. corresponding to amino acids 1-49 of SEQ ID NO:9), a transmembrane domain (e.g. corresponding to amino acids 50-70 of SEQ ID NO:9), and an extracellular domain containing an extracellular stalk (e.g. corresponding to amino acids 71-187 of SEQ ID NO:9), and a globular head (corresponding to amino acids 188-602 of SEQ ID NO:9). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors eprhin B2 and ephrin B3, but is dispensable for membrane fusion (Brandel-Tretheway et al. Journal of Virology. 2019. 93(13)e00577-19). In particular embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described further below, a re-targeted lipid particle can contain heterologous G and F proteins from different species.

In some embodiments, a henipavirus G protein molecule described herein comprises an amino acid sequence of Table 5 (e.g., any of SEQ ID NOS: 8-12), or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 100, 200, 300, 400, 500, or 600 amino acids in length. For instance, in some embodiments, a henipavirus G protein molecule described herein comprises an amino acid sequence having at least 80% identity to any amino acid sequence of Table 5. In some embodiments, a henipavirus G protein molecule described herein comprises an amino acid sequence having at least 80% identity to SEQ ID NO: 8. In some embodiments, a henipavirus G protein molecule described herein comprises an amino acid sequence having at least 80% identity to SEQ ID NO: 9. In some embodiments, a henipavirus G protein molecule described herein comprises an amino acid sequence having at least 80% identity to SEQ ID NO: 10. In some embodiments, a henipavirus G protein molecule described herein comprises an amino acid sequence having at least 80% identity to SEQ ID NO: 11. In some embodiments, a henipavirus G protein molecule described herein comprises an amino acid sequence having at least 80% identity to SEQ ID NO: 12. In some embodiments, a nucleic acid sequence described herein encodes an amino acid sequence of Table 5, or an amino acid sequence having at least 80%, 85%, 90%, 95%, 96%, 97%, 98%, or 99% sequence identity thereto, or an amino acid sequence having at least 80%, 85%, 90%, 95%, 96%, 97%, 98%, or 99% sequence identity to a portion of the sequence, e.g., a portion of 40, 50, 60, 80, 100, 200, 300, 400, 500, or 600 amino acids in length.

In particular embodiments, the G protein or functionally active variant or biologically active portion is a protein that retains fusogenic activity in conjunction with a Henipavirus F protein, such as an F protein set forth in Table 4 (e.g. NiV-F or HeV-F). Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NOs: 8-12; such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 800 of the level or degree of fusogenic activity of the corresponding wild-type G protein, such as at least or at least about 8500 of the level or degree of fusogenic activity of the corresponding wild-type G protein, such as at least or at least about 9000 of the level or degree of fusogenic activity of the corresponding wild-type G protein, such as at least or at least about 9500 of the level or degree of fusogenic activity of the corresponding wild-type G protein, such as at least or at least about 1000% of the level or degree of fusogenic activity of the corresponding wild-type G protein, or such as at least or at least about 1200% of the level or degree of fusogenic activity of the corresponding wild-type G protein.

TABLE 5
Henipavirus protein G sequences. Column 1, Genbank ID includes the Genbank ID
of the whole genome sequence of the virus that is the centroid sequence of the cluster.
Column 2, nucleotides of CDS provides the nucleotides corresponding to the CDS of the
gene in the whole genome. Column 3, Full Gene Name, provides the full name of the gene
including Genbank ID, virus species, strain, and protein name. Column 4, Sequence,
provides the amino acid sequence of the gene. Column 5, SEQ ID number.

| Genbank | Nucleotides | | | SEQ |
|---|---|---|---|---|
| ID | of CDS | Full sequence ID | Sequence | ID |
| AF017149 | 8913-10727 | gb: AF017149\| | MMADSKLVSLNNNLSGKIKDQGKVIKNYYGTMDIKKINDG | 8 |
| | | Organism: | LLDSKILGAFNTVIALLGSIIIIVMNIMIIQNYTRTTDNQALIK | |
| | | Hendra virus\| | ESLQSVQQQIKALTDKIGTEIGPKVSLIDTSSTITIPANIGLLG | |
| | | Strain Name: | SKISQSTSSINENVNDKCKFTLPPLKIHECNISCPNPLPFREY | |
| | | UNKNOWN- | RPISQGVSDLVGLPNQICLQKTTSTILKPRLISYTLPINTREGV | |
| | | AF017149\| | CITDPLLAVDNGFFAYSHLEKIGSCTRGIAKQRIIGVGEVLDR | |
| | | Protein Name: | GDKVPSMFMTNVWTPPNPSTIHHCSSTYHEDFYYTLCAVS | |
| | | glycoprotein\| | HVGDPILNSTSWTESLSLIRLAVRPKSDSGDYNQKYIAITKV | |
| | | Gene Symbol: | ERGKYDKVMPYGPSGIKQGDTLYFPAVGFLPRTEFQYNDS | |
| | | G | NCPIIHCKYSKAENCRLSMGVNSKSHYILRSGLLKYNLSLGG | |
| | | | DIILQFIEIADNRLTIGSPSKIYNSLGQPVFYQASYSWDTMIK | |
| | | | LGDVDTVDPLRVQWRNNSVISRPGQSQCPRFNVCPEVC | |
| | | | WEGTYNDAFLIDRLNWVSAGVYLNSNQTAENPVFAVFKD | |
| | | | NEILYQVPLAEDDTNAQKTITDCFLLENVIWCISLVEIYDTG | |
| | | | DSVIRPKLFAVKIPAQCSES | |
| AF212302 | 8943-10751 | gb: AF212302\| | MPAENKKVRFENTTSDKGKIPSKVIKSYYGTMDIKKINEGLL | 9 |
| | | Organism: | DSKILSAFNTVIALLGSIVIIVMNIMIIQNYTRSTDNQAVIKD | |
| | | Nipah virus\| | ALQGIQQQIKGLADKIGTEIGPKVSLIDTSSTITIPANIGLLGS | |
| | | Strain Name: | KISQSTASINENVNEKCKFTLPPLKIHECNISCPNPLPFREYR | |
| | | UNKNOWN- | PQTEGVSNLVGLPNNICLQKTSNQILKPKLISYTLPVVGQSG | |
| | | AF212302\| | TCITDPLLAMDEGYFAYSHLERIGSCSRGVSKQRIIGVGEVL | |
| | | Protein Name: | DRGDEVPSLFMTNVWTPPNPNTVYHCSAVYNNEFYYVLC | |
| | | attachment | AVSTVGDPILNSTYWSGSLMMTRLAVKPKSNGGGYNQH | |
| | | glycoprotein\| | QLALRSIEKGRYDKVMPYGPSGIKQGDTLYFPAVGFLVRTE | |
| | | Gene Symbol: | FKYNDSNCPITKCQYSKPENCRLSMGIRPNSHYILRSGLLKY | |
| | | G | NLSDGENPKVVFIEISDQRLSIGSPSKIYDSLGQPVFYQASFS | |
| | | | WDTMIKFGDVLTVNPLVVNWRNNTVISRPGQSQCPRENT | |
| | | | CPEICWEGVYNDAFLIDRINWISAGVFLDSNQTAENPVFTV | |
| | | | FKDNEILYRAQLASEDTNAQKTITNCFLLKNKIWCISLVEIYD | |
| | | | TGDNVIRPKLFAVKIPEQCT | |
| JQ001776 | 8170-10275 | gb: JQ001776: | MLSQLQKNYLDNSNQQGDKMNNPDKKLSVNFNPLELDK | 10 |
| | | 8170-10275\| | GQKDLNKSYYVKNKNYNVSNLLNESLHDIKFCIYCIFSLLIIITI | |
| | | Organism: | INIITISIVITRLKVHEENNGMESPNLQSIQDSLSSLTNMINTE | |
| | | Cedar virus\| | ITPRIGILVTATSVTLSSSINYVGTKTNQLVNELKDYITKSCGF | |
| | | Strain Name: | KVPELKLHECNISCADPKISKSAMYSTNAYAELAGPPKIFCK | |
| | | CG1a\| | SVSKDPDFRLKQIDYVIPVQQDRSICMNNPLLDISDGFFTYI | |
| | | Protein Name: | HYEGINSCKKSDSFKVLLSHGEIVDRGDYRPSLYLLSSHYHPY | |
| | | attachment | SMQVINCVPVTCNQSSFVFCHISNNTKTLDNSDYSSDEYYI | |
| | | glycoprotein\| | TYFNGIDRPKTKKIPINNMTADNRYIHFTFSGGGGVCLGEE | |
| | | Gene Symbol: | FIIPVTTVINTDVFTHDYCESFNCSVQTGKSLKEICSESLRSPT | |
| | | G | NSSRYNLNGIMIISQNNMTDFKIQLNGITYNKLSFGSPGRL | |
| | | | SKTLGQVLYYQSSMSWDTYLKAGFVEKWKPFTPNWMNN | |
| | | | TVISRPNQGNCPRYHKCPEICYGGTYNDIAPLDLGKDMYVS | |
| | | | VILDSDQLAENPEITVFNSTTILYKERVSKDELNTRSTTTSCFL | |
| | | | FLDEPWCISVLETNRFNGKSIRPEIYSYKIPKYC | |
| NC_025256 | 9117-11015 | gb: NC_025256: | MPQKTVEFINMNSPLERGVSTLSDKKTLNQSKITKQGYFGL | 11 |
| | | 9117-11015\| | GSHSERNWKKQKNQNDHYMTVSTMILEILVVLGIMENLIV | |
| | | Organism: | LTMVYYQNDNINQRMAELTSNITVLNLNLNQLTNKIQREII | |
| | | Bat | PRITLIDTATTITIPSAITYILATLTTRISELLPSINQKCEFKTPTL | |
| | | Paramyxovirus | VLNDCRINCTPPLNPSDGVKMSSLATNLVAHGPSPCRNFS | |
| | | Eid_hel/GH-M74a/ | SVPTIYYYRIPGLYNRTALDERCILNPRLTISSTKFAYVHSEYD | |
| | | GHA/2009\| | KNCTRGFKYYELMTFGEILEGPEKEPRMFSRSFYSPTNAVN | |
| | | Strain Name: | YHSCTPIVTVNEGYFLCLECTSSDPLYKANLSNSTFHLVILRH | |
| | | BatPV/Eid_hel/ | NKDEKIVSMPSFNLSTDQEYVQIIPAEGGGTAESGNLYFPCI | |
| | | GH-M74a/GHA/2009\| | GRLLHKRVTHPLCKKSNCSRTDDESCLKSYYNQGSPQHQV | |
| | | Protein Name: | VNCLIRIRNAQRDNPTWDVITVDLTNTYPGSRSRIFGSFSKP | |
| | | glycoprotein\| | MLYQSSVSWHTLLQVAEITDLDKYQLDWLDTPYISRPGGS | |
| | | Gene Symbol: | ECPFGNYCPTVCWEGTYNDVYSLTPNNDLFVTVYLKSEQV | |
| | | G | AENPYFAIFSRDQILKEFPLDAWISSARTTTISCFMFNNEIW | |
| | | | CIAALEITRLNDDIIRPIYYSFWLPTDCRTPYPHTGKMTRVPL | |
| | | | RSTYNY | |
| NC_025352 | 8716-11257 | gb: NC_025352: | MATNRDNTITSAEVSQEDKVKKYYGVETAEKVADSISGNK | 12 |
| | | 8716-11257\| | VFILMNTLLILTGAIITITLNITNLTAAKSQQNMLKIIQDDVN | |
| | | Organism : | AKLEMFVNLDQLVKGEIKPKVSLINTAVSVSIPGQISNLQTK | |
| | | Mojiang virus\| | FLQKYVYLEESITKQCTCNPLSGIFPTSGPTYPPTDKPDDDTT | |
| | | Strain Name: | DDDKVDTTIKPIEYPKPDGCNRTGDHFTMEPGANFYTVPN | |
| | | Tongguan1\| | LGPASSNSDECYTNPSFSIGSSIYMFSQEIRKTDCTAGEILSI | |
| | | Protein Name: | QIVLGRIVDKGQQGPQASPLLVWAVPNPKIINSCAVAAGD | |
| | | attachment | EMGWVLCSVTLTAASGEPIPHMFDGFWLYKLEPDTEVVSY | |
| | | glycoprotein\| | RITGYAYLLDKQYDSVFIGKGGGIQKGNDLYFQMYGLSRN | |
| | | Gene Symbol: | RQSFKALCEHGSCLGTGGGGYQVLCDRAVMSFGSEESLIT | |
| | | G | NAYLKVNDLASGKPVIIGQTFPPSDSYKGSNGRMYTIGDKY | |
| | | | GLYLAPSSWNRYLRFGITPDISVRSTTWLKSQDPIMKILSTC | |
| | | | TNTDRDMCPEICNTRGYQDIFPLSEDSEYYTYIGITPNNGG | |
| | | | TKNFVAVRDSDGHIASIDILQNYYSITSATISCFMYKDEIWCI | |
| | | | AITEGKKQKDNPQRIYAHSYKIRQMCYNMKSATVTVGNA | |
| | | | KNITIRRY | |

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 9-12.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOS: 9-12. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is NiV-G or a functionally active variant or biologically active portion thereof and binds to Ephrin B2 or Ephrin B3. In some aspects, the NiV-G has the sequence of amino acids set forth in any of SEQ ID NOs:9-12, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs:9-12 and retains binding to Eprhin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type NiV-G, such as set forth in SEQ ID NOs:9-12.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:9.

In some embodiments, the G protein is a mutant G protein containing one or more amino acid substitutions selected from the group consisting of E501A, W504A, Q530A and E533A with reference to numbering set forth in SEQ ID NO:9. In some embodiments, the G protein is a mutant G protein that contains one or more amino acid substitutions elected from the group consisting of E501A, W504A, Q530A and E533A with reference to SEQ ID NO: 9 and is a biologically active portion thereof containing an N-terminal truncation.

In some embodiments, the G protein is a mutant G protein containing one or more amino acid substitutions selected from the group consisting of E501A, W504A, Q530A and E533A with reference to numbering set forth in SEQ ID NO: 9. In some embodiments, the G protein is a mutant G protein that contains one or more amino acid substitutions elected from the group consisting of E501A, W504A, Q530A and E533A with reference to SEQ ID NO: 9 and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO: 9), 6 contiguous amino acid residues at or near the N-terminus of the wild-type NiV-G protein (SEQ ID NO: 9), 7 contiguous amino acid residues at or near the N-terminus of the wild-type NiV-G protein (SEQ ID NO: 9), 8 contiguous amino acid residues at or near the N-terminus of the wild-type NiV-G protein (SEQ ID NO: 9), 9 contiguous amino acid residues at or near the N-terminus of the wild-type NiV-G protein (SEQ ID NO: 9), up to 10 contiguous amino acid residues at or near the N-terminus of the wild-type NiV-G protein (SEQ ID NO: 9), 11 contiguous amino acid residues at or near the N-terminus of the wild-type NiV-G protein (SEQ ID NO: 9), 12 contiguous amino acid residues at or near the N-terminus of the wild-type NiV-G protein (SEQ ID NO: 9), 13 contiguous amino acid residues at or near the N-terminus of the wild-type NiV-G protein (SEQ ID NO: 9), 14 contiguous amino acid residues at or near the N-terminus of the wild-type NiV-G protein (SEQ ID NO: 9), up to 15 contiguous amino acid residues at or near the N-terminus of the wild-type NiV-G protein (SEQ ID NO: 9), 16 contiguous amino acid residues at or near the N-terminus of the wild-type NiV-G protein (SEQ ID NO: 9), 17 contiguous amino acid residues at or near the N-terminus of the wild-type NiV-G protein (SEQ ID NO: 9), 18 contiguous amino acid residues at or near the N-terminus of the wild-type NiV-G protein (SEQ ID NO: 9), 19 contiguous amino acid residues at or near the N-terminus of the wild-type NiV-G protein (SEQ ID NO: 9), up to 20 contiguous amino acid residues at or near the N-terminus of the wild-type NiV-G protein (SEQ ID NO: 9), 21 contiguous amino acid residues at or near the N-terminus of the wild-type NiV-G protein (SEQ ID NO: 9), 22 contiguous amino acid residues at or near the N-terminus of the wild-type NiV-G protein (SEQ ID NO: 9), 23 contiguous amino acid residues at or near the N-terminus of the wild-type NiV-G protein (SEQ ID NO: 9), 24 contiguous amino acid residues at or near the N-terminus of the wild-type NiV-G protein (SEQ ID NO: 9), up to 25 contiguous amino acid residues at or near the N-terminus of the wild-type NiV-G protein (SEQ ID NO: 9), 26 contiguous amino acid residues at or near the N-terminus of the wild-type NiV-G protein (SEQ ID NO: 9), 27 contiguous amino acid residues at or near the N-terminus of the wild-type NiV-G protein (SEQ ID NO: 9), 28 contiguous amino acid residues at or near the N-terminus of the wild-type NiV-G protein (SEQ ID NO: 9), 29 contiguous amino acid residues at or near the N-terminus of the wild-type NiV-G protein (SEQ ID NO: 9), up to 30 contiguous amino acid residues at or near the N-terminus of the wild-type NiV-G protein (EQ ID NO: 9), up to 31 contiguous amino acid residues at or near the N-terminus of the wild-type NiV-G protein (SEQ ID NO: 9), 32 contiguous amino acid residues at or near the N-terminus of the wild-type NiV-G protein (SEQ ID NO: 9), 33 contiguous amino acid residues at or near the N-terminus of the wild-type NiV-G protein (SEQ ID NO: 9), 34 contiguous amino acid residues at or near the N-terminus of the wild-type NiV-G protein (SEQ ID NO: 9), 35 contiguous amino acid residues at or near the N-terminus of the wild-type NiV-G protein (SEQ ID NO: 9), up to 36 contiguous amino acid residues at or near the N-terminus of the wild-type NiV-G protein (EQ ID NO: 9), up to 37 contiguous amino acid residues at or near the N-terminus of the wild-type NiV-G protein (EQ ID NO: 9), up to 38 contiguous amino acid residues at or near the N-terminus of the wild-type NiV-G protein (EQ ID NO: 9), up to 39 contiguous amino acid residues at or near the N-terminus of the wild-type NiV-G protein (EQ ID NO: 9), or up to 40 contiguous amino acid residues at or near the N-terminus of the wild-type NiV-G protein (EQ ID NO: 9).

In some embodiments, the NiV-G protein is encoded by a nucleotide sequence that encodes the sequence set forth in SEQ ID NO: 18. In some embodiments, the NiV-G protein is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 18. In some embodiments, the mutant NiV-G protein has the amino acid sequence set forth in SEQ ID NO: 18 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18. In particular embodiments, the G protein has the sequence of amino acids set forth in SEQ ID NO: 18.

In some embodiments, the NiV-F protein has an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17, and the NiV-G protein has an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18. In some embodiments, the NiV-F protein has the amino acid sequence set forth in SEQ ID NO:17, and the NiV-G protein has the amino acid sequence set forth in SEQ ID NO: 18.

### Other Proteins

In some embodiments, the fusogen may include a pH dependent protein, a homologue thereof, a fragment thereof, and a protein fusion comprising one or more proteins or fragments thereof. Fusogens may mediate membrane fusion at the cell surface or in an endosome or in another cell-membrane bound space.

In some embodiments, the fusogen includes a EFF-1, AFF-1, gap junction protein, e.g., a connexin (such as Cn43, GAP43, CX43) (DOI: 10.1021/jacs.6b05191), other tumor connection proteins, a homologue thereof, a fragment thereof, a variant thereof, and a protein fusion comprising one or more proteins or fragments thereof.

### Modifications to Protein Fusogens

Protein fusogens or viral envelope proteins (e.g., a henipavirus G protein molecule) may be re-targeted by mutating amino acid residues in a fusion protein or a targeting protein (e.g. the hemagglutinin protein). In some embodiments the fusogen is randomly mutated. In some embodiments the fusogen is rationally mutated. In some embodiments the fusogen is subjected to directed evolution. In some embodiments the fusogen is truncated and only a subset of the peptide is used in the retroviral vector or fusosome. For example, amino acid residues in the measles hemagglutinin protein may be mutated to alter the binding properties of the protein, redirecting fusion (doi:10.1038/nbt942, Molecular Therapy vol. 16 no. 8, 1427-1436 Aug. 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

Protein fusogens (e.g., a henipavirus G protein molecule) may be re-targeted by covalently conjugating a targeting-moiety to the fusion protein or targeting protein (e.g. the hemagglutinin protein). For instance, a G protein may be linked to a targeting moiety (e.g. an antibody or aan antigen-binding fragment). In some embodiments, the fusogen and targeting moiety are covalently conjugated by expression of a chimeric protein comprising the fusogen linked to the targeting moiety. A target includes any peptide (e.g. a receptor) that is displayed on a target cell. In some examples the target is expressed at higher levels on a target cell than non-target cells. For example, single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). For example, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). For example, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). A targeting protein can also include, e.g., an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Protein fusogens may be re-targeted by non-covalently conjugating a targeting moiety to the fusion protein or targeting protein (e.g. the hemagglutinin protein). For example, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). Altered and non-altered fusogens may be displayed on the same retroviral vector or fusosome (doi: 10.1016/j.biomaterials.2014.01.051).

A targeting moiety may comprise a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE® s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR® s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the single domain antibody is attached to the C-terminus of the G protein or biologically active portion thereof. In some embodiments, the N-terminus of the single domain antibody is exposed on the exterior surface of the lipid bilayer. In some embodiments, the N-terminus of the single domain antibody binds to a cell surface molecule of a target cell. In some embodiments, the single domain antibody specifically binds to a cell surface molecule present on a target cell. In some embodiments, the cell surface molecule is a protein, glycan, lipid or low molecular weight molecule.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein.

Retroviral vectors or fusosomes may display targeting moieties that are not conjugated to protein fusogens in order to redirect the fusion activity towards a cell that is bound by the targeting moiety, or to affect homing.

The targeting moiety added to the retroviral vector or fusosome may be modulated to have different binding strengths. For example, scFvs and antibodies with various binding strengths may be used to alter the fusion activity of the retroviral vector or fusosome towards cells that display high or low amounts of the target antigen (doi:10.1128/JVI.01415-07, doi:10.1038/cgt.2014.25, DOI: 10.1002/jgm.1151). For example DARPins with different affinities may be used to alter the fusion activity of the retroviral vector or fusosome towards cells that display high or low amounts of the target antigen (doi:10.1038/mt.2010.298). Targeting moieties may also be modulated to target different regions on the target ligand, which will affect the fusion rate with cells displaying the target (doi: 10.1093/protein/gzv005).

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes,

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+ T cell, a CD4+ Tcell, a CD8+ T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the cell surface molecule is any one of CD8, CD4, asialoglycoprotein receptor 2 (ASGR2), transmembrane 4 L6 family member 5 (TM4SF5), low density lipoprotein receptor (LDLR) or asialoglycoprotein 1 (ASGR1).

In some embodiments, the G protein or functionally active variant or biologically active portion thereof is linked directly to the sdAb variable domain. In some embodiments, the targeted envelope protein is a fusion protein that has the following structure: (N′-single domain antibody-C′)—(C′-G protein-N′).

In some embodiments, the G protein or functionally active variant or biologically active portion thereof is linked indirectly via a linker to the the sdAb variable domain. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain. In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:19), GGGGGS (SEQ ID NO:20) or combinations thereof. In some embodiments, the polypeptide linker has the sequence (GGS)n, wherein n is 1 to 10. In some embodiments, the polypeptide linker has the sequence (GGGGS)n, (SEQ ID NO:21) wherein n is 1 to 10. In some embodiments, the polypeptide linker has the sequence (GGGGGS)n (SEQ ID NO:22), wherein n is 1 to 6.

In some embodiments, protein fusogens can be altered to reduce immunoreactivity, e.g., as described herein. For instance, protein fusogens may be decorated with molecules that reduce immune interactions, such as PEG (DOI: 10.1128/JVI.78.2.912-921.2004). Thus, in some embodiments, the fusogen comprises PEG, e.g., is a PEGylated polypeptide. Amino acid residues in the fusogen that are targeted by the immune system may be altered to be unrecognized by the immune system (doi: 10.1016/j.virol.2014.01.027, doi:10.1371/journal.pone.0046667). In some embodiments, the protein sequence of the fusogen is altered to resemble amino acid sequences found in humans (humanized). In some embodiments, the protein sequence of the fusogen is changed to a protein sequence that binds NMC complexes less strongly. In some embodiments, the protein fusogens are derived from viruses or organisms that do not infect humans (and which humans have not been vaccinated against), increasing the likelihood that a patient's immune system is naïve to the protein fusogens (e.g., there is a negligible humoral or cell-mediated adaptive immune response towards the fusogen) (doi:10.1006/mthe.2002.0550, doi:10.1371/journal.ppat.1005641, doi:10.1038/gt.2011.209, DOI 10.1182/blood-2014-02-558163). In some embodiments, glycosylation of the fusogen may be changed to alter immune interactions or reduce immunoreactivity. Without wishing to be bound by theory, in some embodiments, a protein fusogen derived from a virus or organism that do not infect humans does not have a natural fusion targets in patients, and thus has high specificity.

### Lipid Fusogens

In some embodiments, the retroviral vector or fusosome can comprise, e.g., in addition to an F and G protein described herein, one or more fusogenic lipids, such as saturated fatty acids. In some embodiments, the saturated fatty acids have between 10-14 carbons. In some embodiments, the saturated fatty acids have longer-chain carboxylic acids. In some embodiments, the saturated fatty acids are mono-esters.

In some embodiments, the retroviral vector or fusosome can comprise one or more unsaturated fatty acids. In some embodiments, the unsaturated fatty acids have between C16 and C18 unsaturated fatty acids. In some embodiments, the unsaturated fatty acids include oleic acid, glycerol mono-oleate, glycerides, diacylglycerol, modified unsaturated fatty acids, and any combination thereof.

Without wishing to be bound by theory, in some embodiments negative curvature lipids promote membrane fusion. In some embodiments, the retroviral vector or fusosome comprises one or more negative curvature lipids, e.g., exogenous negative curvature lipids, in the membrane. In embodiments, the negative curvature lipid or a precursor thereof is added to media comprising source cells, retroviral vector, or fusosome. In embodiments, the source cell is engineered to express or overexpress one or more lipid synthesis genes. The negative curvature lipid can be, e.g., diacylglycerol (DAG), cholesterol, phosphatidic acid (PA), phosphatidylethanolamine (PE), or fatty acid (FA).

Without wishing to be bound by theory, in some embodiments positive curvature lipids inhibit membrane fusion. In some embodiments, the retroviral vector or fusosome comprises reduced levels of one or more positive curvature lipids, e.g., exogenous positive curvature lipids, in the membrane. In embodiments, the levels are reduced by inhibiting synthesis of the lipid, e.g., by knockout or knockdown of a lipid synthesis gene, in the source cell. The positive curvature lipid can be, e.g., lysophosphatidylcholine (LPC), phosphatidylinositol (PtdIns), lysophosphatidic acid (LPA), lysophosphatidylethanolamine (LPE), or monoacylglycerol (MAG).

### Chemical Fusogens

In some embodiments, the retroviral vector or fusosome may be treated with fusogenic chemicals. In some embodiments, the fusogenic chemical is polyethylene glycol (PEG) or derivatives thereof.

In some embodiments, the chemical fusogen induces a local dehydration between the two membranes that leads to unfavorable molecular packing of the bilayer. In some embodiments, the chemical fusogen induces dehydration of an area near the lipid bilayer, causing displacement of aqueous molecules between two membranes and allowing interaction between the two membranes together.

In some embodiments, the chemical fusogen is a positive cation. Some nonlimiting examples of positive cations include Ca2+, Mg2+, Mn2+, Zn2+, La3+, Sr3+, and H+.

In some embodiments, the chemical fusogen binds to the target membrane by modifying surface polarity, which alters the hydration-dependent intermembrane repulsion.

In some embodiments, the chemical fusogen is a soluble lipid soluble. Some nonlimiting examples include oleoylglycerol, dioleoylglycerol, trioleoylglycerol, and variants and derivatives thereof.

In some embodiments, the chemical fusogen is a water-soluble chemical. Some nonlimiting examples include polyethylene glycol, dimethyl sulphoxide, and variants and derivatives thereof.

In some embodiments, the chemical fusogen is a small organic molecule. A nonlimiting example includes n-hexyl bromide.

In some embodiments, the chemical fusogen does not alter the constitution, cell viability, or the ion transport properties of the fusogen or target membrane.

In some embodiments, the chemical fusogen is a hormone or a vitamin. Some nonlimiting examples include abscisic acid, retinol (vitamin A1), a tocopherol (vitamin E), and variants and derivatives thereof.

In some embodiments, the retroviral vector or fusosome comprises actin and an agent that stabilizes polymerized actin. Without wishing to be bound by theory, stabilized actin in a retroviral vector or fusosome can promote fusion with a target cell. In embodiments, the agent that stabilizes polymerized actin is chosen from actin, myosin, biotin-streptavidin, ATP, neuronal Wiskott-Aldrich syndrome protein (N-WASP), or formin. See, e.g., Langmuir. 2011 Aug. 16; 27(16):10061-71 and Wen et al., Nat Commun. 2016 Aug. 31; 7. In embodiments, the retroviral vector or fusosome comprises exogenous actin, e.g., wild-type actin or actin comprising a mutation that promotes polymerization. In embodiments, the retroviral vector or fusosome comprises ATP or phosphocreatine, e.g., exogenous ATP or phosphocreatine.

### Small Molecule Fusogens

In some embodiments, the retroviral vector or fusosome may be treated with fusogenic small molecules. Some nonlimiting examples include halothane, nonsteroidal anti-inflammatory drugs (NSAIDs) such as meloxicam, piroxicam, tenoxicam, and chlorpromazine.

In some embodiments, the small molecule fusogen may be present in micelle-like aggregates or free of aggregates.

### Positive Target Cell-Specific Regulatory Element

In some embodiments, a fusosome nucleic acid described herein comprises a positive target cell-specific regulatory element such as a tissue-specific promoter, a tissue-specific enhancer, a tissue-specific splice site, a tissue-specific site extending half-life of an RNA or protein, a tissue-specific mRNA nuclear export promoting site, a tissue-specific translational enhancing site, or a tissue-specific post-translational modification site. Additional positive target cell-specific regulatory elements are described, for instance, in International Application WO2019/222403, which is hereby incorporated by reference in its entirety.

In particular embodiments, a fusosome nucleic acid described herein comprises control elements, e.g., capable of directing, increasing, regulating, or controlling the transcription or expression of an operatively linked polynucleotide in a cell-specific manner.

In particular embodiments, fusosome nucleic acids comprise one or more expression control sequences that are specific to particular cells, cell types, or cell lineages e.g., target cells; that is, expression of polynucleotides operatively linked to an expression control sequence specific to particular cells, cell types, or cell lineages is expressed in target cells and not (or at a lower level) in non-target cells. In particular embodiments, a fusosome nucleic acid can include exogenous, endogenous, or heterologous control sequences such as promoters and/or enhancers.

In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. In embodiments, an enhancer comprises a segment of DNA which contains sequences capable of providing enhanced transcription and in some instances can function independent of orientation relative to another control sequence. An enhancer can function cooperatively or additively with promoters and/or other enhancer elements. In some embodiments, a promoter/enhancer segment of DNA contains sequences capable of providing both promoter and enhancer functions. In some embodiments, a control sequence is a ubiquitous expression control sequence.

In some embodiments, the promoter is a tissue-specific promoter, e.g., a promoter that drives expression in liver cells, e.g., hepatocytes, liver sinusoidal endothelial cells, cholangiocytes, stellate cells, liver-resident antigen-presenting cells (e.g., Kupffer Cells), liver-resident immune lymphocytes (e.g., T cell, B cell, or NK cell), or portal fibroblasts.

### Non-Target Cell-Specific Regulatory Element

In some embodiments, the non-target cell specific regulatory element comprises a tissue-specific miRNA recognition sequence, tissue-specific protease recognition site, tissue-specific ubiquitin ligase site, tissue-specific transcriptional repression site, or tissue-specific epigenetic repression site. Additional non-target cell specific regulatory elements are described, for instance, in International Application WO2019/222403, which is hereby incorporated by reference in its entirety. In some embodiments, a non-target cell comprises an endogenous miRNA. The fusosome nucleic acid (e.g., the gene encoding the exogenous agent) may comprise a recognition sequence for that miRNA. Thus, if the fusosome nucleic acid enters the non-target cell, the miRNA can downregulate expression of the exogenous agent. This helps achieve additional specificity for the target cell versus non-target cells.

In some embodiments, the miRNA is a small non-coding RNAs of 20-22 nucleotides, typically excised from \tilde{ } 70 nucleotide foldback RNA precursor structures known as pre-miRNAs. miRNAs (e.g., naturally occurring miRNAs or artificially designed miRNAs) can specifically target any mRNA sequence. In one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR.

Hundreds of distinct miRNA genes are differentially expressed during development and across tissue types. Molecular analysis has shown that miRNAs have distinct expression profiles in different tissues. Computational methods have been used to analyze the expression of approximately 7,000 predicted human miRNA targets. The data suggest that miRNA expression broadly contributes to tissue specificity of mRNA expression in many human tissues. (See Sood et al. 2006 PNAS USA 103(8):2746-51.)

Thus, an miRNA-based approach may be used for restricting expression of the exogenous agent to a target cell population by silencing exogenous agent expression in non-target cell types by using endogenous microRNA species. In some embodiments, the fusosome nucleic acid comprises one or more of (e.g., a plurality of) tissue-specific miRNA recognition sequences. In some embodiments, the tissue-specific miRNA recognition sequence is about 20-25, 21-24, or 23 nucleotides in length. In embodiments, the tissue-specific miRNA recognition sequence has perfect complementarity to an miRNA present in a non-target cell. In some embodiments, the exogenous agent does not comprise GFP, e.g., does not comprise a fluorescent protein, e.g., does not comprise a reporter protein. In some embodiments, the off-target cells are not hematopoietic cell and/or the miRNA is not present in hematopoietic cells.

In some embodiments, a method herein comprises tissue-specific expression of an exogenous agent in a target cell comprising contacting a plurality of fusosome nucleic acids comprising a nucleotide encoding the exogenous agent and at least one tissue-specific microRNA (miRNA) target sequence with a plurality of cells comprising target cells and non-target cells, wherein the exogenous agent is preferentially expressed in, e.g., restricted, to the target cell. In embodiments, the fusosome nucleic acid comprises at least one miRNA recognition sequence operably linked to a nucleotide sequence having a corresponding miRNA in a non-target cell, e.g., a hematopoietic progenitor cell (HSPC), hematopoietic stem cell (HSC), which prevents or reduces expression of the nucleotide sequence in the non-target cell but not in a target cell, e.g., differentiated cell. In some embodiments, the fusosome nucleic acid comprises at least one miRNA sequence target for a miRNA which is present in an effective amount (e.g., concentration of the endogenous miRNA is sufficient to reduce or prevent expression of a transgene) in the non-target cell, and comprises a transgene. In embodiments, the miRNA used in this system is strongly expressed in non-target cells, such as HSPC and HSC, but not in differentiated progeny of e.g. the myeloid and lymphoid lineage, preventing or reducing expression of a transgene in sensitive stem cell populations, while maintaining expression and therapeutic efficacy in the target cells.

### Immune Modulation

In some embodiments, a retroviral vector or fusosome described herein comprises elevated CD47. See, e.g., U.S. Pat. No. 9,050,269, which is herein incorporated by reference in its entirety. In some embodiments, a retroviral vector or fusosome described herein comprises elevated Complement Regulatory protein. See, e.g., ES2627445T3 and U.S. Pat. No. 6,790,641, each of which is incorporated herein by reference in its entirety. In some embodiments, a retroviral vector or fusosome described herein lacks or comprises reduced levels of an MHC protein, e.g., an MHC-1 class 1 or class II. See, e.g., US20170165348, which is herein incorporated by reference in its entirety.

Sometimes retroviral vectors or fusosomes are recognized by the subject's immune system. In the case of enveloped viral vector particles (e.g., retroviral vector particles), membrane-bound proteins that are displayed on the surface of the viral envelope may be recognized and the viral particle itself may be neutralised. Furthermore, on infecting a target cell, the viral envelope becomes integrated with the cell membrane and as a result viral envelope proteins may become displayed on or remain in close association with the surface of the cell. The immune system may therefore also target the cells which the viral vector particles have infected. Both effects may lead to a reduction in the efficacy of exogenous agent delivery by viral vectors.

A viral particle envelope typically originates in a membrane of the source cell. Therefore, membrane proteins that are expressed on the cell membrane from which the viral particle buds may be incorporated into the viral envelope.

### The Immune Modulating Protein CD47

The internalization of extracellular material into cells is commonly performed by a process called endocytosis (Rabinovitch, 1995, Trends Cell Biol. 5(3):85-7; Silverstein, 1995, Trends Cell Biol. 5(3):141-2; Swanson et al., 1995, Trends Cell Biol. 5(3):89-93; Allen et al., 1996, J. Exp. Med. 184(2):627-37). Endocytosis may fall into two general categories: phagocytosis, which involves the uptake of particles, and pinocytosis, which involves the uptake of fluid and solutes.

Professional phagocytes have been shown to differentiate from non-self and self, based on studies with knockout mice lacking the membrane receptor CD47 (Oldenborg et al., 2000, Science 288(5473):2051-4). CD47 is a ubiquitous member of the Ig superfamily that interacts with the immune inhibitory receptor SIRPα (signal regulatory protein) found on macrophages (Fujioka et al., 1996, Mol. Cell. Biol. 16(12):6887-99; Veillette et al., 1998, J. Biol. Chem. 273(35):22719-28; Jiang et al., 1999, J. Biol. Chem. 274(2):559-62). Although CD47-SIRPα interactions appear to deactivate autologous macrophages in mouse, severe reductions of CD47 (perhaps 90%) are found on human blood cells from some Rh genotypes that show little to no evidence of anemia (Mouro-Chanteloup et al., 2003, Blood 101(1):338-344) and also little to no evidence of enhanced cell interactions with phagocytic monocytes (Arndt et al., 2004, Br. J. Haematol. 125(3):412-4).

In some embodiments, a retroviral vector or fusosome (e.g., a viral particle having a radius of less than about 1 μm, less than about 400 nm, or less than about 150 nm), comprises at least a biologically active portion of CD47, e.g., on an exposed surface of the retroviral vector or fusosome. In some embodiments, the retroviral vector (e.g., lentivirus) or fusosome includes a lipid coat. In embodiments, the amount of the biologically active CD47 in the retroviral vector or fusosome is between about 20-250, 20-50, 50-100, 100-150, 150-200, or 200-250 molecules/μm². In some embodiments, the CD47 is human CD47.

A method described herein can comprise evading phagocytosis of a particle by a phagocytic cell. The method may include expressing at least one peptide including at least a biologically active portion of CD47 in a retroviral vector or fusosome so that, when the retroviral vector or fusosome comprising the CD47 is exposed to a phagocytic cell, the viral particle evades phacocytosis by the phagocytic cell, or shows decreased phagocytosis compared to an otherwise similar unmodified retroviral vector or fusosome. In some embodiments, the half-life of the retroviral vector or fusosome in a subject is extended compared to an otherwise similar unmodified retroviral vector or fusosome.

### MHC Deletion

The major histocompatibility complex class I (MHC-I) is a host cell membrane protein that can be incorporated into viral envelopes and, because it is highly polymorphic in nature, it is a major target of the body's immune response (McDevitt H. O. (2000) Annu. Rev. Immunol. 18: 1-17). MHC-I molecules exposed on the plasma membrane of source cells can be incorporated in the viral particle envelope during the process of vector budding. These MHC-I molecules derived from the source cells and incorporated in the viral particles can in turn be transferred to the plasma membrane of target cells. Alternatively, the MHC-I molecules may remain in close association with the target cell membrane as a result of the tendency of viral particles to absorb and remain bound to the target cell membrane.

The presence of exogenous MHC-I molecules on or close to the plasma membrane of transduced cells may elicit an alloreactive immune response in subjects. This may lead to immune-mediated killing or phagocytosis of transduced cells either upon ex vivo gene transfer followed by administration of the transduced cells to the subject, or upon direct in vivo administration of the viral particles. Furthermore, in the case of in vivo administration of MHC-I bearing viral particles into the bloodstream, the viral particles may be neutralised by pre-existing MHC-I specific antibodies before reaching their target cells.

Accordingly, in some embodiments, a source cell is modified (e.g., genetically engineered) to decrease expression of MHC-I on the surface of the cell. In embodiments, the source comprises a genetically engineered disruption of a gene encoding β2-microglobulin (β2M). In embodiments, the source cell comprises a genetically engineered disruption of one or more genes encoding an MHC-I α chain. The cell may comprise genetically engineered disruptions in all copies of the gene encoding β2-microglobulin. The cell may comprise genetically engineered disruptions in all copies of the genes encoding an MHC-I α chain. The cell may comprise both genetically engineered disruptions of genes encoding β2-microglobulin and genetically engineered disruptions of genes encoding an MHC-I α chain. In some embodiments, the retroviral vector or fusosome comprises a decreased number of surface-exposed MHC-I molecules. The number of surface-exposed MHC-I molecules may be decreased such that the immune response to the MHC-I is decreased to a therapeutically relevant degree. In some embodiments, the enveloped viral vector particle is substantially devoid of surface-exposed MHC-I molecules.

### HLA-G E Overexpression

In some embodiments, a retroviral vector or fusosome displays on its envelope a tolerogenic protein, e.g., an ILT-2 or ILT-4 agonist, e.g., HLA-E or HLA-G or any other ILT-2 or ILT-4 agonist. In some embodiments, a retroviral vector or fusosome has increased expression of HLA-E, HLA-G, ILT-2 or ILT-4 compared to a reference retrovirus, e.g., an unmodified retrovirus otherwise similar to the retrovirus.

In some embodiments, a retrovirus composition has decreased MHC Class I compared to an unmodified retrovirus and increased HLA-G compared to an unmodified retrovirus.

In some embodiments, the retroviral vector or fusosome has an increase in expression of HLA-G or HLA-E, e.g., an increase in expression of 1%, 5%, 10%, 20%, 30%, 40%, 50%, 60%, 70%, 80%, 90%, or more of HLA-G or HLA-E, compared to a reference retrovirus, e.g., an unmodified retrovirus otherwise similar to the retrovirus, wherein expression of HLA-G or HLA-E is assayed in vitro using flow cytometry, e.g., FACS.

In some embodiments, the retrovirus with increased HLA-G expression demonstrates reduced immunogenicity, e.g., as measured by reduced immune cell infiltration, in a teratoma formation assay.

### Complement Regulatory Proteins

Complement activity is normally controlled by a number of complement regulatory proteins (CRPs). These proteins prevent spurious inflammation and host tissue damage. One group of proteins, including CD55/decay accelerating factor (DAF) and CD46/membrane cofactor protein (MCP), inhibits the classical and alternative pathway C3/C5 convertase enzymes. Another set of proteins including CD59 regulates MAC assembly. CRPs have been used to prevent rejection of xenotransplanted tissues and have also been shown to protect viruses and viral vectors from complement inactivation.

Membrane resident complement control factors include, e.g., decay-accelerating factor (DAF) or CD55, factor H (FH)-like protein-1 (FHL-1), C4b-binding protein (C4BP), Complement receptor 1 (CD35), membrane cofactor protein (MCP) or CD46, and CD59 (protectin) (e.g., to prevent the formation of membrane attack complex (MAC) and protect cells from lysis).

### Albumin Binding Protein

In some embodiments the lentivirus binds albumin. In some embodiments the lentivirus comprises on its surface a protein that binds albumin. In some embodiments the lentivirus comprises on its surface an albumin binding protein. In some embodiments the albumin binding protein is streptococcal Albumin Binding protein. In some embodiments the albumin binding protein is streptococcal Albumin Binding Domain.

### Expression of Non-Fusogen Proteins on the Lentiviral Envelope

In some embodiments the lentivirus is engineered to comprise one or more proteins on its surface. In some embodiments the proteins affect immune interactions with a subject. In some embodiments the proteins affect the pharmacology of the lentivirus in the subject. In some embodiments the protein is a receptor. In some embodiments the protein is an agonist. In some embodiments the protein is a signaling molecule. In some embodiments, the protein on the lentiviral surface comprises an anti-CD3 antibody (e.g., OKT3) or IL7.

In some embodiments, a mitogenic transmembrane protein and/or a cytokine-based transmembrane protein is present in the source cell, which can be incorporated into the retrovirus when it buds from the source cell membrane. The mitogenic transmembrane protein and/or a cytokine-based transmembrane protein can be expressed as a separate cell surface molecule on the source cell rather than being part of the viral envelope glycoprotein.

In some embodiments of any of the aspects described herein, the retroviral vector, fusosome, or pharmaceutical composition is substantially non-immunogenic. Immunogenicity can be quantified, e.g., as described herein.

In some embodiments, a retroviral vector or fusosome fuses with a target cell to produce a recipient cell. In some embodiments, a recipient cell that has fused to one or more retroviral vectors or fusosomes is assessed for immunogenicity. In embodiments, a recipient cell is analyzed for the presence of antibodies on the cell surface, e.g., by staining with an anti-IgM antibody. In other embodiments, immunogenicity is assessed by a PBMC cell lysis assay. In embodiments, a recipient cell is incubated with peripheral blood mononuclear cells (PBMCs) and then assessed for lysis of the cells by the PBMCs. In other embodiments, immunogenicity is assessed by a natural killer (NK) cell lysis assay. In embodiments, a recipient cell is incubated with NK cells and then assessed for lysis of the cells by the NK cells. In other embodiments, immunogenicity is assessed by a CD8+ T-cell lysis assay. In embodiments, a recipient cell is incubated with CD8+ T-cells and then assessed for lysis of the cells by the CD8+ T-cells.

In some embodiments, the retroviral vector or fusosome comprises elevated levels of an immunosuppressive agent (e.g., immunosuppressive protein) as compared to a reference retroviral vector or fusosome, e.g., one produced from an unmodified source cell otherwise similar to the source cell, or a HEK293 cell. In some embodiments, the elevated level is at least 5%, 10%, 20%, 30%, 40%, 50%, 60%, 70%, 80%, 90%, 2-fold, 3-fold, 5-fold, 10-fold, 20-fold, 50-fold, or 100-fold. In some embodiments, the retroviral vector or fusosome comprises an immunosuppressive agent that is absent from the reference cell. In some embodiments, the retroviral vector or fusosome comprises reduced levels of an immunostimulatory agent (e.g., immunostimulatory protein) as compared to a reference retroviral vector or fusosome, e.g., one produced from an unmodified source cell otherwise similar to the source cell, or a HEK293 cell. In some embodiments, the reduced level is at least 5%, 10%, 20%, 30%, 40%, 50%, 60%, 70%, 80%, 90%, 95%, 98%, or 99% compared to the reference retroviral vector or fusosome. In some embodiments, the immunostimulatory agent is substantially absent from the retroviral vector or fusosome.

In some embodiments, the retroviral vector or fusosome, or the source cell from which the retroviral vector or fusosome is derived, has one, two, three, four, five, six, seven, eight, nine, ten, eleven, twelve, or more of the following characteristics:
a. less than 50%, 40%, 30%, 20%, 15%, 10%, or 5% or lesser expression of MHC class I or MHC class II, compared to a reference retroviral vector or fusosome, e.g., an unmodified retroviral vector or fusosome from a source cell otherwise similar to the source cell, or a HeLa cell, or a HEK293 cell;
b. less than 50%, 40%, 30%, 20%, 15%, 10%, or 5% or lesser expression of one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference retroviral vector or fusosome, e.g., an unmodified retroviral vector or fusosome from a cell otherwise similar to the source cell, or a HEK cell, or a reference cell described herein;
c. expression of surface proteins which suppress macrophage engulfment e.g., CD47, e.g., detectable expression by a method described herein, e.g., more than 1.5-fold, 2-fold, 3-fold, 4-fold, 5-fold, 10-fold, or more expression of the surface protein which suppresses macrophage engulfment, e.g., CD47, compared to a reference retroviral vector or fusosome, e.g., an unmodified retroviral vector or fusosome from a cell otherwise similar to the source cell, a Jurkat cell, or a HEK293 cell;
d. expression of soluble immunosuppressive cytokines, e.g., IL-10, e.g., detectable expression by a method described herein, e.g., more than 1.5-fold, 2-fold, 3-fold, 4-fold, 5-fold, 10-fold, or more expression of soluble immunosuppressive cytokines, e.g., IL-10, compared to a reference retroviral vector or fusosome, e.g., an unmodified retroviral vector or fusosome from a cell otherwise similar to the source cell, or a HEK293 cell;
e. expression of soluble immunosuppressive proteins, e.g., PD-L1, e.g., detectable expression by a method described herein, e.g., more than 1.5-fold, 2-fold, 3-fold, 4-fold, 5-fold, 10-fold, or more expression of soluble immunosuppressive proteins, e.g., PD-L1, compared to a reference retroviral vector or fusosome, e.g., an unmodified retroviral vector or fusosome from a cell otherwise similar to the source cell, or a HEK293 cell;
f. less than 50%, 40%, 30%, 20%, 15%, 10%, or 5% or lesser expression of soluble immune stimulating cytokines, e.g., IFN-gamma or TNF-α, compared to a reference retroviral vector or fusosome, e.g., an unmodified retroviral vector or fusosome from a cell otherwise similar to the source cell, or a HEK293 cell or a U-266 cell;
g. less than 50%, 40%, 30%, 20%, 15%, 10%, or 5% or lesser expression of endogenous immune-stimulatory antigen, e.g., Zg16 or Hormad1, compared to a reference retroviral vector or fusosome, e.g., an unmodified retroviral vector or fusosome from a cell otherwise similar to the source cell, or a HEK293 cell or an A549 cell, or a SK-BR-3 cell;
h. expression of, e.g., detectable expression by a method described herein, HLA-E or HLA-G, compared to a reference retroviral vector or fusosome, e.g., an unmodified retroviral vector or fusosome from a cell otherwise similar to the source cell, a HEK293 cell, or a or a Jurkat cell;
i. surface glycosylation profile, e.g., containing sialic acid, which acts to, e.g., suppress NK cell activation;
j. less than 50%, 40%, 30%, 20%, 15%, 10%, or 5% or lesser expression of TCRα/β, compared to a reference retroviral vector or fusosome, e.g., an unmodified retroviral vector or fusosome from a cell otherwise similar to the source cell, a HEK293 cell, or a Jurkat cell;
k. less than 50%, 40%, 30%, 20%, 15%, 10%, or 5% or lesser expression of ABO blood groups, compared to a reference retroviral vector or fusosome, e.g., an unmodified retroviral vector or fusosome from a cell otherwise similar to the source cell, a HEK293 cell, or a HeLa cell;
l. less than 50%, 40%, 30%, 20%, 15%, 10%, or 5% or lesser expression of Minor Histocompatibility Antigen (MHA), compared to a reference retroviral vector or fusosome, e.g., an unmodified retroviral vector or fusosome from a cell otherwise similar to the source cell, a HEK293 cell, or a Jurkat cell; or
m. has less than 10%, 9%, 8%, 7%, 6%, 5%, 4%, 3%, 2%, 1%, or less, of mitochondrial MHAs, compared to a reference retroviral vector or fusosome e.g., an unmodified retroviral vector or fusosome from a cell otherwise similar to the source cell, a HEK293 cell, or a Jurkat cell, or has no detectable mitochondrial MHAs.

In embodiments, the co-stimulatory protein is 4-1BB, B7, SLAM, LAG3, HVEM, or LIGHT, and the reference cell is HDLM-2. In some embodiments, the co-stimulatory protein is BY-H3 and the reference cell is HeLa. In some embodiments, the co-stimulatory protein is ICOSL or B7-H4, and the reference cell is SK-BR-3. In some embodiments, the co-stimulatory protein is ICOS or OX40, and the reference cell is MOLT-4. In some embodiments, the co-stimulatory protein is CD28, and the reference cell is U-266. In some embodiments, the co-stimulatory protein is CD30L or CD27, and the reference cell is Daudi.

In some embodiments, the retroviral vector, fusosome, or pharmaceutical composition does not substantially elicit an immunogenic response by the immune system, e.g., innate immune system. In embodiments, an immunogenic response can be quantified, e.g., as described herein. In some embodiments, the an immunogenic response by the innate immune system comprises a response by innate immune cells including, but not limited to NK cells, macrophages, neutrophils, basophils, eosinophils, dendritic cells, mast cells, or gamma/delta T cells. In some embodiments, an immunogenic response by the innate immune system comprises a response by the complement system which includes soluble blood components and membrane bound components.

In some embodiments, the retroviral vector, fusosome, or pharmaceutical composition does not substantially elicit an immunogenic response by the immune system, e.g., adaptive immune system. In some embodiments, an immunogenic response by the adaptive immune system comprises an immunogenic response by an adaptive immune cell including, but not limited to a change, e.g., increase, in number or activity of T lymphocytes (e.g., CD4 T cells, CD8 T cells, and or gamma-delta T cells), or B lymphocytes. In some embodiments, an immunogenic response by the adaptive immune system includes increased levels of soluble blood components including, but not limited to a change, e.g., increase, in number or activity of cytokines or antibodies (e.g., IgG, IgM, IgE, IgA, or IgD).

In some embodiments, the retroviral vector, fusosome, or pharmaceutical composition is modified to have reduced immunogenicity. In some embodiments, the retroviral vector, fusosome, or pharmaceutical composition has an immunogenicity less than 5%, 10%, 20%, 30%, 40%, or 50% lesser than the immunogenicity of a reference retroviral vector or fusosome, e.g., an unmodified retroviral vector or fusosome from a cell otherwise similar to the source cell, a HEK293 cell, or a Jurkat cell.

In some embodiments of any of the aspects described herein, the retroviral vector, fusosome, or pharmaceutical composition is derived from a source cell, e.g., a mammalian cell, having a modified genome, e.g., modified using a method described herein, to reduce, e.g., lessen, immunogenicity. Immunogenicity can be quantified, e.g., as described herein.

In some embodiments, the retroviral vector, fusosome, or pharmaceutical composition is derived from a mammalian cell depleted of, e.g., with a knock out of, one, two, three, four, five, six, seven or more of the following:
a. MHC class I, MHC class II or MHA;
b. one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4;
c. soluble immune-stimulating cytokines e.g., IFN-gamma or TNF-α;
d. endogenous immune-stimulatory antigen, e.g., Zg16 or Hormad1;
e. T-cell receptors (TCR);
f. The genes encoding ABO blood groups, e.g., ABO gene;
g. transcription factors which drive immune activation, e.g., NFkB;
h. transcription factors that control MHC expression e.g., class II trans-activator (CIITA), regulatory factor of the Xbox 5 (RFX5), RFX-associated protein (RFXAP), or RFX ankyrin repeats (RFXANK; also known as RFXB); or
i. TAP proteins, e.g., TAP2, TAP1, or TAPBP, which reduce MHC class I expression.

In some embodiments, the retroviral vector or fusosome is derived from a source cell with a genetic modification which results in increased expression of an immunosuppressive agent, e.g., one, two, three or more of the following (e.g., wherein before the genetic modification the cell did not express the factor):
a. surface proteins which suppress macrophage engulfment, e.g., CD47; e.g., increased expression of CD47 compared to a reference retroviral vector or fusosome, e.g., an unmodified retroviral vector or fusosome from a cell otherwise similar to the source cell, a HEK293 cell, or a Jurkat cell;
b. soluble immunosuppressive cytokines, e.g., IL-10, e.g., increased expression of IL-10 compared to a reference retroviral vector or fusosome, e.g., an unmodified retroviral vector or fusosome from a cell otherwise similar to the source cell, a HEK293 cell, or a Jurkat cell;
c. soluble immunosuppressive proteins, e.g., PD-1, PD-L1, CTLA4, or BTLA; e.g., increased expression of immunosuppressive proteins compared to a reference retroviral vector or fusosome, e.g., an unmodified retroviral vector or fusosome from a cell otherwise similar to the cell source, a HEK293 cell, or a Jurkat cell;
d. a tolerogenic protein, e.g., an ILT-2 or ILT-4 agonist, e.g., HLA-E or HLA-G or any other endogenous TLT-2 or ILT-4 agonist, e.g., increased expression of HLA-E, HLA-G, ILT-2 or ILT-4 compared to a reference retroviral vector or fusosome, e.g., an unmodified retroviral vector or fusosome from a cell otherwise similar to the source cell, a HEK293 cell, or a Jurkat cell; or
e. surface proteins which suppress complement activity, e.g., complement regulatory proteins, e.g. proteins that bind decay-accelerating factor (DAY, CD55), e.g. factor H (FH)-like protein-1 (FHL-1), e.g. C4b-binding protein (C4BP), e.g. complement receptor 1 (CD35), e.g. Membrane cofactor protein (MCP, CD46), eg. Profectin (CD59), e.g. proteins that inhibit the classical and alternative complement pathway CD/C5 convertase enzymes, e.g. proteins that regulate MAC assembly; e.g. increased expression of a complement regulatory protein compared to a reference retroviral vector or fusosome, e.g. an unmodified retroviral vector or fusosome from a cell otherwise similar to the source cell, a HEK293 cell, or a Jurkat cell.

In some embodiments, the increased expression level is at least 5%, 10%, 20%, 30%, 40%, 50%, 60%, 70%, 80%, 90%, 2-fold, 3-fold, 5-fold, 10-fold, 20-fold, 50-fold, or 100-fold higher as compared to a reference retroviral vector or fusosome.

In some embodiments, the retroviral vector or fusosome is derived from a source cell modified to have decreased expression of an immunostimulatory agent, e.g., one, two, three, four, five, six, seven, eight or more of the following:
a. less than 50%, 40%, 30%, 20%, 15%, 10%, or 5% or lesser expression of MHC class I or MHC class II, compared to a reference retroviral vector or fusosome, e.g., an unmodified retroviral vector or fusosome from a cell otherwise similar to the source cell, a HEK293 cell, or a HeLa cell;
b. less than 50%, 40%, 30%, 20%, 15%, 10%, or 5% or lesser expression of one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference retroviral vector or fusosome, e.g., an unmodified retroviral vector or fusosome from a cell otherwise similar to the source cell, a HEK293 cell, or a reference cell described herein;
c. less than 50%, 40%, 30%, 20%, 15%, 10%, or 5% or lesser expression of soluble immune stimulating cytokines, e.g., IFN-gamma or TNF-a, compared to a reference retroviral vector or fusosome, e.g., an unmodified retroviral vector or fusosome from a cell otherwise similar to the source cell, a HEK293 cell, or a U-266 cell;
d. less than 50%, 40%, 30%, 20%, 15%, 10%, or 5% or lesser expression of endogenous immune-stimulatory antigen, e.g., Zg16 or Hormad1, compared to a reference retroviral vector or fusosome, e.g., an unmodified retroviral vector or fusosome from a cell otherwise similar to the source cell, a HEK293 cell, or an A549 cell or a SK-BR-3 cell;
e. less than 50%, 40%, 30%, 20%, 15%, 10%, or 5% or lesser expression of T-cell receptors (TCR) compared to a reference retroviral vector or fusosome, e.g., an unmodified retroviral vector or fusosome from a cell otherwise similar to the source cell, a HEK293 cell, or a Jurkat cell;
f. less than 50%, 40%, 30%, 20%, 15%, 10%, or 5% or lesser expression of ABO blood groups, compared to a reference retroviral vector or fusosome, e.g., an unmodified retroviral vector or fusosome from a cell otherwise similar to the source cell, a HEK293 cell, or a HeLa cell;
g. less than 50%, 40%, 30%, 20%, 15%, 10%, or 5% or lesser expression of transcription factors which drive immune activation, e.g., NFkB; compared to a reference retroviral vector or fusosome, e.g., an unmodified retroviral vector or fusosome from a cell otherwise similar to the source cell, a HEK293 cell, or a Jurkat cell
h. less than 50%, 40%, 30%, 20%, 15%, 10%, or 5% or lesser expression of transcription factors that control MHC expression, e.g., class II trans-activator (CIITA), regulatory factor of the Xbox 5 (RFX5), RFX-associated protein (RFXAP), or RFX ankyrin repeats (RFXANK; also known as RFXB) compared to a reference retroviral vector or fusosome, e.g., an unmodified retroviral vector or fusosome from a cell otherwise similar to the source cell, a HEK293 cell, or a Jurkat cell; or
i. less than 50%, 40%, 30%, 20%, 15%, 10%, or 5% or lesser expression of TAP proteins, e.g., TAP2, TAP1, or TAPBP, which reduce MHC class I expression compared to a reference retroviral vector or fusosome, e.g., an unmodified retroviral vector or fusosome from a cell otherwise similar to the source cell, a HEK293 cell, or a HeLa cell.

In some embodiments, a retroviral vector, fusosome, or pharmaceutical composition derived from a mammalian cell, e.g., a HEK293, modified using shRNA expressing lentivirus to decrease MHC Class I expression, has lesser expression of MHC Class I compared to an unmodified retroviral vector or fusosome, e.g., a retroviral vector or fusosome from a cell (e.g., mesenchymal stem cell) that has not been modified. In some embodiments, a retroviral vector or fusosome derived from a mammalian cell, e.g., a HEK293, modified using lentivirus expressing HLA-G to increase expression of HLA-G, has increased expression of HLA-G compared to an unmodified retroviral vector or fusosome, e.g., from a cell (e.g., a HEK293) that has not been modified.

In some embodiments, the retroviral vector, fusosome, or pharmaceutical composition is derived from a source cell, e.g., a mammalian cell, which is not substantially immunogenic, wherein the source cells stimulate, e.g., induce, T-cell IFN-gamma secretion, at a level of 0 pg/mL to >0 pg/mL, e.g., as assayed in vitro, by IFN-gamma ELISPOT assay.

In some embodiments, the retroviral vector, fusosome, or pharmaceutical composition is derived from a source cell, e.g., a mammalian cell, wherein the mammalian cell is from a cell culture treated with an immunosuppressive agent, e.g., a glucocorticoid (e.g., dexamethasone), cytostatic (e.g., methotrexate), antibody (e.g., Muromonab (OKT3)-CD3), or immunophilin modulator (e.g., Ciclosporin or rapamycin).

In some embodiments, the retroviral vector, fusosome, or pharmaceutical composition is derived from a source cell, e.g., a mammalian cell, wherein the mammalian cell comprises an exogenous agent, e.g., a therapeutic agent.

In some embodiments, the retroviral vector, fusosome, or pharmaceutical composition is derived from a source cell, e.g., a mammalian cell, wherein the mammalian cell is a recombinant cell.

In some embodiments, the retroviral vector, fusosome, or pharmaceutical is derived from a mammalian cell genetically modified to express viral immunoevasins, e.g., hCMV US2, or US11.

In some embodiments, the surface of the retroviral vector or fusosome, or the surface of the source cell, is covalently or non-covalently modified with a polymer, e.g., a biocompatible polymer that reduces immunogenicity and immune-mediated clearance, e.g., PEG.

In some embodiments, the surface of the retroviral vector or fusosome, or the surface of the source cell is covalently or non-covalently modified with a sialic acid, e.g., a sialic acid comprising glycopolymers, which contain NK-suppressive glycan epitopes.

In some embodiments, the surface of the retroviral vector or fusosome, or the surface of the source cell is enzymatically treated, e.g., with glycosidase enzymes, e.g., α-N-acetylgalactosaminidases, to remove ABO blood groups

In some embodiments, the surface of the retroviral vector or fusosome, or the surface of the source cell is enzymatically treated, to give rise to, e.g., induce expression of, ABO blood groups which match the recipient's blood type.

### Parameters for Assessing Immunogenicity

In some embodiments, the retroviral vector or fusosome is derived from a source cell, e.g., a mammalian cell which is not substantially immunogenic, or modified, e.g., modified using a method described herein, to have a reduction in immunogenicity. Immunogenicity of the source cell and the retroviral vector or fusosome can be determined by any of the assays described herein.

In some embodiments, the retroviral vector or fusosome has an increase, e.g., an increase of 1%, 5%, 10%, 20%, 30%, 40%, 50%, 60%, 70%, 80%, 90%, or more, in in vivo graft survival compared to a reference retroviral vector or fusosome, e.g., an unmodified retroviral vector or fusosome from a cell otherwise similar to the source cell.

In some embodiments, the retroviral vector or fusosome has a reduction in immunogenicity as measured by a reduction in humoral response following one or more implantation of the retroviral vector or fusosome into an appropriate animal model, e.g., an animal model described herein, compared to a humoral response following one or more implantation of a reference retroviral vector or fusosome, e.g., an unmodified retroviral vector or fusosome from a cell otherwise similar to the source cell, into an appropriate animal model, e.g., an animal model described herein. In some embodiments, the reduction in humoral response is measured in a serum sample by an anti-cell antibody titre, e.g., anti-retroviral or anti-fusosome antibody titre, e.g., by ELISA. In some embodiments, the serum sample from animals administered the retroviral vector or fusosome has a reduction of 1%, 5%, 10%, 20%, 30%, 40%, 50%, 60%, 70%, 80%, 90%, or more of an anti-retroviral or anti-fusosome antibody titer compared to the serum sample from animals administered an unmodified retroviral vector or fusosome. In some embodiments, the serum sample from animals administered the retroviral vector or fusosome has an increased anti-retroviral or anti-fusosome antibody titre, e.g., increased by 1%, 2%, 5%, 10%, 20%, 30%, or 40% from baseline, e.g., wherein baseline refers to serum sample from the same animals before administration of the retroviral vector or fusosome.

In some embodiments, the retroviral vector or fusosome has a reduction in macrophage phagocytosis, e.g., a reduction of 1%, 5%, 10%, 20%, 30%, 40%, 50%, 60%, 70%, 80%, 90%, or more in macrophage phagocytosis compared to a reference retroviral vector or fusosome, e.g., an unmodified retroviral vector or fusosome from a cell otherwise similar to the source cell, wherein the reduction in macrophage phagocytosis is determined by assaying the phagocytosis index in vitro. In some embodiments, the retroviral vector or fusosome has a phagocytosis index of 0, 1, 10, 100, or more, when incubated with macrophages in an in vitro assay of macrophage phagocytosis.

In some embodiments, the source cell or recipient cell has a reduction in cytotoxicity mediated cell lysis by PBMCs, e.g., a reduction of 1%, 5%, 10%, 20%, 30%, 40%, 50%, 60%, 70%, 80%, 90%, or more in cell lysis compared to a reference cell, e.g., an unmodified cell otherwise similar to the source cell, or a recipient cell that received an unmodified retroviral vector or fusosome, or a mesenchymal stem cells. In embodiments, the source cell expresses exogenous HLA-G.

In some embodiments, the source cell or recipient cell has a reduction in NK-mediated cell lysis, e.g., a reduction of 1%, 5%, 10%, 20%, 30%, 40%, 50%, 60%, 70%, 80%, 90%, or more in NK-mediated cell lysis compared to a reference cell, e.g., an unmodified cell otherwise similar to the source cell, or a recipient cell that received an unmodified retroviral vector or fusosome, wherein NK-mediated cell lysis is assayed in vitro, by a chromium release assay or europium release assay.

In some embodiments, the source cell or recipient cell has a reduction in CD8+ T-cell mediated cell lysis, e.g., a reduction of 1%, 5%, 10%, 20%, 30%, 40%, 50%, 60%, 70%, 80%, 90%, or more in CD8 T cell mediated cell lysis compared to a reference cell, e.g., an unmodified cell otherwise similar to the source cell, or a recipient cell that received an unmodified retroviral vector or fusosome, wherein CD8 T cell mediated cell lysis is assayed in vitro.

In some embodiments, the source cell or recipient cell has a reduction in CD4+ T-cell proliferation and/or activation, e.g., a reduction of 1%, 5%, 10%, 20%, 30%, 40%, 50%, 60%, 70%, 80%, 90%, or more compared to a reference cell, e.g., an unmodified cell otherwise similar to the source cell, or a recipient cell that received an unmodified retroviral vector or fusosome, wherein CD4 T cell proliferation is assayed in vitro (e.g. co-culture assay of modified or unmodified mammalian source cell, and CD4+ T-cells with CD3/CD28 Dynabeads).

In some embodiments, the retroviral vector or fusosome causes a reduction in T-cell IFN-gamma secretion, e.g., a reduction of 1%, 5%, 10%, 20%, 30%, 40%, 50%, 60%, 70%, 80%, 90%, or more in T-cell IFN-gamma secretion compared to a reference retroviral vector or fusosome, e.g., an unmodified retroviral vector or fusosome from a cell otherwise similar to the source cell, wherein T-cell IFN-gamma secretion is assayed in vitro, e.g., by IFN-gamma ELISPOT.

In some embodiments, the retroviral vector or fusosome causes a reduction in secretion of immunogenic cytokines, e.g., a reduction of 1%, 5%, 10%, 20%, 30%, 40%, 50%, 60%, 70%, 80%, 90%, or more in secretion of immunogenic cytokines compared to a reference retroviral vector or fusosome, e.g., an unmodified retroviral vector or fusosome from a cell otherwise similar to the source cell, wherein secretion of immunogenic cytokines is assayed in vitro using ELISA or ELISPOT.

In some embodiments, the retroviral vector or fusosome results in increased secretion of an immunosuppressive cytokine, e.g., an increase of 1%, 5%, 10%, 20%, 30%, 40%, 50%, 60%, 70%, 80%, 90%, or more in secretion of an immunosuppressive cytokine compared to a reference retroviral vector or fusosome, e.g., an unmodified retroviral vector or fusosome from a cell otherwise similar to the source cell, wherein secretion of the immunosuppressive cytokine is assayed in vitro using ELISA or ELISPOT.

In some embodiments, the retroviral vector or fusosome has an increase in expression of HLA-G or HLA-E, e.g., an increase in expression of 1%, 5%, 10%, 20%, 30%, 40%, 50%, 60%, 70%, 80%, 90%, or more of HLA-G or HLA-E, compared to a reference retroviral vector or fusosome, e.g., an unmodified retroviral vector or fusosome from a cell otherwise similar to the source cell, wherein expression of HLA-G or HLA-E is assayed in vitro using flow cytometry, e.g., FACS. In some embodiments, the retroviral vector or fusosome is derived from a source cell which is modified to have an increased expression of HLA-G or HLA-E, e.g., compared to an unmodified cell, e.g., an increased expression of 1%, 5%, 10%, 20%, 30%, 40%, 50%, 60%, 70%, 80%, 90%, or more of HLA-G or HLA-E, wherein expression of HLA-G or HLA-E is assayed in vitro using flow cytometry, e.g., FACS. In some embodiments, the retroviral vector or fusosome derived from a modified cell with increased HLA-G expression demonstrates reduced immunogenicity.

In some embodiments, the retroviral vector or fusosome has or causes an increase in expression of T cell inhibitor ligands (e.g. CTLA4, PD1, PD-L1), e.g., an increase in expression of 1%, 5%, 10%, 20%, 30%, 40%, 50%, 60%, 70%, 80%, 90%, or more of T cell inhibitor ligands as compared to a reference retroviral vector or fusosome, e.g., an unmodified retroviral vector or fusosome from a cell otherwise similar to the source cell, wherein expression of T cell inhibitor ligands is assayed in vitro using flow cytometry, e.g., FACS.

In some embodiments, the retroviral vector or fusosome has a decrease in expression of co-stimulatory ligands, e.g., a decrease of 1%, 5%, 10%, 20%, 30%, 40%, 50%, 60%, 70%, 80%, 90%, or more in expression of co-stimulatory ligands compared to a reference retroviral vector or fusosome, e.g., an unmodified retroviral vector or fusosome from a cell otherwise similar to the source cell, wherein expression of co-stimulatory ligands is assayed in vitro using flow cytometry, e.g., FACS.

In some embodiments, the retroviral vector or fusosome has a decrease in expression of MHC class I or MHC class II, e.g., a decrease in expression of 1%, 5%, 10%, 20%, 30%, 40%, 50%, 60%, 70%, 80%, 90%, or more of MHC Class I or MHC Class II compared to a reference retroviral vector or fusosome, e.g., an unmodified retroviral vector or fusosome from a cell otherwise similar to the source cell or a HeLa cell, wherein expression of MHC Class I or II is assayed in vitro using flow cytometry, e.g., FACS.

In some embodiments, the retroviral vector or fusosome is derived from a cell source, e.g., a mammalian cell source, which is substantially non-immunogenic. In some embodiments, immunogenicity can be quantified, e.g., as described herein. In some embodiments, the mammalian cell source comprises any one, all or a combination of the following features:
a. wherein the source cell is obtained from an autologous cell source; e.g., a cell obtained from a recipient who will be receiving, e.g., administered, the retroviral vector or fusosome;
b. wherein the source cell is obtained from an allogeneic cell source which is of matched, e.g., similar, gender to a recipient, e.g., a recipient described herein who will be receiving, e.g., administered; the retroviral vector or fusosome;
c. wherein the source cell is obtained is from an allogeneic cell source is which is HLA matched with a recipient's HLA, e.g., at one or more alleles;
d. wherein the source cell is obtained is from an allogeneic cell source which is an HLA homozygote;
e. wherein the source cell is obtained is from an allogeneic cell source which lacks (or has reduced levels compared to a reference cell) MHC class I and II; or
f. wherein the source cell is obtained is from a cell source which is known to be substantially non-immunogenic including but not limited to a stem cell, a mesenchymal stem cell, an induced pluripotent stem cell, an embryonic stem cell, a sertoli cell, or a retinal pigment epithelial cell.

In some embodiments, the subject to be administered the retroviral vector or fusosome has, or is known to have, or is tested for, a pre-existing antibody (e.g., IgG or IgM) reactive with a retroviral vector or fusosome. In some embodiments, the subject to be administered the retroviral vector or fusosome does not have detectable levels of a pre-existing antibody reactive with the retroviral vector or fusosome. Tests for the antibody are described.

In some embodiments, a subject that has received the retroviral vector or fusosome has, or is known to have, or is tested for, an antibody (e.g., IgG or IgM) reactive with a retroviral vector or fusosome. In some embodiments, the subject that received the retroviral vector or fusosome (e.g., at least once, twice, three times, four times, five times, or more) does not have detectable levels of antibody reactive with the retroviral vector or fusosome. In embodiments, levels of antibody do not rise more than 1%, 2%, 5%, 10%, 20%, or 50% between two timepoints, the first timepoint being before the first administration of the retroviral vector or fusosome, and the second timepoint being after one or more administrations of the retroviral vector or fusosome. Tests for the antibody are described.

### Exogenous Agents

In some embodiments, a retroviral vector, fusosome, or pharmaceutical composition described herein encodes an exogenous agent.

In embodiments, an exogenous agent is a cargo that is exogenous relative to the source cell (hereinafter also called “agent” or “payload”). In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent is a nucleic acid that encodes a protein. The protein can be any protein as is desired for targeted delivery to a target cell. In some embodiments, the protein is a therapeutic agent or a diagnostic agent. In some embodiments, the protein is an antigen receptor for targeting cells expressed by or associated with a disease or condition, for instance a chimeric antigen receptor (CAR) or a T cell receptor (TCR). Reference to the coding sequence of a nucleic acid encoding the protein also is referred to herein as a payload gene. In some embodiments, the exogenous agent or the nucleic acid encoding the exogenous agent are present in the lumen of the fusosome.

In some embodiments, the exogenous agent or cargo comprises or encodes a cytosolic protein. In some embodiments the exogenous agent or cargo comprises or encodes a membrane protein. In some embodiments, the exogenous agent or cargo comprises or encodes a therapeutic agent. In some embodiments, the therapeutic agent is chosen from one or more of a protein, e.g., an enzyme, a transmembrane protein, a receptor, an antibody; a nucleic acid, e.g., DNA, a chromosome (e.g. a human artificial chromosome), RNA, mRNA, siRNA, miRNA, or a small molecule.

In embodiments, the exogenous agent is present at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In embodiments, the fusosome has an altered, e.g., increased or decreased level of one or more endogenous molecule, e.g., protein or nucleic acid (e.g., in some embodiments, endogenous relative to the source cell, and in some embodiments, endogenous relative to the target cell), e.g., due to treatment of the source cell, e.g., mammalian source cell with a siRNA or gene editing enzyme. In embodiments, the endogenous molecule is present at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In embodiments, the endogenous molecule (e.g., an RNA or protein) is present at a concentration of at least 1, 2, 3, 4, 5, 10, 20, 50, 100, 500, 103, 5.0×103, 104, 5.0×104, 105, 5.0×105, 106, 5.0×106, 1.0×107, 5.0×107, or 1.0×108, greater than its concentration in the source cell. In embodiments, the endogenous molecule (e.g., an RNA or protein) is present at a concentration of at least 1, 2, 3, 4, 5, 10, 20, 50, 100, 500, 103, 5.0×103, 104, 5.0×104, 105, 5.0×105, 106, 5.0×106, 1.0×107, 5.0×107, or 1.0×108 less than its concentration in the source cell.

In some embodiments, the fusosome delivers to a target cell at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the fusosome. In some embodiments, the fusosome that fuses with the target cell(s) delivers to the target cell an average of at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the fusosomes that fuse with the target cell(s). In some embodiments, the fusosome composition delivers to a target tissue at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the fusosome compositions.

In some embodiments, the exogenous agent or cargo is not expressed naturally in the cell from which the targeted lipid particle is derived. In some embodiments, the exogenous agent or cargo is expressed naturally in the cell from which the targeted lipid particle is derived. In some embodiments, the exogenous agent or cargo is loaded into the targeted lipid particle via expression in the cell from which the fusosome is derived (e.g. expression from DNA or mRNA introduced via transfection, transduction, or electroporation). In some embodiments, the exogenous agent or cargo is expressed from DNA integrated into the genome or maintained episosomally. In some embodiments, expression of the exogenous agent or cargo is constitutive. In some embodiments, expression of the exogenous agent or cargo is induced. In some embodiments, expression of the exogenous agent or cargo is induced immediately prior to generating the targeted lipid particle. In some embodiments, expression of the exogenous agent or cargo is induced at the same time as expression of the fusogen.

In some embodiments, the exogenous agent or cargo is loaded into the fusosome via electroporation into the fusosome itself or into the cell from which the fusosome is derived. In some embodiments, the exogenous agent or cargo is loaded into the lipid particle via transfection (e.g., of a DNA or mRNA encoding the cargo) into the fusosome itself or into the cell from which the fusosome is derived.

### Exemplary Exogenous Agents

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell.

In some embodiments, the exogenous agent comprises a nucleic acid, e.g., RNA, intron(s), exon(s), mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid or a mutant nucleic acid. In some embodiments the nucleic acid is a fusion or chimera of multiple nucleic acid sequences.

In some embodiments, the exogenous agent comprises a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein or a mutant protein. In some embodiments the protein is a fusion or chimeric protein.

In some embodiments, the exogenous agent or cargo may include one or more nucleic acid sequences, one or more polypeptides, a combination of nucleic acid sequences and/or polypeptides, one or more organelles, and any combination thereof. In some embodiments, the exogenous agent or cargo may include one or more cellular components. In some embodiments, the exogenous agent or cargo includes one or more cytosolic and/or nuclear components.

In some embodiments, the exogenous agent or cargo includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograrnming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In some embodiments, the exogenous agent or cargo may include a nucleic acid. For example, the exogenous agent or cargo may comprise RNA to enhance expression of an endogenous protein, or a siRNA or miRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the exogenous agent or cargo is a nucleic acid that targets a transcriptional activator that modulate structure or function in the target cells.

In some embodiments, the exogenous agent or cargo is or encodes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein. In some embodiments, the protein is a mutant protein. In some embodiments the protein is a fusion or chimeric protein.

In some embodiments, the exogenous agent or cargo is a small molecule, e.g., ions (e.g. Ca2+, Cl—, Fe2+), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent or cargo includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the exogenous agent or cargo includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, networks of organelles, and any combination thereof.

In some embodiments, the exogenous agent is or encodes a cytosolic protein, e.g., a protein that is produced in the recipient cell and localizes to the recipient cell cytoplasm. In some embodiments, the exogenous agent is or encodes a secreted protein, e.g., a protein that is produced and secreted by the recipient cell. In some embodiments, the exogenous agent is or encodes a nuclear protein, e.g., a protein that is produced in the recipient cell and is imported to the nucleus of the recipient cell. In some embodiments, the exogenous agent is or encodes an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

In some embodiments, the exogenous agent is capable of being delivered to a hepatocyte or liver cell. In some embodiments, the exogenous agents or cargo can be delivered to treat a disease or disorder in a hepatocyte or liver cell.

In some embodiments, the exogenous agent is encoded by a gene from among OTC, CPS1, NAGS, BCKDHA, BCKDHB, DBT, DLD, MUT, MMAA, MMAB, MMACHC, MMADHC, MCEE, PCCA, PCCB, UGT1A1, ASS1, PAH, PAL, ATP8B1, ABCB11, ABCB4, TJP2, IVD, GCDH, ETFA, ETFB, ETFDH, ASL, D2HGDH, HMGCL, MCCC1, MCCC2, ABCD4, HCFC1, LNBRD1, ARG1, SLC25A15, SLC25A13, ALAD, CPOX, HMBS, PPOX, BTD, HLCS, PC, SLC7A7, CPT2, ACADM, ACADS, ACADVL, AGL, G6PC, GBE1, PHKA1, PHKA2, PHKB, PHKG2, SLC37A4, PMM2, CBS, FAH, TAT, GALT, GALK1, GALE, G6PD, SLC3A1, SLC7A9, MTHFR, MTR, MTRR, ATP7B, HPRT1, HJV, HAMP, JAG1, TTR, AGXT, LIPA, SERPINGI, HSD17B4, UROD, HFE, LPL,GRHPR, HOGA1, LDLR, ACAD8, ACADSB, ACAT1, ACSF3, ASPA, AUH, DNAJC19, ETHE1, FBP1, FTCD, GSS, HIBCH, IDH2, L2HGDH, MLYCD, OPA3, OPLAH, OXCT1, POLG, PPM1K, SERAC1, SLC25A1, SUCLA2, SUCLG1, TAZ, AGK, CLPB, TMEM70, ALDH18A1, OAT, CA5A, GLUD1, GLUL, UMPS, SLC22A5, CPT1A, HADHA, HADH, SLC52A1, SLC52A2, SLC52A3, HADHB, GYS2, PYGL, SLC2A2, ALG1, ALG2, ALG3, ALG6, ALG8, ALG9, ALG11, ALG12, ALG13, ATP6VOA2, B3GLCT, CHST14, COG1, COG2, COG4, COG5, COG6, COG7, COG8, DOLK, DHDDS, DPAGT1, DPM1, DPM2, DPM3, G6PC3, GFPT1, GMPPA, GMPPB, MAGT1, MAN1B1, MGAT2, MOGS, MPDU1, MPI, NGLY1, PGM1, PGM3, RFT1, SEC23B, SLC35A1, SLC35A2, SLC35C1, SSR4, SRD5A3, TMEM165, TRIP11, TUSC3, ALG14, B4GALT1, DDOST, NUS1, RPN2, SEC23A, SLC35A3, ST3GAL3, STT3A, STT3B, AGA, ARSA, ARSB, ASAH1, ATP13A2, CLN3, CLN5, CLN6, CLN8, CTNS, CTSA, CTSD, CTSF, CTSK, DNAJC5, FUCA1, GAA, GALC, GALNS, GLA, GLB1, GM2A, GNPTAB, GNPTG, GNS, GRN, GUSB, HEXA, HEXB, HGSNAT, HYAL1, IDS, IDUA, KCTD7, LAMP2, MAN2B1, MANBA, MCOLN1, MFSD8, NAGA, NAGLU, NEU1, NPC1, NPC2, SGSH, PPT1, PSAP, SLC17A5, SMPD1, SUMF1, TPP1, AHCY, GNMT, MAT1A, GCH1, PCBD1, PTS, QDPR, SPR, DNAJC12, ALDH4A1, PRODH, HPD, GBA, HGD, AMN, CD320, CUBN, GIF, TCN1, TCN2, PREPL, PHGDH, PSAT1, PSPH, AMT,GCSH, GLDC, LIAS, NFU1, SLC6A9, SLC2A1, ATP7A, APIS1, CP, SLC33A1, PEX7, PHYH, AGPS, GNPAT, ABCD1, ACOX1, PEX1, PEX2, PEX3, PEX5, PEX6, PEX10, PEX12, PEX13, PEX14, PEX16, PEX19, PEX26, AMACR, ADA, ADSL, AMPD1, GPHN, MOCOS, MOCS1, PNP, XDH, SUOX, OGDH, SLC25A19, DHTKD1, SLC13A5, FH, DLAT, MPC1, PDHA1, PDHB, PDHX, PDP1, ABCC2, SLCO1B1, SLCO1B3, HFE2, ADAMTS13, PYGM, COL1A2, TNFRSF11B, TSC1, TSC2, DHCR7, PGK1, VLDLR, KYNU, F5, C3, COL4A1, CFH, SLC12A2, GK, SFTPC, CRTAP, P3H1, COL7A1, PKLR, TALDO1, TF, EPCAM, VHL, GC, SERPINA1, ABCC6, F8, F9, ApoB, PCSK9, LDLRAP1,ABCG5, ABCG8, LCAT, SPINK5, or GNE.

In some embodiments, the exogenous agent is encoded by a gene from among OTC, CPS1, NAGS, BCKDHA, BCKDHB, DBT, DLD, MUT, MMAA, MMAB, MMACHC, MMADHC, MCEE, PCCA, PCCB, UGT1A1, ASS1, PAL, PAH, ATP8B1, ABCB11, ABCB4, TJP2, IVD, GCDH, ETFA, ETFB, ETFDH, ASL, D2HGDH, HMGCL, MCCC1, MCCC2, ABCD4, HCFC1, LMBRD1, ARG1, SLC25A15, SLC25A13, ALAD, CPOX, HMBS, PPOX, BTD, HLCS, PC, SLC7A7, CPT2, ACADM, ACADS, ACADVL, AGL, G6PC, GBE1, PHKA1, PHKA2, PHKB, PHKG2, SLC37A4, PMM2, CBS, FAH, TAT, GALT, GALK1, GALE, G6PD, SLC3A1, SLC7A9, MTHFR, MTR, MTRR, ATP7B, HPRT1, HJV, HAMP, JAG1, TTR, AGXT, LIPA, SERPINGI, HSD17B4, UROD, HFE, LPL, GRHPR, HOGA1, or LDLR. In some embodiments, the exogenous agent is the enzyme phenylalanine ammonia lyase (PAL).

In some embodiments, the exogenous agents or cargo can be delivered to treat and disease or indication listed in Table 5A. In some embodiments, the indications are specific for a liver cell or hepatocyte.

In some embodiments, the exogenous cargo comprises a protein of Table 5A below. In some embodiments, the exogenous agent comprises the wild-type human sequence of any of the proteins of Table 5A, a functional fragment thereof (e.g., an enzymatically active fragment thereof), or a functional variant thereof. In some embodiments, the exogenous agent comprises an amino acid sequence having at least 70%, 75%, 80%, 85, 90%, 95%, 96%, 97%, 98%, or 99% identity to an amino acid sequence of Table 5A. In some embodiments, the payload gene encoding an exogenous agent encodes an amino acid sequence having at least 70%, 75%, 80%, 85%, 90%, 95%, 96%, 97%, 98%, or 99% identity to an amino acid sequence of Table 5A. In some embodiments, the payload gene encoding an exogenous agent has a nucleic acid sequence having at least 70%, 75%, 80%, 85%, 90%, 95%, 96%, 97%, 98%, or 99% identity to a nucleic acid sequence of Table 5A.

TABLE 5A
Exemplary Exogenous Cargo

| | | | Ensembl | | |
|---|---|---|---|---|---|
| | | | Gene(s) | | |
| | | | Accession | Uniprot | |
| | | Entrez | Number | Protein(s) | |
| | Disease/ | Accession | (ENSG0000 + | Accession | |
| Gene | Disorder | Number | number shown) | Number | Category |
| OTC | ornithine | 5009 | 0036473 | P00480 | Urea cycle |
| | transcarbamylase | | | | disorder |
| | (OTC) | | | | |
| | deficiency | | | | |
| CPS1 | carbamoyl | 1373 | 0021826 | P31327, | Urea cycle |
| | phosphate | | | Q6PEK7, | disorder |
| | synthetase I | | | B7ZAW0, | |
| | (CPSI) | | | A0A024R454 | |
| | deficiency | | | | |
| NAGS | N-acetylglutamate | 162417 | 0161653 | Q8N159, | Urea cycle |
| | synthase | | | Q2NKP2 | disorder |
| | (NAGS) | | | | |
| | deficiency | | | | |
| BCKDHA | maple syrup | 593 | 0248098 | A0A024R0K3, | Organic |
| | urine disease | | | P12694, | acidemia |
| | (MSUD); | | | Q59EI3 | |
| | Classic Maple | | | | |
| | Syrup Urine | | | | |
| | Disease | | | | |
| | (CMSUD) | | | | |
| BCKDHB | maple syrup | 594 | 0083123 | A0A140VKB3, | Organic |
| | urine disease | | | P21953, | acidemia |
| | (MSUD); | | | B4E2N3, | |
| | Classic Maple | | | B7ZB80 | |
| | Syrup Urine | | | | |
| | Disease | | | | |
| | (CMSUD) | | | | |
| DBT | maple syrup | 1629 | 0137992 | P11182 | Organic |
| | urine disease | | | | acidemia |
| | (MSUD); | | | | |
| | Classic Maple | | | | |
| | Syrup Urine | | | | |
| | Disease | | | | |
| | (CMSUD) | | | | |
| DLD | maple syrup | 1738 | 0091140 | A0A024R713, | Urea cycle |
| | urine disease | | | P09622, | disorder |
| | (MSUD) | | | | |
| | Dihydrolipoamide | | | E9PEX6 | |
| | dehydrogenase | | | | |
| | deficiency | | | | |
| MUT | methylmalonic | 4594 | 0146085 | A0A024RD8 | Organic |
| | acidemia due to | | | B2R6K1, | acidemia |
| | methylmalonyl- | | | P22033 | |
| | CoA mutase | | | | |
| | deficiency | | | | |
| MMAA | cobalamin A | 166785 | 0151611 | Q8IVH4 | Organic |
| | deficiency | | | | acidemia |
| | (methylmalonic | | | | |
| | acidemia) | | | | |
| MMAB | cobalamin B | 326625 | 0139428 | Q96EY8 | Organic |
| | deficiency | | | | acidemia |
| | (methylmalonic | | | | |
| | acidemia) | | | | |
| MMACHC | cobalamin C | 25974 | 0132763 | A0A0C4DGU2, | Organic |
| | deficiency | | | Q9Y4U1 | acidemia |
| | (methylmalonic | | | | |
| | acidemia); | | | | |
| | Methylmalonic | | | | |
| | Acidemia with | | | | |
| | Homocystinuria | | | | |
| MMADHC | cobalamin D | 27249 | 0168288 | Q9H3L0 | Organic |
| | deficiency | | | | acidemia |
| | (methylmalonic | | | | |
| | acidemia); | | | | |
| | Methylmalonic | | | | |
| | Acidemia with | | | | |
| | Homocystinuria; | | | | |
| | Homocystinuria; | | | | |
| | Cobalamin C | | | | |
| | Deficiency | | | | |
| MCEE | methylmalonic | 84693 | 0124370 | Q96PE7 | Organic |
| | acidemia; | | | | acidemia |
| | Cobalamin D | | | | |
| | Deficiency | | | | |
| PCCA | propionic | 5095 | 0175198 | P05165 | Organic |
| | acidemia | | | | acidemia |
| PCCB | propionic | 5096 | 0114054 | P05166 | Organic |
| | acidemia | | | | acidemia |
| UGT1A1 | Crigler-Najjar | 54658 | 0241635 | P22309, | |
| | syndrome type 1 | | | Q5DT03 | |
| | Crigler-Najjar | | | | |
| | syndrome type | | | | |
| | 2, Gilbert | | | | |
| | syndrome | | | | |
| ASS1 | citrullinemia | 445 | 0130707 | P00966, | Urea cycle |
| | type I | | | Q5T6L4 | disorder |
| PAH | Phenylalanine | 5053 | 0171759 | A0A024RBG4, | Aminoacidopathy |
| | hydroxylase | | | P00439 | |
| | deficiency | | | | |
| PAL | Phenylalanine | | | | Aminoacidopathy |
| | hydroxylase | | | | |
| | deficiency | | | | |
| ATP8B1 | Progressive | 5205 | 0081923 | O43520 | |
| | familial | | | | |
| | intrahepatic | | | | |
| | cholestasis | | | | |
| | Type 1 | | | | |
| ABCB11 | Progressive | 8647 | 0073734, | O95342 | |
| | familial | | 0276582 | | |
| | intrahepatic | | | | |
| | cholestasis | | | | |
| | Type 2; | | | | |
| | Progressive | | | | |
| | Familial | | | | |
| | Intrahepatic | | | | |
| | Cholestasis | | | | |
| | Type 3 | | | | |
| ABCB4 | Progressive | 5244 | 0005471 | P21439 | |
| | familial | | | | |
| | intrahepatic | | | | |
| | cholestasis | | | | |
| | Type 3; | | | | |
| | Progressive | | | | |
| | Familial | | | | |
| | Intrahepatic | | | | |
| | Cholestasis | | | | |
| | Type 2 | | | | |
| TJP2 | Progressive | 9414 | 0119139 | B7Z2R3, | |
| | familial | | | Q9UDY2, | |
| | intrahepatic | | | B7Z954 | |
| | cholestasis | | | | |
| | Type 4 | | | | |
| IVD | isovaleric | 3712 | 0128928 | P26440, | Organic |
| | acidemia (IVD) | | | A0A0A0MT83 | acidemia |
| GCDH | glutaric | 2639 | 0105607 | A0A024R7F9, | Organic |
| | acidemia type I | | | Q92947 | acidemia |
| ETFA | multiple | 2108 | 0140374 | A0A0S2Z3L0, | Organic |
| | acyl-CoA | | | P13804 | acidemia |
| | dehydrogenase | | | | |
| | deficiency | | | | |
| | (a.k.a. glutaric | | | | |
| | aciduria type II) | | | | |
| ETFB | multiple | 2109 | 0105379 | P38117 | Organic |
| | acyl-CoA | | | | acidemia |
| | dehydrogenase | | | | |
| | deficiency | | | | |
| | (a.k.a. glutaric | | | | |
| | aciduria type II) | | | | |
| ETFDH | multiple | 2110 | 0171503 | B4DEQ0, | Organic |
| | acyl-CoA | | | Q16134 | acidemia |
| | dehydrogenase | | | | |
| | deficiency | | | | |
| | (a.k.a. glutaric | | | | |
| | aciduria type II) | | | | |
| ASL | argininosuccinate | 435 | 0126522 | A0A024RDL8, | Urea cycle |
| | lyase (ASL) | | | P04424, | disorder |
| | deficiency | | | A0A0S2Z316 | |
| D2HGDH | D-2- | 728294 | 0180902 | B3KSR6, | Organic |
| | hydroxyglutaric | | | B4E3K7, | acidemia |
| | aciduria type I | | | B5MCV2, | |
| | | | | Q8N465 | |
| HMGCL | 3-hydroxy-3- | 3155 | 0117305 | P35914 | Organic |
| | methylglutaryl- | | | | academia |
| | CoA lyase | | | | Urea cycle |
| | (3HMG) | | | | disorder |
| | deficiency | | | | |
| MCCC1 | 3-methylcrotonyl- | 56922 | 0078070 | Q68D27, | Organic |
| | CoA carboxylase | | | Q96RQ3, | acidemia |
| | (3MCC) | | | A0A0S2Z693, | |
| | deficiency | | | E9PHF7 | |
| MCCC2 | 3-methylcrotonyl- | 64087 | 0131844, | A0A140VK29, | Organic |
| | CoA carboxylase | | 0281742, | Q9HCC0 | acidemia |
| | (3MCC) deficiency | | 0275300 | | |
| ABCD4 | methylmalonic | 5826 | 0119688 | A0A024R6B9, | Organic |
| | acidemia with | | | O14678, | acidemia |
| | homocystinuria | | | A0A024R6C8 | |
| HCFC1 | methylmalonic | 3054 | 0172534 | P51610, | Organic |
| | acidemia with | | | A6NEM2 | acidemia |
| | homocystinuria | | | | |
| LMBRD1 | methylmalonic | 55788 | 0168216 | Q9NUN5 | Organic |
| | acidemia with | | | | acidemia |
| | homocystinuria | | | | |
| ARG1 | arginase | 383 | 0118520 | P05089 | Urea cycle |
| | (ARG1) | | | | disorder |
| | deficiency | | | | |
| SLC25A15 | hyperammonemia- | 10166 | 0102743 | Q9Y619 | Urea cycle |
| | hyperornithinemia- | | | | disorder |
| | homocitrullinuria | | | | |
| | (HHH) | | | | |
| | syndrome | | | | |
| SLC25A13 | citrin deficiency | 10165 | 0004864 | Q9UJS0 | Urea cycle |
| | citrullinemia | | | | disorder |
| | type II | | | | |
| ALAD | Acute Hepatic | 210 | 0148218 | P13716 | Porphyria |
| | porphyria | | | | |
| CPOX | Acute Hepatic | 1371 | 0080819 | P36551 | Porphyria |
| | porphyria | | | | |
| HMBS | Acute Hepatic | 3145 | 0256269, | P08397 | Porphyria |
| | porphyria; | | 0281702 | | |
| | Acute | | | | |
| | Intermittent | | | | |
| | Porphyria | | | | |
| PPOX | Acute Hepatic | 5498 | 0143224 | P50336, | Porphyria |
| | porphyria | | | B4DY76 | |
| BTD | Biotinidase | 686 | 0169814 | P43251 | Organic |
| | Deficiency | | | | acidemia |
| HLCS | Holocarboxylase | 3141 | 0159267 | P50747 | Organic |
| | Synthetase | | | | acidemia |
| | Deficiency | | | | |
| PC | Pyruvate | 5091 | 0173599 | P11498 | Urea cycle |
| | Carboxylase | | | A0A024R5C5 | disorder |
| | Deficiency | | | | |
| SLC7A7 | Lysinuric | 9056 | 0155465 | Q9UM01 | Urea cycle |
| | Protein | | | A0A0S2Z502 | disorder |
| | Intolerance | | | | |
| CPT2 | Carnitine | 1376 | 0157184 | P23786 | Fatty Acid |
| | Palmitoyltransferase | | | A0A140VK13 | Oxidation |
| | Type II | | | A0A1B0GTB8 | |
| | (CPT II) | | | | |
| | Deficiency | | | | |
| ACADM | Medium Chain | 34 | 0117054 | P11310 | Fatty Acid |
| | Acyl-CoA | | | A0A0S2Z366, | Oxidation |
| | Dehydrogenase | | | B7Z911, | |
| | (MCAD) | | | Q5HYG7, | |
| | Deficiency | | | Q5T4U5, | |
| | | | | B4DJE7 | |
| ACADS | Short Chain | 35 | 0122971 | P16219 | Fatty acid |
| | Acyl-CoA | | | E5KSD5, | oxidation |
| | (SCAD) | | | B4DUH1, | |
| | Dehydrogenase | | | E9PE82 | |
| | Deficiency | | | | |
| ACADVL | Very Long | 37 | 0072778 | P49748 | Fatty acid |
| | Chain Acyl- | | | B3KPA6 | oxidation |
| | CoA | | | | |
| | Dehydrogenase | | | | |
| | (VLCAD) | | | | |
| | Deficiency | | | | |
| AGL | GSD III (Cori/ | 178 | 0162688 | P35573 | Liver |
| | Forbe Disease | | | A0A0S2A4E4 | glycogen |
| | or Debrancher) | | | | storage |
| | | | | | disorder |
| G6PC | GSDIa (Von | 2538 | 0131482 | P35575 | Liver |
| | Gierke Disease) | | | | glycogen |
| | | | | | storage |
| | | | | | disorder |
| GBE1 | GSD IV | 2632 | 0114480 | Q04446 | Liver |
| | (Andersen | | | Q59ET0 | glycogen |
| | Disease, | | | | storage |
| | Brancher | | | | disorder |
| | Enzyme) | | | | |
| PHKA1 | GSD IXa | 5255 | 0067177 | P46020 | |
| PHKA2 | GSD IXa | 0044446 | 5256 | P46019 | Liver |
| | | 5256 | 0044446 | | glycogen |
| | | | | | storage |
| | | | | | disorder |
| PHKB | GSD IXb | 5257 | 0102893 | Q93100 | Liver |
| | | | | | glycogen |
| | | | | | storage |
| | | | | | disorder |
| PHKG2 | GSD IXc | 5261 | 0156873 | P15735 | Liver |
| | | | | | glycogen |
| | | | | | storage |
| | | | | | disorder |
| SLC37A4 | GSDIb. c, d | 2542 | 0281500 | O43826 | Liver |
| | | | 0137700 | A0A024R3H9, | glycogen |
| | | | | A8K0S7, | storage |
| | | | | A0A024R3L1, | disorder |
| | | | | B4DUH2 | |
| PMM2 | PMM2-CDG | 5373 | 0140650 | O15305, | Glycosylation |
| | | | | A0A0S2Z4J6, | disorder |
| | | | | Q59F02 | |
| CBS | Cystathionine | 102724560, | 0160200 | P35520, | Aminoacidopathy |
| | Beta-Synthase | 875 | | P0DN79, | |
| | Deficiency | | | Q9NTF0, | |
| | (Classic | | | B7Z2D6 | |
| | Homocystinuria); | | | | |
| | Homocystinuria | | | | |
| FAH | Tyrosinemia | 2184 | 0103876 | P16930 | Aminoacidopathy |
| | Type I | | | | |
| TAT | Tyrosinemia | 6898 | 0198650 | P17735, | Aminoacidopathy |
| | Type II | | | A0A140VKB7 | |
| | Tyrosinemia | | | | |
| | Type III | | | | |
| GALT | Galactosemia | 2592 | 0213930 | P07902, | Carbohydrate |
| | due to | | | A0A0S2Z3Y7, | disorder |
| | galactose-1- | | | B2RAT6 | |
| | phosphate | | | | |
| | uridylyltranserase | | | | |
| | (GALT) | | | | |
| | deficiency | | | | |
| GALK1 | Galactosemia | 2584 | 0108479 | P51570 | Carbohydrate |
| | | | | | disorder |
| GALE | Galactosemia | 2582 | 0117308 | Q14376 | Carbohydrate |
| | | | | | disorder |
| G6PD | Glucose-6- | 2539 | 0160211 | P11413 | Carbohydrate |
| | Phosphate | | | | disorder |
| | Dehydrogenase | | | | |
| | (G6PD) | | | | |
| | Deficiency | | | | |
| SLC3A1 | Cystinuria | 6519 | 0138079 | Q07837, | Aminoacidopathy |
| | | | | A0A0S2Z4E1, | |
| | | | | B8ZZK1 | |
| SLC7A9 | Cystinuria | 11136 | 0021488 | P82251 | Aminoacidopathy |
| MTHFR | Homocystinuria | 4524 | 0177000 | P42898, | Aminoacidopathy |
| | | | | Q59GJ6, | |
| | | | | Q81U67 | |
| MTR | Homocystinuria | 4548 | 0116984 | Q99707 | Aminoacidopathy |
| MTRR | Homocystinuria | 4552 | 0124275 | Q9UBK8 | Aminoacidopathy |
| ATP7B | Wilson Disease | 540 | 0123191 | P35670, | Metal |
| | Copper | | | A0A024RDX3, | transport |
| | Metabolism | | | B7ZLR4, | disorder |
| | Disorder | | | B7ZLR3, | |
| | | | | E7ET55 | |
| HPRT1 | Lesch-Nyhan | 3251 | 0165704 | P00492, | Purine |
| | Syndrome | | | A0A140VJL3 | Metabolism |
| | Purine | | | | Disorder |
| | Metabolism | | | | |
| | Disorder | | | | |
| HJV | Hemochromatosis, | 148738 | 0168509 | Q6ZVN8 | |
| | Type 2A | | | | |
| HAMP | Hemochromatosis | 57817 | 0105697 | P81172 | |
| | Type 2B: | | | | |
| | Primary | | | | |
| | Hemochromatosis | | | | |
| JAG1 | Alagille | 182 | 0101384 | P78504, | |
| | Syndrome 1 | | | Q99740 | |
| TTR | Familial TTR | 7276 | 0118271 | P02766, | |
| | Amyloidoisis; | | | E9KL36 | |
| | Familial | | | | |
| | amyloid | | | | |
| | polyneuropathy | | | | |
| AGXT | Primary | 189 | 0172482 | P21549 | |
| | Hyperoxaluria | | | | |
| | Type I | | | | |
| LIPA | Lysosomal Acid | 3988 | 0107798 | P38571 | Lyososomal |
| | Lipase | | | A0A0A0MT32 | storage |
| | Deficiency | | | | disorder |
| SERPING1 | Hereditary | 710 | 0149131 | P05155, | |
| | Angioedma | | | A0A0S2Z4J1, | |
| | | | | B2R659, | |
| | | | | E7EWE5, | |
| | | | | B3KSP2, | |
| | | | | G5E9S2 | |
| HSD17B4 | D-Bifunctional | 3295 | 0133835 | P51659 | Peroxisomal |
| | Protein | | | | disorders |
| | Deficiency | | | | |
| | X-linked | | | | |
| | Adrenoleukodys | | | | |
| | trophy | | | | |
| UROD | Porphyria | 7389 | 0126088 | P06132 | |
| | Cutanea Tarda | | | | |
| HFE | Porphyria | 3077 | 0010704 | Q30201 | |
| | Cutanea Tarda | | | | |
| LPL | Lipoprotein | 4023 | 0175445 | P06858, | |
| | Lipase | | | A0A1BIRVA9 | |
| | Deficiency | | | | |
| | (hyperlipoprote | | | | |
| | inemia type Ia; | | | | |
| | Buerger-Gruetz | | | | |
| | syndrome, or | | | | |
| | Familial | | | | |
| | hyperchylomicronemia) | | | | |
| GRHPR | Primary | 9380 | 0137106 | Q9UBQ7 | |
| | Hyperoxaluria | | | | |
| | Type II | | | | |
| HOGA1 | Primary | 112817 | 0241935 | Q86XE5 | |
| | Hyperoxaluria | | | | |
| | Type III | | | | |
| LDLR | Homozygous | 3949 | 0130164 | P01130, | |
| | Familial | | | A0A024R7D5 | |
| | Hypercholestero | | | | |
| | lemia | | | | |
| ACAD8 | isobutyryl-CoA | 27034 | 0151498 | Q9UKU7 | Organic |
| | dehydrogenase | | | | acidemia |
| | (IBD) | | | | |
| | deficiency | | | | |
| ACADSB | short-branched | 36 | 0196177 | P45954, | Organic |
| | chain acyl-CoA | | | A0A0S2Z3P9 | acidemia |
| | dehydrogenase | | | | |
| | (SBCAD) | | | | |
| | deficiency | | | | |
| ACAT1 | beta- | 38 | 0075239 | A0A140VJX1, | Organic |
| | ketothiolase | | | P24752 | acidemia |
| | deficiency | | | | |
| ACSF3 | combined | 197322 | 0176715 | Q4G176, | Organic |
| | malonic and | | | F5H5A1 | acidemia |
| | methylmalonic | | | | |
| | aciduria | | | | |
| ASPA | Canavan disease | 443 | 0108381 | P45381, | Organic |
| | | | | Q6FH48 | acidemia |
| AUH | 3-methylglutaconic | 549 | 0148090 | Q13825, | Organic |
| | acidemia type I | | | B4DYI6 | acidemia |
| DNAJC19 | dilated | 131118 | 0205981 | Q96DA6, | Organic |
| | cardiomyopathy | | | A0AOS2Z5X1 | acidemia |
| | with ataxia | | | | |
| | syndrome | | | | |
| | (causes 3- | | | | |
| | methylglutaconic | | | | |
| | aciduria) | | | | |
| ETHE1 | ethylmalonic | 23474 | 0105755 | A0A0S2Z580, | Organic |
| | encephalopathy | | | O95571, | acidemia |
| | | | | A0A0S2Z5N8, | |
| | | | | A0A0S2Z5B3, | |
| | | | | B2RCZ7 | |
| FBP1 | fructose 1,6- | 2203 | 0165140 | P09467, | Organic |
| | Bisphosphatase | | | Q2TU34 | acidemia |
| | deficiency | | | | |
| FTCD | glutamate | 10841 | 0160282, | O95954 | Organic |
| | formiminotransferase | | 0281775 | | acidemia |
| | deficiency | | | | |
| | (FIGLU | | | | |
| GSS | glutathione | 2937 | 0100983 | P48637, | Organic |
| | synthetase | | | V9HWJ1 | acidemia |
| | deficiency | | | | |
| HIBCH | 3-hyroxyisobutyry | 26275 | 0198130 | A0A140VJL0, | Organic |
| | 1-CoA hydrolase | | | Q6NVY1 | acidemia |
| | deficiency | | | | |
| IDH2 | D-2- | 3418 | 0182054 | P48735, | Organic |
| | hydroxyglutaric | | | B4DSZ6 | acidemia |
| | aciduria type II | | | | |
| L2HGDH | L-2- | 79944 | 0087299 | Q9H9P8 | Organic |
| | hydroxyglutaric | | | | acidemia |
| | aciduria | | | | |
| MLYCD | malonic | 23417 | 0103150 | O95822 | Organic |
| | acidemia | | | | acidemia |
| OPA3 | Costeff | 80207 | 0125741 | Q9H6K4, | Organic |
| | syndrome/ | | | B4DK77 | acidemia |
| | 3-methylglutaconic | | | | |
| | aciduria type III | | | | |
| OPLAH | 5-oxoprolinase | 26873 | 0178814 | O14841 | Organic |
| | deficiency | | | | acidemia |
| OXCT1 | SCOT deficiency | 5019 | 0083720 | A0A024R040, | Organic |
| | | | | P55809 | acidemia |
| POLG | 3-methylglutaconic | 5428 | 0140521 | E5KNU5, | Organic |
| | aciduria | | | P54098 | acidemia |
| PPM1K | maple syrup | 152926 | 0163644 | Q8N3J5 | Organic |
| | urine disease | | | | acidemia |
| | (MSUD), | | | | |
| | variant type | | | | |
| SERAC1 | Megdel | 84947 | 0122335 | Q96JX3 | Organic |
| | Syndrome | | | | acidemia |
| SLC25A1 | D,L-2- | 6576 | 0100075 | D9HTE9, | Organic |
| | hydroxyglutaric | | | B4DP62, | acidemia |
| | aciduria | | | P53007 | |
| SUCLA2 | succinate-CoA | 8803 | 0136143 | E5KS60, | Organic |
| | ligase | | | Q9P2R7, | acidemia |
| | deficiency, | | | Q9Y4T0 | |
| | methylmalonic | | | | |
| | aciduria | | | | |
| SUCLG1 | succinate-CoA | 8802 | 0163541 | P53597 | Organic |
| | ligase | | | | acidemia |
| | deficiency, | | | | |
| | methylmalonic | | | | |
| | aciduria | | | | |
| TAZ | Barth syndrome | 6901 | 0102125 | A0A0S2Z4K0, | Organic |
| | | | | Q16635, | acidemia |
| | | | | A6XNE1, | |
| | | | | A0A0S2Z4E6, | |
| | | | | A0A0S2Z4K9 | |
| | | | | A0A0S2Z4F4 | |
| AGK | 3-methylglutaconic | 55750 | 0006530, | A4D1U5, | Organic |
| | aciduria | | 0262327 | Q53H12 | acidemia |
| CLPB | 3-methylglutaconic | 81570 | 0162129 | Q9H078, | Organic |
| | aciduria | | | A0A140VK11 | acidemia |
| TMEM70 | 3-methylglutaconic | 54968 | 0175606 | Q9BUB7 | Organic |
| | aciduria | | | | acidemia |
| ALDH18A1 | ALDH18A1- | 5832 | 0059573 | P54886 | Urea cycle |
| | related cutis | | | | disorder |
| | laxa | | | | |
| OAT | gyrate atrophy | 4942 | 0065154 | A0A140VJQ4, | Urea cycle |
| | (OAT) | | | P04181 | disorder |
| CA5A | carbonic | 763 | 0174990 | P35218 | Urea cycle |
| | anhydrase | | | | disorder |
| | deficiency | | | | |
| GLUD1 | glutamate | 2746 | 0148672 | P00367, | Urea cycle |
| | dehydrogenase | | | E9KL48 | disorder |
| | deficiency | | | | |
| GLUL | glutamine | 2752 | 0135821 | A8YXX4, | Urea cycle |
| | synthetase | | | P15104 | disorder |
| | deficienc | | | | |
| UMPS | Orotic Aciduria | 7372 | 0114491 | A8K5J1, | Urea cycle |
| | | | | P11172 | disorder |
| SLC22A5 | carnitine- | 6584 | 0197375 | O76082 | Fatty acid |
| | acylcarnitine | | | | oxidation |
| | translocase | | | | |
| | (CACT) | | | | |
| | deficiency | | | | |
| CPTIA | carnitine | 1374 | 0110090 | P50416, | Fatty acid |
| | palmitoyltransfe | | | A0A024R5F4, | oxidation |
| | rase type I | | | B2RAQ8, | |
| | (CPT I) | | | Q8WZ48 | |
| | deficiency | | | | |
| HADHA | long chain 3- | 3030 | 0084754 | E9KL44, | Fatty acid |
| | hydroxyacyl- | | | P40939 | oxidation |
| | CoA | | | | |
| | dehydrogenase | | | | |
| | (LCHAD) | | | | |
| | deficiency | | | | |
| HADH | medium/short | 3033 | 0138796 | Q16836, | Fatty acid |
| | chain acyl-CoA | | | B3KTT6 | oxidation |
| | dehydrogenase | | | | |
| | (M/SCHAD) | | | | |
| | deficiency | | | | |
| SLC52A1 | Riboflavin | 55065 | 0132517 | Q9NWF4 | Fatty acid |
| | transporter | | | | oxidation |
| | deficiency | | | | |
| SLC52A2 | Riboflavin | 79581 | 0185803 | Q9HAB3 | Fatty acid |
| | transporter | | | | oxidation |
| | deficiency | | | | |
| SLC52A3 | Riboflavin | 113278 | 0101276 | K0A6P4, | Fatty acid |
| | transporter | | | Q9NQ40 | oxidation |
| | deficiency | | | | |
| HADHB | Trifunctional | 3032 | 0138029 | P55084, | Fatty acid |
| | protein | | | F5GZQ3 | oxidation |
| | deficiency | | | | |
| GYS2 | GSD 0 | 2998 | 0111713 | P54840 | Liver |
| | (Glycogen | | | | glycogen |
| | synthase, liver | | | | storage |
| | isoform) | | | | disorder |
| PYGL | GSD VI (Hers | 5836 | 0100504 | P06737 | Liver |
| | disease) | | | | glycogen |
| | | | | | storage |
| | | | | | disorder |
| SLC2A2 | Fanconi-Bickel | 6514 | 0163581 | P11168, | Liver |
| | syndrome | | | Q6PAU8 | glycogen |
| | | | | | storage |
| | | | | | disorder |
| ALG1 | ALG1-CDG | 56052 | 0033011 | Q9BT22 | Glycosylation |
| | | | | | disorder |
| ALG2 | ALG2- | 85365 | 0119523 | A0A024R184, | Glycosylation |
| | associated | | | Q9H553 | disorder |
| | myasthenic | | | | |
| | syndrome | | | | |
| ALG3 | ALG3-CDG | 10195 | 0214160 | Q92685, | Glycosylation |
| | | | | C9J7S5 | disorder |
| ALG6 | ALG6-CDG | 29929 | 0088035 | Q9Y672 | Glycosylation |
| | | | | | disorder |
| ALG8 | ALG8-CDG | 79053 | 0159063 | Q9BVK2, | Glycosylation |
| | | | | A0A024R5K5 | disorder |
| ALG9 | ALG9-CDG | 79796 | 0086848 | Q9H6U8 | Glycosylation |
| | | | | | disorder |
| ALG11 | ALG11-CDG | 440138 | 0253710 | Q2TAA5 | Glycosylation |
| | | | | | disorder |
| ALG12 | ALG12-CDG | 79087 | 0182858 | A0A024R4V6, | Glycosylation |
| | | | | Q9BV10 | disorder |
| ALG13 | ALG13-CDG | 79868 | 0101901 | Q9NP73, | Glycosylation |
| | | | | A0A087WX43, | disorder |
| | | | | A0A087WT15 | |
| ATP6V0A2 | ATP6V0A2- | 23545 | 0185344 | Q9Y487 | Glycosylation |
| | associated cutis | | | | disorder |
| | laxa | | | | |
| B3GLCT | B3GLCT-CDG | 145173 | 0187676 | Q6Y288 | Glycosylation |
| | | | | | disorder |
| CHST14 | CHST14-CDG | 113189 | 0169105 | Q8NCH0 | Glycosylation |
| | | | | | disorder |
| COG1 | COG1-CDG | 9382 | 0166685 | Q8WTW3 | Glycosylation |
| | | | | | disorder |
| COG2 | COG2-CDG | 22796 | 0135775 | Q14746, | Glycosylation |
| | | | | B1ALW7 | disorder |
| COG4 | COG4-CDG | 25839 | 0103051 | A0A0A0MS45, | Glycosylation |
| | | | | Q8N8L9, | disorder |
| | | | | Q9H9E3, | |
| | | | | J3KNI1 | |
| COG5 | COG5-CDG | 10466 | 0164597, | Q9UP83 | Glycosylation |
| | | | 0284369 | | disorder |
| COG6 | COG6-CDG | 57511 | 0133103 | A0A140VJG7, | Glycosylation |
| | | | | Q9Y2V7, | disorder |
| | | | | A0A024RDW5 | |
| COG7 | COG7-CDG | 91949 | 0168434 | A0A0S2Z652, | Glycosylation |
| | | | | P83436 | disorder |
| COG8 | COG8-CDG | 84342 | 0272617 | A0A024R6Z6, | Glycosylation |
| | | | | Q96MW5 | disorder |
| DOLK | DOLK-CDG | 22845 | 0175283 | A0A0S2Z597, | Glycosylation |
| | | | | Q9UPQ8 | disorder |
| DHDDS | DHDDS-CDG | 79947 | 0117682 | Q86SQ9 | Glycosylation |
| | | | | | disorder |
| DPAG | DPAGTI-CDG | 1798 | 0172269 | A0A024R3H8, | Glycosylation |
| | | | | Q9H3H5 | disorder |
| DPM1 | DPM1-CDG | 8813 | 0000419 | O60762, | Glycosylation |
| | | | | Q5QPK2, | disorder |
| | | | | A0A0S2Z4Y5 | |
| DPM2 | DPM2-CDG | 8818 | 0136908 | O94777 | Glycosylation |
| | | | | | disorder |
| DPM3 | DPM3-CDG | 54344 | 0179085 | A0A140VJI4, | Glycosylation |
| | | | | Q9P2X0, | disorder |
| | | | | Q86TM7 | |
| G6PC3 | Congenital | 92579 | 0141349 | Q9BUM1 | Glycosylation |
| | neutropenia | | | | disorder |
| GFPT1 | Congenital | 2673 | 0198380 | Q06210 | Glycosylation |
| | myasthenic | | | | disorder |
| | syndrome | | | | |
| GMPPA | GMPPA-CDG | 29926 | 0144591 | A0A024R482, | Glycosylation |
| | | | | Q96IJ6 | disorder |
| GMPPB | Congenital | 29925 | 0173540 | Q9Y5P6 | Glycosylation |
| | muscular dystrophy, | | | | disorder |
| | congenital myasthenic | | | | |
| | syndrome, and | | | | |
| | dystroglycanopathy | | | | |
| MAGT1 | MAGT1-CDG; | 84061 | 0102158 | A0A087WU53, | Glycosylation |
| | X-linked | | | Q9H0U3 | disorder |
| | immunodeficiency | | | | |
| | with magnesium | | | | |
| | defect, Epstein-Barr | | | | |
| | virus infection and | | | | |
| | neoplasia (XMEN) | | | | |
| | syndrome | | | | |
| MAN1B1 | MAN1B1-CDG | 11253 | 0177239 | Q9UKM7 | Glycosylation |
| | | | | | disorder |
| MGAT2 | MGAT2-CDG | 4247 | 0168282 | Q10469 | Glycosylation |
| | | | | | disorder |
| MOGS | MOGS-CDG | 7841 | 0115275 | Q13724, | Glycosylation |
| | | | | Q58F09 | disorder |
| MPDU1 | MPDU1-CDG | 9526 | 0129255 | J3QW43, | Glycosylation |
| | | | | O75352, | disorder |
| | | | | A0A0S2Z4W8, | |
| | | | | B4DLH7 | |
| MPI | MPI-CDG | 4351 | 0178802 | H3BPP3, | Glycosylation |
| | | | | Q8NHZ6, | disorder |
| | | | | B4DW50, | |
| | | | | F5GX71, | |
| | | | | P34949, | |
| | | | | H3BPB8 | |
| NGLY1 | NGLY1-CDG | 55768 | 0151092 | Q96IV0 | Glycosylation |
| | | | | | disorder |
| PGM1 | PGM1-CDG | 5236 | 0079739 | B7Z6C2, | Glycosylation |
| | | | | P36871, | disorder |
| | | | | B4DDQ8 | |
| PGM3 | PGM3-CDG | 5238 | 0013375 | O95394, | Glycosylation |
| | | | | A0A087WT27 | disorder |
| RFT1 | RFT1-CDG | 91869 | 0163933 | Q96AA3 | Glycosylation |
| | | | | | disorder |
| SEC23B | SEC23B-CDG | 10483 | 0101310 | Q15437, | Glycosylation |
| | | | | B4DJW8 | disorder |
| SLC35A1 | SLC35A1-CDG | 10559 | 0164414 | P78382 | Glycosylation |
| | | | | | disorder |
| SLC35A2 | SLC35A2-CDG | 7355 | 0102100 | P78381, | Glycosylation |
| | | | | A6NFI1, | disorder |
| | | | | A6NKM8, | |
| | | | | B4DE15 | |
| SLC35C1 | SLC35C1-CDG | 55343 | 0181830 | Q96A29, | Glycosylation |
| | | | | B3KQH0 | disorder |
| SSR4 | SSR4-CDG | 6748 | 0180879 | P51571 | Glycosylation |
| | | | | | disorder |
| SRD5A3 | SRD5A3-CDG | 79644 | 0128039 | Q9H8P0 | Glycosylation |
| | | | | | disorder |
| TMEM165 | TMEM165-CDG | 55858 | 0134851 | Q9HC07 | Glycosylation |
| | | | | | disorder |
| TRIP11 | TRIP11-CDG | 9321 | 0100815 | Q15643 | Glycosylation |
| | | | | | disorder |
| TUSC3 | TUSC3-CDG | 799] | 0104723 | Q13454 | Glycosylation |
| | | | | | disorder |
| ALG14 | ALG14-CDG | 199857 | 0172339 | Q96F25 | Glycosylation |
| | | | | | disorder |
| B4GALT1 | B4GALT1-CDG | 2683 | 0086062 | P15291, | Glycosylation |
| | | | | W6MEN3 | disorder |
| DDOST | DDOST-CDG | 1650 | 0244038 | A0A024RAD5, | Glycosylation |
| | | | | P39656 | disorder |
| NUS1 | NUS1-CDG | 116150 | 0153989 | Q96E22 | Glycosylation |
| | | | | | disorder |
| RPN2 | RPN2-CDG | 6185 | 0118705 | P04844 | Glycosylation |
| | | | | | disorder |
| SEC23A | SEC23A-CDG | 10484 | 0100934 | Q15436 | Glycosylation |
| | | | | | disorder |
| SLC35A3 | SLC35A3-CDG | 23443 | 0117620 | Q9Y2D2, | Glycosylation |
| | | | | A0A1W2PRT7, | disorder |
| | | | | A0A1W2PSD1, | |
| | | | | A0A1W2PQL8 | |
| ST3GAL3 | ST3GAL3-CDG | 6487 | 0126091 | Q11203 | Glycosylation |
| | | | | | disorder |
| STT3A | STT3A-CDG | 3703 | 0134910 | P46977 | Glycosylation |
| | | | | | disorder |
| STT3B | STT3B-CDG | 201595 | 0163527 | Q8TCJ2 | Glycosylation |
| | | | | | disorder |
| AGA | Aspartylglucosaminuria | 175 | 0038002 | P20933 | Lyososo mal |
| | | | | | storage |
| | | | | | disorder |
| ARSA | Metachromatic | 410 | 0100299 | A0A0C4DFZ2, | Lyososomal |
| | leukodystrophy | | | B4DVI5, | storage |
| | | | | P15289 | disorder |
| ARSB | Mucopolysacch | 411 | 0113273 | A0A024RAJ9, | Lyososomal |
| | aridosis type VI | | | P15848, | storage |
| | | | | A8K4A0 | disorder |
| ASAH1 | Farber disease | 427 | 0104763 | A8K0B6, | Lyososomal |
| | | | | Q13510, | storage |
| | | | | Q53H01 | disorder |
| ATP13A2 | Neuronal ceroid | 23400 | 0159363 | Q8N4D4, | Lyososomal |
| | lipofuscinosis 12 | | | Q9NQ11, | storage |
| | (CLN12), | | | Q8NBS1 | disorder |
| | Kufor-Rakeb | | | | |
| | syndrome | | | | |
| | (KRS) | | | | |
| CLN3 | Neuronal ceroid | 1201 | 0188603, | A0A024QZB8, | Lyososomal |
| | lipofuscinosis 3 | | 0261832 | Q13286, | storage |
| | (CLN3) | | | B4DMY6, | disorder |
| | | | | Q2TA70, | |
| | | | | B4DFF3 | |
| CLN5 | Neuronal ceroid | 1203 | 0102805 | A0A024R644, | Lyososomal |
| | lipofuscinosis 5 | | | O75503 | storage |
| | (CLN5) | | | | disorder |
| CLN6 | Neuronal ceroid | 54982 | 0128973 | A0A024R601, | Lyososomal |
| | lipofuscinosis 6 | | | Q9NWW5 | storage |
| | (CLN6) | | | | disorder |
| CLN8 | Neuronal ceroid | 2055 | 0182372, | A0A024QZ57, | Lyososomal |
| | lipofuscinosis 8 | | 0278220 | Q9UBY8 | storage |
| | (CLN8) | | | | disorder |
| CTNS | cystinosis | 1497 | 0040531 | A0A0S2Z319, | Lyososomal |
| | | | | O60931, | storage |
| | | | | A0A0S2Z3K3 | disorder |
| CTSA | Galactosialidosis | 5476 | 0064601 | P10619, | Lyososomal |
| | | | | X6R8A1, | storage |
| | | | | B4E324, | disorder |
| | | | | X6R5C5 | |
| CTSD | Neuronal ceroid | 1509 | 0117984 | P07339, | Lyososomal |
| | lipofuscinosis | | | V9HWI3 | storage |
| | 10 (CLN10) | | | | disorder |
| CTSF | Neuronal ceroid | 8722 | 0174080 | Q9UBX1 | Lyososomal |
| | lipofuscinosis | | | | storage |
| | 13 (CLN13) | | | | disorder |
| CTSK | Pycnodysostosis | 1513 | 0143387 | P43235 | Lyososomal |
| | | | | | storage |
| | | | | | disorder |
| DNAJC5 | Neuronal ceroid | 80331 | 0101152 | Q6AHX3, | Lyososomal |
| | lipofuscinosis 4 | | | Q9H3Z4 | storage |
| | (CLN4) | | | | disorder |
| FUCA1 | Fucosidosis | 2517 | 0179163 | P04066, | Lyososomal |
| | | | | B5MDC5 | storage |
| | | | | | disorder |
| GAA | Pompe disease | 2548 | 0171298 | P10253 | Lyososomal |
| | | | | | storage |
| | | | | | disorder |
| GALC | Krabbe disease | 2581 | 0054983 | A0A0A0MQV0, | Lyososomal |
| | | | | P54803 | storage |
| | | | | | disorder |
| GALNS | Mucopolysaccharidosis | 2588 | 0141012 | P34059, | Lyososomal |
| | type IVa | | | Q96I49, | storage |
| | | | | Q6YL38 | disorder |
| GLA | Fabry disease | 2717 | 0102393 | P06280, | Lyososomal |
| | | | | Q53Y83 | storage |
| | | | | | disorder |
| GLB1 | GM1 | 2720 | 0170266 | P16278, | Lyososomal |
| | gangliosidosis, | | | B7Z6Q5 | storage |
| | Mucopolysacch | | | | disorder |
| | aridosis IVb | | | | |
| GM2A | GM2- | 2760 | 0196743 | P17900 | Lyososomal |
| | gangliosidosis, | | | | storage |
| | AB variant | | | | disorder |
| GNPTAB | Mucolipidosis | 79158 | 0111670 | Q3T906 | Lyososomal |
| | type II alpha/beta, | | | | storage |
| | Mucolipidosis III | | | | disorder |
| | alpha/beta | | | | |
| GNPTG | Mucolipidosis III | 84572 | 0090581 | Q9UJJ9 | Lyososomal |
| | gamma | | | | storage |
| | | | | | disorder |
| GNS | Mucopolysacch | 2799 | 0135677 | A0A024RBC5, | Lyososomal |
| | aridosis | | | P15586, | storage |
| | type IIID | | | Q7Z3X3 | disorder |
| GRN | Neuronal ceroid | 2896 | 0030582 | P28799 | Lyososomal |
| | lipofuscinosis 11 | | | | storage |
| | (CLN11), | | | | disorder |
| | frontotemporal | | | | |
| | dementia | | | | |
| GUSB | Mucopolysacch | 2990 | 0169919 | P08236 | Lyososomal |
| | aridosis type VII | | | | storage |
| | | | | | disorder |
| HEXA | Tay-Sachs | 3073 | 0213614 | A0A0S2Z3W3, | Lyososomal |
| | disease | | | P06865, | storage |
| | | | | B4DVA7, | disorder |
| | | | | H3BP20 | |
| HEXB | Sandhoff | 3074 | 0049860 | A0A024RAJ6, | Lyososomal |
| | diseaase | | | P07686, | storage |
| | | | | Q5URX0 | disorder |
| HGSNAT | Mucopolysacch | 138050 | 0165102 | Q68CP4, | Lyososomal |
| | aridosis type IIIC | | | Q8IVU6 | storage |
| | | | | | disorder |
| HYAL1 | Mucopolysaccharidosis | 3373 | 0114378 | A0A024R2X3, | Lyososomal |
| | type IX | | | Q12794, | storage |
| | | | | B3KUI5, | disorder |
| | | | | A0A0S2Z3Q0 | |
| IDS | Mucopolysaccharidosis | 3423 | 0010404 | P22304, | Lyososomal |
| | type II | | | B4DGD7 | storage |
| | | | | | disorder |
| IDUA | Mucopolysaccharidosis | 3425 | 0127415 | P35475 | Lyososomal |
| | type I | | | | storage |
| | | | | | disorder |
| KCTD7 | Neuronal ceroid | 154881 | 0243335 | Q96MP8, | Lyososomal |
| | lipofuscinosis 14 | | | A0A024RDN7 | storage |
| | (CLN14) | | | | disorder |
| LAMP2 | Danon disease | 3920 | 0005893 | P13473 | Lyososomal |
| | | | | | storage |
| | | | | | disorder |
| MAN2B1 | alpha-mannosidosis | 4125 | 0104774 | O00754, | Lyososomal |
| | | | | A8K6A7 | storage |
| | | | | | disorder |
| MANBA | beta-mannosidosis | 4126 | 0109323 | O00462 | Lyososomal |
| | | | | | storage |
| | | | | | disorder |
| MCOLN1 | Mucolipidosis | 57192 | 0090674 | Q9GZU1 | Lyososomal |
| | type IV | | | | storage |
| | | | | | disorder |
| MFSD8 | Neuronal ceroid | 256471 | 0164073 | Q8NHS3 | Lyososomal |
| | lipofuscinosis 7 | | | | storage |
| | (CLN7) | | | | disorder |
| NAGA | Schindler | 4668 | 0198951 | A0A024RIQ5, | Lyososomal |
| | disease | | | P17050 | storage |
| | | | | | disorder |
| NAGLU | Mucopolysacch | 4669 | 0108784 | A0A140VJE4, | Lyososomal |
| | aridosis IIIB | | | P54802 | storage |
| | | | | | disorder |
| NEU1 | Mucolipidosis | 4758 | 0204386, 0227315, | Q5JQI0, | Lyososomal |
| | type I, | | 0227129, 0223957, | Q99519 | storage |
| | Sialidosis I | | 0234846, 0184494, | | disorder |
| | | | 0228691, 0234343 | | |
| NPC1 | Niemann-Pick | 4864 | 0141458 | O15118 | Lyososomal |
| | type C | | | | storage |
| | | | | | disorder |
| NPC2 | Niemann-Pick | 10577 | 0119655 | A0A024R6C0, | Lyososomal |
| | type C | | | P61916, | storage |
| | | | | G3V3E8 | disorder |
| SGSH | Mucopolysaccharidosis | 6448 | 0181523 | P51688 | Lyososomal |
| | IIIA | | | | storage |
| | | | | | disorder |
| PPT1 | Neuronal ceroid | 5538 | 0131238 | P50897 | Lyososomal |
| | lipofuscinosis 1 | | | | storage |
| | (CLN1) | | | | disorder |
| PSAP | Prosaposin | 5660 | 0197746 | P07602, | Lyososomal |
| | deficiency, | | | A0A024QZQ2 | storage |
| | SapA deficiency | | | | disorder |
| | (Krabbe variant), | | | | |
| | SapB deficiency | | | | |
| | (MLD variant), | | | | |
| | SapC deficiency | | | | |
| | (Gaucher variant) | | | | |
| SLC17A5 | Infantile sialic | 26503 | 0119899 | Q9NRA2 | Lyososomal |
| | acid storage | | | | storage |
| | disease, Salla | | | | disorder |
| | disease | | | | |
| SMPD1 | Niemann Pick | 6609 | 0166311 | P17405, | Lyososomal |
| | types A and B | | | Q59EN6, | storage |
| | | | | E9LUE8, | disorder |
| | | | | Q8IUN0, | |
| | | | | E9LUE9 | |
| SUMF1 | Multiple | 285362 | 0144455 | Q8NBK3 | Lyososomal |
| | sulfatase | | | | storage |
| | deficiency | | | | disorder |
| TPP1 | Neuronal ceroid | 1200 | 0166340 | O14773 | Lyososomal |
| | lipofuscinosis 2 | | | | storage |
| | (CLN2) | | | | disorder |
| AHCY | Hypermethioninemia | 191 | 0101444 | P23526, | Aminoacidophaty |
| | | | | Q1RMG2 | |
| GNMT | Hypermethioninemia | 27232 | 0124713 | A0A0S2Z5F2, | Aminoacidophaty |
| | | | | Q14749, | |
| | | | | V9HW60 | |
| MATIA | Hypermethioninemia | 4143 | 0151224 | Q00266 | Aminoacidophaty |
| GCH1 | BH4 cofactor | 2643 | 0131979 | A0A024R642 | Aminoacidophaty |
| | deficiency | | | P30793, | |
| | | | | Q8IZH9 | |
| PCBD1 | BH4 cofactor | 5092 | 0166228 | P61457 | Aminoacidophaty |
| | deficiency | | | | |
| PTS | BH4 cofactor | 5805 | 0150787 | Q03393 | Aminoacidophaty |
| | deficiency | | | | |
| QDPR | BH4 cofactor | 5860 | 0151552 | A0A140VKA9, | Aminoacidophaty |
| | deficiency | | | P09417 | |
| SPR | BH4 cofactor | 6697 | 0116096 | P35270 | Aminoacidophaty |
| | deficiency | | | | |
| DNAJC12 | Phenylalanine, | 56521 | 0108176 | Q6IAH1, | Aminoacidophaty |
| | tyrosine, and | | | Q9UKB3 | |
| | tryptophan | | | | |
| | hydroxylases | | | | |
| | heat shock | | | | |
| | co-chaperone | | | | |
| | deficiency | | | | |
| ALDH4A1 | Hyperprolinemia | 8659 | 0159423 | P30038, | Aminoacidophaty |
| | | | | A0A024RAD8 | |
| PRODH | Hyperprolinemia | 5625 | 0100033 | O43272 | Aminoacidophaty |
| HPD | Tyrosinemia | 3242 | 0158104 | P32754 | Aminoacidophaty |
| | type II | | | | |
| GBA | Gaucher disease | 2629 | 0177628, | A0A068F658, | |
| | | | 0262446 | P04062, | |
| | | | | B7Z6S9 | |
| HGD | Alkaptonuria | 3081 | 0113924 | Q93099, | |
| | | | | B3KW64 | |
| AMN | Combined | 81693 | 0166126 | Q9BXJ7, | Organic |
| | Methylmalonic | | | B3KP64 | acidemia |
| | Acidemia and | | | | |
| | Homocystinuria | | | | |
| CD320 | Combined | 51293 | 0167775 | Q9NPF0 | Organic |
| | Methylmalonic | | | | acidemia |
| | Acidemia and | | | | |
| | Homocystinuria | | | | |
| CUBN | Combined | 8029 | 0107611 | O60494 | Organic |
| | Methylmalonic | | | | acidemia |
| | Acidemia and | | | | |
| | Homocystinuria | | | | |
| GIF | Combined | 2694 | 0134812 | P27352 | Organic |
| | Methylmalonic | | | | acidemia |
| | Acidemia and | | | | |
| | Homocystinuria | | | | |
| TCN1 | Combined | 6947 | 0134827 | P20061 | Organic |
| | Methylmalonic | | | | acidemia |
| | Acidemia and | | | | |
| | Homocystinuria | | | | |
| TCN2 | Combined | 6948 | 0185339 | P20062 | Organic |
| | Methylmalonic | | | | acidemia |
| | Acidemia and | | | | |
| | Homocystinuria | | | | |
| PREPL | Cystinuria | 9581 | 0138078 | Q4J6C6 | Aminoacidophaty |
| PHGDH | Disorders of | 26227 | 0092621 | O43175 | Aminoacidophaty |
| | Serine | | | | |
| | Biosynthesis | | | | |
| PSAT1 | Disorders of | 29968 | 0135069 | A0A024R280, | Aminoacidophaty |
| | Serine | | | Q9Y617, | |
| | Biosynthesis | | | A0A024R222 | |
| PSPH | Disorders of | 5723 | 0146733 | A0A024RDL3, | Aminoacidophaty |
| | Serine | | | P78330 | |
| | Biosynthesis | | | | |
| AMT | Glycine | 275 | 0145020 | A0A024R2U7, | Aminoacidophaty |
| | Encephalopathy | | | P48728 | |
| GCSH | Glycine | 2653 | 0140905 | P23434 | Aminoacidophaty |
| | Encephalopathy | | | | |
| GLDC | Glycine | 2731 | 0178445 | P23378 | Aminoacidophaty |
| | Encephalopathy | | | | |
| LIAS | Glycine | 11019 | 0121897 | O43766, | Aminoacidophaty |
| | Encephalopathy | | | Q6P5Q6, | |
| | | | | B4E0L7, | |
| | | | | A0A024R9W0. | |
| | | | | A0A1W2PQE9, | |
| | | | | A0A1X7SBR7 | |
| NFU1 | Glycine | 27247 | 0169599 | Q9UMS0 | Aminoacidophaty |
| | Encephalopathy | | | | |
| SLC6A9 | Glycine | 6536 | 0196517 | P48067, | Aminoacidophaty |
| | Encephalopathy | | | B7Z3W8, | |
| | | | | B7Z589 | |
| SLC2A1 | Glucose | 6513 | 0117394 | P11166, | Carbohydrate |
| | Transporter | | | Q59GX2 | disorder |
| | Type 1 | | | | |
| | Deficiency | | | | |
| ATP7A | ATP7A-Related | 538 | 0165240 | B4DRW0, | Metal |
| | Disorders Copper | | | Q04656, | transport |
| | | | | Q762B6 | disorder |
| | Metabolism | | | | |
| | Disorder | | | | |
| APIS1 | Copper | 1174 | 0106367 | A0A024QYT6, | Metal |
| | Metabolism | | | P61966 | transport |
| | Disorder | | | | disorder |
| CP | Copper | 1356 | 0047457 | A5PL27, | Metal |
| | Metabolism | | | P00450 | transport |
| | Disorder | | | | disorder |
| SLC33A1 | Copper | 9197 | 0169359 | O00400 | Metal |
| | Metabolism | | | | transport |
| | Disorder | | | | disorder |
| PEX7 | Adult Refsum | 5191 | 0112357 | O00628, | Peroxisomal |
| | Disease | | | Q6FGN1 | disorders |
| | Rhizomelic | | | | |
| | Chondrodysplasia | | | | |
| | Punctata | | | | |
| | Spectrum | | | | |
| PHYH | Adult Refsum | 5264 | 0107537 | O14832 | Peroxisomal |
| | Disease | | | | disorders |
| AGPS | Rhizomelic | 8540 | 0018510 | O00116, | Peroxisomal |
| | Chondrodysplasia | | | B7Z3Q4 | disorders |
| | Punctata | | | | |
| | Spectrum | | | | |
| GNPAT | Rhizomelic | 8443 | 0116906 | O15228 | Peroxisomal |
| | Chondrodysplasia | | | | disorders |
| | Punctata | | | | |
| | Spectrum | | | | |
| ABCD1 | X-linked | 215 | 0101986 | P33897 | Peroxisomal |
| | Adrenoleukodystrophy | | | | disorders |
| ACOX1 | X-linked | 51 | 0161533 | Q15067 | Peroxisomal |
| | Adrenoleukodystrophy | | | | disorders |
| PEX1 | X-linked | 5189 | 0127980 | O43933, | Peroxisomal |
| | Adrenoleukodystrophy | | | A0A0C4DG33, | disorders |
| | | | | B4DER6 | |
| PEX2 | X-linked | 5828 | 0164751 | P28328 | Peroxisomal |
| | Adrenoleukodystrophy | | | | disorders |
| PEX3 | X-linked | 8504 | 0034693 | P56589 | Peroxisomal |
| | Adrenoleukodystrophy | | | | disorders |
| PEX5 | X-linked | 5830 | 0139197 | A0A0S2Z480, | Peroxisomal |
| | Adrenoleukodystrophy | | | P50542, | disorders |
| | | | | B4DR50, | |
| | | | | A0A0S2Z4F3, | |
| | | | | A0A0S2Z4H1, | |
| | | | | B4E0T2 | |
| PEX6 | X-linked | 5190 | 0124587 | A0A024RD09, | Peroxisomal |
| | Adrenoleukodystrophy | | | Q13608 | disorders |
| PEX10 | X-linked | 5192 | 0157911 | A0A024R068, | Peroxisomal |
| | Adrenoleukodystrophy | | | O60683, | disorders |
| | | | | A0A024R0A4 | |
| PEX12 | X-linked | 5193 | 0108733 | O00623 | Peroxisomal |
| | Adrenoleukodystrophy | | | | disorders |
| PEX13 | X-linked | 5194 | 0162928 | Q92968 | Peroxisomal |
| | Adrenoleukodystrophy | | | | disorders |
| PEX14 | X-linked | 5195 | 0142655 | O75381 | Peroxisomal |
| | Adrenoleukodystrophy | | | | disorders |
| PEX16 | X-linked | 9409 | 0121680 | Q9Y5Y5 | Peroxisomal |
| | Adrenoleukodystrophy | | | | disorders |
| PEX19 | X-linked | 5824 | 0162735 | P40855, | Peroxisomal |
| | Adrenoleukodystrophy | | | A0A0S2Z497 | disorders |
| PEX26 | X-linked | 55670 | 0215193 | A0A024R100, | Peroxisomal |
| | Adrenoleukodystrophy | | | Q7Z412, | disorders |
| | | | | A0A0S2Z5M7, | |
| | | | | Q7Z2D7 | |
| AMACR | Zellweger | 23600 | 0242110 | Q9UHK6 | Peroxisomal |
| | Spectrum | | | | disorders |
| | Disorder | | | | |
| ADA | Purine | 100 | 0196839 | A0A0S2Z381, | Purine |
| | Metabolism | | | P00813, | Metabolism |
| | Disorder | | | F5GWI4 | Disorder |
| ADSL | Purine | 158 | 0239900 | P30566, | Purine |
| | Metabolism | | | X5D8S6, | Metabolism |
| | Disorder | | | X5D7W4, | Disorder |
| | | | | A0A1B0GWJ0 | |
| AMPD1 | Purine | 270 | 0116748 | P23109 | Purine |
| | Metabolism | | | | Metabolism |
| | Disorder | | | | Disorder |
| GPHN | Purine | 10243 | 0171723 | Q9NQX3 | Purine |
| | Metabolism | | | | Metabolism |
| | Disorder | | | | Disorder |
| MOCOS | Purine | 55034 | 0075643 | Q96EN8 | Purine |
| | Metabolism | | | | Metabolism |
| | Disorder | | | | Disorder |
| MOCS1 | Purine | 4337 | 0124615 | A0A024RD17, | Purine |
| | Metabolism | | | Q9NZB8 | Metabolism |
| | Disorder | | | | Disorder |
| PNP | Purine | 4860 | 0198805 | P00491 | Purine |
| | Metabolism | | | V9HWH6 | Metabolism |
| | Disorder | | | | Disorder |
| XDH | Purine | 7498 | 0158125 | P47989 | Purine |
| | Metabolism | | | | Metabolism |
| | Disorder | | | | Disorder |
| SUOX | Purine | 6821 | 0139531 | A0A024RB79, | Purine |
| | Metabolism | | | P51687 | Metabolism |
| | Disorder | | | | Disorder |
| OGDH | 2-Ketoglutarate | 4967 | 0105953 | A0A140VJQ5, | PYRUVATE |
| | Dehydrogenase | | | Q02218, | METABOLISM |
| | Deficiency | | | B4E3E9, | AND |
| | | | | E9PCR7, | TRICARBOXYLIC |
| | | | | E9PDF2 | ACID |
| | | | | | CYCLE |
| | | | | | DEFECT |
| SLC25A19 | 2-Ketoglutarate | 60386 | 0125454 | Q5JPC1, | PYRUVATE |
| | Dehydrogenase | | | Q9HC21 | METABOLISM |
| | Deficiency | | | | AND |
| | | | | | TRICARBOXYLIC |
| | | | | | ACID |
| | | | | | CYCLE |
| | | | | | DEFECT |
| DHTKD1 | 2-Ketoglutarate | 55526 | 0181192 | Q96HY7 | PYRUVATE |
| | Dehydrogenase | | | | METABOLISM |
| | Deficiency | | | | AND |
| | | | | | TRICARBOXYLIC |
| | | | | | ACID |
| | | | | | CYCLE |
| | | | | | DEFECT |
| SLC13A5 | Citrate | 284111 | 0141485 | Q68D44, | PYRUVATE |
| | Transporter | | | Q86YT5 | METABOLISM |
| | Deficiency | | | | AND |
| | | | | | TRICARBOXYLIC |
| | | | | | ACID |
| | | | | | CYCLE |
| | | | | | DEFECT |
| FH | Fumarase | 2271 | 0091483 | A0A0S2Z4C3, | PYRUVATE |
| | Deficiency | | | P07954 | METABOLISM |
| | | | | | AND |
| | | | | | TRICARBOXYLIC |
| | | | | | ACID |
| | | | | | CYCLE |
| | | | | | DEFECT |
| DLAT | Pyruvate | 1737 | 0150768 | P10515, | PYRUVATE |
| | Dehydrogenase | | | Q86YI5 | METABOLISM |
| | Deficiency | | | | AND |
| | | | | | TRICARBOXYLIC |
| | | | | | ACID |
| | | | | | CYCLE |
| | | | | | DEFECT |
| MPC1 | Pyruvate | 51660 | 0060762 | Q5TI65, | PYRUVATE |
| | Dehydrogenase | | | Q9Y5U8 | METABOLISM |
| | Deficiency | | | | AND |
| | | | | | TRICARBOXYLIC |
| | | | | | ACID |
| | | | | | CYCLE |
| | | | | | DEFECT |
| PDHA1 | Pyruvate | 5160 | 0131828 | A0A024RBX9, | PYRUVATE |
| | Dehydrogenase | | | P08559 | METABOLISM |
| | Deficiency | | | | AND |
| | | | | | TRICARBOXYLIC |
| | | | | | ACID |
| | | | | | CYCLE |
| | | | | | DEFECT |
| PDHB | Pyruvate | 5162 | 0168291 | P11177 | PYRUVATE |
| | Dehydrogenase | | | | METABOLISM |
| | Deficiency | | | | AND |
| | | | | | TRICARBOXYLIC |
| | | | | | ACID |
| | | | | | CYCLE |
| | | | | | DEFECT |
| PDHX | Pyruvate | 8050 | 0110435 | O00330 | PYRUVATE |
| | Dehydrogenase | | | | METABOLISM |
| | Deficiency | | | | AND |
| | | | | | TRICARBOXYLIC |
| | | | | | ACID |
| | | | | | CYCLE |
| | | | | | DEFECT |
| PDP1 | Pyruvate | 54704 | 0164951 | Q9POJ1, | PYRUVATE |
| | Dehydrogenase | | | Q6PIN1, | METABOLISM |
| | Deficiency | | | A0A024R9C0 | AND |
| | | | | | TRICARBOXYLIC |
| | | | | | ACID |
| | | | | | CYCLE |
| | | | | | DEFECT |
| ABCC2 | Dubin-Johnson | 1244 | 0023839 | Q92887 | |
| | syndrome | | | | |
| SLCO1B1 | Rotor Syndrome | 10599 | 0134538 | A0A024RAU7, | |
| | | | | Q05CV5, | |
| | | | | Q9Y6L6 | |
| SLCO1B3 | Rotor Syndrome | 28234 | 0111700 | B3KP78, | |
| | | | | Q9NPD5 | |
| HFE2 | Hemochromatosis, | 148738 | 0168509 | Q6ZVN8, | |
| | type 2A | | | A8K466, | |
| | | | | A0A024R4F5 | |
| ADAMTS13 | Congenital | 11093 | 0160323, | Q76LX8 | |
| | thrombotic | | 0281244 | | |
| | thrombocytopenic | | | | |
| | purpura due to | | | | |
| | ADAMTS-13 | | | | |
| | deficiency | | | | |
| PYGM | McArdle's | 5837 | 0068976 | P11217 | |
| | Disease | | | | |
| COL1A2 | Ehlers-Danlos | 1278 | 0164692 | A0A0S2Z3H5, | |
| | syndrome, | | | P08123 | |
| | cardiac valvular | | | | |
| | type | | | | |
| TNFRS | Juvenile Paget's | 4982 | 0164761 | O00300 | |
| F11B | disease | | | | |
| TSC1 | Tuberous | 7248 | 0165699 | Q86WV8, | |
| | sclerosis | | | Q92574, | |
| | | | | X5D9D2, | |
| | | | | Q32NF0 | |
| TSC2 | Tuberous | 7249 | 0103197 | P49815, | |
| | sclerosis | | | X5D7Q2, | |
| | | | | B3KWH7, | |
| | | | | Q5HYF7, | |
| | | | | H3BMQ0, | |
| | | | | X5D2U8 | |
| DHCR7 | Smith-Lemli- | 1717 | 0172893 | A0A024R5F7, | |
| | Opitz Syndrome | | | Q9UBM7 | |
| PGK1 | D-glycericacidemia | 5230 | 0102144 | P00558, | |
| | | | | V9HWF4 | |
| VLDLR | Dysequilibrium | 7436 | 0147852 | P98155, | |
| | syndrome | | | Q5VVF5 | |
| KYNU | Encephalopathy | 8942 | 0115919 | Q16719 | |
| | due to | | | | |
| | hydroxykynureninuria | | | | |
| F5 | Factor V | 2153 | 0198734 | P12259 | |
| | deficiency | | | | |
| C3 | Atypical | 718 | 0125730 | B4DR57, | |
| | hemolytic | | | P01024, | |
| | uremic | | | V9HWA9 | |
| | syndrome with | | | | |
| | C3 anomaly | | | | |
| COL4A1 | Autosomal | 1282 | 0187498 | A5PKV2, | |
| | dominant familial | | | F5H5K0, | |
| | hematuria- | | | P02462 | |
| | retinal arteriolar | | | | |
| | tortuosity- | | | | |
| | contractures | | | | |
| CFH | Atypical | 3075 | 0000971 | A0A024R962, | |
| | hemolytic | | | P08603, | |
| | uremic | | | A0A0D9SG88 | |
| | syndrome | | | | |
| SLC12A2 | Bartter | 6558 | 0064651 | P55011, | |
| | syndrome type I | | | Q53ZR1, | |
| | (neonatal) | | | B7ZM24 | |
| GK | Glycerol kinase | 2710 | 0198814 | B4DH54, | |
| | deficiency | | | P32189 | |
| SFTPC | Chronic | 6440 | 0168484 | A0A0A0MTC9, | |
| | respiratory | | | P11686, | |
| | distress with | | | A0A0S2Z4Q0, | |
| | surfactant | | | E5RI64 | |
| | metabolism | | | | |
| | deficiency | | | | |
| CRTAP | Osteogenesis | 10491 | 0170275 | O75718 | |
| | Imperfecta VII | | | | |
| P3H1 | Osteogenesis | 64175 | 0117385 | Q32P28 | |
| | Imperfecta VIII | | | | |
| COL7A1 | Autosomal | 1294 | 0114270 | Q02388, | |
| | recessive | | | Q59F16 | |
| | dystrophic | | | | |
| | epidermolysis | | | | |
| | bullosa | | | | |
| PKLR | Pyruvate Kinase | 5313 | 0143627 | P30613 | |
| | deficiency | | | | |
| TALD01 | Transaldolase | 6888 | 0177156 | A0A140VK56, | |
| | deficiency | | | P37837 | |
| TF | Atransferrinemia | 7018 | 0091513 | A0PJA6, | |
| | (familial | | | P02787, | |
| | hypotransferrinemia) | | | Q06AH7 | |
| EPCAM | Intestinal | 4072 | 0119888 | P16422 | |
| | epithelial | | | | |
| | dysplasia | | | | |
| VHL | Familial | 7428 | 0134086 | A0A024R2F2, | |
| | erythrocytosis | | | P40337, | |
| | type 2; von | | | A0A0S2Z4K1 | |
| | Hippel Lindau | | | | |
| | disease | | | | |
| GC | Vitamin D | 2638 | 0145321 | P02774 | |
| | deficiency | | | | |
| SERP1NA1 | Alpha-1 | 5265 | 0197249, | E9KL23, | |
| | antitrypsin | | 0277377 | P01009 | |
| | deficiency | | | | |
| ABCC6 | Pseudoxanthoma | 368 | 0091262, | O95255 | |
| | elasticum | | 0275331 | | |
| F8 | Hemophilia A | 2157 | 0185010 | P00451 | |
| F9 | Hemophilia B | 2158 | 0101981 | P00740 | |
| ApoB | Familial | 338 | 0084674 | P04114 | |
| | hypercholesterolemia | | | | |
| PCSK9 | Familial | 255738 | 0169174 | Q8NBP7 | |
| | hypercholesterolemia | | | | |
| LDLR | Familial | 26119 | 0157978 | B3KR97, | |
| AP1 | hypercholesterolemia | | | Q5SW96 | |
| ABCG5 | Sitosterolemia | 64240 | 0138075 | Q9H222 | |
| ABCG8 | Sitosterolemia | 64241 | 0143921 | Q9H221 | |
| LCAT | Lecithin | 3931 | 0213398 | A0A140VK24, | |
| | cholesterol | | | P04180 | |
| | acyltransferase | | | | |
| | deficiency | | | | |
| SPINK5 | Netherton | 11005 | 0133710 | Q9NQ38 | |
| | syndrome | | | | |
| GNE | Inclusion body | 10020 | 0159921 | Q9Y223 | |
| | myopathy 2 | | | | |

In some embodiments, the exogenous cargo comprises a protein of OTC. In some embodiments, the exogenous agent comprises the wild-type human sequence of OTC, a functional fragment thereof (e.g., an enzymatically active fragment thereof), or a functional variant thereof. In some embodiments, the exogenous agent comprises an amino acid sequence having at least 70%, 75%, 80%, 85%, 90%, 95%, 96%, 97%, 98%, or 99%, identity to SEQ ID NO 23. In some embodiments, the payload gene encoding an exogenous agent encodes an amino acid sequence having at least 70%, 75%, 80%, 85%, 90%, 95%, 96%, 97%, 98%, or 99%, identity to a SEQ TD NO. 23. In some embodiments, the payload gene encoding an exogenous agent has a nucleic acid sequence having at least 70%, 75%, 80%, 85%, 90%, 95%, 96%, 97%, 98%, or 99%, identity to a SEQ TD NO. 23.

In some embodiments, the exogenous cargo comprises a protein of LDLR. In some embodiments, the exogenous agent comprises the wild-type human sequence of LDLR, a functional fragment thereof (e.g., an enzymatically active fragment thereof), or a functional variant thereof. In some embodiments, the exogenous agent comprises an amino acid sequence having at least 70%, 75%, 80%, 85%, 90%, 95%, 96%, 97%, 98%, or 99%, identity to SEQ ID NO 24. In some embodiments, the payload gene encoding an exogenous agent encodes an amino acid sequence having at least 70%, 75%, 80%, 85%, 90%, 95%, 96%, 97%, 98%, or 99%, identity to a SEQ ID NO. 24. In some embodiments, the payload gene encoding an exogenous agent has a nucleic acid sequence having at least 70%, 75%, 80%, 85%, 90%, 95%, 96%, 97%, 98%, or 99%, identity to a SEQ ID NO. 24.

In some embodiments, the fusosome or lentiviral vector contains an exogenous agent that is capable of targeting a T cell. In some embodiments, the exogenous agent capable of targeting a T cell is a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, a CAR antigen binding domain is or comprises an antibody or antigen-binding portion thereof. In some embodiments, a CAR antigen binding domain is or comprises an scFv or Fab. In some embodiments a CAR antigen binding domain comprises an scFv or Fab fragment of a T-cell alpha chain antibody; T-cell R chain antibody; T-cell 7 chain antibody; T-cell 6 chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR binding domain binds to a cell surface antigen of a cell. In some embodiments, a cell surface antigen is characteristic of one type of cell. In some embodiments, a cell surface antigen is characteristic of more than one type of cell.

In some embodiments, the antigen binding domain of the CAR targets an antigen characteristic of a T cell. In some embodiments, the antigen characteristic of a T cell is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, the antigen binding domain of the CAR targets an antigen characteristic of a disorder. In some embodiments, the disease or disorder is associates with CD4+ T cells. In some embodiments, the disease or disorder is associated with CD8+ T cells.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

In some embodiments, the CAR comprises at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSFIB); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof, and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof, (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof; (iii) a 4-1BB domain, or a CD134 domain, or functional variant thereof; and (iv) a cytokine or costimulatory ligand transgene.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Examples of exemplary components of a CAR are described in Table 5B. In provided aspects, the sequences of each component in a CAR can include any combination listed in Table 5B.

TABLE 5B
CAR components and Exemplary Sequences

| Component | Sequence | SEQ ID NO |
|---|---|---|
| Extracellular binding domain | | |
| Anti-CD19 scFv (FMC63) | DIQMTQTTSSLSASLGDRVTISCRASQDISKYLN | 25 |
| | WYQQKPDGTVKLLIYHTSRLHSGVPSRFSGSGSG | |
| | TDYSLTISNLEQEDIATYFCQQGNTLPYTFGGGT | |
| | KLEITGSTSGSGKPGSGEGSTKGEVKLQESGPGL | |
| | VAPSQSLSVTCTVSGVSLPDYGVSWIRQPPRKGL | |
| | EWLGVIWGSETTYYNSALKSRLTIIKDNSKSQVF | |
| | LKMNSLQTDDTAIYYCAKHYYYGGSYAMDYW | |
| | GQGTSVTVSS | |
| Anti-CD19 scFv (FMC63) | DIQMTQTTSSLSASLGDRVTISCRASQDISKYLN | 26 |
| | WYQQKPDGTVKLLIYHTSRLHSGVPSRFSGSGSG | |
| | TDYSLTISNLEQEDIATYFCQQGNTLPYTFGGGT | |
| | KLEITGGGGSGGGGSGGGGSEVKLQESGPGLVA | |
| | PSQSLSVTCTVSGVSLPDYGVSWIRQPPRKGLEW | |
| | LGVIWGSETTYYNSALKSRLTIIKDNSKSQVFLK | |
| | MNSLQTDDTAIYYCAKHYYYGGSYAMDYWGQ | |
| | GTSVTVSS | |
| Spacer (e.g. hinge) | | |
| IgG4 Hinge | ESKYGPPCPPCP | 27 |
| CD8 Hinge | TTTPAPRPPTPAPTIASQPLSLRPE | 28 |
| CD28 | IEVMYPPPYLDNEKSNGTIIHVKGKHLCPSPLFPG | 29 |
| | PSKP | |
| Transmembrane | | |
| CD8 | ACRPAAGGAVHTRGLDFACDIYIWAPLAGTCGV | 30 |
| | LLLSLVITLYC | |
| CD28 | FWVLVVVGGVLACYSLLVTVAFIIFWV | 31 |
| CD28 | FWVLVVVGGVLACYSLLVTVAFIIFWV | 32 |
| Costimulatory domain | | |
| CD28 | RSKRSRLLHSDYMNMTPRRPGPTRKHYQPYAPP | 33 |
| | RDFAAYRS | |
| 4-1BB | KRGRKKLLYIFKQPFMRPVQTTQEEDGCSCRFPE | 34 |
| | EEEGGCEL | |
| Primary Signaling Domain | | |
| CD3zeta | RVKFSRSADAPAYQQGQNQLYNELNLGRREEYD | 35 |
| | VLDKRRGRDPEMGGKPRRKNPQEGLYNELQKD | |
| | KMAEAYSEIGMKGERRRGKGHDGLYQGLSTAT | |
| | KDTYDALHMQALPPR | |
| CD3zeta | RVKFSRSADAPAYKQGQNQLYNELNLGRREEYD | 36 |
| | VLDKRRGRDPEMGGKPRRKNPQEGLYNELQKD | |
| | KMAEAYSEIGMKGERRRGKGHDGLYQGLSTAT | |
| | KDTYDALHMQALPPR | |

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets. In addition to the CARs described herein, various chimeric antigen receptors and nucleotide sequences encoding the same are known and would be suitable for fusosomal delivery and reprogramming of target cells in vivo and in vitro as described herein. See, e.g., WO2013040557; WO2012079000; WO2016030414; Smith T, et al., Nature Nanotechnology. 2017.(DOI. 10.1038/NNANO.2017.57), the disclosures of which are herein incorporated by reference in their entirety.

In some embodiments a targeted lipid particle comprising a CAR or a nucleic acid encoding a CAR (e.g., a DNA, a gDNA, a cDNA, an RNA, a pre-MRNA, an mRNA, an miRNA, an siRNA, etc.) is delivered to a target cell. In some embodiments the target cell is an effector cell, e.g., a cell of the immune system that expresses one or more Fc receptors and mediates one or more effector functions. In some embodiments, a target cell may include, but may not be limited to, one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including but not limited to humans, mice, rats, rabbits, and monkeys.

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

In some embodiments, the exogenous agent is associated with a disease of hematopoetic stem cells (HSC). In some embodiments, the exogenous agent comprises a protein of Table 5C below. In some embodiments, the exogenous agent comprises the wild-type human sequence of any of the proteins of Table 5C, a functional fragment thereof (e.g., an enzymatically active fragment thereof), or a functional variant thereof. In some embodiments, the exogenous agent comprises an amino acid sequence having at least 70%, 75%, 80%, 85%, 90%, 95%, 96%, 97%, 98%, or 99%, identity to an amino acid sequence of Table 5C, e.g., a Uniprot Protein Accession Number sequence of Table 5C or an amino acid sequence of Table 5C. In some embodiments, the payload gene encodes an amino acid sequence having at least 70%, 75%, 80%, 85%, 90%, 95%, 96%, 97%, 98%, or 99%, identity to an amino acid sequence of Table 5C. In some embodiments, the payload gene has a nucleic acid sequence having at least 70%, 75%, 80%, 85%, 90%, 95%, 96%, 97%, 98%, or 99%, identity to a nucleic acid sequence of Table 5C, e.g., an Ensemble Gene Accession Number of Table 5C.

TABLE 5C
Exemplary exogenous agents associated with
HSC disorders

| | | Ensembl | | |
|---|---|---|---|---|
| | | Gene(s) | | |
| | | Accession | | |
| | | Number | Uniprot | |
| | Entrez | (ENSG0000+ | Protein(s) | |
| | Accession | number | Accession | Disease/ |
| Gene | Number | shown) | Number | Disorder |
| ADA | 100 | 0196839 | P00813 | ADA |
| | | | | SCID |
| IL2RG | 3561 | 0147168 | P31785 | X-Linked |
| | | | | SCID |
| JAK3 | 3718 | 0105639 | P52333 | Jak-3 |
| | | | | SCID |
| IL7R | 3575 | 0168685 | P16871 | IL7R |
| | | | | SCID |
| HBB | 3043 | 0244734 | P68871 | Thalassemia |
| | | | | Major; |
| | | | | Sickle |
| | | | | Cell |
| | | | | Disease |
| F8 | 2157 | 0185010 | P00451 | Hemophilia |
| | | | | A |
| F9 | 2158 | 0101981 | P00740 | Hemophilia |
| | | | | B |
| WAS | 7454 | 0015285 | P42768 | Wiskott- |
| | | | | Aldrich |
| | | | | Syndrome |
| CYBA | 1535 | 0051523 | P13498 | Chronic |
| | | | | Granulom |
| | | | | atous |
| | | | | Disease |
| CYBB | 1536 | 0165168 | P04839 | Chronic |
| | | | | Granulom |
| | | | | atous |
| | | | | Disease |
| NCF1 | 653361 | 0158517 | P14598 | Chronic |
| | | | | Granulom |
| | | | | atous |
| | | | | Disease |
| NCF2 | 4688 | 0116701 | P19878 | Chronic |
| | | | | Granulom |
| | | | | atous |
| | | | | Disease |
| NCF4 | 4689 | 0100365 | Q15080 | Chronic |
| | | | | Granulom |
| | | | | atous |
| | | | | Disease |
| UROS | 7390 | 0188690 | P10746 | Gunther |
| | | | | Disease |
| TCIRG1 | 10312 | 0110719 | Q13488 | Malignant |
| | | | | Infantile |
| | | | | Osteoporosis |
| CLCN7 | 1186 | 0103249 | P51798 | Malignant |
| | | | | Infantile |
| | | | | Osteoporosis |
| MPL | 4352 | 0117400 | P40238 | Congenital |
| | | | | Amegakaryocytic |
| | | | | Thrombocytopenia |
| ITGA2B | 3674 | 0005961 | P08514 | Glanzmann's |
| | | | | Thrombasthenia |
| ITGB3 | 3690 | 0259207 | P05106 | Glanzmann's |
| | | | | Thrombasthenia |
| ITGB2 | 3689 | 0160255 | P05107 | Leukocyte |
| | | | | Adhesion |
| | | | | Deficiency |
| PKLR | 5313 | 0143627 | P30613 | Pyruvate |
| | | | | Kinase |
| | | | | Deficiency |
| SLC25A38 | 54977 | 0144659 | Q96DW6 | Autosomal |
| | | | | Recessive |
| | | | | Sideroblastic |
| | | | | Anemia |
| RAG1 | 5896 | 0166349 | P15918 | Rag 1 |
| | | | | Deficiency |
| RAG2 | 5897 | 0175097 | P55895 | Rag 2 |
| | | | | Deficiency |
| FANCA | 2175 | 0187741 | O15360 | Fanconi |
| | | | | Anemia |
| FANCC | 2176 | 0158169 | Q00597 | Fanconi |
| | | | | Anemia |
| FANCG | 2189 | 0221829 | O15287 | Fanconi |
| | | | | Anemia |
| ABCD1 | 215 | 0101986 | P33897 | X-Linked |
| | | | | Adrenoleukodystrophy |

In some embodiments, the exogenous agent is associated with a disease of lysosomal storage. In some embodiments, the exogenous agent comprises a protein of Table 5D below. In some embodiments, the exogenous agent comprises the wild-type human sequence of any of the proteins of Table 5D, a functional fragment thereof (e.g., an enzymatically active fragment thereof), or a functional variant thereof. In some embodiments, the exogenous agent comprises an amino acid sequence having at least 70%, 75%, 80%, 85%, 90%, 95%, 96%, 97%, 98%, or 99%, identity to an amino acid sequence of Table 5D, e.g., a Uniprot Protein Accession Number sequence of Table 5D or an amino acid sequence of Table 5D. In some embodiments, the payload gene encodes an amino acid sequence having at least 70%, 75%, 80%, 85%, 90%, 95%, 96%, 97%, 98%, or 99%, identity to an amino acid sequence of Table 5D. In some embodiments, the payload gene has a nucleic acid sequence having at least 70%, 75%, 80%, 85%, 90%, 95%, 96%, 97%, 98%, or 99%, identity to a nucleic acid sequence of Table 5D, e.g., an Ensemble Gene Accession Number of Table 5D.

TABLE 5D
Exogenous agents associate with Lysosomal
stoarage diseases

| | | Ensembl | | |
|---|---|---|---|---|
| | | Gene(s) | | |
| | | Accession | | |
| | | Number | Uniprot | |
| | Entrez | (ENSG0000+ | Protein(s) | |
| | Accession | number | Accession | Disease/ |
| Gene | Number | shown) | Number | Disorder |
| MAN2B1 | 4125 | 0104774 | O00754 | Alpha- |
| | | | | mannosidosis |
| AGA | 175 | 0038002 | P20933 | Aspartylguco |
| | | | | saminuria |
| LYST | 1130 | 0143669 | Q99698 | Chediak- |
| | | | | Higashi |
| | | | | Syndrome |
| CTNS | 1497 | 0040531 | O60931 | Cystinosis |
| LAMP2 | 3920 | 0005893 | P13473 | Danon |
| | | | | Disease |
| GLA | 2717 | 0102393 | P06280 | Fabry |
| | | | | Disease |
| CTSA | 5476 | 0064601 | P10619 | Galactosialidosis |
| GBA | 2629 | 0177628 | P04062 | Gaucher |
| | | | | Disease |
| GAA | 2548 | 0171298 | P10253 | Pompe |
| | | | | Disease |
| IDS | 3423 | 0010404 | P22304 | Hunter |
| | | | | Disease |
| IDUA | 3425 | 0127415 | P35475 | Hurler |
| | | | | Disease |
| ISSD | 26503 | 0119899 | Q9NRA2 | Infantile Free |
| | | | | Sialic Acid |
| | | | | Storage |
| | | | | Disease |
| ARSB | 411 | 0113273 | P15848 | Maroteaux- |
| | | | | Lamy |
| GALNS | 2588 | 0141012 | P34059 | Morquio |
| | | | | Type A |
| GLB1 | 2720 | 0170266 | P16278 | Morquio |
| | | | | Type B |
| NEU1 | 4758 | 0204386 | Q99519 | Mucolipidosis |
| | | | | Type I |
| GNPTA | 79158 | 0111670 | Q3T906 | Mucolipidosis |
| | | | | Type II |
| SUMF1 | 285362 | 0144455 | Q8NBK3 | Multiple |
| | | | | Sulfatase |
| | | | | Deficiency |
| SMPD1 | 6609 | 0166311 | P17405 | Niemann- |
| | | | | Pick Disease |
| | | | | Type A; |
| | | | | Niemann- |
| | | | | Pick Disease |
| | | | | Type B |
| NPC1 | 4864 | 0141458 | O15118 | Niemann- |
| | | | | Pick Disease |
| | | | | Type C |
| NPC2 | 10577 | 0119655 | P61916 | Niemann- |
| | | | | Pick Disease |
| | | | | Type C |
| CTSK | 1513 | 0143387 | P43235 | Pycnodystosis |
| GNS | 2799 | 0135677 | P15586 | Sanfilippo |
| | | | | Syndrome |
| | | | | Type A |
| HGSNAT | 138050 | 0165102 | Q68CP4 | Sanfilippo |
| | | | | Syndrome |
| | | | | Type B |
| NAGLU | 4669 | 0108784 | P54802 | Sanfilippo |
| | | | | Syndrome |
| | | | | Type C |
| SGSH | 6448 | 0181523 | P51688 | Sanfilippo |
| | | | | Syndrome |
| | | | | Type D |
| NAGA | 4668 | 0198951 | P17050 | Schindler |
| | | | | Disease |
| | | | | Types I and II |
| GUSB | 2990 | 0169919 | P08236 | Sly Disease |
| PSAP | 5660 | 0197746 | P07602 | Sphinoglipido |
| | | | | sis- |
| | | | | Encephalopathy |
| LAL | 3988 | 0107798 | P38571 | Wolman |
| | | | | Disease |

### Fusogen Receptors and Methods of Preventin2 Source Cell Fusion

In some embodiments, a source cell is modified (e.g., using siRNA, miRNA, shRNA, genome editing, or other methods) to have reduced expression (e.g., no expression) of a fusogen receptor that binds a fusogen expressed by the source cell. In some embodiments, the fusogen is a re-targeted fusogen, e.g., the fusogen may comprise a target-binding domain, e.g., an antibody, e.g., an scFv. In some embodiments, the fusogen receptor is bound by the antibody.

### Insulator Elements

In some embodiments, a fusosome nucleic acid further comprises one or more insulator elements, e.g., an insulator element described herein. Insulators elements may contribute to protecting lentivirus-expressed sequences, e.g., therapeutic polypeptides, from integration site effects, which may be mediated by cis-acting elements present in genomic DNA and lead to deregulated expression of transferred sequences (e.g., position effect; see, e.g., Burgess-Beusse et al, 2002, Proc. Natl. Acad. Sci., USA, 99: 16433; and Zhan et al, 2001, Hum. Genet., 109:471) or deregulated expression of endogenous sequences adjacent to the transferred sequences. In some embodiments, transfer vectors comprise one or more insulator element the 3′ LTR and upon integration of the provirus into the host genome, the provirus comprises the one or more insulators at the 5′ LTR and/or 3′ LTR, by virtue of duplicating the 3′ LTR. Suitable insulators include, but are not limited to, the chicken β-globin insulator (see Chung et al, 1993. Cell 74:505; Chung et al, 1997. N4S 94:575; and Bell et al., 1999. Cell 98:387, incorporated by reference herein) or an insulator from a human β-globin locus, such as chicken HS4. In some embodiments the insulator binds CCCTC binding factor (CTCF). In some embodiments the insulator is a barrier insulator. In some embodiments the insulator is an enhancer-blocking insulator. See, e.g., Emery et al., Human Gene Therapy, 2011, and in Browning and Trobridge, Biomedicines, 2016, both of which are included in their entirety by reference.

In some embodiments, insulators in the retroviral nucleic acid reduce genotoxicity in recipient cells. Genotoxicity can be measured, e.g., as described in Cesana et al, “Uncovering and dissecting the genotoxicity of self-inactivating lentiviral vectors in vivo” Mol Ther. 2014 April; 22(4):774-85. doi: 10.1038/mt.2014.3. Epub 2014 Jan. 20.

Pharmaceutical Compositions and Methods of Making them

In some embodiments, one or more transducing units of a fusosome or retroviral vector are administered to the subject. In some embodiments, at least 1, 10, 100, 1000, 10⁴, 10⁵, 10⁶, 10⁷, 10⁸, 10⁹, 10¹⁰, 10¹¹, 10¹², 10¹³, or 10¹⁴, transducing units per kg are administered to the subject. In some embodiments at least 1, 10, 100, 1000, 10⁴, 10⁵, 10⁶, 10⁷, 10⁸, 10⁹, 10¹⁰, 10¹¹, 10¹², 10¹³, or 10¹⁴, transducing units per target cell per ml of blood are administered to the subject.

### Concentration and Purification of Lentivirus

In some embodiments, a fusosome formulation described herein can be produced by a process comprising one or more of, e.g., all of, the following steps (i) to (vi), e.g., in chronological order:
(i) culturing cells that produce the fusosome;
(ii) harvesting the fusosome containing supernatant;
(iii) optionally clarifying the supernatant;
(iv) purifying the fusosome to give a fusosome preparation;
(v) optionally filter-sterilization of the fusosome preparation; and
(vi) concentrating the fusosome preparation to produce the final bulk product.

In some embodiments the process does not comprise the clarifying step (iii). In other embodiments the process does include the clarifying step (iii). In some embodiments, step (vi) is performed using ultrafiltration, or tangential flow filtration, more preferably hollow fiber ultrafiltration. In some embodiments, the purification method in step (iv) is ion exchange chromatography, e.g., anion exchange chromatography. In some embodiments, the filter-sterilisation in step (v) is performed using a 0.22 μm or a 0.2 μm sterilising filter. In some embodiments, step (iii) is performed by filter clarification. In some embodiments, step (iv) is performed using a method or a combination of methods selected from chromatography, ultrafiltration/diafiltration, or centrifugation. In some embodiments, the chromatography method or a combination of methods is selected from ion exchange chromatography, hydrophobic interaction chromatography, size exclusion chromatography, affinity chromatography, reversed phase chromatography, and immobilized metal ion affinity chromatography. In some embodiments, the centrifugation method is selected from zonal centrifugation, isopycnic centrifugation and pelleting centrifugation. In some embodiments, the ultrafiltration/diafiltration method is selected from tangential flow diafiltration, stirred cell diafiltration and dialysis. In some embodiments, at least one step is included into the process to degrade nucleic acid to improve purification. In some embodiments, said step is nuclease treatment.

In some embodiments, concentration of the vectors is done before filtration. In some embodiments, concentration of the vectors is done after filtration. In some embodiments, concentration and filtrations steps are repeated.

In some embodiments, the final concentration step is performed after the filter-sterilisation step. In some embodiments, the process is a large scale-process for producing clinical grade formulations that are suitable for administration to humans as therapeutics. In some embodiments, the filter-sterilisation step occurs prior to a concentration step. In some embodiments, the concentration step is the final step in the process and the filter-sterilisation step is the penultimate step in the process. In some embodiments, the concentration step is performed using ultrafiltration, preferably tangential flow filtration, more preferably hollow fiber ultrafiltration. In some embodiments, the filter-sterilisation step is performed using a sterilising filter with a maximum pore size of about 0.22 μm. In another preferred embodiment the maximum pore size is 0.2 μm

In some embodiments, the vector concentration is less than or equal to about 4.6×10¹¹ RNA genome copies per ml of preparation prior to filter-sterilisation. The appropriate concentration level can be achieved through controlling the vector concentration using, e.g. a dilution step, if appropriate. Thus, in some embodiments, a retroviral vector preparation is diluted prior to filter sterilisation.

Clarification may be done by a filtration step, removing cell debris and other impurities. Suitable filters may utilize cellulose filters, regenerated cellulose fibers, cellulose fibers combined with inorganic filter aids (e.g. diatomaceous earth, perlite, fumed silica), cellulose filters combined with inorganic filter aids and organic resins, or any combination thereof, and polymeric filters (examples include but are not limited to nylon, polypropylene, polyethersulfone) to achieve effective removal and acceptable recoveries. A multiple stage process may be used. An exemplary two or three-stage process would consist of a coarse filter(s) to remove large precipitate and cell debris followed by polishing second stage filter(s) with nominal pore sizes greater than 0.2 micron but less than 1 micron. The optimal combination may be a function of the precipitate size distribution as well as other variables. In addition, single stage operations employing a relatively small pore size filter or centrifugation may also be used for clarification. More generally, any clarification approach including but not limited to dead-end filtration, microfiltration, centrifugation, or body feed of filter aids (e.g. diatomaceous earth) in combination with dead-end or depth filtration, which provides a filtrate of suitable clarity to not foul the membrane and/or resins in the subsequent steps, will be acceptable to use in the clarification step of the present invention.

In some embodiments, depth filtration and membrane filtration is used. Commercially available products useful in this regard are for instance mentioned in WO 03/097797, p. 20-21. Membranes that can be used may be composed of different materials, may differ in pore size, and may be used in combinations. They can be commercially obtained from several vendors. In some embodiments, the filter used for clarification is in the range of 1.2 to 0.22 μm. In some embodiments, the filter used for clarification is either a 1.2/0.45 m filter or an asymmetric filter with a minimum nominal pore size of 0.22 m

In some embodiments, the method employs nuclease to degrade contaminating DNA/RNA, i.e. mostly host cell nucleic acids. Exemplary nucleases suitable for use in the present invention include Benzonase® Nuclease (EP 0229866) which attacks and degrades all forms of DNA and RNA (single stranded, double stranded linear or circular) or any other DNase and/or RNase commonly used within the art for the purpose of eliminating unwanted or contaminating DNA and/or RNA from a preparation. In preferred embodiments, the nuclease is Benzonase® Nuclease, which rapidly hydrolyzes nucleic acids by hydrolyzing internal phosphodiester bonds between specific nucleotides, thereby reducing the size of the polynucleotides in the vector containing supernatant. Benzonase® Nuclease can be commercially obtained from Merck KGaA (code W214950). The concentration in which the nuclease is employed is preferably within the range of 1-100 units/ml.

In some embodiments, the vector suspension is subjected to ultrafiltration (sometimes referred to as diafiltration when used for buffer exchange) at least once during the process, e.g. for concentrating the vector and/or buffer exchange. The process used to concentrate the vector can include any filtration process (e.g., ultrafiltration (UF)) where the concentration of vector is increased by forcing diluent to be passed through a filter in such a manner that the diluent is removed from the vector preparation whereas the vector is unable to pass through the filter and thereby remains, in concentrated form, in the vector preparation. UF is described in detail in, e.g., Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A suitable filtration process is Tangential Flow Filtration (“TFF”) as described in, e.g., MILLIPORE catalogue entitled “Pharmaceutical Process Filtration Catalogue” pp. 177-202 (Bedford, Mass., 1995/96). TFF is widely used in the bioprocessing industry for cell harvesting, clarification, purification and concentration of products including viruses. The system is composed of three distinct process streams: the feed solution, the permeate and the retentate. Depending on application, filters with different pore sizes may be used. In some embodiments, the retentate contains the product (lentiviral vector).The particular ultrafiltration membrane selected may have a pore size sufficiently small to retain vector but large enough to effectively clear impurities. Depending on the manufacturer and membrane type, for retroviral vectors nominal molecular weight cutoffs (NMWC) between 100 and 1000 kDa may be appropriate, for instance membranes with 300 kDa or 500 kDa NMWC. The membrane composition may be, but is not limited to, regenerated cellulose, polyethersulfone, polysulfone, or derivatives thereof. The membranes can be flat sheets (also called flat screens) or hollow fibers. A suitable UF is hollow fibre UF, e.g., filtration using filters with a pore size of smaller than 0.1 m. Products are generally retained, while volume can be reduced through permeation (or be kept constant during diafiltration by adding buffer with the same speed as the speed with which the permeate, containing buffer and impurities, is removed at the permeate side).

The two most widely used geometries for TFF in the biopharmaceutical industry are plate & frame (flat screens) and hollow fiber modules. Hollow fiber units for ultrafiltration and microfiltration were developed by Amicon and Ramicon in the early 1970s (Cheryan, M. Ultrafiltration Handbook), even though now there are multiple vendors including Spectrum and GE Healthcare. The hollow fiber modules consist of an array of self-supporting fibers with a dense skin layer. Fiber diameters range from 0.5 mm-3 mm. In certain embodiments, hollow fibers are used for TFF. In certain embodiments, hollow fibers of 500 kDa (0.05 m) pore size are used. Ultrafiltration may comprise diafiltration (DF). Microsolutes can be removed by adding solvent to the solution being ultrafiltered at a rate equal to the UF rate. This washes microspecies from the solution at a constant volume, purifying the retained vector.

UF/DF can be used to concentrate and/or buffer exchange the vector suspensions in different stages of the purification process. The method can utilize a DF step to exchange the buffer of the supernatant after chromatography or other purification steps, but may also be used prior to chromatography.

In some embodiments, the eluate from the chromatography step is concentrated and further purified by ultrafiltration-diafiltration. During this process the vector is exchanged into formulation buffer. Concentration to the final desired concentration can take place after the filter-sterilisation step. After said sterile filtration, the filter sterilised substance is concentrated by aseptic UF to produce the bulk vector product.

In embodiments, the ultrafiltration/diafiltration may be tangential flow diafiltration, stirred cell diafiltration and dialysis.

Purification techniques tend to involve the separation of the vector particles from the cellular milieu and, if necessary, the further purification of the vector particles. One or more of a variety of chromatographic methods may be used for this purification. Ion exchange, and more particularly anion exchange, chromatography is a suitable method, and other methods could be used. A description of some chromatographic techniques is given below.

Ion-exchange chromatography utilises the fact that charged species, such as biomolecules and viral vectors, can bind reversibly to a stationary phase (such as a membrane, or else the packing in a column) that has, fixed on its surface, groups that have an opposite charge. There are two types of ion exchangers. Anion exchangers are stationary phases that bear groups having a positive charge and hence can bind species with a negative charge. Cation exchangers bear groups with a negative charge and hence can bind species with positive charge. The pH of the medium has an influence on this, as it can alter the charge on a species. Thus, for a species such as a protein, if the pH is above the pI, the net charge will be negative, whereas below the pI, the net charge will be positive.

Displacement (elution) of the bound species can be effected by the use of suitable buffers. Thus commonly the ionic concentration of the buffer is increased until the species is displaced through competition of buffer ions for the ionic sites on the stationary phase. An alternative method of elution entails changing the pH of the buffer until the net charge of the species no longer favours biding to the stationary phase. An example would be reducing the pH until the species assumes a net positive charge and will no longer bind to an anion exchanger.

Some purification can be achieved if impurities are uncharged, or else if they bear a charge of opposite sign to that of the desired species, but the same sign to that on the ion exchanger. This is because uncharged species and those having a charge of the same sign to that an ion exchanger, will not normally bind. For different bound species, the strength of the binding varies with factors such as the charge density and the distribution of charges on the various species. Thus by applying an ionic or pH gradient (as a continuous gradient, or as a series of steps), the desired species might be eluted separately from impurities.

Size exclusion chromatography is a technique that separates species according to their size. Typically it is performed by the use of a column packed with particles having pores of a well-defined size. For the chromatographic separation, particles are chosen that have pore sizes that are appropriate with regard to the sizes of the species in the mixture to be separated. When the mixture is applied, as a solution (or suspension, in the case of a virus), to the column and then eluted with buffer, the largest particles will elute first as they have limited (or no) access to the pores. Smaller particles will elute later as they can enter the pores and hence take a longer path through the column. Thus in considering the use of size exclusion chromatography for the purification of viral vectors, it would be expected that the vector would be eluted before smaller impurities such as proteins.

Species, such as proteins, have on their surfaces, hydrophobic regions that can bind reversibly to weakly hydrophobic sites on a stationary phase. In media having a relatively high salt concentration, this binding is promoted. Typically in HIC the sample to be purified is bound to the stationary phase in a high salt environment. Elution is then achieved by the application of a gradient (continuous, or as a series of steps) of decreasing salt concentration. A salt that is commonly used is ammonium sulphate. Species having differing levels of hydrophobicity will tend to be eluted at different salt concentrations and so the target species can be purified from impurities. Other factors, such as pH, temperature and additives to the elution medium such as detergents, chaotropic salts and organics can also influence the strength of binding of species to HIC stationary phases. One, or more, of these factors can be adjusted or utilised to optimise the elution and purification of product.

Viral vectors have on their surface, hydrophobic moieties such as proteins, and thus HIC could potentially be employed as a means of purification.

Like HIC, RPC separates species according to differences in their hydrophobicities. A stationary phase of higher hydrophobicity than that employed in HIC is used. The stationary phase often consists of a material, typically silica, to which are bound hydrophobic moieties such as alkyl groups or phenyl groups. Alternatively the stationary phase might be an organic polymer, with no attached groups. The sample-containing the mixture of species to be resolved is applied to the stationary phase in an aqueous medium of relatively high polarity which promotes binding. Elution is then achieved by reducing the polarity of the aqueous medium by the addition of an organic solvent such as isopropanol or acetonitrile. Commonly a gradient (continuous, or as a series of steps) of increasing organic solvent concentration is used and the species are eluted in order of their respective hydrophobicities.

Other factors, such as the pH of the elution medium, and the use of additives, can also influence the strength of binding of species to RPC stationary phases. One, or more, of these factors can be adjusted or utilised to optimise the elution and purification of product. A common additive is trifluororacetic acid (TFA). This suppresses the ionisation of acidic groups such as carboxyl moieties in the sample. It also reduces the pH in the eluting medium and this suppresses the ionisation of free silanol groups that may be present on the surface of stationary phases having a silica matrix. TFA is one of a class of additives known as ion pairing agents. These interact with ionic groups, present on species in the sample, that bear an opposite charge. The interaction tends to mask the charge, increasing the hydrophobicity of the species. Anionic ion pairing agents, such as TFA and pentafluoropropionic acid interact with positively charged groups on a species. Cationic ion pairing agents such, as triethylamine, interact with negatively charged groups.

Viral vectors have on their surface, hydrophobic moieties such as proteins, and thus RPC, potentially, could be employed as a means of purification.

Affinity chromatography utilises the fact that certain ligands that bind specifically with biomolecules such as proteins or nucleotides, can be immobilised on a stationary phase. The modified stationary phase can then be used to separate the relevant biomolecule from a mixture. Examples of highly specific ligands are antibodies, for the purification of target antigens and enzyme inhibitors for the purification of enzymes. More general interactions can also be utilised such as the use of the protein A ligand for the isolation of a wide range of antibodies.

Typically, affinity chromatography is performed by application of a mixture, containing the species of interest, to the stationary phase that has the relevant ligand attached. Under appropriate conditions this will lead to the binding of the species to the stationary phase. Unbound components are then washed away before an eluting medium is applied. The eluting medium is chosen to disrupt the binding of the ligand to the target species. This is commonly achieved by choice of an appropriate ionic strength, pH or by the use of substances that will compete with the target species for ligand sites. For some bound species, a chaotropic agent such as urea is used to effect displacement from the ligand. This, however, can result in irreversible denaturation of the species.

Viral vectors have on their surface, moieties such as proteins, that might be capable of binding specifically to appropriate ligands. This means that, potentially, affinity chromatography could be used in their isolation.

Biomolecules, such as proteins, can have on their surface, electron donating moieties that can form coordinate bonds with metal ions. This can facilitate their binding to stationary phases carrying immobilised metal ions such as Ni²⁺, Cu²⁺, Zn²⁺ or Fe³⁺. The stationary phases used in IMAC have chelating agents, typically nitriloacetic acid or iminodiacetic acid covalently attached to their surface and it is the chelating agent that holds the metal ion. It is necessary for the chelated metal ion to have at least one coordination site left available to form a coordinate bond to a biomolecule. Potentially there are several moieties on the surface of biomolecules that might be capable of bonding to the immobilised metal ion. These include histidine, tryptophan and cysteine residues as well as phosphate groups. For proteins, however, the predominant donor appears to be the imidazole group of the histidine residue. Native proteins can be separated using IMAC if they exhibit suitable donor moieties on their surface. Otherwise IMAC can be used for the separation of recombinant proteins bearing a chain of several linked histidine residues.

Typically, IMAC is performed by application of a mixture, containing the species of interest, to the stationary phase. Under appropriate conditions this will lead to the coordinate bonding of the species to the stationary phase. Unbound components are then washed away before an eluting medium is applied. For elution, gradients (continuous, or as a series of steps) of increasing salt concentration or decreasing pH may be used. Also a commonly used procedure is the application of a gradient of increasing imidazole concentration. Biomolecules having different donor properties, for example having histidine residues in differing environments, can be separated by the use of gradient elution.

Viral vectors have on their surface, moieties such as proteins, that might be capable of binding to IMAC stationary phases. This means that, potentially, IMAC could be used in their isolation.

Suitable centrifugation techniques include zonal centrifugation, isopycnic ultra and pelleting centrifugation.

Filter-sterilisation is suitable for processes for pharmaceutical grade materials. Filter-sterilisation renders the resulting formulation substantially free of contaminants. The level of contaminants following filter-sterilisation is such that the formulation is suitable for clinical use. Further concentration (e.g. by ultrafiltration) following the filter-sterilisation step may be performed in aseptic conditions. In some embodiments, the sterilising filter has a maximum pore size of 0.22 m.

The fusosomes or retroviral vectors herein can also be subjected to methods to concentrate and purify a lentiviral vector using flow-through ultracentrifugation and high-speed centrifugation, and tangential flow filtration. Flow through ultracentrifugation can be used for the purification of RNA tumor viruses (Toplin et al, Applied Microbiology 15:582-589, 1967; Burger et al., Journal of the National Cancer Institute 45: 499-503, 1970). Flow-through ultracentrifugation can be used for the purification of Lentiviral vectors. This method can comprise one or more of the following steps. For example, a lentiviral vector can be produced from cells using a cell factory or bioreactor system. A transient transfection system can be used or packaging or producer cell lines can also similarly be used. A pre-clarification step prior to loading the material into the ultracentrifuge could be used if desired. Flow-through ultracentrifugation can be performed using continuous flow or batch sedimentation. The materials used for sedimentation are, e.g.: Cesium chloride, potassium tartrate and potassium bromide, which create high densities with low viscosity although they are all corrosive. CsCl is frequently used for process development as a high degree of purity can be achieved due to the wide density gradient that can be created (1.0 to 1.9 g/cm³). Potassium bromide can be used at high densities, e.g., at elevated temperatures, such as 25° C., which may be incompatible with stability of some proteins. Sucrose is widely used due to being inexpensive, non-toxic and can form a gradient suitable for separation of most proteins, sub-cellular fractions and whole cells. Typically the maximum density is about 1.3 g/cm³. The osmotic potential of sucrose can be toxic to cells in which case a complex gradient material can be used, e.g. Nycodenz. A gradient can be used with 1 or more steps in the gradient. An embodiment is to use a step sucrose gradient. The volume of material can be from 0.5 liters to over 200 liters per run. The flow rate speed can be from 5 to over 25 liters per hour. A suitable operating speed is between 25,000 and 40,500 rpm producing a force of up to 122,000×g. The rotor can be unloaded statically in desired volume fractions. An embodiment is to unload the centrifuged material in 100 ml fractions. The isolated fraction containing the purified and concentrated Lentiviral vector can then be exchanged in a desired buffer using gel filtration or size exclusion chromatography. Anionic or cationic exchange chromatography could also be used as an alternate or additional method for buffer exchange or further purification. In addition, Tangential Flow Filtration can also be used for buffer exchange and final formulation if required. Tangential Flow Filtration (TFF) can also be used as an alternative step to ultra or high speed centrifugation, where a two step TFF procedure would be implemented. The first step would reduce the volume of the vector supernatant, while the second step would be used for buffer exchange, final formulation and some further concentration of the material. The TFF membrane can have a membrane size of between 100 and 500 kilodaltons, where the first TFF step can have a membrane size of 500 kilodaltons, while the second TFF can have a membrane size of between 300 to 500 kilodaltons. The final buffer should contain materials that allow the vector to be stored for long term storage.

In embodiments, the method uses either cell factories that contains adherent cells, or a bioreactor that contains suspension cells that are either transfected or transduced with the vector and helper constructs to produce lentiviral vector. Non limiting examples or bioreactors, include the Wave bioreactor system and the Xcellerex bioreactors. Both are disposable systems. However non-disposable systems can also be used. The constructs can be those described herein, as well as other lentiviral transduction vectors. Alternatively the cell line can be engineered to produce Lentiviral vector without the need for transduction or transfection. After transfection, the lentiviral vector can be harvested and filtered to remove particulates and then is centrifuged using continuous flow high speed or ultra centrifugation. A preferred embodiment is to use a high speed continuous flow device like the JCF-A zonal and continuous flow rotor with a high speed centrifuge. Also preferably is the use of Contifuge Stratus centrifuge for medium scale Lentiviral vector production. Also suitable is any continuous flow centrifuge where the speed of centrifugation is greater than 5,000×g RCF and less than 26,000×g RCF. Preferably, the continuous flow centrifugal force is about 10,500×g to 23,500×g RCF with a spin time of between 20 hours and 4 hours, with longer centrifugal times being used with slower centrifugal force. The lentiviral vector can be centrifuged on a cushion of more dense material (a non limiting example is sucrose but other reagents can be used to form the cushion and these are well known in the art) so that the Lentiviral vector does not form aggregates that are not filterable, as sometimes occurs with straight centrifugation of the vector that results in a viral vector pellet. Continuous flow centrifugation onto a cushion allows the vector to avoid large aggregate formation, yet allows the vector to be concentrated to high levels from large volumes of transfected material that produces the Lentiviral vector. In addition, a second less-dense layer of sucrose can be used to band the Lentiviral vector preparation. The flow rate for the continuous flow centrifuge can be between 1 and 100 ml per minute, but higher and lower flow rates can also be used. The flow rate is adjusted to provide ample time for the vector to enter the core of the centrifuge without significant amounts of vector being lost due to the high flow rate. If a higher flow rate is desired, then the material flowing out of the continuous flow centrifuge can be re-circulated and passed through the centrifuge a second time. After the virus is concentrated using continuous flow centrifugation, the vector can be further concentrated using Tangential Flow Filtration (TFF), or the TFF system can be simply used for buffer exchange. A non-limiting example of a TFF system is the Xampler cartridge system that is produced by GB-Healthcare. Preferred cartridges are those with a MW cut-off of 500,000 MW or less. Preferably a cartridge is used with a MW cut-off of 300,000 MW. A cartridge of 100,000 MW cut-off can also be used. For larger volumes, larger cartridges can be used and it will be easy for those in the art to find the right TFF system for this final buffer exchange and/or concentration step prior to final fill of the vector preparation. The final fill preparation may contain factors that stabilize the vector-sugars are generally used and are known in the art.

### Protein Content

In some embodiments the fusosome includes various source cell genome-derived proteins, exogenous proteins, and viral-genome derived proteins. In some embodiments the retroviral particle contains various ratios of source cell genome-derived proteins to viral-genome-derived proteins, source cell genome-derived proteins to exogenous proteins, and exogenous proteins to viral-genome derived proteins.

In some embodiments, the viral-genome derived proteins are GAG polyprotein precursor, HIV-1 Integrase, POL polyprotein precursor, Capsid, Nucleocapsid, β17 matrix, β6, β2, VPR, Vif.

In some embodiments, the source cell-derived proteins are Cyclophilin A, Heat Shock 70 kD, Human Elongation Factor-1 Alpha (EF-1R), Histones H1, H2A, H3, H4, beta-globin, Trypsin Precursor, Parvulin, Glyceraldehyde-3-phosphate dehydrogenase, Lck, Ubiquitin, SUMO-1, CD48, Syntenin-1, Nucleophosmin, Heterogeneous nuclear ribonucleoproteins C1/C2, Nucleolin, Probable ATP-dependent helicase DDX48, Matrin-3, Transitional ER ATPase, GTP-binding nuclear protein Ran, Heterogeneous nuclear ribonucleoprotein U, Interleukin enhancer binding factor 2, Non-POU domain containing octamer binding protein, RuvB like 2, HSP 90-b, HSP 90-a, Elongation factor 2, D-3-phosphoglycerate dehydrogenase, a-enolase, C-1-tetrahydrofolate synthase, cytoplasmic, Pyruvate kinase, isozymes M1/M2, Ubiquitin activating enzyme E1, 26S protease regulatory subunit S10B, 60S acidic ribosomal protein P2, 60S acidic ribosomal protein P0, 40S ribosomal protein SA, 40S ribosomal protein S2, 40S ribosomal protein S3, 60S ribosomal protein L4, 60S ribosomal protein L3, 40S ribosomal protein S3a, 40S ribosomal protein S7, 60S ribosomal protein L7a, 60S acidic ribosomal protein L31, 60S ribosomal protein L10a, 60S ribosomal protein L6, 26S proteasome non-ATPase regulatory subunit 1, Tubulin b-2 chain, Actin, cytoplasmic 1, Actin, aortic smooth muscle, Tubulin a-ubiquitous chain, Clathrin heavy chain 1, Histone H2B.b, Histone H4, Histone H3.1, Histone H3.3, Histone H2A type 8, 26S protease regulatory subunit 6A, Ubiquitin-4, RuvB like 1, 26S protease regulatory subunit 7, Leucyl-tRNA synthetase, cytoplasmic, 60S ribosomal protein L19, 26S proteasome non-ATPase regulatory subunit 13, Histone H2B.F, U5 small nuclear ribonucleoprotein 200 kDa helicase, Poly[ADP-ribose]polymerase-1, ATP-dependent DNA helicase II, DNA replication licensing factor MCCC5, Nuclease sensitive element binding protein 1, ATP-dependent RNA helicase A, Interleukin enhancer binding factor 3, Transcription elongation factor B polypeptide 1, Pre-mRNA processing splicing factor 8, Staphylococcal nuclease domain containing protein 1, Programmed cell death 6-interacting protein, Mediator of RNA polymerase II transcription subunit 8 homolog, Nucleolar RNA helicase II, Endoplasmin, DnaJ homolog subfamily A member 1, Heat shock 70 kDa protein 1L, T-complex protein 1 e subunit, GNE-like protein 1, Serotransferrin, Fructose bisphosphate aldolase A, Inosine-5′monophosphate dehydrogenase 2, 26S protease regulatory subunit 6B, Fatty acid synthase, DNA-dependent protein kinase catalytic subunit, 40S ribosomal protein 517, 60S ribosomal protein L7, 60S ribosomal protein L12, 60S ribosomal protein L9, 40S ribosomal protein S8, 40S ribosomal protein S4 X isoform, 60S ribosomal protein L11, 26S proteasome non-ATPase regulatory subunit 2, Coatomer a subunit, Histone H2A.z, Histone H1.2, Dynein heavy chain cytosolic. See: Saphire et al., Journal of Proteome Research, 2005, and Wheeler et al., Proteomics Clinical Applications, 2007.

In some embodiments the fusosome is pegylated.

### Particle Size

In some embodiments the median fusosome diameter is between 10 and 1000 nM, 25 and 500 nm 40 and 300 nm, 50 and 250 nm, 60 and 225 nm, 70 and 200 nm, 80 and 175 nm, or 90 and 150 nm.

In some embodiments, 90% of the fusosomes fall within 50% of the median diameter. In some embodiments, 90% of the fusosomesfall within 25% of the median diameter. In some embodiments, 90% of the fusosomesfall within 20% of the median diameter. In some embodiments, 90% of the fusosomesfall within 15% of the median diameter. In some embodiments, 90% of the fusosomesfall within 10% of the median diameter.

### Indications and Uses

In some embodiments, the fusosome or pharmaceutical compositions thereof as described herein can be administered to a subject, e.g. a mammal, e.g. a human. In some aspects, provided herein are fusosomes, or pharmaceutical compositions, such as any as described herein, that can be administered to a subject, e.g., a mammal, e.g., a human. In such embodiments, the subject may be at risk of, may have a symptom of, or may be diagnosed with or identified as having, a particular disease or condition (e.g., a disease or condition described herein). In one embodiment, the subject has cancer. In one embodiment, the subject has an infectious disease. In some embodiments, the fusosome, e.g. retroviral vectors or particles, contains nucleic acid sequences encoding an exogenous agent for treating the disease or condition in the subject. For example, the exogenous agent is one that targets or is specific for a protein of a neoplastic cells and the fusosome is administered to a subject for treating a tumor or cancer in the subject. In another example, the exogenous agent is an inflammatory mediator or immune molecule, such as a cytokine, and the fusosome is administered to a subject for treating any condition in which it is desired to modulate (e.g. increase) the immune response, such as a cancer or infectious disease.

Thus, also provided, in some aspects, are methods of administering and uses, such as therapeutic and prophylactic uses, of the provided fusosomes, e.g., retroviral vectors and particles, such as lentiviral vectors and particles, and/or compositions comprising the same. Such methods and uses include therapeutic methods and uses, for example, involving administration of the fusosomes, e.g., retroviral vectors or particles, such as lentiviral vectors or particles, or compositions containing the same, to a subject having a disease, condition, or disorder for delivery of an exogenous agent for treatment of the disease, condition or disorder. In some embodiments, the fusosome (e.g., retroviral vector or particle, such as lentiviral vector or particle) is administered in an effective amount or dose to effect treatment of the disease, condition or disorder. Provided herein are uses of any of the provided fusosomes (e.g. retroviral vector or particle, such as lentiviral vector or particle) in such methods and treatments, and in the preparation of a medicament in order to carry out such therapeutic methods. In some embodiments, the methods are carried out by administering the fusosomes (e.g. retroviral vector or particle, such as lentiviral vector or particle), or compositions comprising the same, to the subject having, having had, or suspected of having the disease or condition or disorder. In some embodiments, the methods thereby treat the disease or condition or disorder in the subject. Also provided herein are use of any of the compositions, such as pharmaceutical compositions provided herein, for the treatment of a disease, condition or disorder associated with a particular gene or protein targeted by or provided by the exogenous agent.

The administration of a pharmaceutical composition described herein may be, for example, by way of oral, inhaled, transdermal or parenteral (including intravenous, intratumoral, intraperitoneal, intramuscular, intracavity, and subcutaneous) administration. The fusosomes may, in some embodiments, be administered alone or formulated as a pharmaceutical composition.

In embodiments, the fusosome composition mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the fusosome composition comprises an exogenous protein), the effect lasts for less than 1, 2, 3, 4, 5, 6, or 7 days, 2, 3, or 4 weeks, or 1, 2, 3, 6, or 12 months.

In embodiments, the fusosome composition described herein is delivered ex-vivo to a cell or tissue, e.g., a human cell or tissue.

The fusosome compositions described herein can, in some embodiments, be administered to a subject, e.g., a mammal, e.g., a human. In certain embodiments, the subject may be at risk of, may have a symptom of, or may be diagnosed with or identified as having, a particular disease or condition (e.g., a disease or condition described herein).

In some embodiments, the source of fusosomes are from the same subject that is administered a fusosome composition. In other embodiments, they are different. For example, the source of fusosomes and recipient tissue may be autologous (from the same subject) or heterologous (from different subjects). In either case, the donor tissue for fusosome compositions described herein may be a different tissue type than the recipient tissue. For example, the donor tissue may be muscular tissue and the recipient tissue may be connective tissue (e.g., adipose tissue). In other embodiments, the donor tissue and recipient tissue may be of the same or different type, but from different organ systems.

In some embodiments, the fusosome is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3:1462 DOI: 10.1038/srep01462). Thus, in some embodiments, the fusosome is co-administered with an inhibitor of sypressyn, e.g., a siRNA or inhibitory antibody.

Compositions described herein may, in some embodiments, be used to similarly modulate the cell or tissue function or physiology of a variety of other organisms, including but not limited to: farm or working animals (horses, cows, pigs, chickens etc.), pet or zoo animals (cats, dogs, lizards, birds, lions, tigers and bears etc.), aquaculture animals (fish, crabs, shrimp, oysters etc.), plants species (trees, crops, ornamentals flowers etc), fermentation species (saccharomyces etc.). Fusosome compositions described herein can be made, in some embodiments, from such non-human sources and administered to a non-human target cell or tissue or subject.

Fusosome compositions can be autologous, allogeneic or xenogeneic to the target.

### Additional Therapeutic Agents

In some embodiments, the fusosome composition is co-administered with an additional agent, e.g., a therapeutic agent, to a subject, e.g., a recipient, e.g., a recipient described herein. In some embodiments, the co-administered therapeutic agent is an immunosuppressive agent, e.g., a glucocorticoid (e.g., dexamethasone), cytostatic (e.g., methotrexate), antibody (e.g., Muromonab-CD3), or immunophilin modulator (e.g., Ciclosporin or rapamycin). In embodiments, the immunosuppressive agent decreases immune mediated clearance of fusosomes. In some embodiments the fusosome composition is co-administered with an immunostimulatory agent, e.g., an adjuvant, an interleukin, a cytokine, or a chemokine.

In some embodiments, the fusosome composition and the immunosuppressive agent are administered at the same time, e.g., contemporaneously administered. In some embodiments, the fusosome composition is administered before administration of the immunosuppressive agent. In some embodiments, the fusosome composition is administered after administration of the immunosuppressive agent.

In some embodiments, the immunosuppressive agent is a small molecule such as ibuprofen, acetaminophen, cyclosporine, tacrolimus, rapamycin, mycophenolate, cyclophosphamide, glucocorticoids, sirolimus, azathriopine, or methotrexate.

In some embodiments, the immunosuppressive agent is an antibody molecule, including but not limited to: muronomab (anti-CD3), Daclizumab (anti-IL12), Basiliximab, Infliximab (Anti-TNFa), or rituximab (Anti-CD20).

In some embodiments, co-administration of the fusosome composition with the immunosuppressive agent results in enhanced persistence of the fusosome composition in the subject compared to administration of the fusosome composition alone. In some embodiments, the enhanced persistence of the fusosome composition in the co-administration is at least 10%, 20%, 30%, 40%, 50%, 60%, 70%, 80%, 90% or longer, compared to persistence of the fusosome composition when administered alone. In some embodiments, the enhanced persistence of the fusosome composition in the co-administration is at least 1, 2, 3, 4, 5, 6, 7, 10, 15, 20, 25, or 30 days or longer, compared to survival of the fusosome composition when administered alone.

### Examples

The Examples below are set forth to aid in the understanding of the inventions, but are not intended to, and should not be construed to, limit its scope in any way.

### Example 1: Elevated Levels of a Cathepsin Molecule LED to Increased Functional Titre of Fusosome on Target Cells

This Example describes the generation of active fusosomes (in particular, pseudotyped lentivirus fusosomes) and the effects of elevated levels of cathepsin L in these fusosome-producer cells on resulting pseudotyped lentivirus titres on CD8 overexpressing target cells. Human embryonic kidney cells (293LX cells) were transfected with the vectors psPAX2, pLenti-GFP, and pCAGGS Niv-CD8/Fd22 to serve as fusosome producer cells. The producer cells were also transfected with 1 pg of pcDNA (No CathL control) or Cathepsin L DNA (CathL). These modified producer cells were harvested at 48 hours. At the time of harvest, GFP, which served as a marker for active production of fusosomes, was detected in large syncytia in producer cells that had elevated levels of the cathepsin L molecule in comparison to the pcDNA transfected control cells (data not shown).

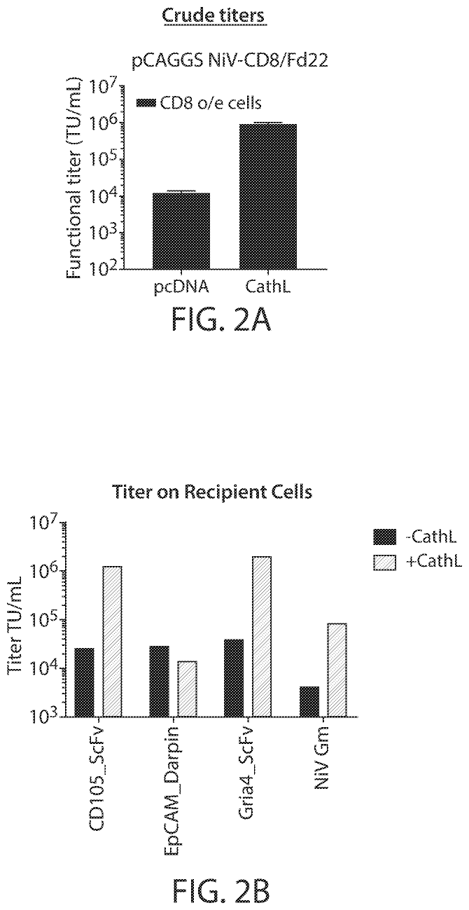

The supernatants containing the pseudotyped lentiviruses were then used to transduce the target CD8 overexpressing cells. Lentiviral vector supernatants were serial diluted in 293LX cell culture media (DMEM with 10% fetal bovine serum) and applied to 293LX cells that were transfected to over-express human CD8A and B for 24 hours. GFP expression was analyzed by flow cytometry 72 hours post-transduction, and the serial dilution point that corresponded to 5-15% GFP-positive cells was used to calculate the lentiviral vector titer. As shown in FIG. 2A, the fusosomes produced in the modified 293LX producer cells with elevated levels of cathepsin L had functional titres of about 1,000,000 TU/mL on CD8 overexpressing cells. The fusosomes generated in control cells transfected with only pcDNA (no elevated cathepsin L) had functional titres of only about 10,000 TU/mL. These results demonstrated that incorporation of elevated levels of cathepsin L in fusosome-producer cells resulted in an approximately 100-fold increase in functional titres on the target cells.

### Example 2: Elevated Levels of a Cathepsin Molecule LED to Increased Functional Titres of Retargeted Fusosomes on Target Cells

This Example describes the production of retargeted fusosomes and the effects of elevated levels of cathepsin L in these retargeted fusosome producer cells on their resulting pseudotyped lentivirus titres on target cells. Human embryonic kidney cells (293LX cells) were transfected with the vectors psPAX2 and pLenti-GFP. To retarget fusosomes with additional binding moieties, these producer cells were also transfected with either NivGm-CD105-ScFv, NivGm-EpCAM-Darpin, or NivGm-Gria4-ScFv. Additionally, producer cells were transfected with pcDNA (No CathL control) or Cathepsin L DNA (CathL). Supernatants from the modified producer cells were harvested at 48 hours. The supernatants containing the pseudotyped lentiviruses were then used to transduce target 293LX cells transfected to over-express CD105, EpCAM, or Gria4 receptors (or mock DNA as control, referred to as − CathL). GFP expression was analyzed by flow cytometry 72 hours post-transduction. As shown in FIG. 2B, the fusosomes targeting CD105 produced in modified cells with elevated levels of cathepsin L had functional titres of at least 1,000,000 TU/mL on target cells, compared to functional titres of slightly above 10,000 TU/mL on target cells generated by fusosomes produced by control cells only transfected with pcDNA (no elevated cathepsin L). Similarly, as also shown in FIG. 2B, the fusosomes targeting Gria4 produced in modified cells with elevated levels of cathepsin L had functional titres greater than 1,000,000 TU/mL on target cells, compared to functional titres of above 10,000 TU/mL on target cells generated by fusosomes produced by control cells transfected with only pcDNA (no elevated cathepsin L). These results indicated that incorporation of elevated levels of cathepsin L in these retargeted fusosome producer cells resulted in at least a 10- to 100-fold increase in functional titres for fusosomes targeted to CD105 and Gria4, in addition to the CD8-targeted fusosomes described in Example 1. This experiment also demonstrated production of non-targeted fusosomes and fusosomes targeted to CD105, EpCAM, Gria4, and CD8.

### Example 3: Elevated Levels of a Cathepsin Molecule Increased Functional Titres of Fusosomes on Activated T Cells

This Example describes the generation of active fusosomes and the effects of elevated levels of cathepsin L in these active fusosome producer cells on resulting pseudotyped lentivirus titres on PanT cells (human T cells negatively selected to remove any CD3-negative cells; obtained from StemCell Tech). PanT cells were thawed and activated with CD3/CD28 and IL-2 for 48 hours prior to transduction with lentiviral vectors via spin-oculation for 90 minutes. To make produced cells, human embryonic kidney cells (293LX cell line) were transfected with the vectors psPAX2 and pLenti-SFFV-eGFP, along with additional vectors and conditions as indicated in Table 6. The pseudotyped lentivirus samples were harvested 48 hours post-transfection.

The pseudotyped lentivirus samples were then concentrated approximately 400× by ultracentrifugation. Both crude and concentrated samples were used to transduce the target cells which included PanT cells, Molt4.8 cells, and 293LX cells that were transiently transfected with hCD8A and B at 24 hours post-transfection (and do not naturally express detectable amounts of CD8). Six days post-transduction, GFP expression was analyzed by flow cytometry.

TABLE 6
Transfection conditions and vectors used for producer fusogens and their
resulting pseudotyped lentivirus samples that were then utilized for
transduction of target cells

| Sample | | | Other | Transfection | |
|---|---|---|---|---|---|
| ID# | Binder | Fusogen | DNA | Type | Media |
| 336 | NivGm-hCD8- | NivFd22 | pcDNA | Xfect Reverse | DMEM + |
| | s1 | | | | 10% FBS |
| 337 | NivGm-hCD8- | NivFd22- | pcDNA | Xfect Reverse | DMEM + |
| | s1 | HA | | | 10% FBS |
| 338 | NivGm-hCD8- | NivFd22 | CathL | Xfect Reverse | DMEM + |
| | s1 | | | | 10% FBS |
| 339 | NivGm-hCD8- | NivFd22- | CathL | Xfect Reverse | DMEM + |
| | s1 | HA | | | 10% FBS |

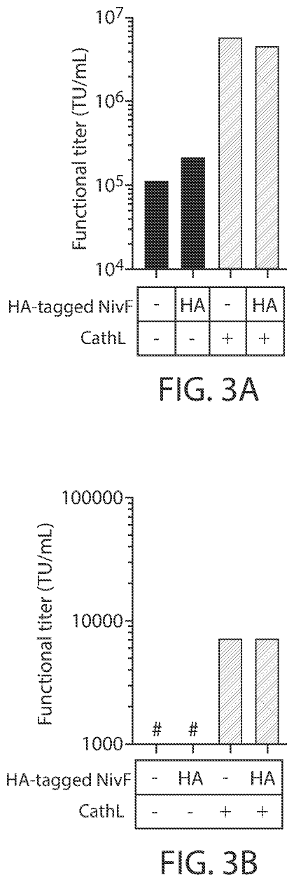

As shown in FIGS. 3A-3B, the CD8 targeting fusosomes produced in modified cells with elevated levels of cathepsin L transfected by Xfect/DMEM had approximately 100-fold higher functional titres on PanT cells than fusosomes produced in control cells transfected with only pcDNA. This approximately 100-fold difference was observed when target cells were transduced with either crude or concentrated pseudotyped lentivirus samples. While data is only shown for PanT cells in FIGS. 3A-3B, similar results were observed with the Molt4.8 target cells and 293LX target cells transiently transfected with hCD8A and B.

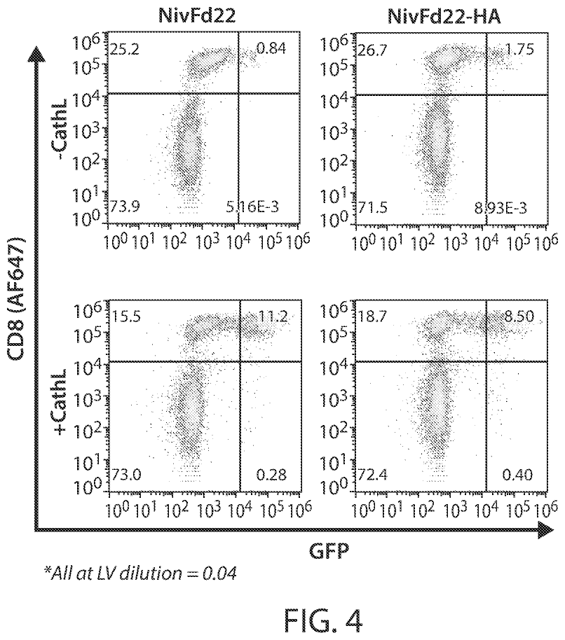

Additionally, as shown in FIG. 4 and Table 7, the number of double-positive CD8 and GFP PanT cells was quantified by flow cytometry. GFP was used as a marker for the active NivFd22-HA tagged or untagged fusogen and thus successful transduction of target cells. The number of CD8+ PanT cells also positive for GFP increased when fusosomes were produced in modified cells with elevated levels of cathepsin L that were transfected by Xfect/DMEM.

TABLE 7
Quantified percent of double positive CD8 and GFP cells by flow
cytometry following transduction of PanT cells with pseudotyped
lentivirus samples from producer cells modified as described
in the table

| | | | | | CD8+/ |
|---|---|---|---|---|---|
| Sample | | Other | Transfection | | GFP + |
| ID # | Fusogen | DNA | Type | Media | Cells (%) |
| 336 | NivFd22 | pcDNA | Xfect Reverse | DMEM + 10% | 0.84 |
| | | | | FBS | |
| 337 | NivFd22- | pcDNA | Xfect Reverse | DMEM + 10% | 1.75 |
| | HA | | | FBS | |
| 338 | NivFd22 | CathL | Xfect Reverse | DMEM + 10% | 11.2 |
| | | | | FBS | |
| 339 | NivFd22- | CathL | Xfect Reverse | DMEM + 10% | 8.5 |
| | HA | | | FBS | |

### Example 4: Elevated Levels of a Cathepsin Molecule Increased Henipavirus F Protein Processing and Decreased Overall Lentiviral Particle Number in Fusosome Producer Cells

This Example describes the effects of elevated levels of cathepsin L in fusosome producer cells on henipavirus F protein processing in said producer cells and the effects of elevated levels of cathepsin on the overall number of resulting pseudotyped lentivirus particles. The CD8 targeted fusosome producer cells in this Example were generated as described in Example 3 and Table 6 of Example 3.

A. Elevated Cathepsin Levels Increased Henipavirus Protein F Processing in Fusosome Producer Cells and their Resulting Pseudotyped Lentiviruses

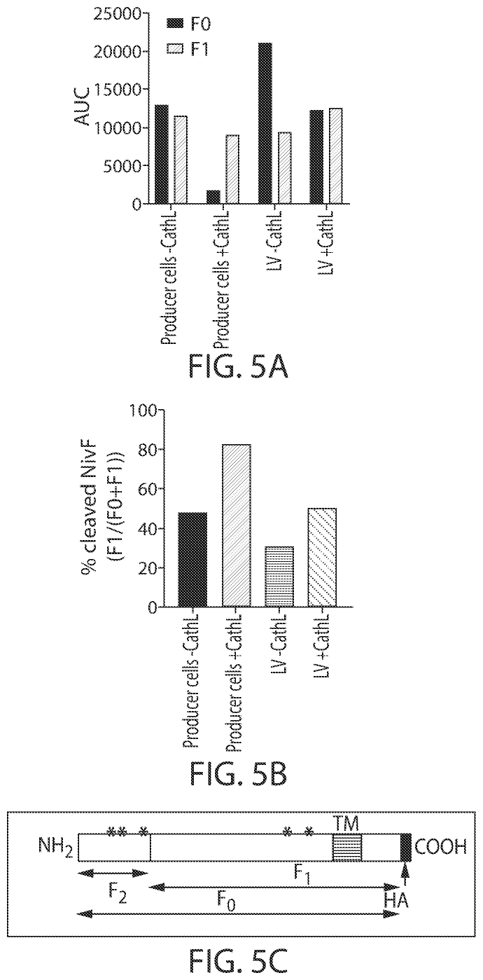

The total amount of inactive (F₀) and active (F₁) henipavirus protein F was quantified 20 using band density on a Western blot that was probed with an anti-HA antibody. The inactive henipavirus protein F (F₀) had a molecular weight of approximately 60 kD and active henipavirus protein F (F₁) had a molecular weight of approximately of approximately 40 kD. When producer cells had elevated levels of cathepsin L, the amount of active henipavirus protein F (F₁) within the producer cell and their respective pseudotyped lentivirus samples remained approximately unchanged, whereas the amount of inactive protein (F₀) decreased, as demonstrated in FIG. 5A. It was confirmed by Western blot using an anti-protein F antibody that this observation was unaffected by presence of the HA tag (data not shown).

Additionally, in FIG. 5B, the percent of active henipavirus protein F to total henipavirus protein F also increased in producer cells and their respective pseudotyped lentivirus samples that were transfected with elevated levels of cathepsin L. Therefore, FIGS. 5A-5B indicated that elevated cathepsin levels increased henipavirus protein F processing in producer cells, as compared to fusosome producer control cells that were transfected with only pcDNA.

B. Modified Producer Cells Transfected with Cathepsin Demonstrated Elevated Levels of the Cathepsin Molecule and the Ability to Process Cathepsin

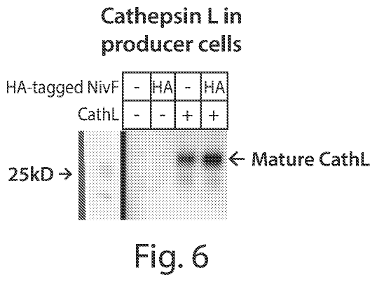

The levels of pro-cathepsin L and mature cathepsin L were evaluated using the same Western blot membrane from Example 4A. The membrane was stripped and re-probed with an anti-Cathepsin L antibody. As observed in FIG. 6, producer cells transfected with cathepsin L using the Xfect/DMEM method showed elevated levels of cathepsin L and also processing of the pro-cathepsin L form (molecular weight: approximately 38-42 kD) to the mature cathepsin L form (molecular weight: approximately 25-35 kD).

### C. Elevated Cathepsin Levels Decreased β24 Levels in Pseudotyped Lentiviral Particles Produced by the Fusosome Producer Cells

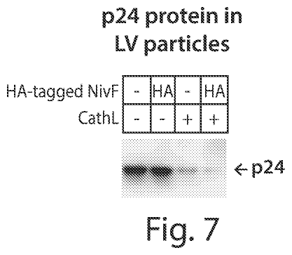

The expression level of β24 in the pseudotyped lentivirus samples produced by the modified producer cells was measured using the same Western blot membrane from Example 4A and 4B. The membrane was stripped and re-probed with an anti-β24 antibody. The β24 antigen is a lentiviral capsid protein and was used here as a marker for lentiviral particles. As demonstrated by FIG. 7, elevated levels of cathepsin L in producer cells decreased β24 expression in their respective pseudotyped lentivirus samples. Therefore, elevated levels of cathepsin in producer cells decreased overall pseudotyped lentivirus particle production, as compared to producer control cells that did not overexpress cathepsin. Whereas elevated levels of cathepsin in producer cells was observed to lead to the formation of fewer overall pseudotyped lentivirus particles, it was observed to generate a greater proportion of active particles. In some embodiments, a pharmaceutical composition comprising a higher proportion of active particles is advantageous for administration to subjects.

### Example 5: Elevated Levels of Cathepsin Decreased Levels of Henipavirus Protein G in Fusosome Producer Cells

This Example describes the effects of elevated levels of cathepsin L in fusosome producer cells on henipavirus G protein expression in the producer cells and their resulting pseudotyped lentivirus samples. The CD8 targeted fusosome producer cells in this Example were generated using the same methods described in Example 3 and Table 6 of Example 3.

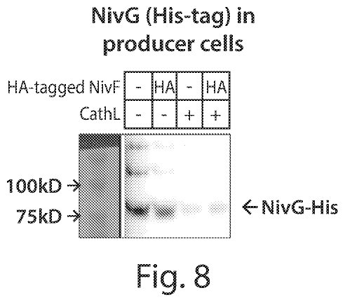

As demonstrated in FIG. 8, Western blot analysis of the levels of henipavirus protein G (His tagged) in producer cells and their resulting pseudotyped lentivirus samples was 5 conducted with an anti-His antibody. Elevated levels of cathepsin L led to decreased cellular expression of henipavirus protein G in the modified fusosome producer cells. These results are consistent with a lower overall number of lentiviral particles being produced, in agreement with the results described in Example 4 above.

SEQUENCE TABLE

| SEQ ID | | |
|---|---|---|
| NO | SEQUENCE | ANNONATION |
| 1 | MDYAFQYVQDNGGLDSEESYPYEATEESCKYNPKYSVA | Exemplary |
| | NDTGFVDIPKQEKALMKAVATVGPISVAIDAGHESFLFY | Cathepsin L1 |
| | KEGIYFEPDCSSEDMDHGVLVVGYGFESTESDNNKYWL | Sequence |
| | VKNSWGEEWGMGGYVKMAKDRRNHCGIASAASYPTV | |
| 2 | MHGNNGHSVPPSKRSETRAPVAPAGCNGGYPAEAWNF | Exemplary |
| | WTRKGLVSGGLYESHVGCRPYSIPPCEHHVNGSRPPCTG | Cathepsin B |
| | EGDTPKCSKICEPGYSPTYKQDKHYGYNSYSVSNSEKDI | Sequence |
| | MAEIYKNGPVEGAFSVYSDFLLYKSGVYQHVTGEMMGG | |
| | HAIRILGWGVENGTPYWLVANSWNTDWGDNGFFKILRG | |
| | QDHCGIESEVVAGIPRTDQYWEKI | |
| 3 | MATQEVRLKCLLCGIIVLVLSLEGLGILHYEKLSKIGL | HeVF |
| | VKGITRKYKIKSNPLTKDIVIKMIPNVSNVSKCTGTV | |
| | MENYKSRLTGILSPIKGAIELYNNNTHDLVGDVKLA | |
| | GVVMAGIAIGIATAAQITAGVALYEAMKNADNINKL | |
| | KSSIESTNEAVVKLQETAEKTVYVLTALQDYINTNLV | |
| | PTIDQISCKQTELALDLALSKYLSDLLFVFGPNLQDPV | |
| | SNSMTIQAISQAFGGNYETLLRTLGYATEDFDDLLES | |
| | DSIAGQIVYVDLSSYYIIVRVYFPILTEIQQAYVQELLP | |
| | VSFNNDNSEWISIVPNFVLIRNTLISNIEVKYCLITKKS | |
| | VICNQDYATPMTASVRECLTGSTDKCPRELVVSSHV | |
| | PRFALSGGVLFANCISVTCQCQTTGRAISQSGEQTLL | |
| | MIDNTTCTTVVLGNIIISLGKYLGSINYNSESIAVGPP | |
| | VYTDKVDISSQISSMNQSLQQSKDYIKEAQKILDTV | |
| | NPSLISMLSMIILYVLSIAALCIGLITFISFVIVEKKRG | |
| | NYSRLDDRQVRPVSNGDLYYIGT | |
| 4 | MSNKRTTVLIIISYTLFYLNNAAIVGFDFDKLNKIGVVQ | CedVF |
| | GRVLNYKIKGDPMTKDLVLKFIPNIVNITECVREPLSRY | |
| | NETVRRLLLPIHNMLGLYLNNTNAKMTGLMIAGVIMG | |
| | GIAIGIATAAQITAGFALYEAKKNTENIQKLTDSIMKTQ | |
| | DSIDKLTDSVGTSILILNKLQTYINNQLVPNLELLSCRQN | |
| | KIEFDLMLTKYLVDLMTVIGPNINNPVNKDMTIQSLSL | |
| | LFDGNYDIMMSELGYTPQDFLDLIESKSITGQIIYVDME | |
| | NLYVVIRTYLPTLIEVPDAQIYEFNKITMSSNGGEYLST | |
| | IPNFILIRGNYMSNIDVATCYMTKASVICNQDYSLPMS | |
| | QNLRSCYQGETEYCPVEAVIASHSPRFALTNGVIFANC | |
| | INTICRCQDNGKTITQNINQFVSMIDNSTCNDVMVDKF | |
| | TIKVGKYMGRKDINNINIQIGPQIIIDKVDLSNEINKMNQ | |
| | SLKDSIFYLREAKRILDSVNISLISPSVQLFLIIISVLSFIIL | |
| | LIIIVYLYCKSKHSYKYNKFIDDPDYYNDYKRERINGKA | |
| | SKSNNIYYVGD | |
| 5 | MALNKNMFSSLFLGYLLVYATTVQSSIHYDSLSKVGVIK | Mojiang virus F |
| | GLTYNYKIKGSPSTKLMVVKLIPNIDSVKNCTQKQYDEY | |
| | KNLVRKALEPVKMAIDTMLNNVKSGNNKYRFAGAIMA | |
| | GVALGVATAATVTAGIALHRSNENAQAIANMKSAIQNT | |
| | NEAVKQLQLANKQTLAVIDTIRGEINNNIIPVINQLSCDTI | |
| | GLSVGIRLTQYYSEIITAFGPALQNPVNTRITIQAISSVEN | |
| | GNFDELLKIMGYTSGDLYEILHSELIRGNIIDVDVDAGY | |
| | IALEIEFPNLTLVPNAVVQELMPISYNIDGDEWVTLVP | |
| | RFVLTRTTLLSNIDTSRCTITDSSVICDNDYALPMSHEL | |
| | IGCLQGDTSKCAREKVVSSYVPKFALSDGLVYANCLN | |
| | TICRCMDTDTPISQSLGATVSLLDNKRCSVYQVGDVLI | |
| | SVGSYLGDGEYNADNVELGPPIVIDKIDIGNQLAGINQ | |
| | TLQEAEDYIEKSEEFLKGVNPSIITLGSMVVLYIFMILI | |
| | AIVSVIALVLSIKLTVKGNVVRQQFTYTQHVPSMENINYV | |
| | SH | |
| 6 | MKKKTDNPTISKRGHNHSRGIKSRALLRETDNYSNG | Bat PVF |
| | LIVENLVRNCHHPSKNNLNYTKTQKRDSTIPYRVEER | |
| | KGHYPKIKHLIDKSYKHIKRGKRRNGHNGNIITIILLL | |
| | ILILKTQMSEGAIHYETLSKIGLIKGITREYKVKGTPS | |
| | SKDIVIKLIPNVTGLNKCTNISMENYKEQLDKILIPIN | |
| | NIIELYANSTKSAPGNARFAGVIIAGVALGVAAAAQIT | |
| | AGIALHEARQNAERINLLKDSISATNNAVAELQEATG | |
| | GIVNVITGMQDYINTNLVPQIDKLQCSQIKTALDISLS | |
| | QYYSEILTVFGPNLQNPVTTSMSIQAISQSFGGNIDLLL | |
| | NLLGYTANDLLDLLESKSITGQITYINLEHYFMVIRV | |
| | YYPIMTTISNAYVQELIKISFNVDGSEWVSLVPSYILIR | |
| | NSYLSNIDISECLITKNSVICRHDFAMPMSYTLKECLTG | |
| | DTEKCPREAVVTSYVPRFAISGGVIYANCLSTTCQCYQ | |
| | TGKVIAQDGSQTLMMIDNQTCSIVRIEEILISTGKYLGS | |
| | QEYNTMHVSVGNPVFTDKLDITSQISNINQSIEQSKFY | |
| | LDKSKAILDKINLNLIGSVPISILFIIAILSLILSIITFVIVM | |
| | IIVRRYNKYTPLINSDPSSRRSTIQDVYIIPNPGEHSIRSAAR | |
| | SIDRDRD | |
| 7 | MVVILDKRCYCNLLILILMISECSVGILHYEKLSKIGLVKG | Nipah F0 |
| | VTRKYKIKSNPLTKDIVIKMIPNVSNMSQCTGSVMENYK | |
| | TRLNGILTPIKGALEIYKNNTHDLVGDVRLAGVIMAGVAI | |
| | GIATAAQITAGVALYEAMKNADNINKLKSSIESTNEAVV | |
| | KLQETAEKTVYVLTALQDYINTNLVPTIDKISCKQTELSL | |
| | DLALSKYLSDLLFVFGPNLQDPVSNSMTIQAISQAFGGNY | |
| | ETLLRTLGYATEDFDDLLESDSITGQIIYVDLSSYYIIVRVY | |
| | FPILTEIQQAYIQELLPVSFNNDNSEWISIVPNFILVRNTLIS | |
| | NIEIGFCLITKRSVICNQDYATPMTNNMRECLTGSTEKCP | |
| | RELVVSSHVPRFALSNGVLFANCISVTCQCQTTGRAISQS | |
| | GEQTLLMIDNTTCPTAVLGNVIISLGKYLGSVNYNSEGIAI | |
| | GPPVFTDKVDISSQISSMNQSLQQSKDYIKEAQRLLDTVN | |
| | PSLISMLSMIILYVLSIASLCIGLITFISFIIVEKKRNTYSRLE | |
| | DRRVRPTSSGDLYYIGT | |
| 8 | MMADSKLVSLNNNLSGKIKDQGKVIKNYYGTMDIKKIN | HeV G |
| | DGLLDSKILGAFNTVIALLGSIIIIVMNIMIIQNYTRTTDNQ | |
| | ALIKESLQSVQQQIKALTDKIGTEIGPKVSLIDTSSTITIPAN | |
| | IGLLGSKISQSTSSINENVNDKCKFTLPPLKIHECNISCPNP | |
| | LPFREYRPISQGVSDLVGLPNQICLQKTTSTILKPRLISYTL | |
| | PINTREGVCITDPLLAVDNGFFAYSHLEKIGSCTRGIAKQR | |
| | IIGVGEVLDRGDKVPSMFMTNVWTPPNPSTIHHCSSTYHE | |
| | DFYYTLCAVSHVGDPILNSTSWTESLSLIRLAVRPKSDSG | |
| | DYNQKYIAITKVERGKYDKVMPYGPSGIKQGDTLYFPAV | |
| | GFLPRTEFQYNDSNCPIIHCKYSKAENCRLSMGVNSKSHY | |
| | ILRSGLLKYNLSLGGDIILQFIEIADNRLTIGSPSKIYNSLGQ | |
| | PVFYQASYSWDTMIKLGDVDTVDPLRVQWRNNSVISRP | |
| | GQSQCPRFNVCPEVCWEGTYNDAFLIDRLNWVSAGVYL | |
| | NSNQTAENPVFAVFKDNEILYQVPLAEDDTNAQKTITDC | |
| | FLLENVIWCISLVEIYDTGDSVIRPKLFAVKIPAQCSES | |
| 9 | MPAENKKVRFENTTSDKGKIPSKVIKSYYGTMDIKKINEG | NIV G |
| | LLDSKILSAFNTVIALLGSIVIIVMNIMIIQNYTRSTDNQAV | |
| | IKDALQGIQQQIKGLADKIGTEIGPKVSLIDTSSTITIPANIG | |
| | LLGSKISQSTASINENVNEKCKFTLPPLKIHECNISCPNPLP | |
| | FREYRPQTEGVSNLVGLPNNICLQKTSNQILKPKLISYTLP | |
| | VVGQSGTCITDPLLAMDEGYFAYSHLERIGSCSRGVSKQ | |
| | RIIGVGEVLDRGDEVPSLFMTNVWTPPNPNTVYHCSAVY | |
| | NNEFYYVLCAVSTVGDPILNSTYWSGSLMMTRLAVKPKS | |
| | NGGGYNQHQLALRSIEKGRYDKVMPYGPSGIKQGDTLY | |
| | FPAVGFLVRTEFKYNDSNCPITKCQYSKPENCRLSMGIRP | |
| | NSHYILRSGLLKYNLSDGENPKVVFIEISDQRLSIGSPSKIY | |
| | DSLGQPVFYQASFSWDTMIKFGDVLTVNPLVVNWRNNT | |
| | VISRPGQSQCPRFNTCPEICWEGVYNDAFLIDRINWISAGV | |
| | FLDSNQTAENPVFTVFKDNEILYRAQLASEDTNAQKTITN | |
| | CFLLKNKIWCISLVEIYDTGDNVIRPKLFAVKIPEQC | |
| 10 | MLSQLQKNYLDNSNQQGDKMNNPDKKLSVNFNPLELDK | CedV G |
| | GQKDLNKSYYVKNKNYNVSNLLNESLHDIKFCIYCIFSLL | |
| | IIITIINIITISIVITRLKVHEENNGMESPNLQSIQDSLSSLTN | |
| | MINTEITPRIGILVTATSVTLSSSINYVGTKTNQLVNELKD | |
| | YITKSCGFKVPELKLHECNISCADPKISKSAMYSTNAYAE | |
| | LAGPPKIFCKSVSKDPDFRLKQIDYVIPVQQDRSICMNNPL | |
| | LDISDGFFTYIHYEGINSCKKSDSFKVLLSHGEIVDRGDYR | |
| | PSLYLLSSHYHPYSMQVINCVPVTCNQSSFVFCHISNNTK | |
| | TLDNSDYSSDEYYITYFNGIDRPKTKKIPINNMTADNRYI | |
| | HFTFSGGGGVCLGEEFIIPVTTVINTDVFTHDYCESFNCSV | |
| | QTGKSLKEICSESLRSPTNSSRYNLNGIMIISQNNMTDFKI | |
| | QLNGITYNKLSFGSPGRLSKTLGQVLYYQSSMSWDTYLK | |
| | AGFVEKWKPFTPNWMNNTVISRPNQGNCPRYHKCPEICY | |
| | GGTYNDIAPLDLGKDMYVSVILDSDQLAENPEITVFNSTT | |
| | ILYKERVSKDELNTRSTTTSCFLFLDEPWCISVLETNRFNG | |
| | KSIRPEIYSYKIPKYC | |
| 11 | MPQKTVEFINMNSPLERGVSTLSDKKTLNQSKITKQGYF | Bat PV G |
| | GLGSHSERNWKKQKNQNDHYMTVSTMILEILVVLGIMF | |
| | NLIVLTMVYYQNDNINQRMAELTSNITVLNLNLNQLTNK | |
| | IQREIIPRITLIDTATTITIPSAITYILATLTTRISELLPSINQKC | |
| | EFKTPTLVLNDCRINCTPPLNPSDGVKMSSLATNLVAHGP | |
| | SPCRNFSSVPTIYYYRIPGLYNRTALDERCILNPRLTISSTK | |
| | FAYVHSEYDKNCTRGFKYYELMTFGEILEGPEKEPRMFS | |
| | RSFYSPTNAVNYHSCTPIVTVNEGYFLCLECTSSDPLYKA | |
| | NLSNSTFHLVILRHNKDEKIVSMPSFNLSTDQEYVQIIPAE | |
| | GGGTAESGNLYFPCIGRLLHKRVTHPLCKKSNCSRTDDES | |
| | CLKSYYNQGSPQHQVVNCLIRIRNAQRDNPTWDVITVDL | |
| | TNTYPGSRSRIFGSFSKPMLYQSSVSWHTLLQVAEITDLD | |
| | KYQLDWLDTPYISRPGGSECPFGNYCPTVCWEGTYNDV | |
| | YSLTPNNDLFVTVYLKSEQVAENPYFAIFSRDQILKEFPLD | |
| | AWISSARTTTISCFMFNNEIWCIAALEITRLNDDIIRPIYYS | |
| | FWLPTDCRTPYPHTGKMTRVPLRSTYNY | |
| 12 | MATNRDNTITSAEVSQEDKVKKYYGVETAEKVADSISGN | Mojiang virus G |
| | KVFILMNTLLILTGAIITITLNITNLTAAKSQQNMLKIIQDD | |
| | VNAKLEMFVNLDQLVKGEIKPKVSLINTAVSVSIPGQISN | |
| | LQTKFLQKYVYLEESITKQCTCNPLSGIFPTSGPTYPPTDK | |
| | PDDDTTDDDKVDTTIKPIEYPKPDGCNRTGDHFTMEPGA | |
| | NFYTVPNLGPASSNSDECYTNPSFSIGSSIYMFSQEIRKTD | |
| | CTAGEILSIQIVLGRIVDKGQQGPQASPLLVWAVPNPKIIN | |
| | SCAVAAGDEMGWVLCSVTLTAASGEPIPHMFDGFWLYK | |
| | LEPDTEVVSYRITGYAYLLDKQYDSVFIGKGGGIQKGND | |
| | LYFQMYGLSRNRQSFKALCEHGSCLGTGGGGYQVLCDR | |
| | AVMSFGSEESLITNAYLKVNDLASGKPVIIGQTFPPSDSYK | |
| | GSNGRMYTIGDKYGLYLAPSSWNRYLRFGITPDISVRSTT | |
| | WLKSQDPIMKILSTCTNTDRDMCPEICNTRGYQDIFPLSE | |
| | DSEYYTYIGITPNNGGTKNFVAVRDSDGHIASIDILQNYY | |
| | SITSATISCFMYKDEIWCIAITEGKKQKDNPQRIYAHSYKI | |
| | RQMCYNMKSATVTVGNAKNITIRRY | |
| 13 | ILHY EKLSKIGLVK GVTRKYKIKS NPLTKDIVIK | Nipah virus NiV-F |
| | MIPNVSNMSQ CTGSVMENYK TRINGILTPI KGALEIYKNN | F0 (aa 27-546) |
| | THDLVGDVRL AGVIMAGVAI GIATAAQITA GVALYEAMKN | |
| | ADNINKLKSS IESTNEAVVK LQETAEKTVY VLTALQDYIN | |
| | TNLVPTIDKI SCKQTELSLD LALSKYLSDL LFVFGPNLQD | |
| | PVSNSMTIQA ISQAFGGNYE TLLRTLGYAT EDFDDLLESD | |
| | SITGQIIYVD LSSYYIIVRV YFPILTEIQQ AYIQELLPVS | |
| | FNNDNSEWIS IVPNFILVRN TLISNIEIGF CLITKRSVIC | |
| | NQDYATPMTN NMRECLTGST EKCPRELVVS SHVPRFALSN | |
| | GVLFANCISV TCQCQTTGRA ISQSGEQTLL MIDNTTCPTA | |
| | VLGNVIISLG KYLGSVNYNS EGIAIGPPVF TDKVDISSQI | |
| | SSMNQSLQQS KDYIKEAQRL LDTVNPSLIS MLSMIILYVL | |
| | SIASLCIGLI TFISFIIVEK KRNTYSRLED RRVRPTSSGD | |
| | LYYIGT | |
| 14 | ILHYEKLSKIGLVKGVTRKYKIKSNPLTKDIVIKMIPNVSNMS | Nipah virus NiV-F |
| | QCTGSVMENYKTRLNGILTPIKGALEIYKNNTHDLVGDVR | F2 (aa 27-109) |
| 15 | LAGVIMAGVAIGIATAAQITAGVALYEAMKNADNINKLKSSIE | Nipah virus NiV F |
| | STNEAVVKLQETAEKTVYVLTALQDYINTNLVPTIDKISCKQT | F1 (aa 110-546) |
| | ELSLDLALSKYLSDLLFVFGPNLQDPVSNSMTIQAISQAFGGN | |
| | YETLLRTLGYATEDFDDLLESDSITGQIIYVDLSSYYIIVRVY | |
| | FPILTEIQQAYIQELLPVSENNDNSEWISIVPNFILVRNTLIS | |
| | NIEIGFCLITKRSVICNQDYATPMTNNMRECLTGSTEKCPREL | |
| | VVSSHVPRFALSNGVLFANCISVTCQCQTTGRAISQSGEQTLL | |
| | MIDNTTCPTAVLGNVIISLGKYLGSVNYNSEGIAIGPPVETDK | |
| | VDISSQISSMNQSLQQSKDYIKEAQRLLDTVNPSLISMLSMII | |
| | LYVLSIASLCIGLITFISFIIVEKKRNTYSRLEDRRVRPTSSG | |
| | DLYYIGT | |
| 16 | MVVILDKRCY CNLLILILMI SECSVG | Signal Seq |
| 17 | MVVILDKRCY CNLLILILMI SECSVGILHY EKLSKIGLVK | NIVF T234 |
| | GVTRKYKIKS NPLTKDIVIK MIPNVSNMSQ CTGSVMENYK | |
| | TRLNGILTPI KGALEIYKNN THDLVGDVRL AGVIMAGVAI | |
| | GIATAAQITA GVALYEAMKN ADNINKLKSS IESTNEAVVK | |
| | LQETAEKTVY VLTALQDYIN TNLVPTIDKI SCKQTELSLD | |
| | LALSKYLSDL LFVEGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK | |
| | KRNT | |
| 18 | MKKINEGLLDSKILSA FNTVIALLGS IVIIVMNIMI | NiVG |
| | IQNYTRSTDN QAVIKDALQG IQQQIKGLAD KIGTEIGPKV | protein attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated |
| | LQKTSNQILK PKLISYTLPV VGQSGTCITD PLLAMDEGYF | and mutated |
| | AYSHLERIGS CSRGVSKQRI IGVGEVLDRG DEVPSLFMTN | (E501A, W504A, |
| | VWTPPNPNTV YHCSAVYNNE FYYVLCAVST VGDPILNSTY | Q530A, E533A) |
| | WSGSLMMTRL AVKPKSNGGG YNQHQLALRS IEKGRYDKVM | NiV G protein |
| | PYGPSGIKQG DTLYFPAVGF LVRTEFKYND SNCPITKCQY | (GcΔ 34) |
| | SKPENCRLSM GIRPNSHYIL RSGLLKYNLS DGENPKVVFI | |
| | EISDQRLSIG SPSKIYDSLG QPVFYQASFS WDTMIKFGDV | |
| | LTVNPLVVNW RNNTVISRPG QSQCPRFNTC PAICAEGVYN | |
| | DAFLIDRINW ISAGVFLDSN ATAANPVFTV FKDNEILYRA | |
| | QLASEDTNAQ KTITNCFLLK NKIWCISLVE IYDTGDNVIR | |
| | PKLFAVKIPE QC | |
| 19 | GGGGS | Linker |
| 20 | GGGGGS | Linker |
| 21 | (GGGGS)n | Linker (N = 1-10) |
| 22 | (GGGGGS)n | Linker (N = 1-6) |
| 23 | MLFNLRILLNNAAFRNGHNFMVRNFRCGQPLQNKVQLK | OTC |
| | GRDLLTLKNFTGEEIKYMLWLSADLKFRIKQKGEYLPLL | |
| | QGKSLGMIFEKRSTRTRLSTETGFALLGGHPCFLTTQDIH | |
| | LGVNESLTDTARVLSSMADAVLARVYKQSDLDTLAKEA | |
| | SIPIINGLSDLYHPIQILADYLTLQEHYSSLKGLTLSWIGDG | |
| | NNILHSIMMSAAKFGMHLQAATPKGYEPDASVTKLAEQ | |
| | YAKENGTKLLLTNDPLEAAHGGNVLITDTWISMGQEEEK | |
| | KKRLQAFQGYQVTMKTAKVAASDWTFLHCLPRKPEEVD | |
| | DEVFY | |
| | SPRSLVFPEAENRKWTIMAVMVSLLTDYSPQLQKPKF | |
| 24 | MGPWGWKLRWTVALLLAAAGTAVGDRCERNEFQCQD | LDLR |
| | GKCISYKWVCDGSAECQDGSDESQETCLSVTCKSGDFSC | |
| | GGRVNRCIPQFWRCDGQVDCDNGSDEQGCPPKTCSQDE | |
| | FRCHDGKCISRQFVCDSDRDCLDGSDEASCPVLTCGPASF | |
| | QCNSSTCIPQLWACDNDPDCEDGSDEWPQRCRGLYVFQ | |
| | GDSSPCSAFEFHCLSGECIHSSWRCDGGPDCKDKSDEENC | |
| | AVATCRPDEFQCSDGNCIHGSRQCDREYDCKDMSDEVG | |
| | CVNVTLCEGPNKFKCHSGECITLDKVCNMARDCRDWSD | |
| | EPIKECGTNECLDNNGGCSHVCNDLKIGYECLCPDGFQL | |
| | VAQRRCEDIDECQDPDTCSQLCVNLEGGYKCQCEEGFQL | |
| | DPHTKACKAVGSIAYLFFTNRHEVRKMTLDRSEYTSLIPN | |
| | LRNVVALDTEVASNRIYWSDLSQRMICSTQLDRAHGVSS | |
| | YDTVISRDIQAPDGLAVDWIHSNIYWTDSVLGTVSVADT | |
| | KGVKRKTLFRENGSKPRAIVVDPVHGFMYWTDWGTPAK | |
| | IKKGGLNGVDIYSLVTENIQWPNGITLDLLSGRLYWVDS | |
| | KLHSISSIDVNGGNRKTILEDEKRLAHPFSLAVFEDKVFW | |
| | TDIINEAIFSANRLTGSDVNLLAENLLSPEDMVLFHNLTQP | |
| | RGVNWCERTTLSNGGCQYLCLPAPQINPHSPKFTCACPD | |
| | GMLLARDMRSCLTEAEAAVATQETSTVRLKVSSTAVRT | |
| | QHTTTRPVPDTSRLPGATPGLTTVEIVTMSHQALGDVAG | |
| | RGNEKKPSSVRALSIVLPIVLLVFLCLGVFLLWKNWRLK | |
| | NINSINFDNPVYQKTTEDEVHICHNQDGYSYPSRQMVSLE | |
| | DDVA | |
| 25 | DIQMTQTTSSLSASLGDRVTISCRASQDISKYLNWYQQKP | Anti-CD19 scFv |
| | DGTVKLLIYHTSRLHSGVPSRFSGSGSGTDYSLTISNLEQE | (FMC63) |
| | DIATYFCQQGNTLPYTFGGGTKLEITGSTSGSGKPGSGEG | |
| | STKGEVKLQESGPGLVAPSQSLSVTCTVSGVSLPDYGVS | |
| | WIRQPPRKGLEWLGVIWGSETTYYNSALKSRLTIIKDNSK | |
| | SQVFLKMNSLQTDDTAIYYCAKHYYYGGSYAMDYWGQ | |
| | GTSVTVSS | |
| 26 | DIQMTQTTSSLSASLGDRVTISCRASQDISKYLNWYQQKP | Anti-CD19 scFv |
| | DGTVKLLIYHTSRLHSGVPSRFSGSGSGTDYSLTISNLEQE | (FMC63) |
| | DIATYFCQQGNTLPYTFGGGTKLEITGGGGSGGGGSGGG | |
| | GSEVKLQESGPGLVAPSQSLSVTCTVSGVSLPDYGVSWIR | |
| | QPPRKGLEWLGVIWGSETTYYNSALKSRLTIIKDNSKSQV | |
| | FLKMNSLQTDDTAIYYCAKHYYYGGSYAMDYWGQGTS | |
| | VTVSS | |
| 27 | ESKYGPPCPPCP | IgG4 Hinge |
| 28 | TTTPAPRPPTPAPTIASQPLSLRPE | CD8 Hinge |
| 29 | IEVMYPPPYLDNEKSNGTIIHVKGKHLCPSPLFPGPSKP | CD28 |
| 30 | ACRPAAGGAVHTRGLDFACDIYIWAPLAGTCGVLLLSLV | CD8 |
| | ITLYC | |
| 31 | FWVLVVVGGVLACYSLLVTVAFIIFWV | CD28 |
| 32 | FWVLVVVGGVLACYSLLVTVAFIIFWV | CD28 |
| 33 | RSKRSRLLHSDYMNMTPRRPGPTRKHYQPYAPPRDFAAY | CD28 |
| | RS | |
| 34 | KRGRKKLLYIFKQPFMRPVQTTQEEDGCSCRFPEEEEGGC | 4-1BB |
| | EL | |
| 35 | RVKFSRSADAPAYQQGQNQLYNELNLGRREEYDVLDKR | CD3zeta |
| | RGRDPEMGGKPRRKNPQEGLYNELQKDKMAEAYSEIGM | |
| | KGERRRGKGHDGLYQGLSTATKDTYDALHMQALPPR | |
| 37 | RVKFSRSADAPAYKQGQNQLYNELNLGRREEYDVLDKR | CD3zeta |
| | RGRDPEMGGKPRRKNPQEGLYNELQKDKMAEAYSEIGM | |
| | KGERRRGKGHDGLYQGLSTATKDTYDALHMQALPPR | |
| 37 | APRSVDWREK GYVTPVKNQG | Uniprot P07711 |
| | QCGSCWAFSATGALEGQMFR KTGRLISLSE | (114-333) |
| | QNLVDCSGPQ GNEGCNGGLM DYAFQYVQDN | |
| | GGLDSEESYP YEATEESCKYNPKYSVANDT | |
| | GFVDIPKQEK ALMKAVATVG PISVAIDAGH | |
| | ESFLFYKEGIYFEPDCSSED MDHGVLVVGY | |
| | GFESTESDNN KYWLVKNSWG EEWGMGGYVK | |
| | MAKDRRNHCG IASAASYPTV | |
| 38 | LPASFDAREQW PQCPTIKEIR DQGSCGSCWA | Uniprot P07858 |
| | FGAVEAISDR ICIHTNAHVS VEVSAEDLLT | |
| | CCGSMCGDGC NGGYPAEAWN FWTRKGLVSG | |
| | GLYESHVGCR PYSIPPCEHH VNGSRPPCTG | |
| | EGDTPKCSKI CEPGYSPTYK QDKHYGYNSY | |
| | SVSNSEKDIM AEIYKNGPVE GAFSVYSDFL | |
| | LYKSGVYQHV TGEMMGGHAI RILGWGVENG | |
| | TPYWLVANSW NTDWGDNGFF KILRGQDHCG | |
| | IESEVVAGIP RTDQYWEKI | |
| 39 | LPASFDAREQW PQCPTIKEIR DQGSCGSCWA | Uniprot P07858 |
| | FGAVEAISDR ICIHTNAHVS VEVSAEDLLT | |
| | CCGSMCGDGC NGGYPAEAWN FWTRKGLVSG | |
| | GLYESHVGCR PYSIPPCEHH VNGSRPPCTG | |
| | EGDTPKCSKI CEPGYSPTYK QDKHYGYNSY | |
| | SVSNSEKDIM AEIYKNGPVE GAFSVYSDFL | |
| | LYKSGVYQHV TGEMMGGHAI RILGWGVENG | |
| | TPYWLVANSW NTDWGDNGFF KILRGQDHCG | |
| | IESEVVAGIP RTD | |

1. A method of producing a plurality of fusosomes, comprising:
(a) providing a modified mammalian producer cell, e.g., a human cell, that comprises:
(i) an elevated level or activity of a mature cathepsin molecule (e.g., cathepsin L or cathepsin B) compared to a corresponding unmodified cell,
(ii) optionally, an exogenous cargo molecule, e.g., a protein or nucleic acid, and
(iii) a henipavirus F protein molecule; and
(iv) a henipavirus G protein molecule,
(b) maintaining (e.g., culturing) the modified mammalian cell under conditions that allow production of a plurality of fusosomes comprising the henipavirus F protein molecule, and the henipavirus G protein molecule.

2. A method of producing a modified mammalian producer cell, the method comprising:
(i) introducing into a mammalian cell a nucleic acid molecule encoding a cathepsin molecule under conditions to increase expression of the mature form of the cathepsin molecule in the mammalian cell;
(ii) optionally, introducing into the mammalian cell an exogenous cargo molecule, e.g., a protein or a nucleic acid;
(iii) introducing into the mammalian cell a henipavirus F protein molecule (e.g., introducing a nucleic acid encoding the henipavirus F protein molecule under conditions suitable for expressing the henipavirus F protein molecule); and
(iv) introducing into the mammalian cell a henipavirus G protein molecule (e.g., introducing a nucleic acid encoding the henipavirus G protein molecule under conditions suitable for expressing the henipavirus G protein molecule),
wherein steps (i)-(iv) can be carried out in any order or one or more of steps (i)-(iv) can be carried out simultaneously.

3. A modified mammalian cell, e.g., a human cell, that comprises:
(i) an elevated level or activity of a mature cathepsin molecule (e.g., cathepsin L or cathepsin B) compared to a corresponding unmodified cell,
(ii) optionally, an exogenous cargo molecule, e.g., a nucleic acid or a protein, e.g., a viral nucleic acid, e.g., a lentiviral nucleic acid, and
(iii) a henipavirus F protein molecule; and
(iv) optionally, a henipavirus G protein molecule.

4. A fusosome comprising:
(a) optionally, an exogenous cargo, e.g., a nucleic acid or protein, e.g., a viral nucleic acid (e.g., a lentiviral nucleic acid);
(b) an active henipavirus F protein molecule_comprising a modified F₁ form that has a C-terminal truncation of up to 30 contiguous amino acids compared to a wild-type henipavirus protein F₁ molecule, wherein at least 33% of henipavirus F protein molecule in the fusosome is active henipavirus F protein; and
(c) a henipavirus G protein molecule.

5. A fusosome comprising:
(a) optionally, an exogenous cargo, e.g., a fusosome nucleic acid, e.g., a viral nucleic acid (e.g., a lentiviral nucleic acid);
(b) a henipavirus F protein molecule, wherein at least 33% of henipavirus F protein molecule in the fusosome is active henipavirus F protein; and
(c) a henipavirus G protein molecule.

6. A pharmaceutical composition comprising a fusosome of claim 4 or claim 5 and optionally a pharmaceutically acceptable excipient.

7. A method of delivering an exogenous cargo (e.g., a fusosome nucleic acid, e.g., a viral nucleic acid, e.g., a lentiviral nucleic acid) to a cell (e.g., in vivo or ex vivo), comprising contacting the cell with a plurality of fusosomes of any of claims 4 or 5, a pharmaceutical composition of claim 6, or fusosomes made by a method of claim 1.

8. A method of delivering an exogenous cargo (e.g., a fusosome nucleic acid, e.g., a viral nucleic acid, e.g., a lentiviral nucleic acid) to a subject, comprising administering to the subject an effective number of fusosomes of any of claims 4 or 5, a pharmaceutical composition of claim 6, or fusosomes made by a method of claim 1.

9. The method, modified cell, fusosome, or pharmaceutical composition of any of the preceding claims, wherein the henipavirus F protein molecule lacks an endocytosis motif.

10. The method, modified cell, fusosome, or pharmaceutical composition of claim 9, wherein the endocytosis motif is a YXXφ motif or the endocytosis motif is a YSRL motif.

11. The method, modified cell, fusosome, or pharmaceutical composition of any of the preceding claims, wherein the cathepsin molecule comprises an amino acid sequence of SEQ ID NO: 1 or SEQ ID NO: 2 or a sequence having at least 80% identity thereto.

12. The method, modified cell, fusosome, or pharmaceutical composition of any of the preceding claims, wherein the cathepsin molecule comprises an amino acid sequence of SEQ ID NO: 37 or SEQ ID NO: 38 or SEQ ID NO: 39 or a sequence having at least 80% identity thereto.

13. The method, modified cell, fusosome, or pharmaceutical composition of any of the preceding claims, wherein the elevated level of the cathepsin molecule comprises at least 50%, or more cathepsin molecule than the amount of endogenous cathepsin L in a corresponding unmodified cell.

14. The method, modified cell, fusosome, or pharmaceutical composition of any of the preceding claims, wherein the fusosome comprises active henipavirus F protein molecule at a level at least 10% greater than an otherwise similar fusosome produced from a cell without the elevated level or activity of a cathepsin molecule.

15. The method, modified cell, fusosome, or pharmaceutical composition of any of the preceding claims, wherein at least 33% of henipavirus F protein molecule in the fusosome is active henipavirus F protein.

16. The method, modified cell, or pharmaceutical composition of any of the preceding claims, wherein the fusosomes have a functional titre of at least about 200,000 TU/mL, e.g., on 293LX cells, e.g., as measured by detection of a GFP reporter in 293 XL cells, e.g., by an assay of Example 1.

17. The method, modified cell, or pharmaceutical composition of any of the preceding claims, wherein the fusosomes have a functional titre of at least about 200,000 TU/mL, e.g., on activated T cells, e.g., primary T cells, e.g., Pan-T cells, e.g., as measured by detection of a GFP reporter in the activated T cells, e.g., by an assay of Example 3.

18. The method, modified cell, or pharmaceutical composition of any of the preceding claims, wherein the produced plurality of fusosomes has a ratio of a titre on target cells to a titre on non-target cells of at least 2:1, e.g., wherein target cells overexpress a protein bound by the henipavirus G protein molecule and the non-target cells are wild-type, e.g., wherein target cells overexpress CD8 and the non-target cells are wild-type, e.g., in an assay of Example 1.

19. The method, modified cell, fusosome, or pharmaceutical composition of any of the preceding claims, wherein the fusosome comprises a level of total henipavirus protein F that is between 70%-130% of the level of total henipavirus protein F comprised by an otherwise similar fusosome produced from a cell without the elevated level or activity of a cathepsin molecule.

20. The method, modified cell, fusosome, or pharmaceutical composition of any of the preceding claims, wherein the henipavirus F protein molecule comprises a wild-type Nipah virus amino acid sequence of SEQ ID NO: 7, or a sequence having at least 80% identity thereto.

21. The method, modified cell, fusosome, or pharmaceutical composition of any of the preceding claims, wherein the henipavirus F protein molecule comprises a henipavirus protein F of Table 4.

22. The method, modified cell, fusosome, or pharmaceutical composition of any of the preceding claims, wherein the henipavirus F protein molecule comprises a truncation of 10-30, 15-30, 10-20, or 20-30 amino acids, e.g., 22 or 25 amino acids, at the C terminus relative to a wild-type henipavirus F protein, e.g., a protein of Table 4, optionally wherein the henipavirus F protein comprises n amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 17, optionally wherein the henipavirus F protein is set forth in SEQ ID NO:17.

23. The method, modified cell, fusosome, or pharmaceutical composition of any of the preceding claims, wherein the henipavirus F protein molecule lacks an endocytic motif, e.g., a YXXφ motif, e.g., a YRSL motif.

24. The method, modified cell, fusosome, or pharmaceutical composition of any of the preceding claims, wherein the henipavirus G protein molecule comprises a wild-type Nipah virus amino acid sequence of SEQ ID NO: 9, or a sequence having at least 80% identity thereto.

25. The method, modified cell, fusosome, or pharmaceutical composition of any of the preceding claims, wherein the henipavirus G protein molecule comprises a truncation of 10-50 amino acids at the N terminus, relative to a wild-type henipavirus G protein, e.g., a protein of Table 5, optionally wherein the henipavirus F protein comprises n amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 18, optionally wherein the henipavirus F protein is set forth in SEQ ID NO:18.

26. The method, modified cell, fusosome, or pharmaceutical composition of any of the preceding claims, wherein the henipavirus G protein molecule is a retargeted henipavirus G protein molecule.

27. The method, modified cell, fusosome, or pharmaceutical composition of any of the preceding claims, wherein the fusosome nucleic acid is a lentiviral nucleic acid.

28. The method, modified cell, fusosome, or pharmaceutical composition of any of the preceding claims, wherein the fusosome nucleic acid encodes a therapeutic payload.

29. The method, modified cell, fusosome, or pharmaceutical composition of any of the preceding claims, wherein the modified cell is a human cell.


SEQUENCE LISTING (39 sequences)

SEQ ID NO: 1 - protein, 151 aa, Artificial Sequence
  Exemplary Cathepsin L1 Sequence
MDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFESTESDNNKYWLVKNSWGEEWGMGGYVKMAKDRRNHCGIASAASYPTV

SEQ ID NO: 2 - protein, 215 aa, Artificial Sequence
  Exemplary Cathepsin B Sequence
MHGNNGHSVPPSKRSETRAPVAPAGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 3 - protein, 546 aa, Artificial Sequence
  HeVF
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 4 - protein, 557 aa, Artificial Sequence
  CedVF
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 5 - protein, 545 aa, Artificial Sequence
  Mojiang virus F
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 6 - protein, 662 aa, Artificial Sequence
  Bat PV F
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 7 - protein, 546 aa, Artificial Sequence
  Nipah F0
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 8 - protein, 604 aa, Artificial Sequence
  HeV G
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 9 - protein, 601 aa, Artificial Sequence
  NiV G
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 10 - protein, 622 aa, Artificial Sequence
  CedV G
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 11 - protein, 632 aa, Artificial Sequence
  Bat PV G
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 12 - protein, 625 aa, Artificial Sequence
  Mojiang virus G
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 13 - protein, 520 aa, Artificial Sequence
  Nipah virus NiV-F F0 (aa 27-546)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 14 - protein, 83 aa, Artificial Sequence
  NiV-F F2 (aa 27-109)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 15 - protein, 437 aa, Artificial Sequence
  NiV F F1 (aa 110-546)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 16 - protein, 26 aa, Artificial Sequence
  Signal Seq
MVVILDKRCYCNLLILILMISECSVG

SEQ ID NO: 17 - protein, 524 aa, Artificial Sequence
  NiVF T234
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 18 - protein, 568 aa, Artificial Sequence
  Variant NiVG
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQC

SEQ ID NO: 19 - protein, 5 aa, Artificial Sequence
  Linker
GGGGS

SEQ ID NO: 20 - protein, 6 aa, Artificial Sequence
  Linker
GGGGGS

SEQ ID NO: 21 - protein, 5 aa, Artificial Sequence
  Linker
  REPEAT (1)...(5): N =1-10
GGGGS

SEQ ID NO: 22 - protein, 6 aa, Artificial Sequence
  Linker
  REPEAT (1)...(6): N=1-6
GGGGGS

SEQ ID NO: 23 - protein, 354 aa, Artificial Sequence
  OTC
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 24 - protein, 860 aa, Artificial Sequence [sequence omitted]

SEQ ID NO: 25 - protein, 245 aa, Artificial Sequence
  Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 242 aa, Artificial Sequence
  Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 27 - protein, 12 aa, Artificial Sequence
  IgG4 Hinge
ESKYGPPCPPCP

SEQ ID NO: 28 - protein, 25 aa, Artificial Sequence
  CD8 Hinge
TTTPAPRPPTPAPTIASQPLSLRPE

SEQ ID NO: 29 - protein, 39 aa, Artificial Sequence
  CD28
IEVMYPPPYLDNEKSNGTIIHVKGKHLCPSPLFPGPSKP

SEQ ID NO: 30 - protein, 44 aa, Artificial Sequence
  CD8
ACRPAAGGAVHTRGLDFACDIYIWAPLAGTCGVLLLSLVITLYC

SEQ ID NO: 31 - protein, 27 aa, Artificial Sequence
  CD28
FWVLVVVGGVLACYSLLVTVAFIIFWV

SEQ ID NO: 32 - protein, 27 aa, Artificial Sequence
  CD28
FWVLVVVGGVLACYSLLVTVAFIIFWV

SEQ ID NO: 33 - protein, 41 aa, Artificial Sequence
  CD28
RSKRSRLLHSDYMNMTPRRPGPTRKHYQPYAPPRDFAAYRS

SEQ ID NO: 34 - protein, 42 aa, Artificial Sequence
  4-1BB
KRGRKKLLYIFKQPFMRPVQTTQEEDGCSCRFPEEEEGGCEL

SEQ ID NO: 35 - protein, 112 aa, Artificial Sequence
  CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 112 aa, Artificial Sequence
  CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 220 aa, Artificial Sequence
  Uniprot P07711
APRSVDWREKGYVTPVKNQGQCGSCWAFSATGALEGQMFRKTGRLISLSEQNLVDCSGPQGNEGCNGGLMDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFESTESDNNKYWLVKNSWGEEWGMGGYVKMAKDRRNHCGIASAASYPTV

SEQ ID NO: 38 - protein, 260 aa, Artificial Sequence
  Uniprot P07858
LPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 39 - protein, 254 aa, Artificial Sequence [sequence omitted]